(19) United States
(12) Patent Application Publication
(10) Pub. No.: US 2025/0092030 A1
(43) Pub. Date: Mar. 20, 2025

(54) ENHANCERS OF PARTICULATE GUANYLYL CYCLASE RECEPTOR A

(71) Applicants: Siobhan Malany, Orlando, FL (US); Satyamaheshwar Peddibhotla, Orlando, FL (US); Mayo Foundation for Medical Education and Research, Rochester, MN (US)

(72) Inventors: Sasantha J. Sangaralingham, Rochester, MN (US); John C. Burnett, JR., St. Anthony, MN (US); Siobhan Malany, Orlando, FL (US); Satyamaheshwar Peddibhotla, Orlando, FL (US)

(21) Appl. No.: 18/763,690

(22) Filed: Jul. 3, 2024

Related U.S. Application Data
(60) Provisional application No. 63/536,615, filed on Sep. 5, 2023.

(51) Int. Cl. C07D 417/12; A61K 31/428; A61K 31/439; A61K 31/454; A61K 31/4545; A61K 31/4709; A61K 31/506; A61K 31/5377; A61K 31/55; C07D 277/82; C07D 417/14

(52) U.S. Cl. CPC C07D 417/12; A61K 31/428; A61K 31/439; A61K 31/454; A61K 31/4545; A61K 31/4709; A61K 31/506; A61K 31/5377; A61K 31/55; C07D 277/82; C07D 417/14

(57) ABSTRACT

In some embodiments, the present disclosure provides a compound of Formula (I):

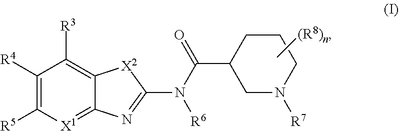

or a pharmaceutically acceptable salt thereof, wherein X¹, X², R³-R⁸, and n are as disclosed herein. In some embodiments, the present disclosure provides a pharmaceutical composition comprising any of the compounds as described herein, or a pharmaceutically acceptable salt thereof, and a pharmaceutically acceptable carrier. In some embodiments, the present disclosure provides a method of modulating particulate guanylyl cyclase receptor A (pGC-A) in a cell, the method comprising contacting the cell with an effective amount of any one of the compounds as described herein, or a pharmaceutically acceptable salt thereof.

### CROSS-REFERENCE TO RELATED APPLICATIONS

This application claims the benefit of U.S. Patent Application Ser. No. 63/536,615, filed on Sep. 5, 2023. The disclosure of the prior application is considered part of (and is incorporated by reference in) the disclosure of this application.

### STATEMENT AS TO FEDERALLY SPONSORED RESEARCH

This invention was made with government support under HL132854 and HTL136340 awarded by National Institutes of Health. The government has certain rights in the invention.

### TECHNICAL FIELD

This invention relates to organic compounds, and more particularly to halobenzo[d]thiazole compounds useful in treating various conditions such as cardiovascular, renal, and metabolic diseases, as well as cancer.

### BACKGROUND

Metabolic disease continues to grow worldwide, representing one of the greatest burdens in human health. Metabolic disease, often referred to as metabolic syndrome, encompasses obesity, type 2 diabetes (T2DM), insulin resistance, hyperlipidemia and hypertension, and represents a global challenge to human health.

Cardiovascular disease (CVD), including myocardial infarction, stroke, and hypertension, also presents a significant socioeconomic burden. CVD remains the leading cause of death in the U.S. The rates of CVD mortality per 100,000 people are currently nearly 400 for women, and nearly 700 for men.

Likewise, renal (kidney) disease is associated with a tremendous economic burden. High-income countries typically spend more than 2-3% of their annual health-care budget on the treatment of end-stage kidney disease, even though those receiving such treatment represent under 0.03% of the total population.

Finally, cancer is one of the leading causes of death in contemporary society. The numbers of new cancer cases and deaths is increasing each year. Currently, cancer incidence is nearly 450 cases of cancer per 100,000 men and women per year, while cancer mortality is nearly 71 cancer deaths per 100,000 men and women per year.

### SUMMARY

Atrial (ANP) and B-type natriuretic peptide (BNP) bind to the particulate guanylyl cyclase receptor A (pGC-A) that is highly expressed in heart, kidney, adrenals, vasculature and adipocytes. Following pGC-A activation, the second messenger 3′, 5′ cyclic guanosine monophosphate (cGMP) is produced resulting in widespread actions, including blood pressure lowering, renal enhancing, cardioprotective, and renin-angiotensin-aldosterone system (RAAS) suppressing properties. Advantageous metabolic actions of pGC-A include lipolysis, browning of adipocytes, stimulation of skeletal muscle energetics and release of adipokines such as adiponectin. The present disclosure is based, at least in part, on the realization that halobenzo[d]thiazole compounds are positive allosteric modulators of pGC-A, and, therefore, are useful in treating cardiovascular, renal, and metabolic diseases. As a further advantage, the compounds of the present disclosure are orally bioavailable.

In a first general aspect, the present disclosure provides a compound of any one of the following Formulae:

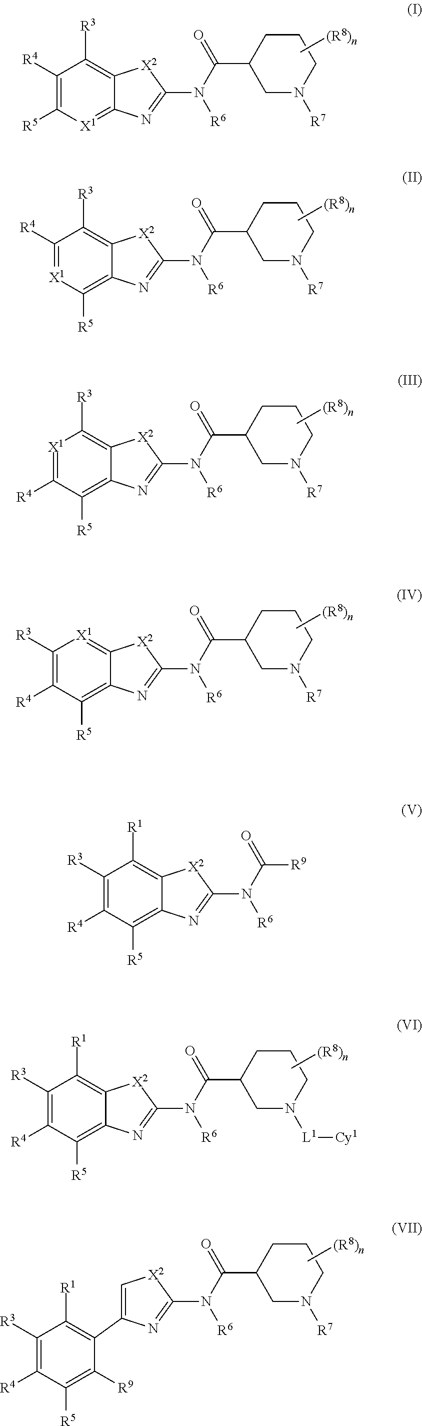

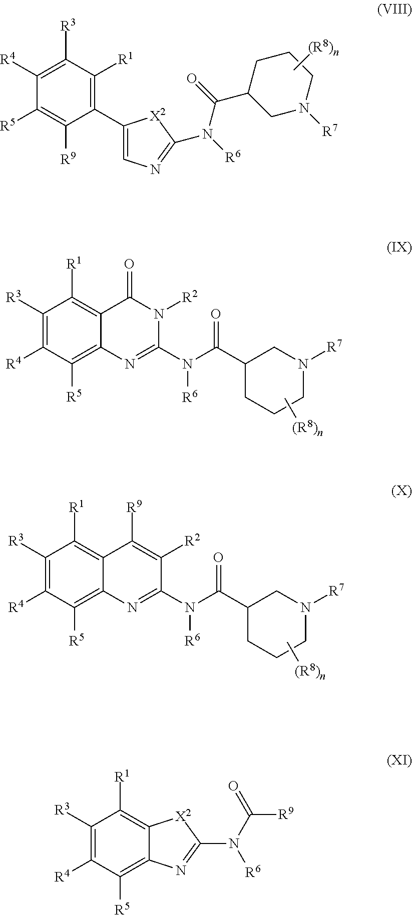

or a pharmaceutically acceptable salt thereof, wherein the R- and X-groups are as described herein.

In a second general aspect, the present disclosure provides a pharmaceutical composition comprising any of the compounds as described herein, or a pharmaceutically acceptable salt thereof, and a pharmaceutically acceptable carrier.

In a third general aspect, the present disclosure provides a method of modulating particulate guanylyl cyclase receptor A (pGC-A) in a cell, the method comprising contacting the cell with an effective amount of any one of the compounds as described herein, or a pharmaceutically acceptable salt thereof.

In a fourth general aspect, the present disclosure provides a method of modulating particulate guanylyl cyclase receptor A (pGC-A) in a subject, the method comprising administering to the subject in need thereof an effective amount of any one of the compounds as described herein, or a pharmaceutically acceptable salt thereof, or the pharmaceutical composition comprising same.

In a fifth general aspect, the present disclosure provides a method of treating or preventing a disease or condition responsive to modulation of a particulate guanylyl cyclase receptor A (pGC-A) in a subject, the method comprising administering to the subject in need thereof a therapeutically effective amount of any one of the compounds as described herein, or a pharmaceutically acceptable salt thereof, or the pharmaceutical composition comprising same. In some embodiments, the disease or condition is selected from metabolic disease, cardiovascular disease, and kidney disease. Suitable examples of these diseases are described herein.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the present application will be apparent from the following detailed description, and from the claims.

### DETAILED DESCRIPTION

Without being bound by a particular theory, it is believed that the heart is a vital endocrine organ that fine-tunes the body's metabolic homeostasis. Atrial natriuretic peptide (ANP) and B-type natriuretic peptide (BNP) are produced in the heart and released from atrial secretory granules, much like insulin is produced and released from pancreatic secretory granules. The molecular target of these two cardiac hormones is the particulate guanylyl cyclase receptor A (pGC-A) which functions via the second messenger cGMP. Among various physiological functions of pGC-A are regulation of blood pressure (BP), reno-enhancing and renoprotective actions, as well as metabolic actions, including lipolysis with production of non-esterified free fatty acids (NEFA) and glycerol, browning of white adipocytes, stimulation of skeletal muscle energetics, and enhancing release of adipokines such as adiponectin. In one example, in a murine model of obesity, over-expression of the pGC-A activating cardiac hormone BNP protected animals from obesity. In addition, GC-A is highly expressed in the heart, kidney, adrenals, vasculature, and adipocytes. While optimally regulating intravascular volume and blood pressure homeostasis, GC-A activation directly mediates organ protection with anti-apoptotic, anti-fibrotic, anti-hypertrophic, vascular endothelial regenerating, lipolytic, aldosterone suppressing, anti-cancer, and tumor suppressive properties.

Population studies investigating the common genetic variants of the ANP (rs5068) and BNP genes (rs1938845) showed that rs5068 and rs1938845 increase circulating ANP or BNP, respectively. Importantly, the elevation of ANP, through rs5068, was associated with protection from obesity and metabolic syndrome, decreased waist circumference, higher HDL levels with reduced BP, and risk for hypertension. While rs5068 is common, only 10% of the population carry this ANP genetic variant, and exhibit the protective phenotype. Hence, approximately 90% of the population has a relative higher risk for metabolic syndrome and hypertension based upon this ANP genetic variation. It was also shown that metabolic protective actions of rs5068 are present in African Americans, which highlights the multiethnic metabolic protection of pGC-A. Furthermore, the BNP gene variant, rs1938845 was found to be associated with reduced risk for type II diabetes mellitus as well as prolonged survival of the diabetes patients. Importantly, in patients with chronic heart failure, twice daily subcutaneously (SQ) administered BNP and subsequently GC-A activation reversed cardiac hypertrophy and improved myocardial function and notably, improved patient symptoms.

Without being bound by a theory, it is believed that the compounds described herein increase pGC-A responsiveness to the endogenous ligands (ANP and BNP), even at reduced levels, by enhancing the pGC-A function in a positive allosteric manner. The compounds within the present claims also exhibit ADME (Absorption, Distribution, Metabolism, and Excretion) properties including solubility, microsomal stability and plasma stability. Certain embodiments of the particulate guanylyl cyclase receptor A (pGC-A) activator compounds, pharmaceutical compositions comprising same, and methods of their use to treat pGC-A associated conditions (alone or in combination with other agents) are described herein.

### Therapeutic Compounds

The compound of this disclosure may be described by reference to any of the following Formulae, or a pharmaceutically acceptable salt thereof.

### Compounds of Formula (I)

In some embodiments, the present disclosure provides a compound of Formula (I):

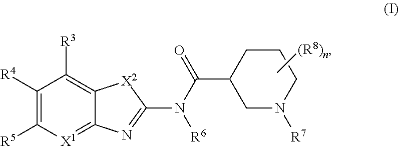

or a pharmaceutically acceptable salt thereof, wherein:
X¹ is selected from N and CR¹;
X² is selected from S, O, and NR²;
R¹ is selected from CN, NO₂, C(O)OR^{a1}, OR^{a1}, NR^{c1}R^{d1}, Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹;
R² is selected from H, C₁₋₃ alkyl, and C₁₋₃ haloalkyl;
R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R₈ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from RCy1; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, the compound of Formula (I) has formula:

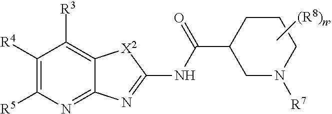

or a pharmaceutically acceptable salt thereof.

In some embodiments, the compound of Formula (I) has formula:

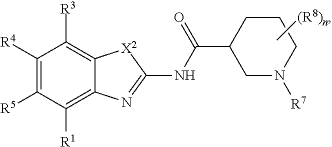

or a pharmaceutically acceptable salt thereof.

In some embodiments, R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R³, R⁴, and R⁵ are each H. In some embodiments, R³, R⁴, and R⁵ are each independently selected from halo, CN, OH, C₁₋₃ alkyl, C₁₋₃ haloalkyl, C₁₋₃ alkoxy, C₁₋₃ haloalkoxy, OR^{a1}—C₁₋₃ alkylene, and NR^{c1}R^{d1}—C₁₋₃ alkyl.

In some embodiments, R³, R⁴, and R⁵ are each independently selected from H, halo, CN, OH, C₁₋₃ alkyl, C₁₋₃ haloalkyl, C₁₋₃ alkoxy, and C₁₋₃ haloalkoxy. In some embodiments, R³ is selected from H, halo, CN, OH, C₁₋₃ alkyl, C₁₋₃ haloalkyl, C₁₋₃ alkoxy, and C₁₋₃ haloalkoxy. In some embodiments, R⁴ is selected from H, halo, CN, OH, C₁₋₃ alkyl, C₁₋₃ haloalkyl, C₁₋₃ alkoxy, and C₁₋₃ haloalkoxy. In some embodiments, R⁵ is selected from H, halo, CN, OH, C₁₋₃ alkyl, C₁₋₃ haloalkyl, C₁₋₃ alkoxy, and C₁₋₃ haloalkoxy. In some embodiments, R³, R⁴, and R⁵ are each independently selected from H, halo, CN, C₁₋₃ alkyl, C₁₋₃ haloalkyl, C₁₋₃ alkoxy, and C₁₋₃ haloalkoxy. In some embodiments, R³, R⁴, and R⁵ are each independently selected from H and halo. In some embodiments, R³ is H or halo. In some embodiments, R⁴ is H or halo. In some embodiments, R⁵ is H or halo.

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, CN, C₁₋₃ alkyl, C₁₋₃ haloalkyl, C₁₋₃ alkoxy, and C₁₋₃ haloalkoxy.

In some embodiments, n is 0, 1, or 2. In some embodiments, n is 0. In some embodiments, n is 1. In some embodiments, n is 2.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S.

In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, the compound of Formula (I) has formula:

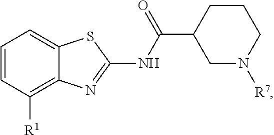

or a pharmaceutically acceptable salt thereof.

In some embodiments, R¹ is selected from CN, NO₂, C(O)OR^{a1}, OR^{a1}, NR^{c1}R^{d1}, Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹.

In some embodiments, R¹ is selected from CN, C(O)OR^{a1}, OR^{a1}, Cy¹, C₁₋₆ alkyl, and C₁₋₆ haloalkyl, wherein said C₁₋₆ alkyl is optionally substituted with Cy¹.

In some embodiments, R¹ is CN. In some embodiments, R¹ is C(O)OR^{a1}. In some embodiments, R¹ is OR^{a1}. In some embodiments, R¹ is Cy¹. In some embodiments, R¹ is C₁₋₆ alkyl (e.g., methyl, ethyl, isopropyl). In some embodiments, R¹ is C₁₋₆ haloalkyl (e.g., trifluormethyl, trichloromethyl). In some embodiments, R¹ is C₁₋₆ alkyl substituted with Cy¹. In some embodiments, R¹ is C(O)O(C₁₋₃ alkyl). In some embodiments, R¹ is CN. In some embodiments, R¹ is C₁₋₃ alkoxy or C₁₋₃ haloalkoxy. In some embodiments, R¹ is C₁₋₆ alkyl or C₁₋₆ haloalkyl. In some embodiments, R¹ is phenyl or phenyl-C₁₋₆ alkylene (e.g., benzyl).

In some embodiments, the compound of Formula (I) has formula:

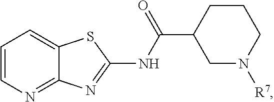

or a pharmaceutically acceptable salt thereof.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is C₁₋₆ alkyl substituted with NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1} In some embodiments, R⁷ is C₁₋₆ alkyl substituted with Cy¹. In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃ alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (I) is selected from any one of the following compounds:

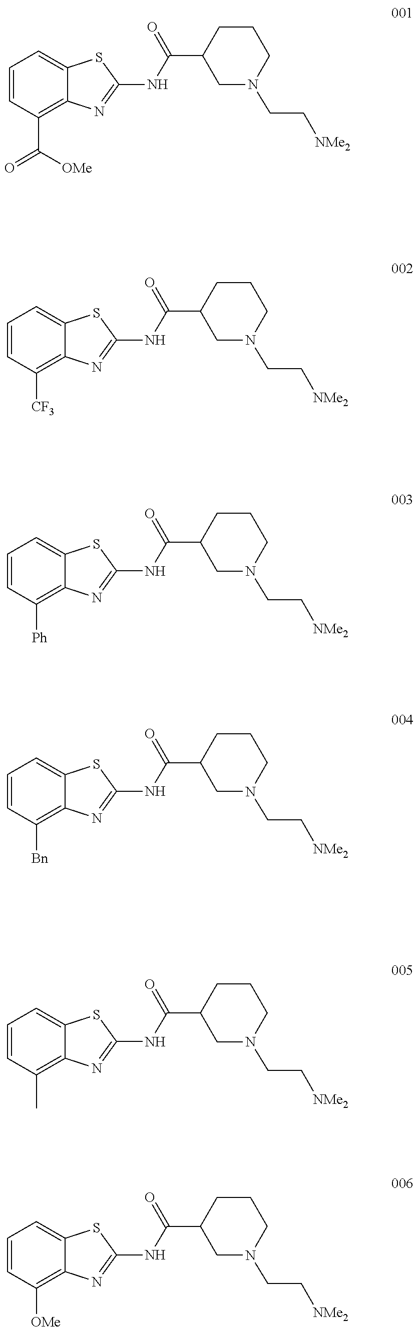 [SMILES: COC(=O)c1cccc2sc(NC(=O)C3CCCN(CCN(C)C)C3)nc12 ; CN(C)CCN1CCCC(C(=O)Nc2nc3c(C(F)(F)F)cccc3s2)C1 ; CN(C)CCN1CCCC(C(=O)Nc2nc3c(-c4ccccc4)cccc3s2)C1 ; CN(C)CCN1CCCC(C(=O)Nc2nc3c(Cc4ccccc4)cccc3s2)C1 ; Cc1cccc2sc(NC(=O)C3CCCN(CCN(C)C)C3)nc12 ; COc1cccc2sc(NC(=O)C3CCCN(CCN(C)C)C3)nc12]

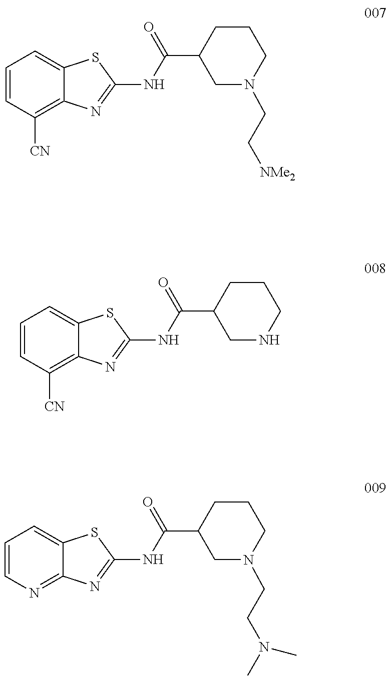 [SMILES: CN(C)CCN1CCCC(C(=O)Nc2nc3c(C#N)cccc3s2)C1 ; N#Cc1cccc2sc(NC(=O)C3CCCNC3)nc12 ; CN(C)CCN1CCCC(C(=O)Nc2nc3ncccc3s2)C1]

or a pharmaceutically acceptable salt thereof.

### Compounds of Formula (HH)

In some embodiments, the present disclosure provides a compound of Formula

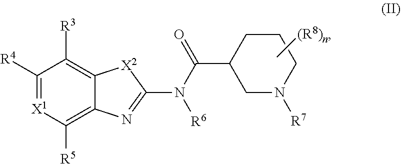

or a pharmaceutically acceptable salt thereof, wherein.
X¹ is selected from N and CR¹;
X² is selected from S, O, and NR²;
R¹ is selected from halo, CN, NO₂, C(O)OR^{a1}, OR^{a1}, NR^{c1}R^{d1}R^{c1}, C₁₋₆ alkyl, and C₁₋₆ haloalkyl, wherein said C₁₋₆ alkyl is optionally substituted with CN, C(O)OR^{a1}, OR^{a1} NR^{c1}R^{d1}, or Cy¹;
R² is selected from H, C₁₋₃ alkyl, and C₁₋₃ haloalkyl;
R³, R⁴, and R⁵ are each independently selected from H, halo, CN, C(O)OR^{a1} OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from RCy1; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, the compound of Formula (II) has formula:

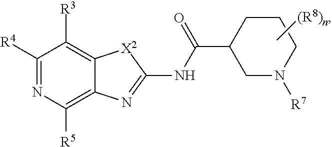

or a pharmaceutically acceptable salt thereof.

In some embodiments, the compound of Formula (II) has formula:

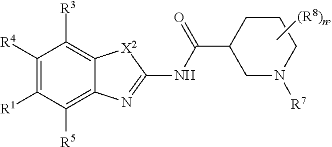

or a pharmaceutically acceptable salt thereof.

In some embodiments, R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R³, R⁴, and R⁵ are each H. In some embodiments, R³, R⁴, and R⁵ are each independently selected from halo, CN, OH, C₁₋₃ alkyl, C₁₋₃ haloalkyl, C₁₋₃ alkoxy, C₁₋₃ haloalkoxy, OR^{a1}—C₁₋₃ alkylene, and NR^{c1}R^{d1}—C₁₋₃ alkyl.

In some embodiments, R³, R⁴, and R⁵ are each independently selected from H, halo, CN, OH, C₁₋₃ alkyl, C₁₋₃ haloalkyl, C₁₋₃ alkoxy, and C₁₋₃ haloalkoxy. In some embodiments, R³ is selected from H, halo, CN, OH, C₁₋₃ alkyl, C₁₋₃ haloalkyl, C₁₋₃ alkoxy, and C₁₋₃ haloalkoxy. In some embodiments, R⁴ is selected from H, halo, CN, OH, C₁₋₃ alkyl, C₁₋₃ haloalkyl, C₁₋₃ alkoxy, and C₁₋₃ haloalkoxy. In some embodiments, R⁵ is selected from H, halo, CN, OH, C₁₋₃ alkyl, C₁₋₃ haloalkyl, C₁₋₃ alkoxy, and C₁₋₃ haloalkoxy. In some embodiments, R³, R⁴, and R⁵ are each independently selected from H, halo, CN, C₁₋₃ alkyl, C₁₋₃ haloalkyl, C₁₋₃ alkoxy, and C₁₋₃ haloalkoxy. In some embodiments, R³, R⁴, and R⁵ are each independently selected from H and halo. In some embodiments, R³ is H or halo. In some embodiments, R⁴ is H or halo. In some embodiments, R⁵ is H or halo.

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, CN, C₁₋₃ alkyl, C₁₋₃ haloalkyl, C₁₋₃ alkoxy, and C₁₋₃ haloalkoxy.

In some embodiments, n is 0, 1, or 2. In some embodiments, n is 0. In some embodiments, n is 1. In some embodiments, n is 2.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S. In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, the compound of Formula (II) has formula:

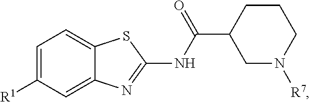

or a pharmaceutically acceptable salt thereof.

In some embodiments, R¹ is selected from CN, NO₂, C(O)OR^{a1}, OR^{a1}, NR^{c1}R^{d1}, Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹.

In some embodiments, R¹ is selected from CN, C(O)OR^{a1}, OR^{a1}, Cy¹, C₁₋₆ alkyl, and C₁₋₆ haloalkyl, wherein said C₁₋₆ alkyl is optionally substituted with Cy¹. In some embodiments, R¹ is selected from halo, C₁₋₆ alkyl, C₁₋₆ haloalkyl, C₁₋₃ alkoxy, and C₁₋₃ haloalkoxy.

In some embodiments, R¹ is CN. In some embodiments, R¹ is C(O)OR^{a1}. In some embodiments, R¹ is OR^{a1}. In some embodiments, R¹ is Cy¹. In some embodiments, R¹ is C₁₋₆ alkyl (e.g., methyl, ethyl, isopropyl). In some embodiments, R¹ is C₁₋₆ haloalkyl (e.g., trifluormethyl, trichloromethyl). In some embodiments, R¹ is C₁₋₆ alkyl substituted with Cy¹. In some embodiments, R¹ is C(O)O(C₁₋₃ alkyl). In some embodiments, R¹ is CN. In some embodiments, R¹ is C₁₋₃ alkoxy or C₁₋₃ haloalkoxy. In some embodiments, R¹ is C₁₋₃ alkoxy. In some embodiments, R¹ is C₁₋₃ haloalkoxy. In some embodiments, R¹ is C₁₋₆ alkyl or C₁₋₆ haloalkyl. In some embodiments, R¹ is phenyl or phenyl-C₁₋₆ alkylene (e.g., benzyl).

In some embodiments, the compound of Formula (II) has formula:

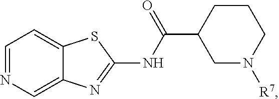

or a pharmaceutically acceptable salt thereof.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is C₁₋₆ alkyl substituted with NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1} In some embodiments, R⁷ is C₁₋₆ alkyl substituted with Cy¹. In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃ alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (II) is selected from any one of the following compounds:

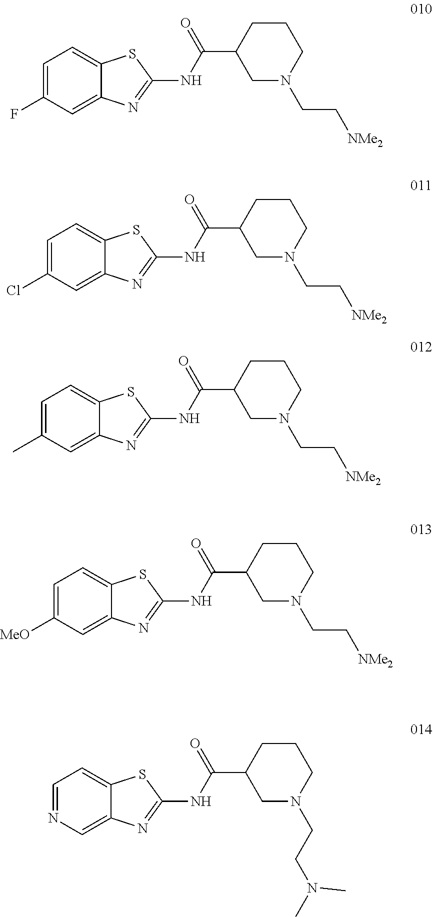 [SMILES: CN(C)CCN1CCCC(C(=O)Nc2nc3cc(F)ccc3s2)C1 ; CN(C)CCN1CCCC(C(=O)Nc2nc3cc(Cl)ccc3s2)C1 ; Cc1ccc2sc(NC(=O)C3CCCN(CCN(C)C)C3)nc2c1 ; COc1ccc2sc(NC(=O)C3CCCN(CCN(C)C)C3)nc2c1 ; CN(C)CCN1CCCC(C(=O)Nc2nc3cnccc3s2)C1]

or a pharmaceutically acceptable salt thereof.

### Compounds of Formula (III)

In some embodiments, the present disclosure provides a compound of Formula (III):

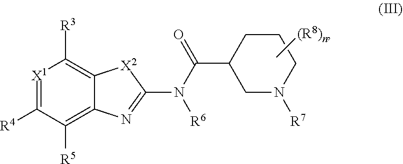

or a pharmaceutically acceptable salt thereof, wherein:
X¹ is selected from N and CR¹;
X² is selected from S, O, and NR²;
R¹ is selected from halo, CN, NO₂, C(O)OR^{a1}, OR^{a1}, N^{c1}R^{d1}, Cy¹, C₁₋₆ alkyl, and C₁₋₆ haloalkyl, wherein said C₁₋₆ alkyl is optionally substituted with CN, C(O)OR^{a1}, OR^{a1} NR^{c1}R^{d1}, or Cy¹;
R² is selected from H, C₁₋₃ alkyl, and C₁₋₃ haloalkyl;
R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from RCy1; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, the compound of Formula (III) has formula:

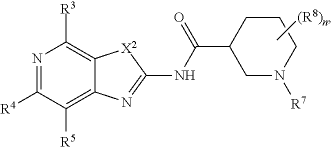

or a pharmaceutically acceptable salt thereof.

In some embodiments, the compound of Formula (III) has formula:

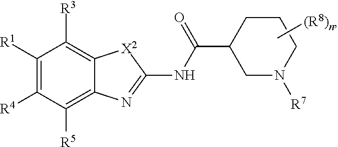

or a pharmaceutically acceptable salt thereof.

In some embodiments, R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R³, R⁴, and R⁵ are each H. In some embodiments, R³, R⁴, and R⁵ are each independently selected from halo, CN, OH, C₁₋₃ alkyl, C₁₋₃ haloalkyl, C₁₋₃ alkoxy, C₁₋₃ haloalkoxy, OR^{a1}—C₁₋₃ alkylene, and NR^{c1}R^{d1}—C₁₋₃ alkyl.

In some embodiments, R³, R⁴, and R⁵ are each independently selected from H, halo, CN, OH, C₁₋₃ alkyl, C₁₋₃ haloalkyl, C₁₋₃ alkoxy, and C₁₋₃ haloalkoxy. In some embodiments, R³ is selected from H, halo, CN, OH, C₁₋₃ alkyl, C₁₋₃ haloalkyl, C₁₋₃ alkoxy, and C₁₋₃ haloalkoxy. In some embodiments, R⁴ is selected from H, halo, CN, OH, C₁₋₃ alkyl, C₁₋₃ haloalkyl, C₁₋₃ alkoxy, and C₁₋₃ haloalkoxy. In some embodiments, R⁵ is selected from H, halo, CN, OH, C₁₋₃ alkyl, C₁₋₃ haloalkyl, C₁₋₃ alkoxy, and C₁₋₃ haloalkoxy. In some embodiments, R³, R⁴, and R⁵ are each independently selected from H, halo, CN, C₁₋₃ alkyl, C₁₋₃ haloalkyl, C₁₋₃ alkoxy, and C₁₋₃ haloalkoxy. In some embodiments, R³, R⁴, and R⁵ are each independently selected from H and halo. In some embodiments, R³ is H or halo. In some embodiments, R⁴ is H or halo. In some embodiments, R⁵ is H or halo.

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, CN, C₁₋₃ alkyl, C₁₋₃ haloalkyl, C₁₋₃ alkoxy, and C₁₋₃ haloalkoxy.

In some embodiments, n is 0, 1, or 2. In some embodiments, n is 0. In some embodiments, n is 1. In some embodiments, n is 2.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S.

In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, the compound of Formula (III) has formula:

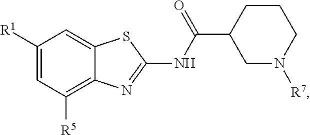

or a pharmaceutically acceptable salt thereof.

In some embodiments, R¹ is selected from CN, NO₂, C(O)OR^{a1}, OR^{a1}, NR^{c1}R^{d1}, Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹.

In some embodiments, R¹ is selected from CN, C(O)OR^{a1}, OR^{a1}, Cy¹, C₁₋₆ alkyl, and C₁₋₆ haloalkyl, wherein said C₁₋₆ alkyl is optionally substituted with Cy¹. In some embodiments, R¹ is selected from halo, C(O)OR^{a1}, OR^{a1}, Cy¹, C₁₋₆ alkyl, and C₁₋₆ haloalkyl, wherein said C₁₋₆ alkyl is optionally substituted with OR^{a1}.

In some embodiments, R¹ is CN. In some embodiments, R¹ is halo. In some embodiments, R¹ is C(O)OR^{a1}. In some embodiments, R¹ is OR^{a1}. In some embodiments, R¹ is Cy¹. In some embodiments, R¹ is C₁₋₆ alkyl (e.g., methyl, ethyl, isopropyl). In some embodiments, R¹ is C₁₋₆ haloalkyl (e.g., trifluormethyl, trichloromethyl). In some embodiments, R¹ is C₁₋₆ alkyl substituted with Cy¹. In some embodiments, R¹ is C(O)O(C₁₋₃ alkyl). In some embodiments, R¹ is C₁₋₃ alkoxy or C₁₋₃ haloalkoxy. In some embodiments, R¹ is C₁₋₆ alkyl or C₁₋₆ haloalkyl. In some embodiments, R¹ is phenyl or phenyl-C₁₋₆ alkylene (e.g., benzyl). In some embodiments, R¹ is C₆₋₁₀ aryl. In some embodiments, R¹ is C₁₋₆ alkyl substituted with C₁₋₃ alkoxy.

In some embodiments, the compound of Formula (III) has formula:

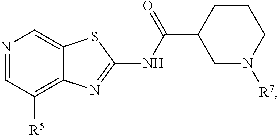

or a pharmaceutically acceptable salt thereof.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is C₁₋₆ alkyl substituted with NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1} In some embodiments, R⁷ is C₁₋₆ alkyl substituted with Cy¹. In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}, —C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, N^{c2}R^{d2}, NR^{c2}C(O)R^{b2} N^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (III) is selected from any one of the following compounds:

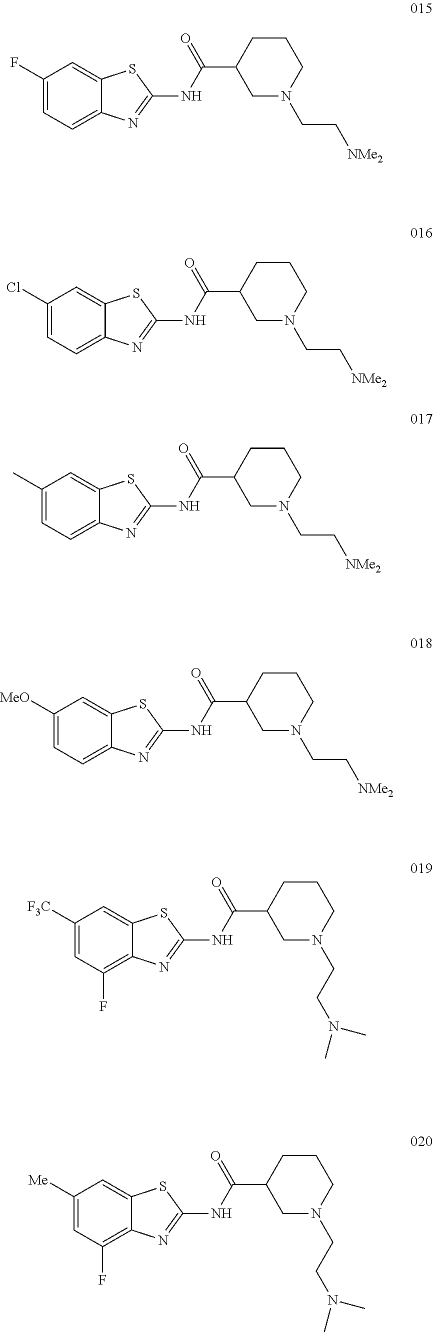 [SMILES: CN(C)CCN1CCCC(C(=O)Nc2nc3ccc(F)cc3s2)C1 ; CN(C)CCN1CCCC(C(=O)Nc2nc3ccc(Cl)cc3s2)C1 ; Cc1ccc2nc(NC(=O)C3CCCN(CCN(C)C)C3)sc2c1 ; COc1ccc2nc(NC(=O)C3CCCN(CCN(C)C)C3)sc2c1 ; CN(C)CCN1CCCC(C(=O)Nc2nc3c(F)cc(C(F)(F)F)cc3s2)C1 ; Cc1cc(F)c2nc(NC(=O)C3CCCN(CCN(C)C)C3)sc2c1]

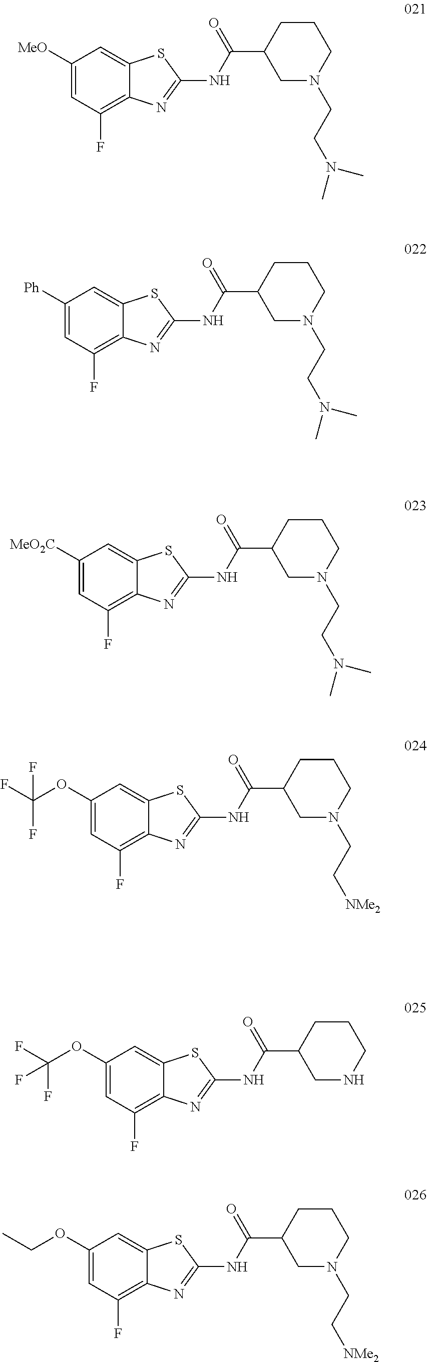

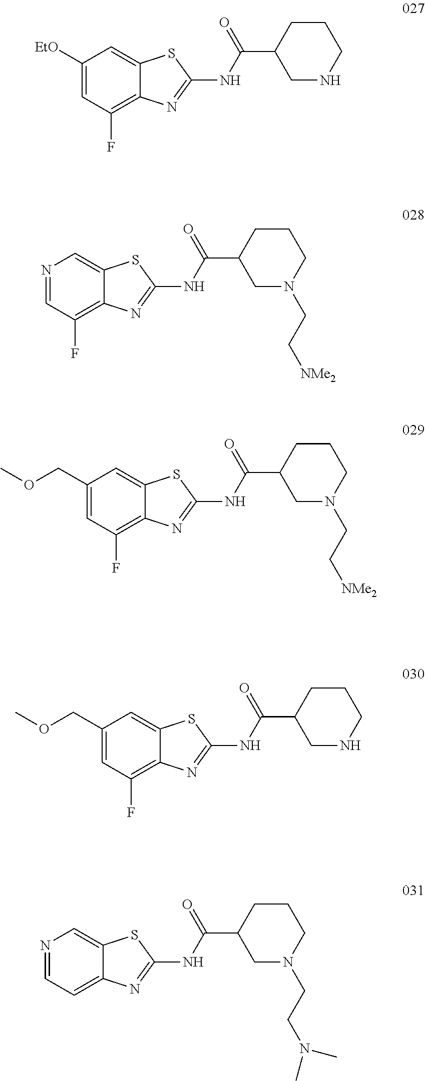 [SMILES: CCOc1cc(F)c2nc(NC(=O)C3CCCNC3)sc2c1 ; CN(C)CCN1CCCC(C(=O)Nc2nc3c(F)cncc3s2)C1 ; COCc1cc(F)c2nc(NC(=O)C3CCCN(CCN(C)C)C3)sc2c1 ; COCc1cc(F)c2nc(NC(=O)C3CCCNC3)sc2c1 ; CN(C)CCN1CCCC(C(=O)Nc2nc3ccncc3s2)C1]

or a pharmaceutically acceptable salt thereof.

### Compounds of Formula (IV)

In some embodiments, the present disclosure provides a compound of Formula (IV):

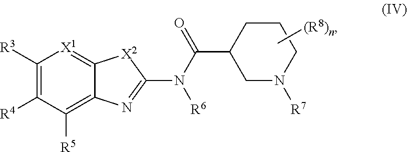

or a pharmaceutically acceptable salt thereof, wherein:
X¹ is selected from N and CR¹;
X² is selected from S, O, and NR²;
R¹ is selected from halo, CN, NO₂, C(O)OR^{a1}, OR^{a1}, N¹R^{d1} Cy¹, C₁₋₆ alkyl, and C₁₋₆ haloalkyl, wherein said C₁₋₆ alkyl is optionally substituted with CN, C(O)OR^{a1}, OR^{a1} NR^{c1}R^{d1}, or Cy¹;
R² is selected from H, C₁₋₃ alkyl, and C₁₋₃ haloalkyl;
R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from RCy1; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, the compound of Formula (IV) has formula:

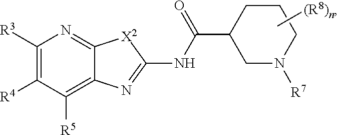

or a pharmaceutically acceptable salt thereof.

In some embodiments, the compound of Formula (IV) has formula:

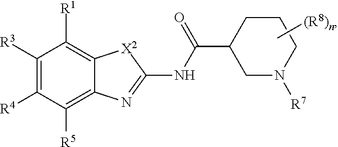

or a pharmaceutically acceptable salt thereof.

In some embodiments, R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R³, R⁴, and R⁵ are each H. In some embodiments, R³, R⁴, and R⁵ are each independently selected from halo, CN, OH, C₁₋₃ alkyl, C₁₋₃ haloalkyl, C₁₋₃ alkoxy, C₁₋₃ haloalkoxy, OR^{a1}—C₁₋₃ alkylene, and NR^{c1}R^{d1}—C₁₋₃ alkyl.

In some embodiments, R³, R⁴, and R⁵ are each independently selected from H, halo, CN, OH, C₁₋₃ alkyl, C₁₋₃ haloalkyl, C₁₋₃ alkoxy, and C₁₋₃ haloalkoxy. In some embodiments, R³ is selected from H, halo, CN, OH, C₁₋₃ alkyl, C₁₋₃ haloalkyl, C₁₋₃ alkoxy, and C₁₋₃ haloalkoxy. In some embodiments, R⁴ is selected from H, halo, CN, OH, C₁₋₃ alkyl, C₁₋₃ haloalkyl, C₁₋₃ alkoxy, and C₁₋₃ haloalkoxy. In some embodiments, R⁵ is selected from H, halo, CN, OH, C₁₋₃ alkyl, C₁₋₃ haloalkyl, C₁₋₃ alkoxy, and C₁₋₃ haloalkoxy. In some embodiments, R³, R⁴, and R⁵ are each independently selected from H, halo, CN, C₁₋₃ alkyl, C₁₋₃ haloalkyl, C₁₋₃ alkoxy, and C₁₋₃ haloalkoxy. In some embodiments, R³, R⁴, and R⁵ are each independently selected from H and halo. In some embodiments, R³ is H or halo. In some embodiments, R⁴ is H or halo. In some embodiments, R⁵ is H or halo.

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, CN, C₁₋₃ alkyl, C₁₋₃ haloalkyl, C₁₋₃ alkoxy, and C₁₋₃ haloalkoxy.

In some embodiments, n is 0, 1, or 2. In some embodiments, n is 0. In some embodiments, n is 1. In some embodiments, n is 2.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S.

In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, the compound of Formula (IV) has formula:

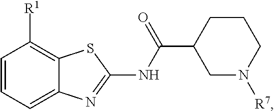

or a pharmaceutically acceptable salt thereof.

In some embodiments, R¹ is selected from CN, NO₂, C(O)OR^{a1}, OR^{a1}, NR^{c}R^{d1}, Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹.

In some embodiments, R¹ is selected from CN, C(O)OR^{a1}, OR^{a1}, Cy¹, C₁₋₆ alkyl, and C₁₋₆ haloalkyl, wherein said C₁₋₆ alkyl is optionally substituted with Cy¹. In some embodiments, R¹ is selected from halo, C(O)OR^{a1}, OR^{a1}, Cy¹, C₁₋₆ alkyl, and C₁₋₆ haloalkyl, wherein said C₁₋₆ alkyl is optionally substituted with OR^{a1}. In some embodiments, R¹ is selected from halo, C₁₋₆ alkyl, C₁₋₆ haloalkyl, C₁₋₃ alkoxy, and C₁₋₃ haloalkoxy.

In some embodiments, R¹ is CN. In some embodiments, R¹ is halo. In some embodiments, R¹ is C(O)OR^{a1}. In some embodiments, R¹ is OR^{a1}. In some embodiments, R¹ is Cy¹. In some embodiments, R¹ is C₁₋₆ alkyl (e.g., methyl, ethyl, isopropyl). In some embodiments, R¹ is C₁₋₆ haloalkyl (e.g., trifluormethyl, trichloromethyl). In some embodiments, R¹ is C₁₋₆ alkyl substituted with Cy¹. In some embodiments, R¹ is C(O)O(C₁₋₃ alkyl). In some embodiments, R¹ is C₁₋₃ alkoxy or C₁₋₃ haloalkoxy. In some embodiments, R¹ is C₁₋₃ alkoxy. In some embodiments, R¹ is C₁₋₃ haloalkoxy. In some embodiments, R¹ is C₁₋₆ alkyl or C₁₋₆ haloalkyl. In some embodiments, R¹ is phenyl or phenyl-C₁₋₆ alkylene (e.g., benzyl). In some embodiments, R¹ is C₆₋₁₀ aryl. In some embodiments, R¹ is C₁₋₆ alkyl substituted with C₁₋₃ alkoxy.

In some embodiments, the compound of Formula (IV) has formula:

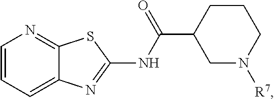

or a pharmaceutically acceptable salt thereof.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is C₁₋₆ alkyl substituted with NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1} In some embodiments, R⁷ is C₁₋₆ alkyl substituted with Cy¹. In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, R^{c2}R^{d2}, NR^{c2}C(O)R^{b2}, NR^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (IV) is selected from any one of the following compounds.

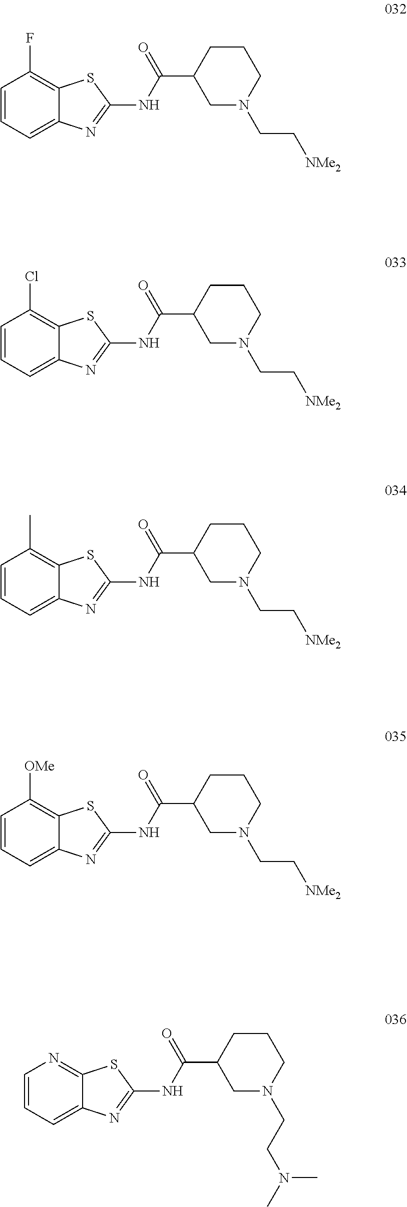 [SMILES: CN(C)CCN1CCCC(C(=O)Nc2nc3cccc(F)c3s2)C1 ; CN(C)CCN1CCCC(C(=O)Nc2nc3cccc(Cl)c3s2)C1 ; Cc1cccc2nc(NC(=O)C3CCCN(CCN(C)C)C3)sc12 ; COc1cccc2nc(NC(=O)C3CCCN(CCN(C)C)C3)sc12 ; CN(C)CCN1CCCC(C(=O)Nc2nc3cccnc3s2)C1]

or a pharmaceutically acceptable salt thereof.

### Compounds of Formula (V)

In some embodiments, the present disclosure provides a compound of Formula (V):

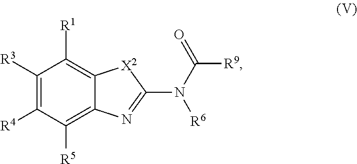

or a pharmaceutically acceptable salt thereof, wherein:
R⁹ is selected from any one of the following groups:

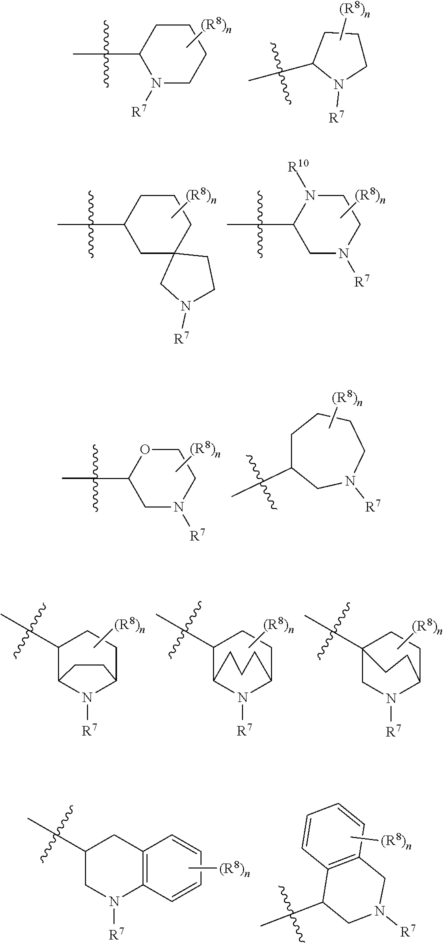

X² is selected from S, O, and NR²;
R² is selected from H, C₁₋₃ alkyl, and C₁₋₃ haloalkyl;
R³ is halo;
R⁵ is halo;
R¹ and R⁴ are each independently selected from H, halo, CN, NO₂, C(O)OR^{a1}, OR^{a1}, NR^{c1}R^{d1}, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
R¹⁰ is selected from H, C₁₋₃ alkyl, C₁₋₃ haloalkyl, C(O)OR^{a1}, C(O)R^{b1}, and C(O)NR^{c1}R^{d1};
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, R³ is F, Cl, or Br. In some embodiments, R⁵ is F, Cl, or Br. In some embodiments, R⁵ is F or Cl, and R³ is F or Cl. In some embodiments, R⁵ is F, and R³ is F. In some embodiments, R⁵ is Cl, and R³ is Cl.

In some embodiments, R¹ and R⁴ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R¹ and R⁴ are each H. In some embodiments, R¹ and R⁴ are each independently selected from H, halo, CN, OH, C₁₋₃ alkyl, C₁₋₃ haloalkyl, C₁₋₃ alkoxy, C₁₋₃ haloalkoxy, OR^{a}i-C₁₋₃ alkylene, and NR^{c1}R^{d1}—C₁₋₃ alkyl. In some embodiments, R¹ and R⁴ are each independently selected from halo, CN, OH, C₁₋₃ alkyl, C₁₋₃ haloalkyl, C₁₋₃ alkoxy, C₁₋₃ haloalkoxy, OR^{a}i-C₁₋₃ alkylene, and NR^{c1}R^{d1}—C₁₋₃ alkyl. In some embodiments, R¹ and R⁴ are each independently selected from H, halo, CN, OH, C₁₋₃ alkyl, C₁₋₃ haloalkyl, C₁₋₃ alkoxy, and C₁₋₃ haloalkoxy. In some embodiments, R¹ and R⁴ are each independently selected from halo, CN, OH, C₁₋₃ alkyl, C₁₋₃ haloalkyl, C₁₋₃ alkoxy, and C₁₋₃ haloalkoxy.

In some embodiments, at least one of R¹ and R⁴ is H. In some embodiments, at least one of R¹ and R⁴ is other than H.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S. In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, the compound of Formula (V) has formula:

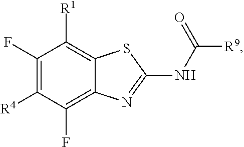

or a pharmaceutically acceptable salt thereof, wherein R⁹ is selected from the group consisting of

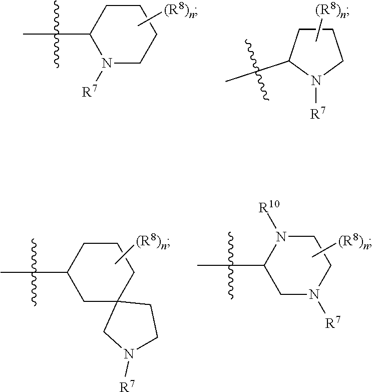

wherein R¹⁰ is selected from H, C₁₋₃ alkyl, C₁₋₃ haloalkyl, and C(O)OR^{a1};

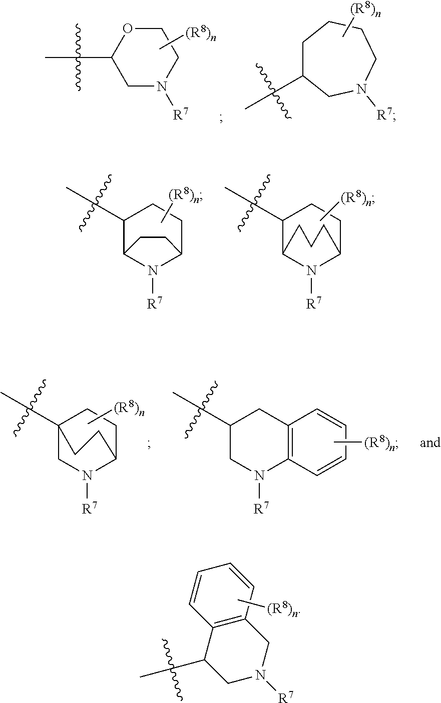

In some embodiments, R⁹ is:

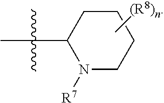

In some embodiments, R⁹ is:

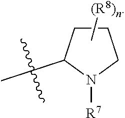

In some embodiments, R⁹ is:

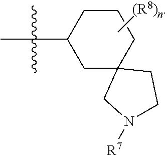

In some embodiments, R⁹ is:

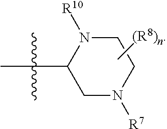

In some embodiments, R¹⁰ is selected from H, C₁₋₃ alkyl, C₁₋₃ haloalkyl, and C(O)OR^{a1}. In some embodiments, R¹⁰ is C(O)OR^{a1}.

In some embodiments, R⁹ is:

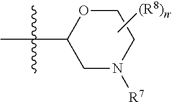

In some embodiments, R⁹ is:

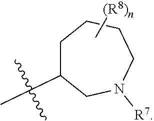

In some embodiments, R⁹ is:

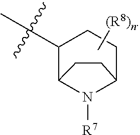

In some embodiments, R⁹ is:

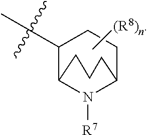

In some embodiments, R⁹ is:

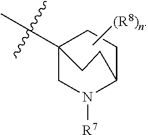

In some embodiments, R⁹ is:

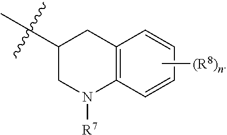

In some embodiments, R⁹ is

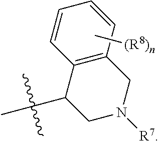

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, CN, C₁₋₃ alkyl, C₁₋₃ haloalkyl, C₁₋₃ alkoxy, and C₁₋₃ haloalkoxy.

In some embodiments, n is 0, 1, or 2. In some embodiments, n is 0. In some embodiments, n is 1. In some embodiments, n is 2.

In some embodiments, the compound of Formula (V) is selected from any one of the following formulae:

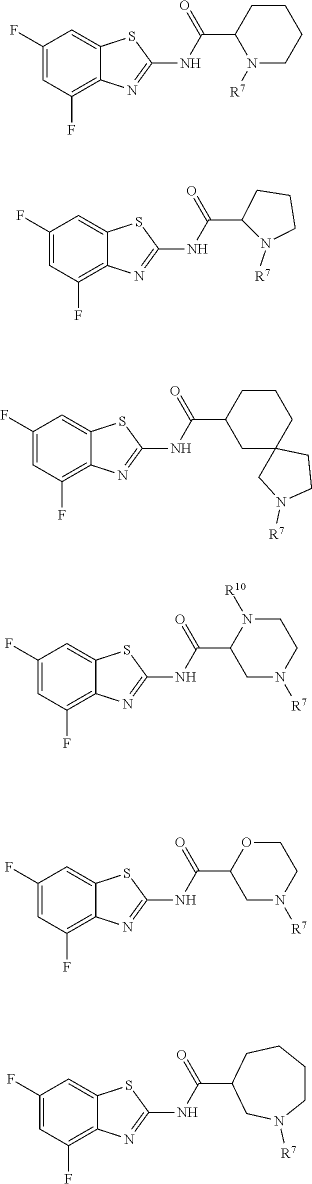

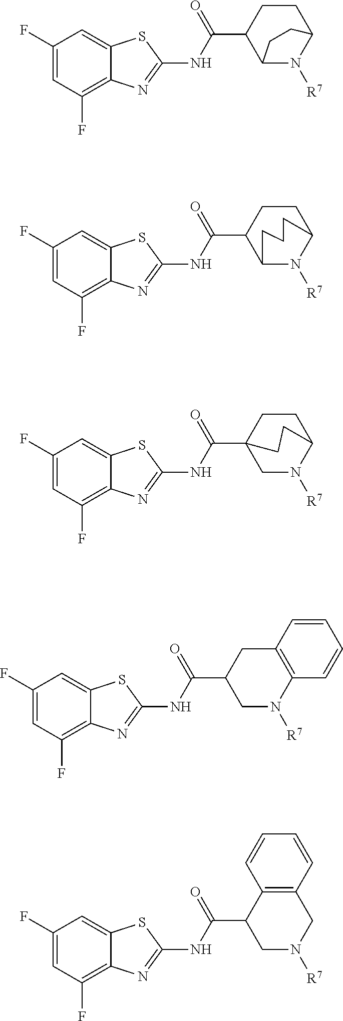

or a pharmaceutically acceptable salt thereof.

In some embodiments, the compound of Formula (V) is

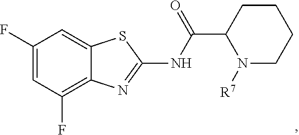

or a pharmaceutically acceptable salt thereof.

In some embodiments, the compound of Formula (V) is

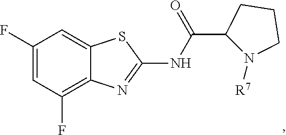

or a pharmaceutically acceptable salt thereof.

In some embodiments, the compound of Formula (V) is

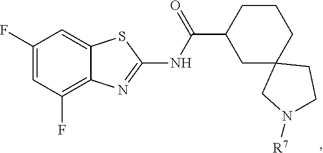

or a pharmaceutically acceptable salt thereof.

In some embodiments, the compound of Formula (V) is

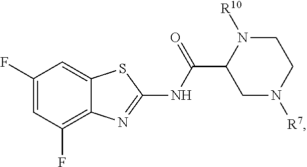

or a pharmaceutically acceptable salt thereof.

In some embodiments, the compound of Formula (V) is

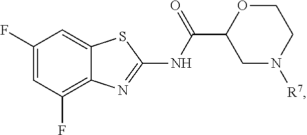

or a pharmaceutically acceptable salt thereof.

In some embodiments, the compound of Formula (V) is

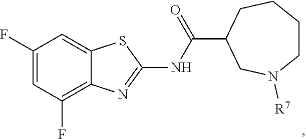

or a pharmaceutically acceptable salt thereof.

In some embodiments, the compound of Formula (V) is

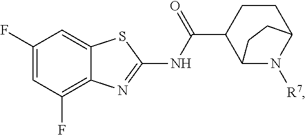

or a pharmaceutically acceptable salt thereof.

In some embodiments, the compound of Formula (V) is

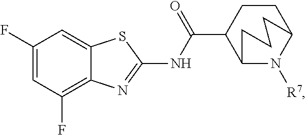

or a pharmaceutically acceptable salt thereof.

In some embodiments, the compound of Formula (V) is

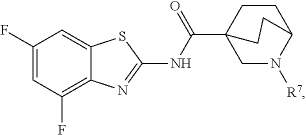

or a pharmaceutically acceptable salt thereof.

In some embodiments, the compound of Formula (V) is

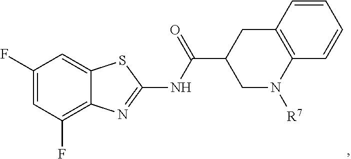

or a pharmaceutically acceptable salt thereof.

In some embodiments, the compound of Formula (V) is

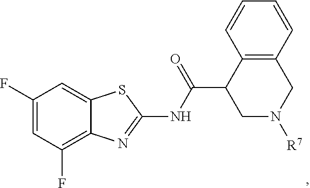

or a pharmaceutically acceptable salt thereof.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹.

In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (V) is selected from any one of the following compounds:

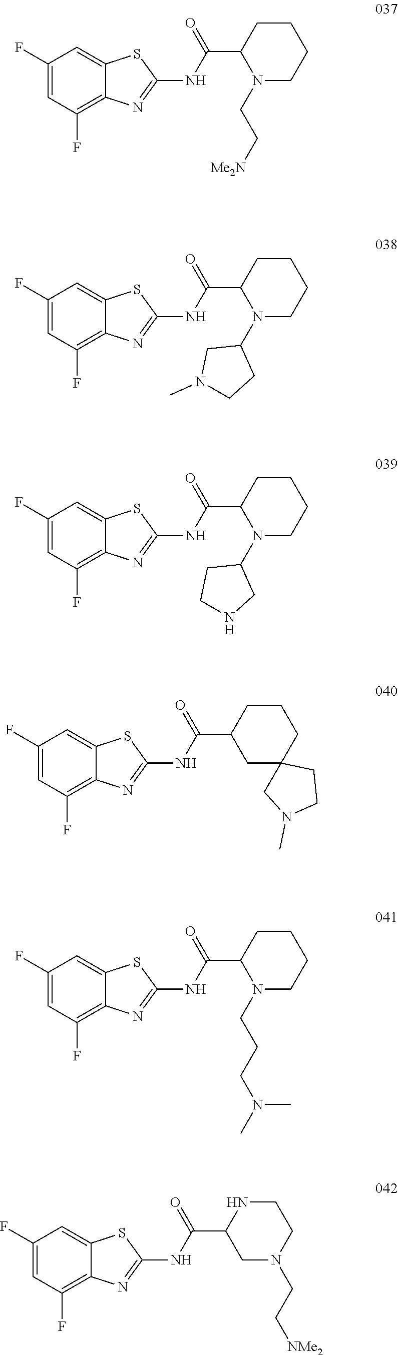 [SMILES: CN(C)CCN1CCCCC1C(=O)Nc1nc2c(F)cc(F)cc2s1 ; CN1CCC(N2CCCCC2C(=O)Nc2nc3c(F)cc(F)cc3s2)C1 ; O=C(Nc1nc2c(F)cc(F)cc2s1)C1CCCCN1C1CCNC1 ; CN1CCC2(CCCC(C(=O)Nc3nc4c(F)cc(F)cc4s3)C2)C1 ; CN(C)CCCN1CCCCC1C(=O)Nc1nc2c(F)cc(F)cc2s1 ; CN(C)CCN1CCNC(C(=O)Nc2nc3c(F)cc(F)cc3s2)C1]

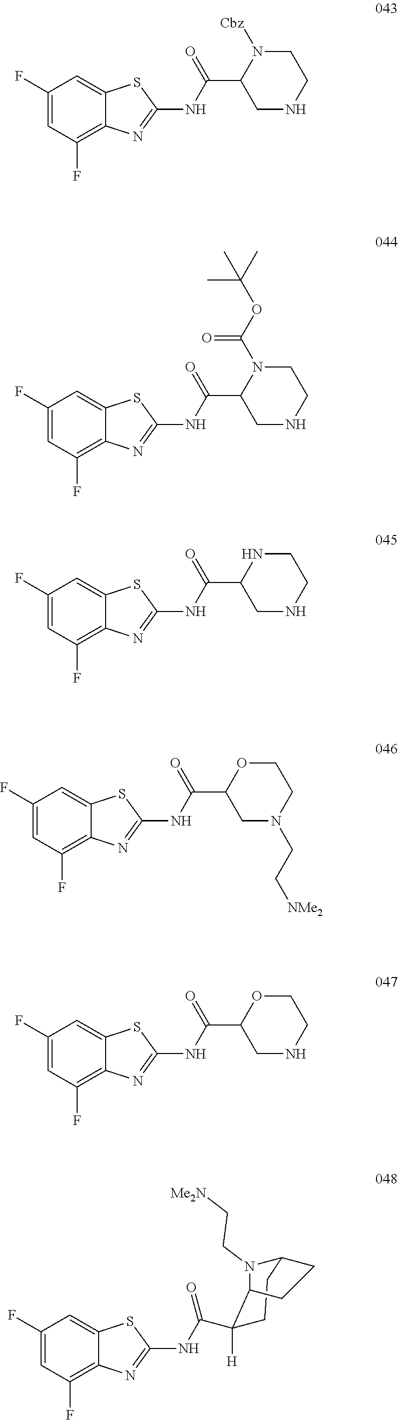 [SMILES: O=C(Nc1nc2c(F)cc(F)cc2s1)C1CNCCN1C(=O)OCc1ccccc1 ; CC(C)(C)OC(=O)N1CCNCC1C(=O)Nc1nc2c(F)cc(F)cc2s1 ; O=C(Nc1nc2c(F)cc(F)cc2s1)C1CNCCN1 ; CN(C)CCN1CCOC(C(=O)Nc2nc3c(F)cc(F)cc3s2)C1 ; O=C(Nc1nc2c(F)cc(F)cc2s1)C1CNCCO1 ; CN(C)CCN1C2CCC(C(=O)Nc3nc4c(F)cc(F)cc4s3)C1CC2]

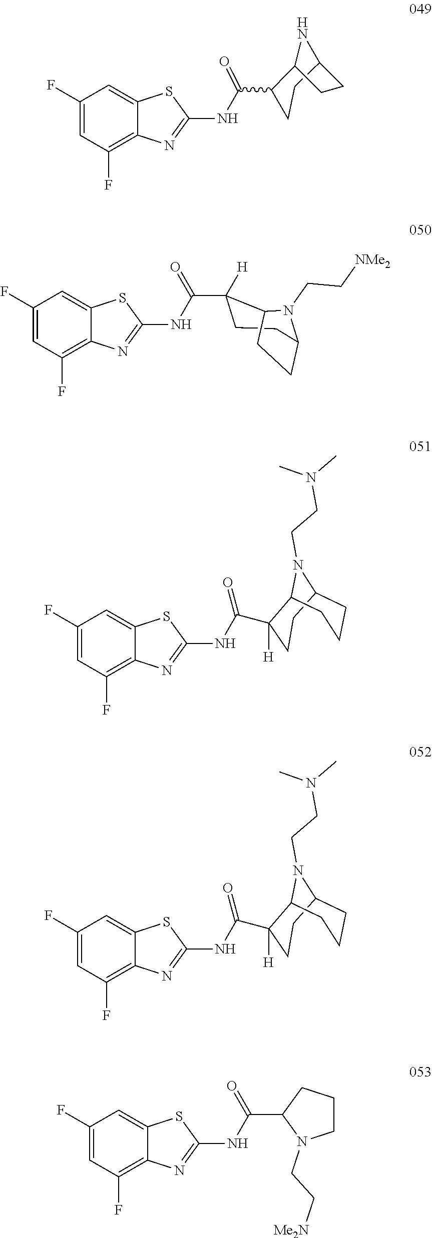 [SMILES: O=C(Nc1nc2c(F)cc(F)cc2s1)C1CCC2CCC1N2 ; CN(C)CCN1C2CCC(C(=O)Nc3nc4c(F)cc(F)cc4s3)C1CC2 ; CN(C)CCN1C2CCCC1C(C(=O)Nc1nc3c(F)cc(F)cc3s1)CC2 ; CN(C)CCN1C2CCCC1C(C(=O)Nc1nc3c(F)cc(F)cc3s1)CC2 ; CN(C)CCN1CCCC1C(=O)Nc1nc2c(F)cc(F)cc2s1]

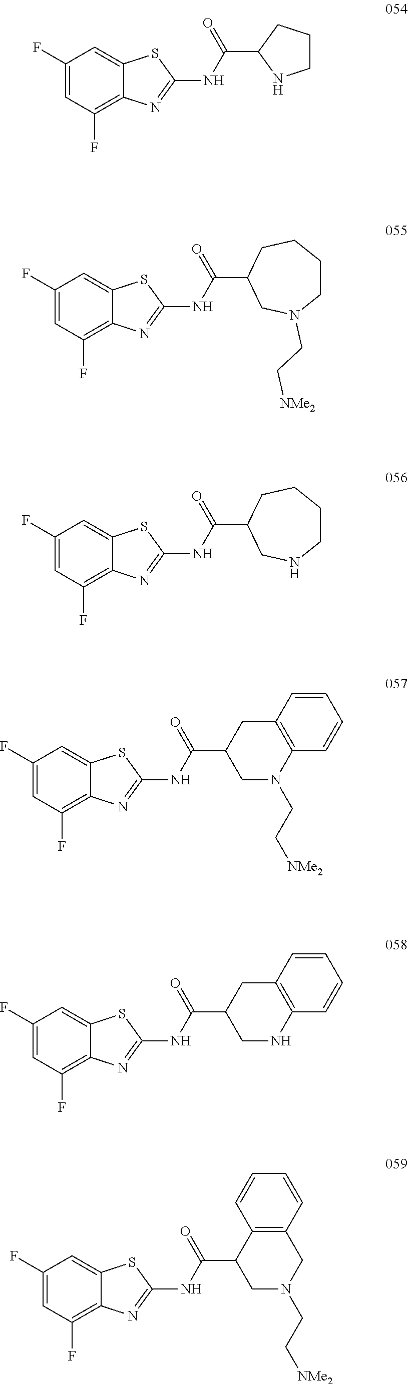 [SMILES: O=C(Nc1nc2c(F)cc(F)cc2s1)C1CCCN1 ; CN(C)CCN1CCCCC(C(=O)Nc2nc3c(F)cc(F)cc3s2)C1 ; O=C(Nc1nc2c(F)cc(F)cc2s1)C1CCCCNC1 ; CN(C)CCN1CC(C(=O)Nc2nc3c(F)cc(F)cc3s2)Cc2ccccc21 ; O=C(Nc1nc2c(F)cc(F)cc2s1)C1CNc2ccccc2C1 ; CN(C)CCN1Cc2ccccc2C(C(=O)Nc2nc3c(F)cc(F)cc3s2)C1]

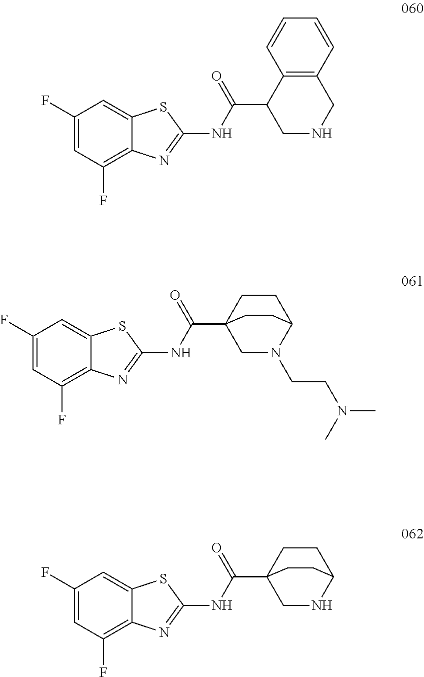 [SMILES: O=C(Nc1nc2c(F)cc(F)cc2s1)C1CNCc2ccccc21 ; CN(C)CCN1CC2(C(=O)Nc3nc4c(F)cc(F)cc4s3)CCC1CC2 ; O=C(Nc1nc2c(F)cc(F)cc2s1)C12CCC(CC1)NC2]

or a pharmaceutically acceptable salt thereof.

### Compounds of Formula (VI)

In some embodiments, the present disclosure provides a compound of Formula

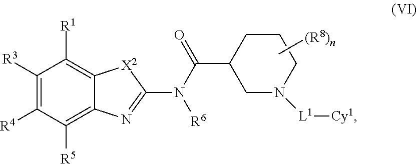

or a pharmaceutically acceptable salt thereof, wherein:
X² is selected from S, O, and NR²;
R² is selected from H, C₁₋₃ alkyl, and C₁₋₃ haloalkyl;
R³ is selected from H and halo;
R⁵ is halo;
R¹ and R⁴ are each independently selected from H, halo, CN, NO₂, C(O)OR^{a1}, OR^{a1}, NR^{c1}R^{d1} C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
L¹ is selected from a bond, C(═O), S(O)₂, and C₁₋₃ alkylene;
Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, R³ is H, F, Cl, or Br. In some embodiments, R³ is H. In some embodiments, R³ is halo. In some embodiments, R³ is F, Cl, or Br. In some embodiments, R⁵ is F, Cl, or Br. In some embodiments, R⁵ is F, Cl, or Br, and R³ is H, F or Cl. In some embodiments, R⁵ is F, Cl, or Br, and R³ is H or F. In some embodiments, R⁵ is F, and R³ is F. In some embodiments, R⁵ is F, and R³ is H. In some embodiments, R⁵ is Br, and R³ is H. In some embodiments, R⁵ is Cl, and R³ is Cl. In some embodiments, R⁵ is Cl, and R³ is F.

In some embodiments, R¹ and R⁴ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R¹ and R⁴ are each H. In some embodiments, R¹ and R⁴ are each independently selected from H, halo, CN, OH, C₁₋₃ alkyl, C₁₋₃ haloalkyl, C₁₋₃ alkoxy, C₁₋₃ haloalkoxy, OR^{a}i-C₁₋₃ alkylene, and NR^{c1}R^{d1}—C₁₋₃ alkyl. In some embodiments, R¹ and R⁴ are each independently selected from halo, CN, OH, C₁₋₃ alkyl, C₁₋₃ haloalkyl, C₁₋₃ alkoxy, C₁₋₃ haloalkoxy, OR^{a}i-C₁₋₃ alkylene, and NR^{c1}R^{d1}—C₁₋₃ alkyl. In some embodiments, R¹ and R⁴ are each independently selected from H, halo, CN, OH, C₁₋₃ alkyl, C₁₋₃ haloalkyl, C₁₋₃ alkoxy, and C₁₋₃ haloalkoxy. In some embodiments, R¹ and R⁴ are each independently selected from halo, CN, OH, C₁₋₃ alkyl, C₁₋₃ haloalkyl, C₁₋₃ alkoxy, and C₁₋₃ haloalkoxy. In some embodiments, at least one of R¹ and R⁴ is H. In some embodiments, at least one of R¹ and R⁴ is other than H.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S. In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, CN, C₁₋₃ alkyl, C₁₋₃ haloalkyl, C₁₋₃ alkoxy, and C₁₋₃ haloalkoxy.

In some embodiments, n is 0, 1, or 2. In some embodiments, n is 0. In some embodiments, n is 1. In some embodiments, n is 2.

In some embodiments, the compound of Formula (VI) is selected from:

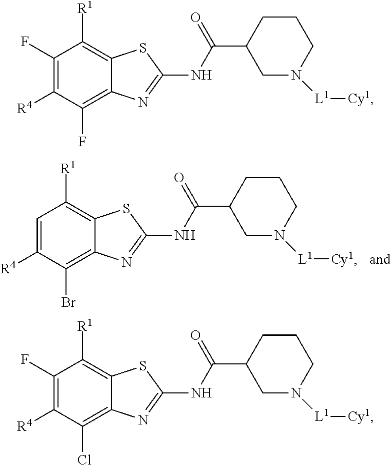

or a pharmaceutically acceptable salt thereof.

In some embodiments, the compound of Formula (VI) is

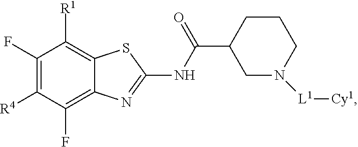

or a pharmaceutically acceptable salt thereof.

In some embodiments, the compound of Formula (VI) is

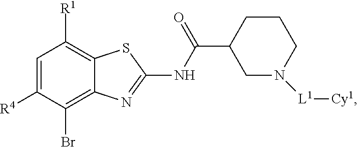

or a pharmaceutically acceptable salt thereof.

In some embodiments, the compound of Formula (VI) is

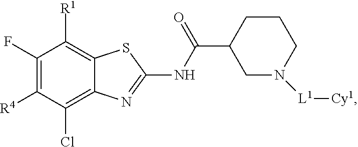

or a pharmaceutically acceptable salt thereof.

In some embodiments, L¹ is C(═O). In some embodiments, L¹ is a bond. In some embodiments, L¹ is C₁₋₃ alkylene. In some embodiments, L¹ is C(═O) or bond. In some embodiments, L¹ is C(═O) or C₁₋₃ alkylene. In some embodiments, L¹ is bond or C₁₋₃ alkylene.

In some embodiments, the compound of Formula (VI) is selected from any one of the following formulae:

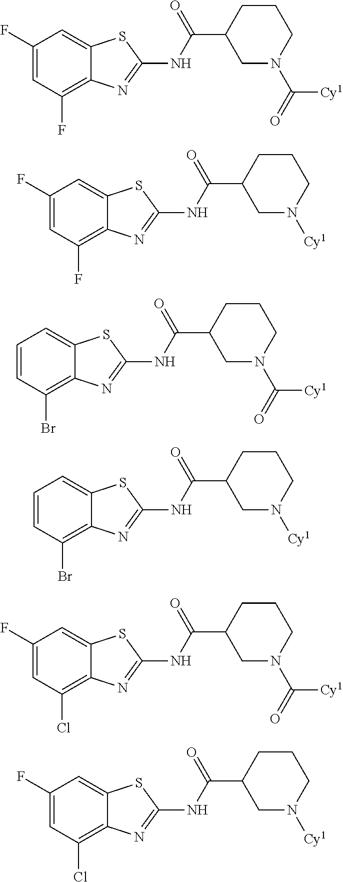

or a pharmaceutically acceptable salt thereof.

In some embodiments, the compound of Formula (VI) is

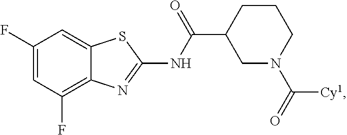

or a pharmaceutically acceptable salt thereof.

In some embodiments, the compound of Formula (VI) is

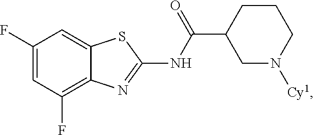

or a pharmaceutically acceptable salt thereof.

In some embodiments, the compound of Formula (VI) is

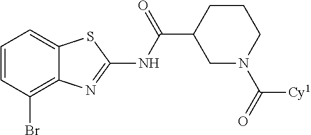

or a pharmaceutically acceptable salt thereof.

In some embodiments, the compound of Formula (VI) is

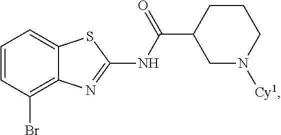

or a pharmaceutically acceptable salt thereof.

In some embodiments, the compound of Formula (VI) is

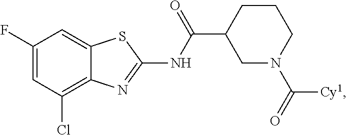

or a pharmaceutically acceptable salt thereof.

In some embodiments, the compound of Formula (VI) is

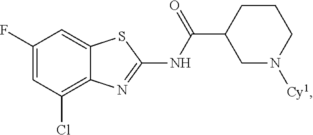

or a pharmaceutically acceptable salt thereof.

In some embodiments, Cy¹ is selected from C₆₋₁₀ aryl, 5-14 membered heteroaryl, and 4-10 membered heterocycloalkyl, each of which is optionally substituted with 1, 2, or 3 substituents independently selected from R^{Cy1}. In some embodiments, Cy¹ is C₆₋₁₀ aryl, optionally substituted with R^{Cy1}. In some embodiments, Cy¹ is 5-14 membered heteroaryl, optionally substituted with R^{Cy1}. In some embodiments, Cy¹ is 4-10 membered heterocycloalkyl, optionally substituted with R^{Cy1}.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, R^{Cy1} is selected from halo, C₁₋₆ alkyl, C₁₋₆ haloalkyl, C₂₋₆ alkenyl, OR^{a2}, C(O)R^{b2}, C(O)NR^{c2}R^{d2}, C(O)OR^{a2}, and NR^{c2}R^{d2}, wherein said C₁₋₆ alkyl is optionally substituted with OR^{a2}, C(O)NR^{c2}R^{d2}, or C(O)OR^{a2}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃ alkylene. In some embodiments, R^{d1} is C(O)OR^{a2}—C₁₋₃ alkylene. In some embodiments, R^{d1} is OR^{a2}—C₁₋₃ alkylene.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (VI) is selected from any one of the following compounds:

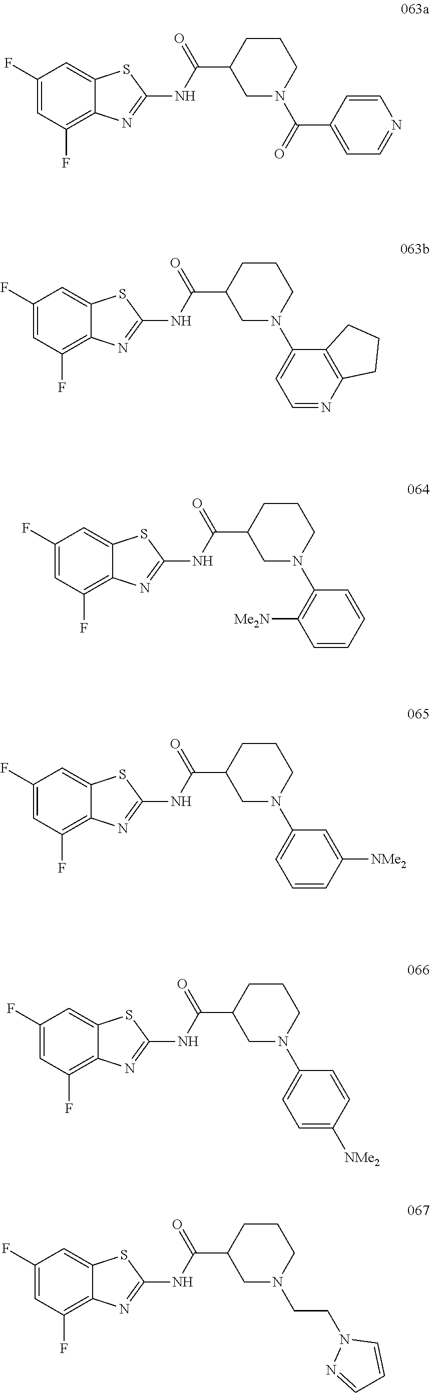 [SMILES: O=C(Nc1nc2c(F)cc(F)cc2s1)C1CCCN(C(=O)c2ccncc2)C1 ; O=C(Nc1nc2c(F)cc(F)cc2s1)C1CCCN(c2ccnc3c2CCC3)C1 ; CN(C)c1ccccc1N1CCCC(C(=O)Nc2nc3c(F)cc(F)cc3s2)C1 ; CN(C)c1cccc(N2CCCC(C(=O)Nc3nc4c(F)cc(F)cc4s3)C2)c1 ; CN(C)c1ccc(N2CCCC(C(=O)Nc3nc4c(F)cc(F)cc4s3)C2)cc1 ; O=C(Nc1nc2c(F)cc(F)cc2s1)C1CCCN(CCn2cccn2)C1]

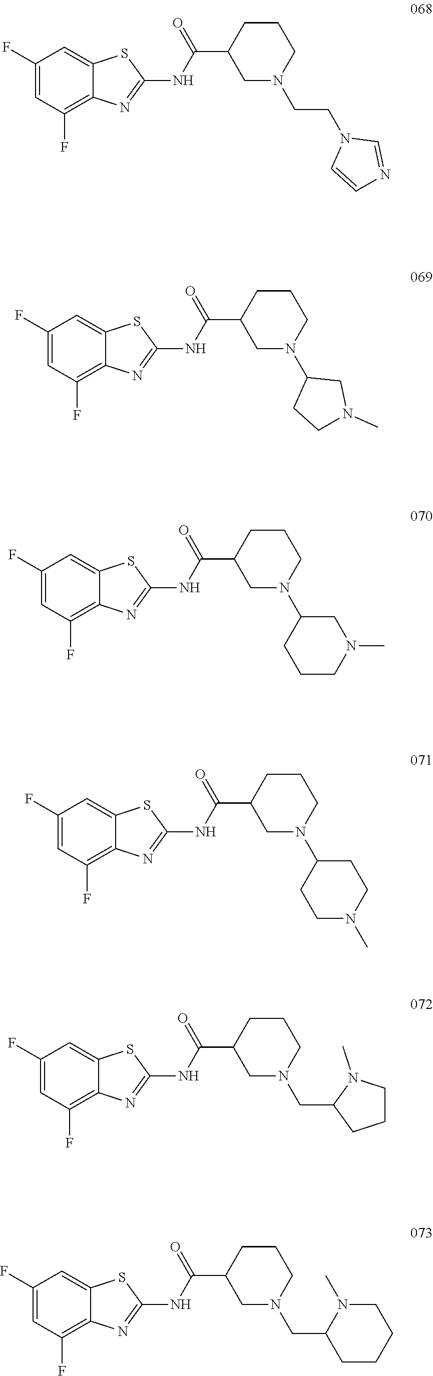 [SMILES: O=C(Nc1nc2c(F)cc(F)cc2s1)C1CCCN(CCn2ccnc2)C1 ; CN1CCC(N2CCCC(C(=O)Nc3nc4c(F)cc(F)cc4s3)C2)C1 ; CN1CCCC(N2CCCC(C(=O)Nc3nc4c(F)cc(F)cc4s3)C2)C1 ; CN1CCC(N2CCCC(C(=O)Nc3nc4c(F)cc(F)cc4s3)C2)CC1 ; CN1CCCC1CN1CCCC(C(=O)Nc2nc3c(F)cc(F)cc3s2)C1 ; CN1CCCCC1CN1CCCC(C(=O)Nc2nc3c(F)cc(F)cc3s2)C1]

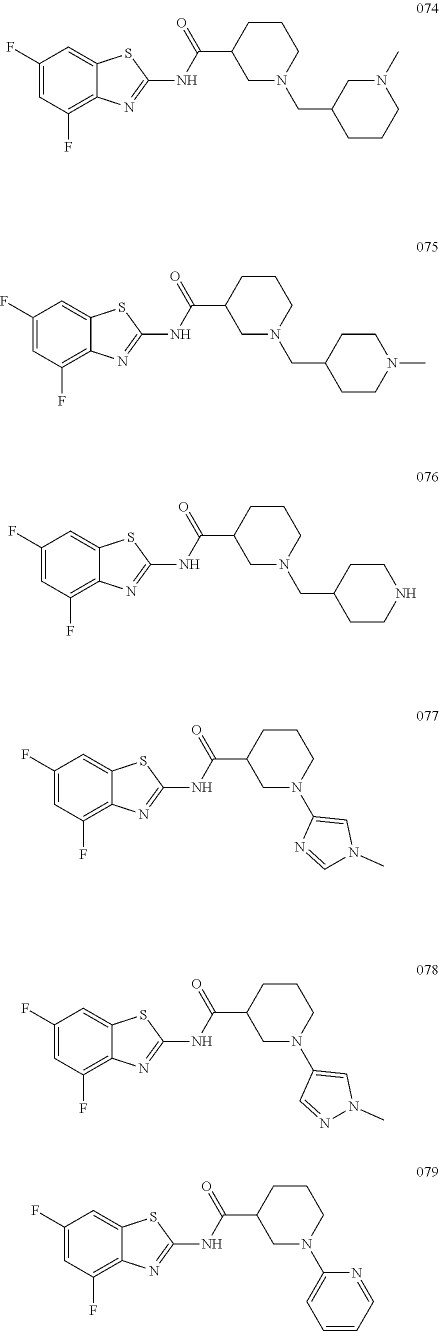 [SMILES: CN1CCCC(CN2CCCC(C(=O)Nc3nc4c(F)cc(F)cc4s3)C2)C1 ; CN1CCC(CN2CCCC(C(=O)Nc3nc4c(F)cc(F)cc4s3)C2)CC1 ; O=C(Nc1nc2c(F)cc(F)cc2s1)C1CCCN(CC2CCNCC2)C1 ; Cn1cnc(N2CCCC(C(=O)Nc3nc4c(F)cc(F)cc4s3)C2)c1 ; Cn1cc(N2CCCC(C(=O)Nc3nc4c(F)cc(F)cc4s3)C2)cn1 ; O=C(Nc1nc2c(F)cc(F)cc2s1)C1CCCN(c2ccccn2)C1]

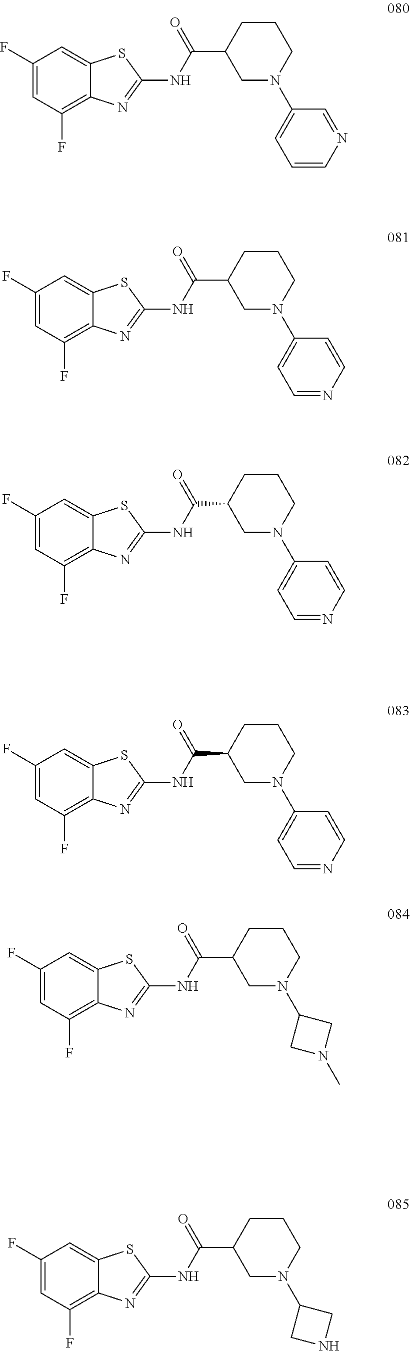 [SMILES: O=C(Nc1nc2c(F)cc(F)cc2s1)C1CCCN(c2cccnc2)C1 ; O=C(Nc1nc2c(F)cc(F)cc2s1)C1CCCN(c2ccncc2)C1 ; O=C(Nc1nc2c(F)cc(F)cc2s1)[C@@H]1CCCN(c2ccncc2)C1 ; O=C(Nc1nc2c(F)cc(F)cc2s1)[C@H]1CCCN(c2ccncc2)C1 ; CN1CC(N2CCCC(C(=O)Nc3nc4c(F)cc(F)cc4s3)C2)C1 ; O=C(Nc1nc2c(F)cc(F)cc2s1)C1CCCN(C2CNC2)C1]

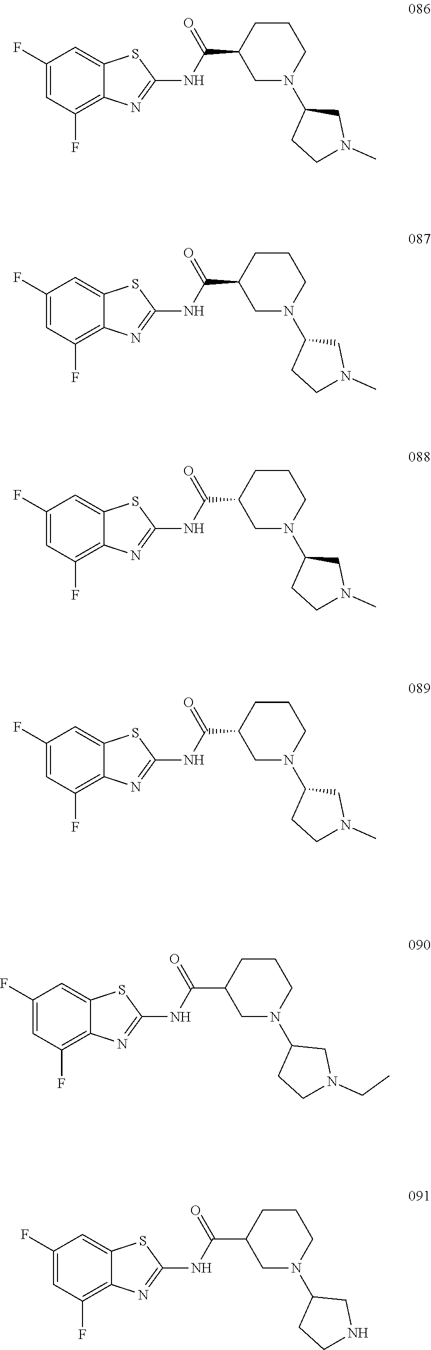 [SMILES: CN1CC[C@@H](N2CCC[C@H](C(=O)Nc3nc4c(F)cc(F)cc4s3)C2)C1 ; CN1CC[C@H](N2CCC[C@H](C(=O)Nc3nc4c(F)cc(F)cc4s3)C2)C1 ; CN1CC[C@@H](N2CCC[C@@H](C(=O)Nc3nc4c(F)cc(F)cc4s3)C2)C1 ; CN1CC[C@H](N2CCC[C@@H](C(=O)Nc3nc4c(F)cc(F)cc4s3)C2)C1 ; CCN1CCC(N2CCCC(C(=O)Nc3nc4c(F)cc(F)cc4s3)C2)C1 ; O=C(Nc1nc2c(F)cc(F)cc2s1)C1CCCN(C2CCNC2)C1]

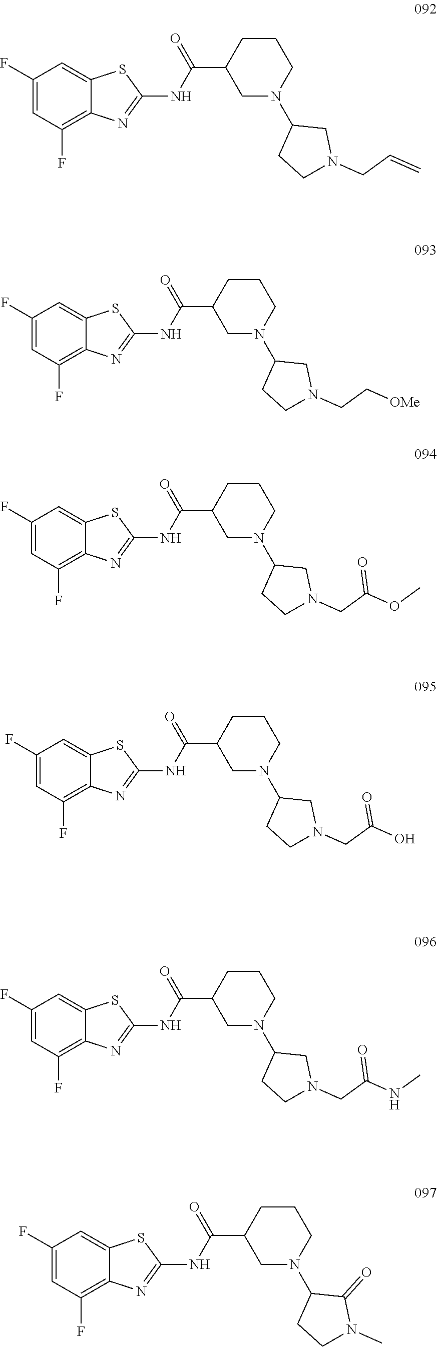 [SMILES: C=CCN1CCC(N2CCCC(C(=O)Nc3nc4c(F)cc(F)cc4s3)C2)C1 ; COCCN1CCC(N2CCCC(C(=O)Nc3nc4c(F)cc(F)cc4s3)C2)C1 ; COC(=O)CN1CCC(N2CCCC(C(=O)Nc3nc4c(F)cc(F)cc4s3)C2)C1 ; O=C(O)CN1CCC(N2CCCC(C(=O)Nc3nc4c(F)cc(F)cc4s3)C2)C1 ; CNC(=O)CN1CCC(N2CCCC(C(=O)Nc3nc4c(F)cc(F)cc4s3)C2)C1 ; CN1CCC(N2CCCC(C(=O)Nc3nc4c(F)cc(F)cc4s3)C2)C1=O]

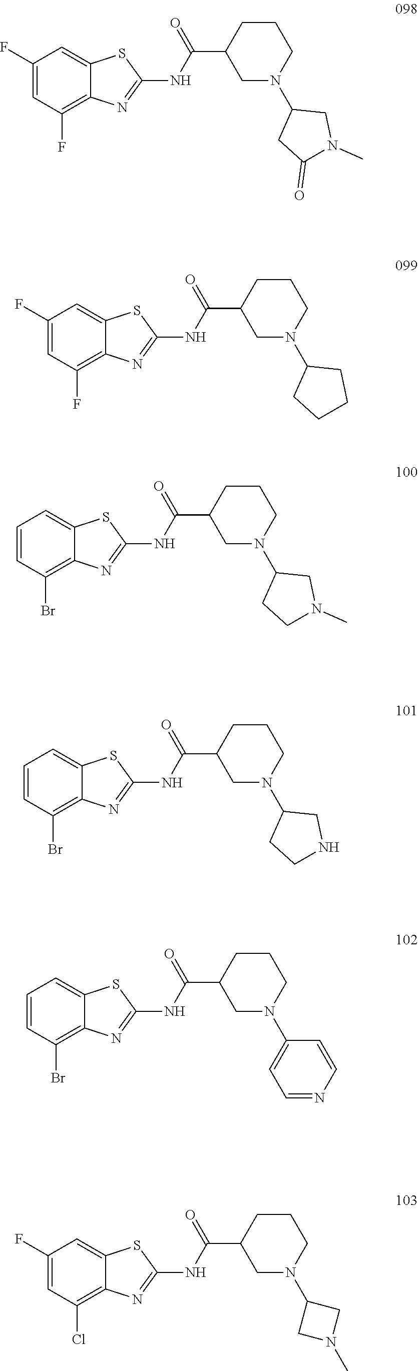 [SMILES: CN1CC(N2CCCC(C(=O)Nc3nc4c(F)cc(F)cc4s3)C2)CC1=O ; O=C(Nc1nc2c(F)cc(F)cc2s1)C1CCCN(C2CCCC2)C1 ; CN1CCC(N2CCCC(C(=O)Nc3nc4c(Br)cccc4s3)C2)C1 ; O=C(Nc1nc2c(Br)cccc2s1)C1CCCN(C2CCNC2)C1 ; O=C(Nc1nc2c(Br)cccc2s1)C1CCCN(c2ccncc2)C1 ; CN1CC(N2CCCC(C(=O)Nc3nc4c(Cl)cc(F)cc4s3)C2)C1]

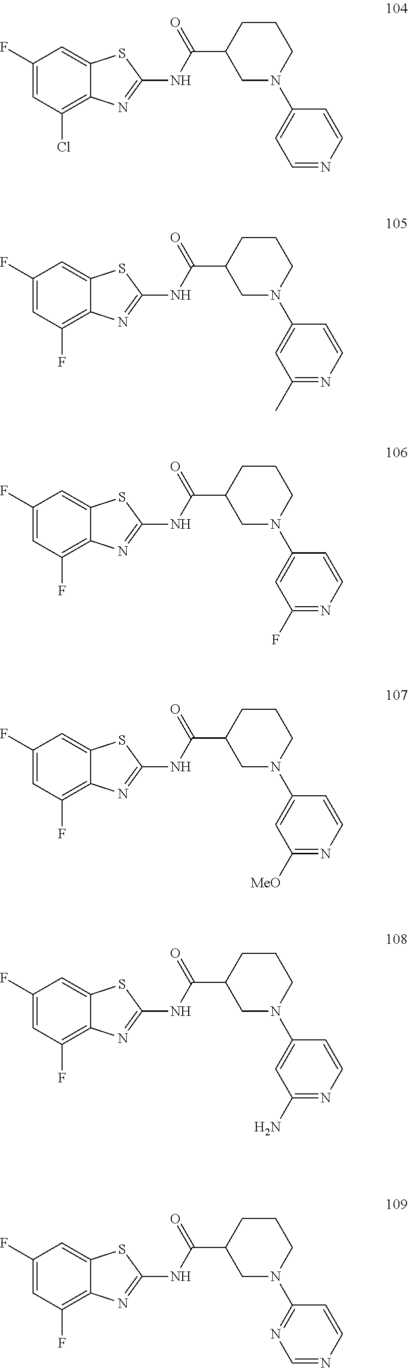 [SMILES: O=C(Nc1nc2c(Cl)cc(F)cc2s1)C1CCCN(c2ccncc2)C1 ; Cc1cc(N2CCCC(C(=O)Nc3nc4c(F)cc(F)cc4s3)C2)ccn1 ; O=C(Nc1nc2c(F)cc(F)cc2s1)C1CCCN(c2ccnc(F)c2)C1 ; COc1cc(N2CCCC(C(=O)Nc3nc4c(F)cc(F)cc4s3)C2)ccn1 ; Nc1cc(N2CCCC(C(=O)Nc3nc4c(F)cc(F)cc4s3)C2)ccn1 ; O=C(Nc1nc2c(F)cc(F)cc2s1)C1CCCN(c2ccncn2)C1]

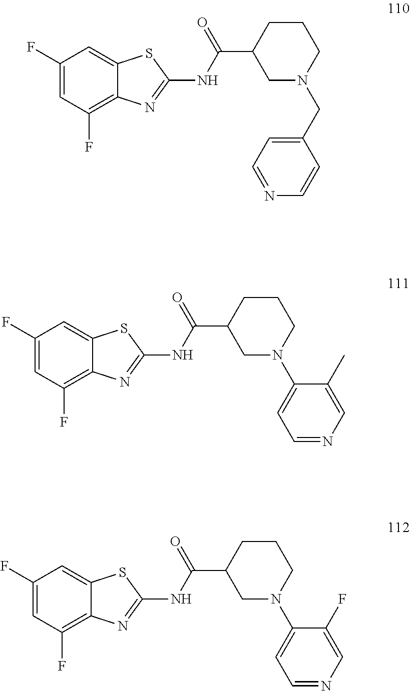 [SMILES: O=C(Nc1nc2c(F)cc(F)cc2s1)C1CCCN(Cc2ccncc2)C1 ; Cc1cnccc1N1CCCC(C(=O)Nc2nc3c(F)cc(F)cc3s2)C1 ; O=C(Nc1nc2c(F)cc(F)cc2s1)C1CCCN(c2ccncc2F)C1]

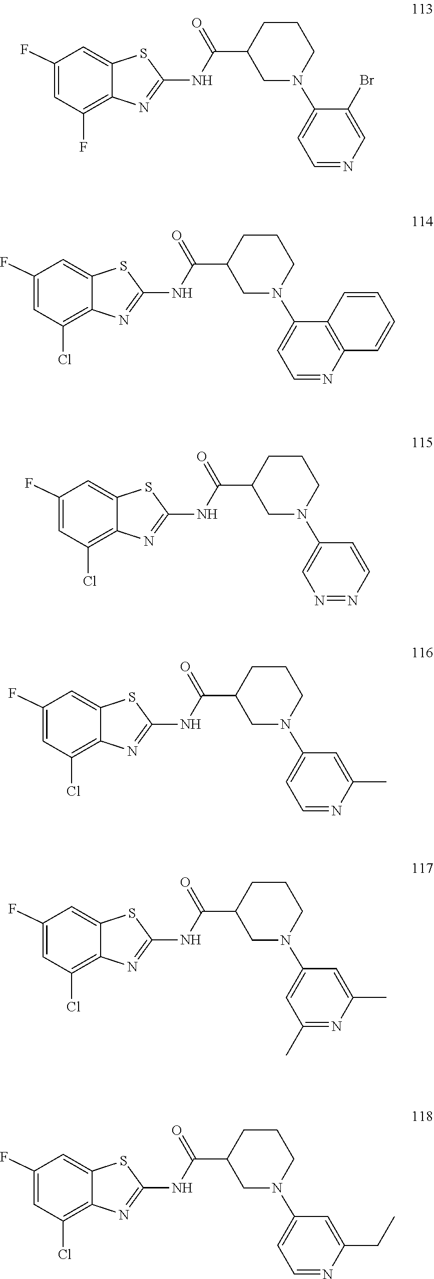 [SMILES: O=C(Nc1nc2c(F)cc(F)cc2s1)C1CCCN(c2ccncc2Br)C1 ; O=C(Nc1nc2c(Cl)cc(F)cc2s1)C1CCCN(c2ccnc3ccccc23)C1 ; O=C(Nc1nc2c(Cl)cc(F)cc2s1)C1CCCN(c2ccnnc2)C1 ; Cc1cc(N2CCCC(C(=O)Nc3nc4c(Cl)cc(F)cc4s3)C2)ccn1 ; Cc1cc(N2CCCC(C(=O)Nc3nc4c(Cl)cc(F)cc4s3)C2)cc(C)n1 ; CCc1cc(N2CCCC(C(=O)Nc3nc4c(Cl)cc(F)cc4s3)C2)ccn1]

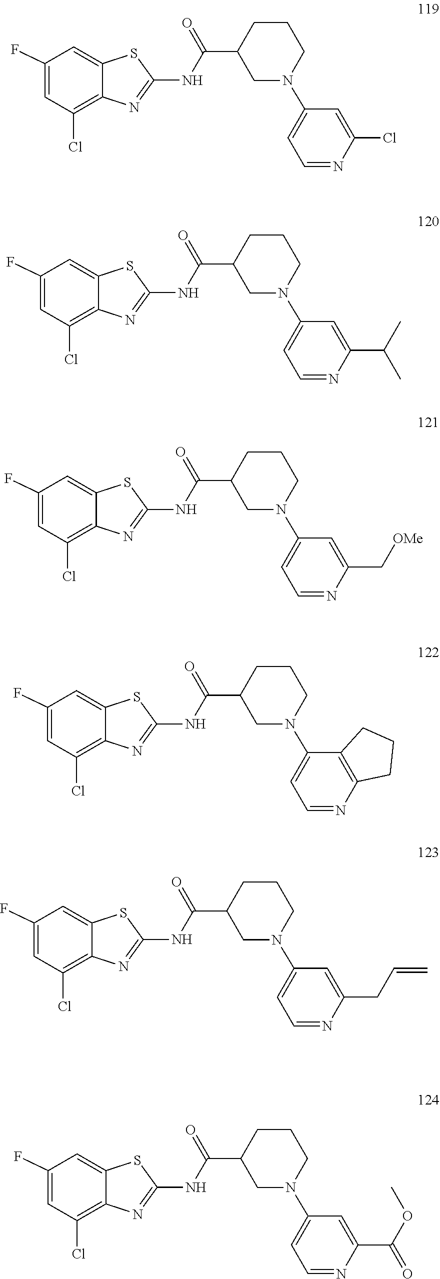 [SMILES: O=C(Nc1nc2c(Cl)cc(F)cc2s1)C1CCCN(c2ccnc(Cl)c2)C1 ; CC(C)c1cc(N2CCCC(C(=O)Nc3nc4c(Cl)cc(F)cc4s3)C2)ccn1 ; COCc1cc(N2CCCC(C(=O)Nc3nc4c(Cl)cc(F)cc4s3)C2)ccn1 ; O=C(Nc1nc2c(Cl)cc(F)cc2s1)C1CCCN(c2ccnc3c2CCC3)C1 ; C=CCc1cc(N2CCCC(C(=O)Nc3nc4c(Cl)cc(F)cc4s3)C2)ccn1 ; COC(=O)c1cc(N2CCCC(C(=O)Nc3nc4c(Cl)cc(F)cc4s3)C2)ccn1]

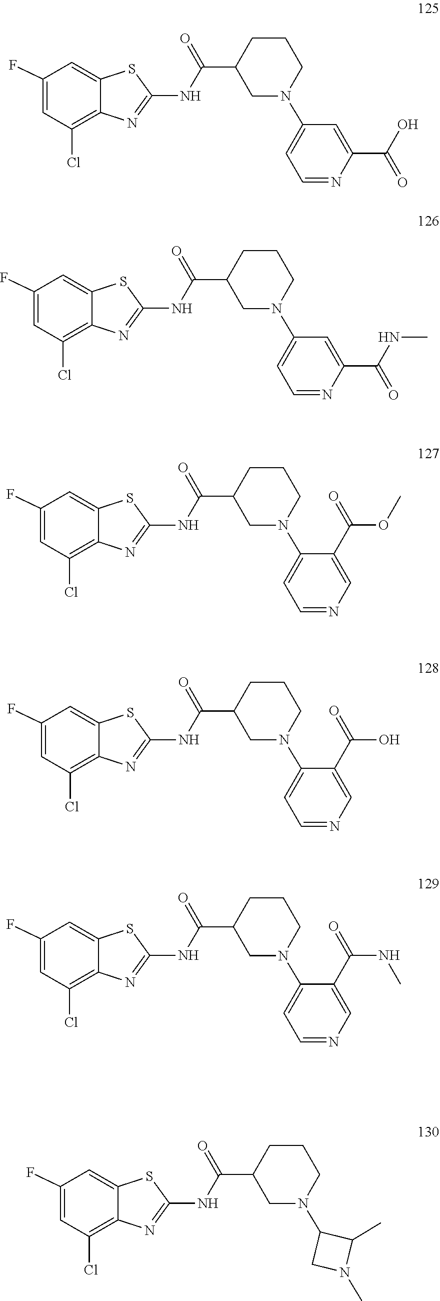 [SMILES: O=C(O)c1cc(N2CCCC(C(=O)Nc3nc4c(Cl)cc(F)cc4s3)C2)ccn1 ; CNC(=O)c1cc(N2CCCC(C(=O)Nc3nc4c(Cl)cc(F)cc4s3)C2)ccn1 ; COC(=O)c1cnccc1N1CCCC(C(=O)Nc2nc3c(Cl)cc(F)cc3s2)C1 ; O=C(O)c1cnccc1N1CCCC(C(=O)Nc2nc3c(Cl)cc(F)cc3s2)C1 ; CNC(=O)c1cnccc1N1CCCC(C(=O)Nc2nc3c(Cl)cc(F)cc3s2)C1 ; CC1C(N2CCCC(C(=O)Nc3nc4c(Cl)cc(F)cc4s3)C2)CN1C]

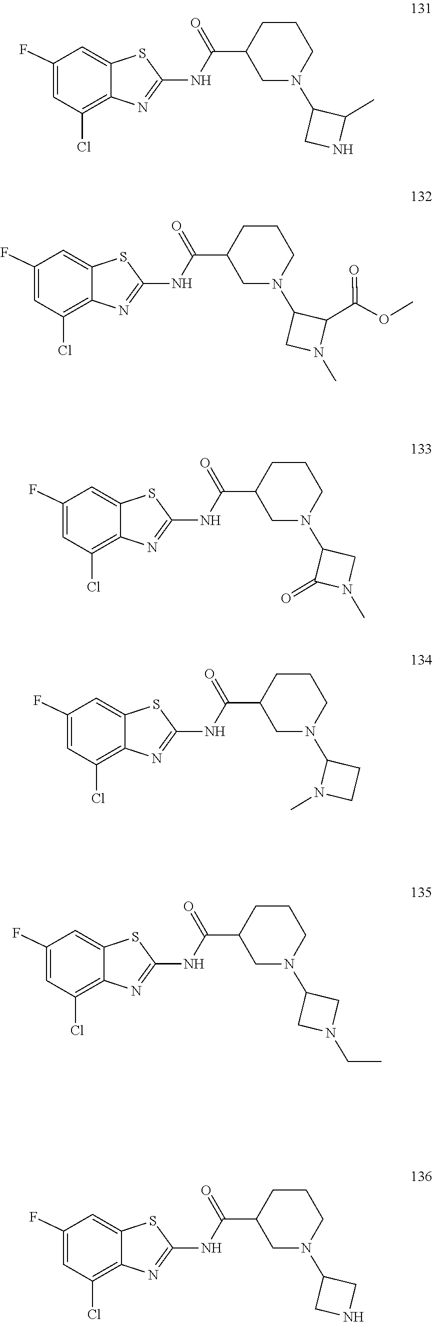 [SMILES: CC1NCC1N1CCCC(C(=O)Nc2nc3c(Cl)cc(F)cc3s2)C1 ; COC(=O)C1C(N2CCCC(C(=O)Nc3nc4c(Cl)cc(F)cc4s3)C2)CN1C ; CN1CC(N2CCCC(C(=O)Nc3nc4c(Cl)cc(F)cc4s3)C2)C1=O ; CN1CCC1N1CCCC(C(=O)Nc2nc3c(Cl)cc(F)cc3s2)C1 ; CCN1CC(N2CCCC(C(=O)Nc3nc4c(Cl)cc(F)cc4s3)C2)C1 ; O=C(Nc1nc2c(Cl)cc(F)cc2s1)C1CCCN(C2CNC2)C1]

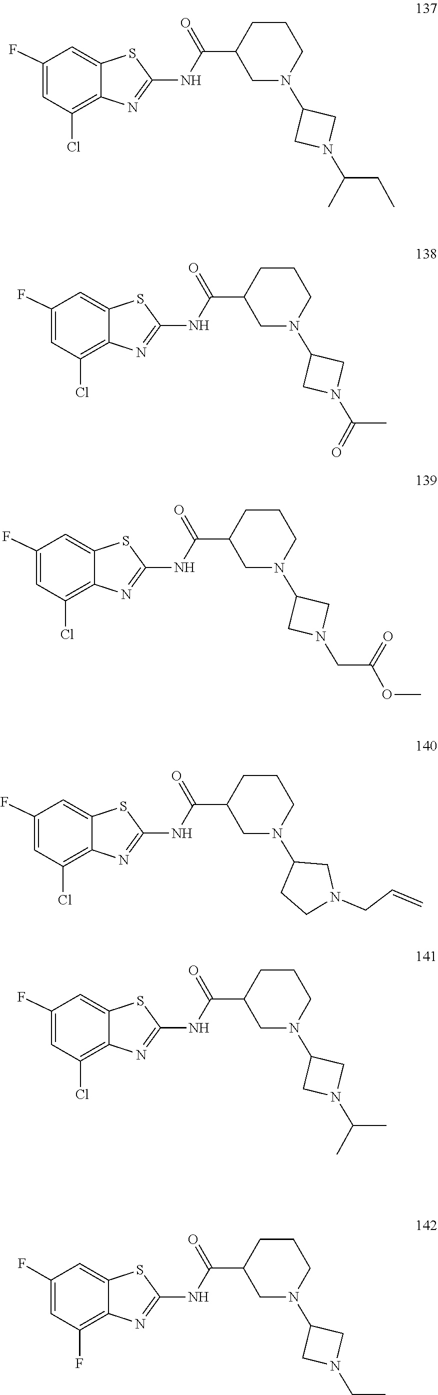 [SMILES: CCC(C)N1CC(N2CCCC(C(=O)Nc3nc4c(Cl)cc(F)cc4s3)C2)C1 ; CC(=O)N1CC(N2CCCC(C(=O)Nc3nc4c(Cl)cc(F)cc4s3)C2)C1 ; COC(=O)CN1CC(N2CCCC(C(=O)Nc3nc4c(Cl)cc(F)cc4s3)C2)C1 ; C=CCN1CCC(N2CCCC(C(=O)Nc3nc4c(Cl)cc(F)cc4s3)C2)C1 ; CC(C)N1CC(N2CCCC(C(=O)Nc3nc4c(Cl)cc(F)cc4s3)C2)C1 ; CCN1CC(N2CCCC(C(=O)Nc3nc4c(F)cc(F)cc4s3)C2)C1]

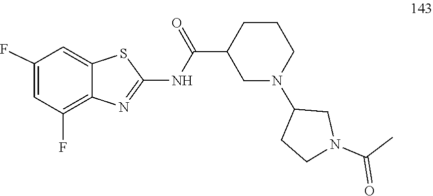 [SMILES: CC(=O)N1CCC(N2CCCC(C(=O)Nc3nc4c(F)cc(F)cc4s3)C2)C1]

or a pharmaceutically acceptable salt thereof.

### Compounds of Formula (VII)

In some embodiments, the present disclosure provides a compound of Formula (VII):

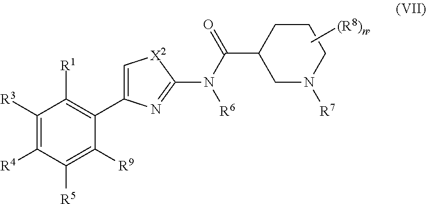

or a pharmaceutically acceptable salt thereof, wherein:
X² is selected from S, O, and NR²;
R² is selected from H, C₁₋₃ alkyl, and C₁₋₃ haloalkyl;
R¹, R³, R⁴, R⁵, and R⁹ are each independently selected from H, halo, CN, NO₂, C(O)OR^{a1}, OR^{a1}, NR^{c1}R^{d1} C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, R¹, R³, R⁴, R⁵, and R⁹ are each independently selected from H, halo, CN, C(O)OR^{a1}, OR^{a1}, NR^{c1}R^{d1} Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, at least one of R¹, R³, R⁴, R⁵, and R⁹ is H. In some embodiments, at least one of R¹, R³, R⁴, R⁵, and R⁹ is halo, CN, NO₂, C(O)OR^{a1}, OR^{a1}, NR^{c1}R^{d1} C₁₋₃ alkyl, or C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R¹, R³, R⁴, R⁵, and R⁹ are each H. In some embodiments, R¹, R³, R⁴, R⁵, and R⁹ are each independently selected from H, halo, OR^{a1}, NR^{c1}R^{d1} and C₁₋₃ alkyl. In some embodiments, R¹, R³, R⁴, R⁵, and R⁹ are each independently selected from halo, OR^{a1}, NR^{c1}R^{d1} and C₁₋₃ alkyl. In some embodiments, R¹, R³, R⁴, R⁵, and R⁹ are each independently selected from halo, C₁₋₃ alkyl, and C₁₋₃ alkoxy.

In some embodiments, R¹ is halo (e.g., F, Cl, or Br). In some embodiments, R¹ is C₁₋₃ alkyl. In some embodiments, R¹ is OR^{a1} (e.g., C₁₋₃ alkoxy or C₁₋₃ haloalkoxy). In some embodiments, R¹ is NR^{c1}R^{d1}. In some embodiments, R³ is halo (e.g., F, Cl, or Br).

In some embodiments, R³ is C₁₋₃ alkyl. In some embodiments, R³ is OR^{a1} (e.g., C₁₋₃ alkoxy or C₁₋₃ haloalkoxy). In some embodiments, R³ is NR^{c1}R^{d1}. In some embodiments, R⁴ is halo (e.g., F, Cl, or Br). In some embodiments, R⁴ is C₁₋₃ alkyl. In some embodiments, R⁴ is OR^{a1} (e.g., C₁₋₃ alkoxy or C₁₋₃ haloalkoxy). In some embodiments, R⁴ is NR^{c1}R^{d1}. In some embodiments, R⁵ is halo (e.g., F, Cl, or Br). In some embodiments, R⁵ is C₁₋₃ alkyl. In some embodiments, R⁵ is OR^{a1} (e.g., C₁₋₃ alkoxy or C₁₋₃ haloalkoxy).

In some embodiments, R⁵ is NR^{c1}R^{d1}. In some embodiments, R⁹ is halo (e.g., F, Cl, or Br). In some embodiments, R⁹ is C₁₋₃ alkyl. In some embodiments, R⁹ is OR^{a1} (e.g., C₁₋₃ alkoxy or C₁₋₃ haloalkoxy). In some embodiments, R⁹ is NR^{c1}R^{d1}. In some embodiments, R¹ is H. In some embodiments, R³ is H. In some embodiments, R⁴ is H. In some embodiments, R⁵ is H. In some embodiments, R⁹ is H.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S.

In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, CN, C₁₋₃ alkyl, C₁₋₃ haloalkyl, C₁₋₃ alkoxy, and C₁₋₃ haloalkoxy.

In some embodiments, n is 0, 1, or 2. In some embodiments, n is 0. In some embodiments, n is 1. In some embodiments, n is 2.

In some embodiments, the compound of Formula (VII) has formula:

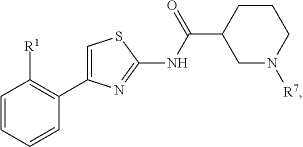

or a pharmaceutically acceptable salt thereof. In some embodiments, R¹ is selected from halo, OR^{a1}, NR^{c1}R^{d1} and C₁₋₃ alkyl. In some embodiments, R¹ is halo, C₁₋₃ alkyl, or C₁₋₃ alkoxy.

In some embodiments, the compound of Formula (VII) has formula:

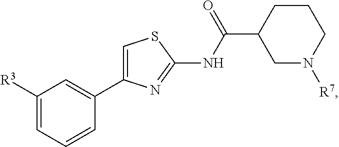

or a pharmaceutically acceptable salt thereof. In some embodiments, R³ is selected from halo, OR^{a1}, NR^{c1}R^{d1}, and C₁₋₃ alkyl. In some embodiments, R³ is halo, C₁₋₃ alkyl, or C₁₋₃ alkoxy.

In some embodiments, the compound of Formula (VII) has formula:

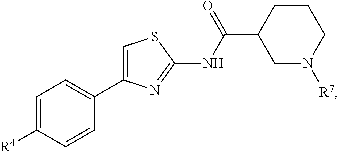

or a pharmaceutically acceptable salt thereof. In some embodiments, R⁴ is selected from halo, OR^{a1}, NR^{c1}R^{d1} and C₁₋₃ alkyl.

In some embodiments, the compound of Formula (VII) has formula:

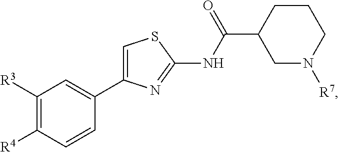

or a pharmaceutically acceptable salt thereof. In some embodiments, R³ and R⁴ are each independently selected from halo, OR^{a1}, NR^{c1}R^{d1} and C₁₋₃ alkyl. In some embodiments, R³ and R⁴ are each independently selected from halo and C₁₋₃ alkoxy.

In some embodiments, the compound of Formula (VII) has formula:

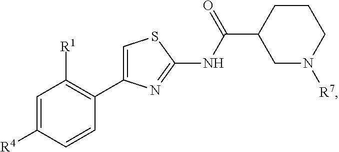

or a pharmaceutically acceptable salt thereof. In some embodiments, R¹ and R⁴ are each independently selected from halo, OR^{a1}, NR^{c1}R^{d1} and C₁₋₃ alkyl. In some embodiments, R¹ and R⁴ are each independently selected from halo and C₁₋₃ alkoxy.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹.

In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is selected from Cy¹, C(═O)Cy¹, and C₁₋₆ alkyl substituted with Cy¹. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃ alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (VII) is selected from any one of the following compounds:

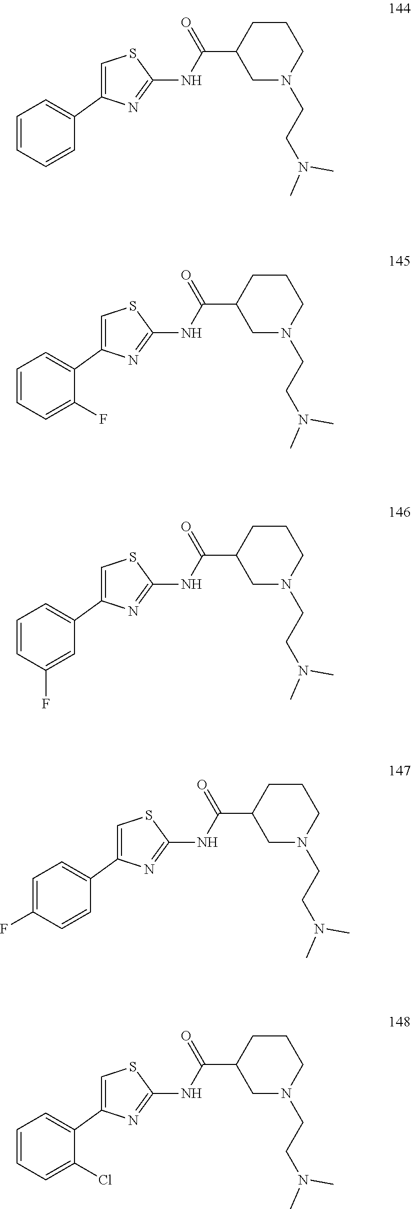 [SMILES: CN(C)CCN1CCCC(C(=O)Nc2nc(-c3ccccc3)cs2)C1 ; CN(C)CCN1CCCC(C(=O)Nc2nc(-c3ccccc3F)cs2)C1 ; CN(C)CCN1CCCC(C(=O)Nc2nc(-c3cccc(F)c3)cs2)C1 ; CN(C)CCN1CCCC(C(=O)Nc2nc(-c3ccc(F)cc3)cs2)C1 ; CN(C)CCN1CCCC(C(=O)Nc2nc(-c3ccccc3Cl)cs2)C1]

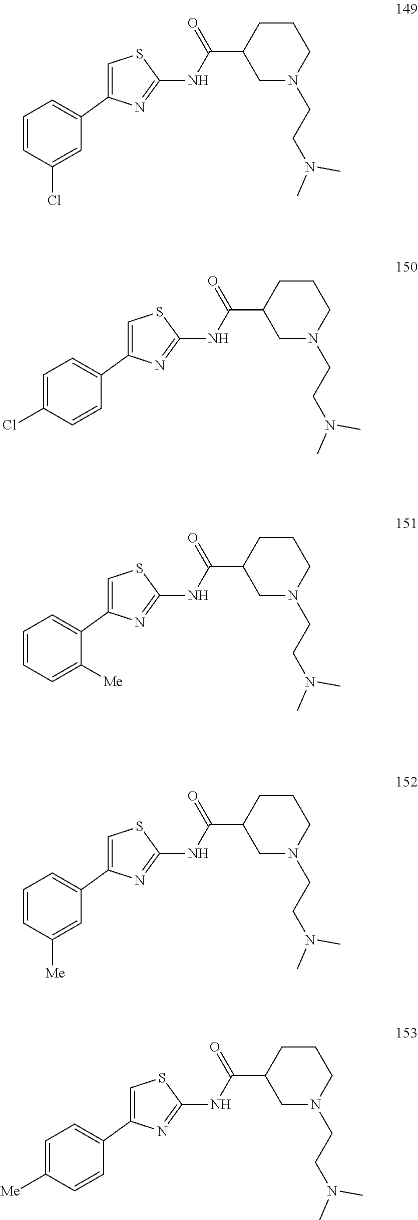 [SMILES: CN(C)CCN1CCCC(C(=O)Nc2nc(-c3cccc(Cl)c3)cs2)C1 ; CN(C)CCN1CCCC(C(=O)Nc2nc(-c3ccc(Cl)cc3)cs2)C1 ; Cc1ccccc1-c1csc(NC(=O)C2CCCN(CCN(C)C)C2)n1 ; Cc1cccc(-c2csc(NC(=O)C3CCCN(CCN(C)C)C3)n2)c1 ; Cc1ccc(-c2csc(NC(=O)C3CCCN(CCN(C)C)C3)n2)cc1]

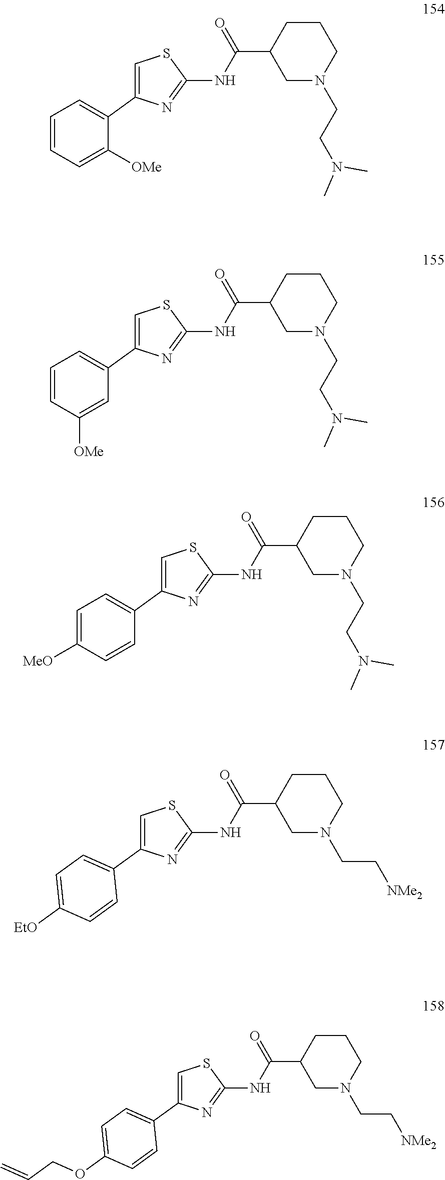

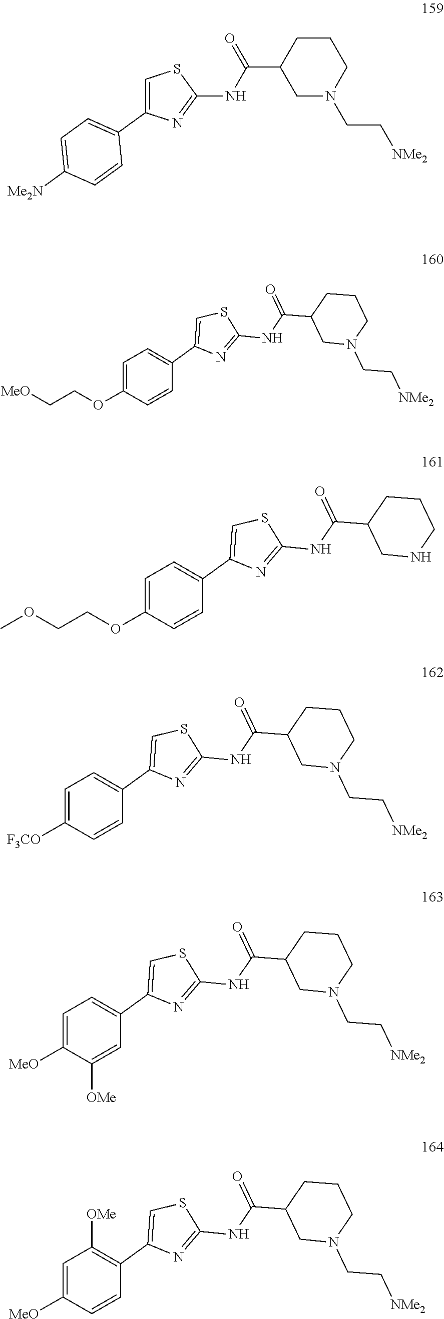 [SMILES: CN(C)CCN1CCCC(C(=O)Nc2nc(-c3ccc(N(C)C)cc3)cs2)C1 ; COCCOc1ccc(-c2csc(NC(=O)C3CCCN(CCN(C)C)C3)n2)cc1 ; COCCOc1ccc(-c2csc(NC(=O)C3CCCNC3)n2)cc1 ; CN(C)CCN1CCCC(C(=O)Nc2nc(-c3ccc(OC(F)(F)F)cc3)cs2)C1 ; COc1ccc(-c2csc(NC(=O)C3CCCN(CCN(C)C)C3)n2)cc1OC ; COc1ccc(-c2csc(NC(=O)C3CCCN(CCN(C)C)C3)n2)c(OC)c1]

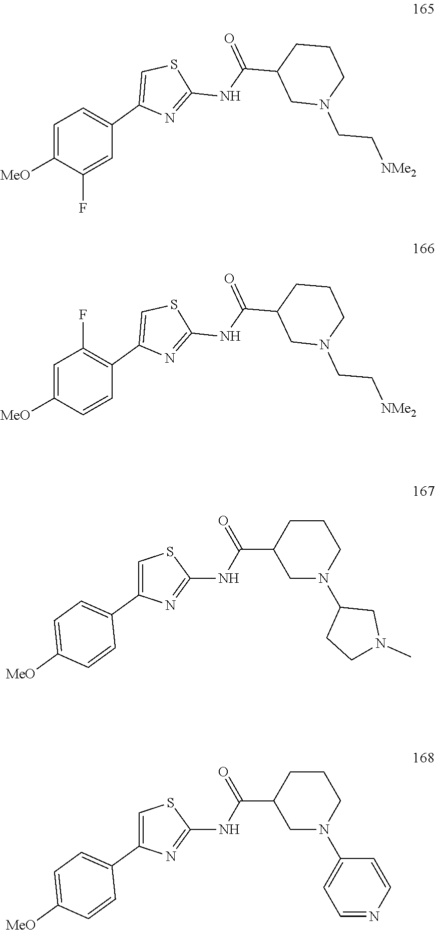 [SMILES: COc1ccc(-c2csc(NC(=O)C3CCCN(CCN(C)C)C3)n2)cc1F ; COc1ccc(-c2csc(NC(=O)C3CCCN(CCN(C)C)C3)n2)c(F)c1 ; COc1ccc(-c2csc(NC(=O)C3CCCN(C4CCN(C)C4)C3)n2)cc1 ; COc1ccc(-c2csc(NC(=O)C3CCCN(c4ccncc4)C3)n2)cc1]

or a pharmaceutically acceptable salt thereof.

### Compounds of Formula (VIII)

In some embodiments, the present disclosure provides a compound of Formula (VIII):

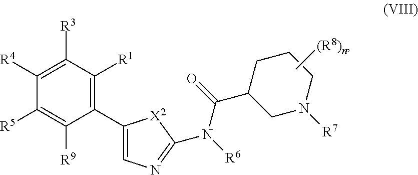

or a pharmaceutically acceptable salt thereof, wherein:
X² is selected from S, O, and NR²;
R² is selected from H, C₁₋₃ alkyl, and C₁₋₃ haloalkyl;
R¹, R³, R⁴, R⁵, and R⁹ are each independently selected from H, halo, CN, NO₂, C(O)OR^{a1}, OR^{a1}, NR^{c1}R^{d1}, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, R¹, R³, R⁴, R⁵, and R⁹ are each independently selected from H, halo, CN, C(O)OR^{a1}, OR^{a1}, NR^{c1}R^{d1} Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, at least one of R¹, R³, R⁴, R⁵, and R⁹ is H. In some embodiments, at least one of R¹, R³, R⁴, R⁵, and R⁹ is halo, CN, NO₂, C(O)OR^{a1}, OR^{a1}, NR^{c1}R^{d1} C₁₋₃ alkyl, or C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R¹, R³, R⁴, R⁵, and R⁹ are each H. In some embodiments, R¹, R³, R⁴, R⁵, and R⁹ are each independently selected from H, halo, OR^{a1}, NR^{c1}R^{d1} and C₁₋₃ alkyl. In some embodiments, R¹, R³, R⁴, R⁵, and R⁹ are each independently selected from halo, OR^{a1}, NR^{c1}R^{d1} and C₁₋₃ alkyl. In some embodiments, R¹, R³, R⁴, R⁵, and R⁹ are each independently selected from halo, C₁₋₃ alkyl, and C₁₋₃ alkoxy.

In some embodiments, R¹ is halo (e.g., F, Cl, or Br). In some embodiments, R¹ is C₁₋₃ alkyl. In some embodiments, R¹ is OR^{a1} (e.g., C₁₋₃ alkoxy or C₁₋₃ haloalkoxy). In some embodiments, R¹ is NR^{c1}R^{d1}. In some embodiments, R³ is halo (e.g., F, Cl, or Br).

In some embodiments, R³ is C₁₋₃ alkyl. In some embodiments, R³ is OR^{a1} (e.g., C₁₋₃ alkoxy or C₁₋₃ haloalkoxy). In some embodiments, R³ is NR^{c1}R^{d1}. In some embodiments, R⁴ is halo (e.g., F, Cl, or Br). In some embodiments, R⁴ is C₁₋₃ alkyl. In some embodiments, R⁴ is OR^{a1} (e.g., C₁₋₃ alkoxy or C₁₋₃ haloalkoxy). In some embodiments, R⁴ is NR^{c1}R^{d1}. In some embodiments, R⁵ is halo (e.g., F, Cl, or Br). In some embodiments, R⁵ is C₁₋₃ alkyl. In some embodiments, R⁵ is OR^{a1} (e.g., C₁₋₃ alkoxy or C₁₋₃ haloalkoxy).

In some embodiments, R⁵ is NR^{c1}R^{d1}. In some embodiments, R⁹ is halo (e.g., F, Cl, or Br). In some embodiments, R⁹ is C₁₋₃ alkyl. In some embodiments, R⁹ is OR^{a1} (e.g., C₁₋₃ alkoxy or C₁₋₃ haloalkoxy). In some embodiments, R⁹ is NR^{c1}R^{d1}. In some embodiments, R¹ is H. In some embodiments, R³ is H. In some embodiments, R⁴ is H. In some embodiments, R⁵ is H. In some embodiments, R⁹ is H.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S. In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, CN, C₁₋₃ alkyl, C₁₋₃ haloalkyl, C₁₋₃ alkoxy, and C₁₋₃ haloalkoxy.

In some embodiments, n is 0, 1, or 2. In some embodiments, n is 0. In some embodiments, n is 1. In some embodiments, n is 2.

In some embodiments, the compound of Formula (VIII) has formula:

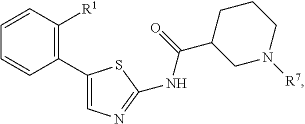

or a pharmaceutically acceptable salt thereof. In some embodiments, R¹ is selected from halo, C₁₋₃ alkyl, and C₁₋₃ alkoxy.

In some embodiments, the compound of Formula (VIII) has formula:

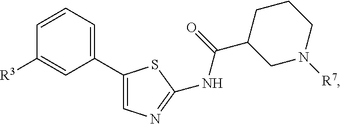

or a pharmaceutically acceptable salt thereof. In some embodiments, R³ is selected from halo, C₁₋₃ alkyl, and C₁₋₃ alkoxy.

In some embodiments, the compound of Formula (VIII) has formula:

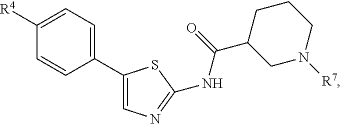

or a pharmaceutically acceptable salt thereof. In some embodiments, R⁴ is selected from halo, C₁₋₃ alkyl, and C₁₋₃ alkoxy.

In some embodiments, the compound of Formula (VIII) has formula:

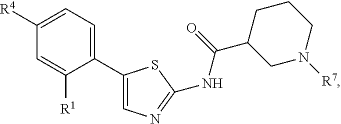

or a pharmaceutically acceptable salt thereof. In some embodiments, R¹ and R⁴ are each independently selected from halo, OR^{a1}, NR^{c1}R^{d1} and C₁₋₃ alkyl. In some embodiments, R¹ and R⁴ are each independently selected from halo and C₁₋₃ alkoxy.

In some embodiments, the compound of Formula (VIII) has formula:

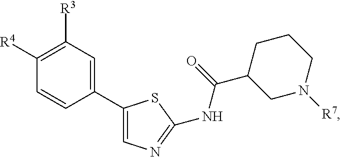

or a pharmaceutically acceptable salt thereof. In some embodiments, R³ and R⁴ are each independently selected from halo, OR^{a1}, NR^{c1}R^{d1} and C₁₋₃ alkyl. In some embodiments, R³ and R⁴ are each independently selected from halo and C₁₋₃ alkoxy.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹.

In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is selected from Cy¹, C(═O)Cy¹, and C₁₋₆ alkyl substituted with Cy¹. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (VIII) is selected from any one of the following compounds:

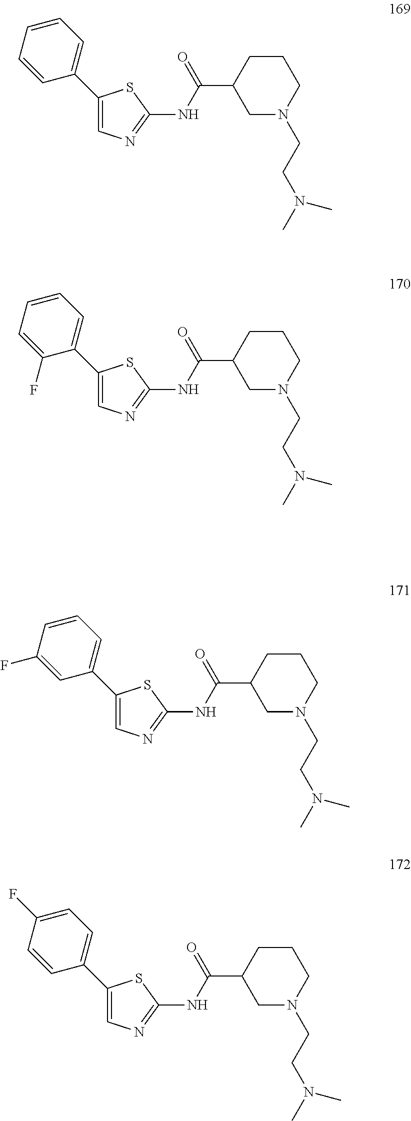 [SMILES: CN(C)CCN1CCCC(C(=O)Nc2ncc(-c3ccccc3)s2)C1 ; CN(C)CCN1CCCC(C(=O)Nc2ncc(-c3ccccc3F)s2)C1 ; CN(C)CCN1CCCC(C(=O)Nc2ncc(-c3cccc(F)c3)s2)C1 ; CN(C)CCN1CCCC(C(=O)Nc2ncc(-c3ccc(F)cc3)s2)C1]

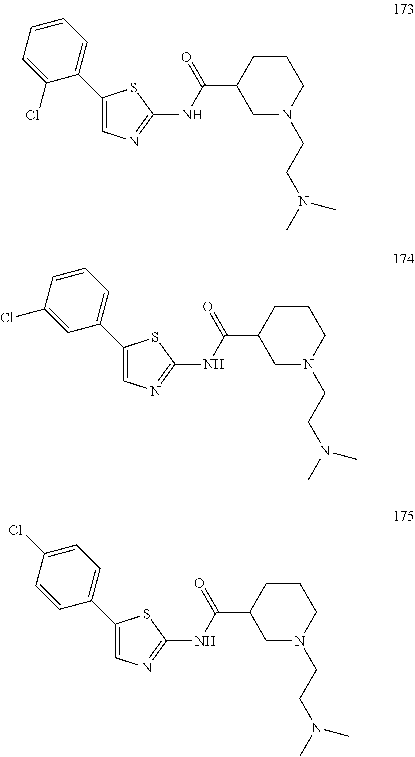 [SMILES: CN(C)CCN1CCCC(C(=O)Nc2ncc(-c3ccccc3Cl)s2)C1 ; CN(C)CCN1CCCC(C(=O)Nc2ncc(-c3cccc(Cl)c3)s2)C1 ; CN(C)CCN1CCCC(C(=O)Nc2ncc(-c3ccc(Cl)cc3)s2)C1]

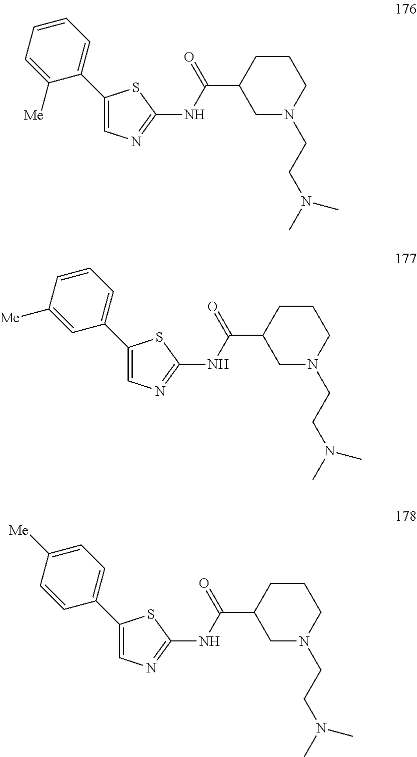 [SMILES: Cc1ccccc1-c1cnc(NC(=O)C2CCCN(CCN(C)C)C2)s1 ; Cc1cccc(-c2cnc(NC(=O)C3CCCN(CCN(C)C)C3)s2)c1 ; Cc1ccc(-c2cnc(NC(=O)C3CCCN(CCN(C)C)C3)s2)cc1]

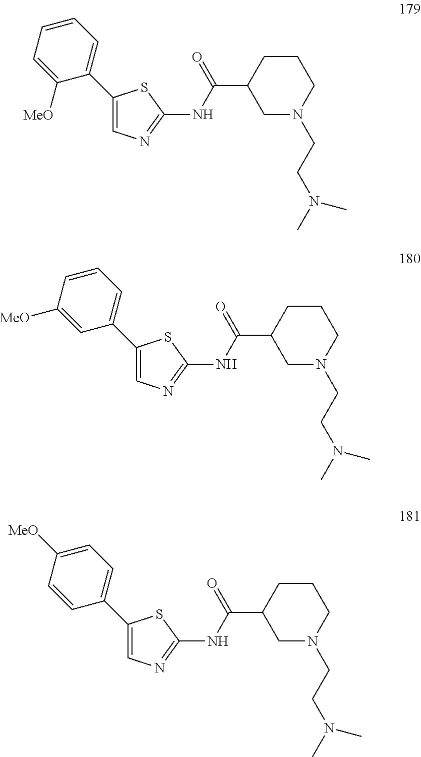 [SMILES: COc1ccccc1-c1cnc(NC(=O)C2CCCN(CCN(C)C)C2)s1 ; COc1cccc(-c2cnc(NC(=O)C3CCCN(CCN(C)C)C3)s2)c1 ; COc1ccc(-c2cnc(NC(=O)C3CCCN(CCN(C)C)C3)s2)cc1]

or a pharmaceutically acceptable salt thereof.

### Compounds of Formula (IX)

In some embodiments, the present disclosure provides a compound of Formula (IX):

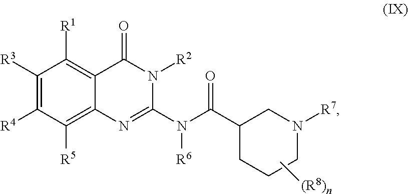

or a pharmaceutically acceptable salt thereof, wherein:
R² is selected from H, C₁₋₃ alkyl, and C₁₋₃ haloalkyl;
R⁶ is selected from H, C₁₋₃ alkyl, and C₁₋₃ haloalkyl;
R¹, R³, R⁴, and R⁵ are each independently selected from H, halo, CN, NO₂, C(O)OR^{a1}, OR^{a1}, NR^{c1}R^{d1}, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, R³ is selected from H and halo, and R⁵ is halo. In some embodiments, R³ is H, F, Cl, or Br. In some embodiments, R³ is H. In some embodiments, R³ is halo. In some embodiments, R³ is F, Cl, or Br. In some embodiments, R⁵ is F, Cl, or Br. In some embodiments, R⁵ is F, Cl, or Br, and R³ is H, F or Cl. In some embodiments, R⁵ is F, Cl, or Br, and R³ is H or F. In some embodiments, R⁵ is F, and R³ is F. In some embodiments, R⁵ is F, and R³ is H. In some embodiments, R⁵ is Br, and R³ is H. In some embodiments, R⁵ is Cl, and R³ is H. In some embodiments, R⁵ is Cl, and R³ is Cl. In some embodiments, R⁵ is Cl, and R³ is F.

In some embodiments, R¹ and R⁴ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{c1} or Cy¹. In some embodiments, R¹ and R⁴ are each H. In some embodiments, R¹ and R⁴ are each independently selected from H, halo, CN, OH, C₁₋₃ alkyl, C₁₋₃ haloalkyl, C₁₋₃ alkoxy, C₁₋₃ haloalkoxy, OR^{a1}—C₁₋₃ alkylene, and NR^{c1}R^{d1}—C₁₋₃ alkyl. In some embodiments, R¹ and R⁴ are each independently selected from halo, CN, OH, C₁₋₃ alkyl, C₁₋₃ haloalkyl, C₁₋₃ alkoxy, C₁₋₃ haloalkoxy, OR^{a1}—C₁₋₃ alkylene, and NR^{c1}R^{d1}—C₁₋₃ alkyl. In some embodiments, R¹ and R⁴ are each independently selected from H, halo, CN, OH, C₁₋₃ alkyl, C₁₋₃ haloalkyl, C₁₋₃ alkoxy, and C₁₋₃ haloalkoxy. In some embodiments, R¹ and R⁴ are each independently selected from halo, CN, OH, C₁₋₃ alkyl, C₁₋₃ haloalkyl, C₁₋₃ alkoxy, and C₁₋₃ haloalkoxy.

In some embodiments, at least one of R¹ and R⁴ is H. In some embodiments, at least one of R¹ and R⁴ is other than H.

In some embodiments, the compound of Formula (IX) has formula:

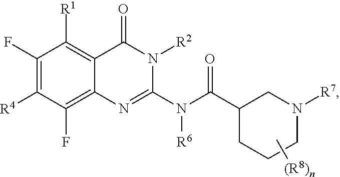

or a pharmaceutically acceptable salt thereof.

In some embodiments, the compound of Formula (IX) has formula:

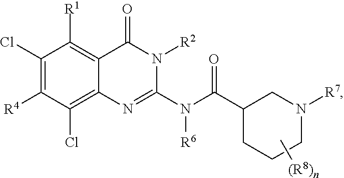

or a pharmaceutically acceptable salt thereof.

In some embodiments, the compound of Formula (IX) has formula:

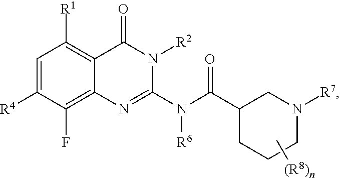

or a pharmaceutically acceptable salt thereof.

In some embodiments, the compound of Formula (IX) has formula:

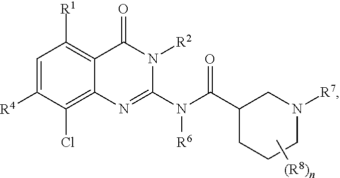

or a pharmaceutically acceptable salt thereof.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl. In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, n is 0, 1, or 2. In some embodiments, n is 0. In some embodiments, n is 1. In some embodiments, n is 2. In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, C₁₋₆ alkyl, C₁₋₆ haloalkyl, C₁₋₆ alkoxy, and C₁₋₆ haloalkoxy.

In some embodiments, the compound of Formula (IX) selected from any one of the following formulae:

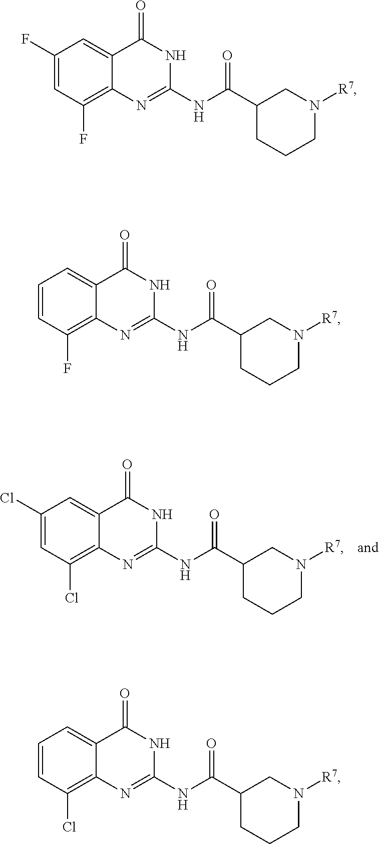

or a pharmaceutically acceptable salt thereof.

In some embodiments, the compound of Formula (IX) has formula:

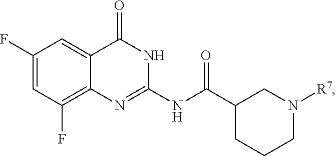

or a pharmaceutically acceptable salt thereof.

In some embodiments, the compound of Formula (IX) has formula:

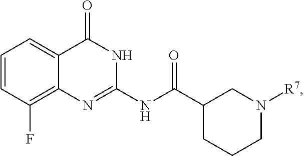

or a pharmaceutically acceptable salt thereof.

In some embodiments, the compound of Formula (IX) has formula:

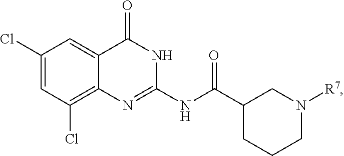

or a pharmaceutically acceptable salt thereof.

In some embodiments, the compound of Formula (IX) has formula:

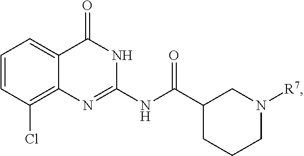

or a pharmaceutically acceptable salt thereof.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹.

In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is selected from Cy¹, C(═O)Cy¹, and C₁₋₆ alkyl substituted with Cy¹. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, N^{c2}R^{d2}, NR^{c2}C(O)R^{b2} N^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (IX) is selected from any one of the following compounds:

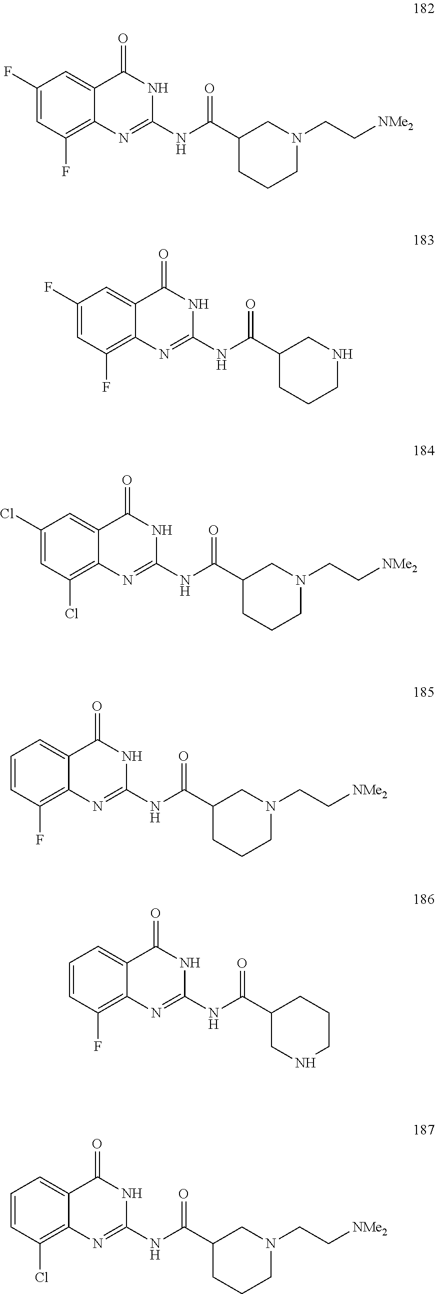 [SMILES: CN(C)CCN1CCCC(C(=O)Nc2nc3c(F)cc(F)cc3c(=O)[nH]2)C1 ; O=C(Nc1nc2c(F)cc(F)cc2c(=O)[nH]1)C1CCCNC1 ; CN(C)CCN1CCCC(C(=O)Nc2nc3c(Cl)cc(Cl)cc3c(=O)[nH]2)C1 ; CN(C)CCN1CCCC(C(=O)Nc2nc3c(F)cccc3c(=O)[nH]2)C1 ; O=C(Nc1nc2c(F)cccc2c(=O)[nH]1)C1CCCNC1 ; CN(C)CCN1CCCC(C(=O)Nc2nc3c(Cl)cccc3c(=O)[nH]2)C1]

or a pharmaceutically acceptable salt thereof.

### Compounds of Formula (X)

In some embodiments, the present disclosure provides a compound of Formula (X):

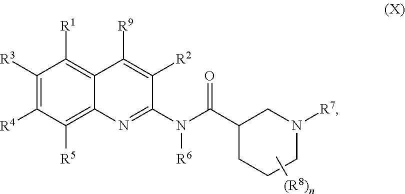

or a pharmaceutically acceptable salt thereof, wherein:
R¹, R², R³, R⁴, R⁵, and R⁹ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{c1} or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, R³ is selected from H and halo, R⁵ is halo. In some embodiments, R³ is selected from H and halo, and R⁵ is halo. In some embodiments, R³ is H, F, Cl, or Br. In some embodiments, R³ is H. In some embodiments, R³ is halo. In some embodiments, R³ is F, Cl, or Br. In some embodiments, R⁵ is F, Cl, or Br. In some embodiments, R⁵ is F, Cl, or Br, and R³ is H, F or Cl. In some embodiments, R⁵ is F, Cl, or Br, and R³ is H or F. In some embodiments, R⁵ is F, and R³ is F. In some embodiments, R⁵ is F, and R³ is H. In some embodiments, R⁵ is Br, and R³ is H. In some embodiments, R⁵ is Cl, and R³ is H. In some embodiments, R⁵ is Cl, and R³ is Cl. In some embodiments, R⁵ is Cl, and R³ is F.

In some embodiments, R¹, R², R⁴, and R⁹ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R¹, R², R⁴, and R⁹ are each H. In some embodiments, R¹, R², R⁴, and R⁹ are each independently selected from H, halo, CN, OH, C₁₋₃ alkyl, C₁₋₃ haloalkyl, C₁₋₃ alkoxy, C₁₋₃ haloalkoxy, OR^{a}i-C₁₋₃ alkylene, and NR^{c1}R^{d1}—C₁₋₃ alkyl. In some embodiments, R¹, R², R⁴, and R⁹ are each independently selected from halo, CN, OH, C₁₋₃ alkyl, C₁₋₃ haloalkyl, C₁₋₃ alkoxy, C₁₋₃ haloalkoxy, OR^{a}i-C₁₋₃ alkylene, and NR^{c1}R^{d1}—C₁₋₃ alkyl. In some embodiments, R¹, R², R⁴, and R⁹ are each independently selected from H, halo, CN, OH, C₁₋₃ alkyl, C₁₋₃ haloalkyl, C₁₋₃ alkoxy, and C₁₋₃ haloalkoxy. In some embodiments, R¹, R², R⁴, and R⁹ are each independently selected from halo, CN, OH, C₁₋₃ alkyl, C₁₋₃ haloalkyl, C₁₋₃ alkoxy, and C₁₋₃ haloalkoxy. In some embodiments, at least one of R¹, R², R⁴, and R⁹ is H. In some embodiments, at least one of R¹, R², R⁴, and R⁹ is other than H.

In some embodiments, R¹ is halo (e.g., F, Cl, or Br). In some embodiments, R¹ is C₁₋₃ alkyl. In some embodiments, R¹ is OR^{a1} (e.g., C₁₋₃ alkoxy or C₁₋₃ haloalkoxy). In some embodiments, R¹ is NR^{c1}R^{d1}. In some embodiments, R² is halo (e.g., F, Cl, or Br).

In some embodiments, R² is C₁₋₃ alkyl. In some embodiments, R² is OR^{a1} (e.g., C₁₋₃ alkoxy or C₁₋₃ haloalkoxy). In some embodiments, R² is NR^{b1}R^{d1}. In some embodiments, R⁴ is halo (e.g., F, Cl, or Br). In some embodiments, R⁴ is C₁₋₃ alkyl. In some embodiments, R⁴ is OR^{a1} (e.g., C₁₋₃ alkoxy or C₁₋₃ haloalkoxy). In some embodiments, R⁴ is NR^{c1}R^{d1}. In some embodiments, R⁹ is C₁₋₃ alkyl. In some embodiments, R⁹ is OR^{a1} (e.g., C₁₋₃ alkoxy or C₁₋₃ haloalkoxy). In some embodiments, R⁹ is NR^{b1}R^{d1}. In some embodiments, R¹ is H. In some embodiments, R² is H. In some embodiments, R⁴ is H. In some embodiments, R⁹ is H.

In some embodiments, the compound of Formula (X) has formula:

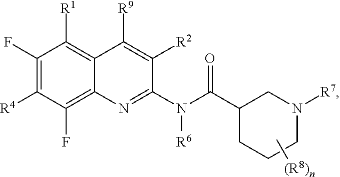

or a pharmaceutically acceptable salt thereof.

In some embodiments, the compound of Formula (X) has formula:

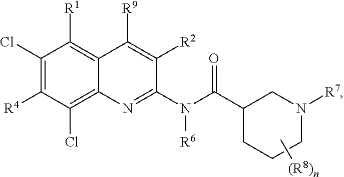

or a pharmaceutically acceptable salt thereof.

In some embodiments, the compound of Formula (X) has formula:

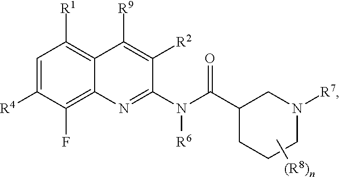

or a pharmaceutically acceptable salt thereof.

In some embodiments, the compound of Formula (X) has formula:

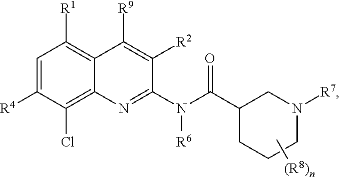

or a pharmaceutically acceptable salt thereof.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, n is 0, 1, or 2. In some embodiments, n is 0. In some embodiments, n is 1. In some embodiments, n is 2.

In some embodiments, R⁸ is selected from halo, CN, OH, NO₂, C₁₋₃ alkyl, C₁₋₃ haloalkyl, C₁₋₃ alkoxy, and C₁₋₃ haloalkoxy. In some embodiments, R⁸ is selected from halo, C₁₋₆ alkyl, C₁₋₆ haloalkyl, C₁₋₆ alkoxy, and C₁₋₆ haloalkoxy.

In some embodiments, the compound of Formula (X) is selected from any one of the following formulae:

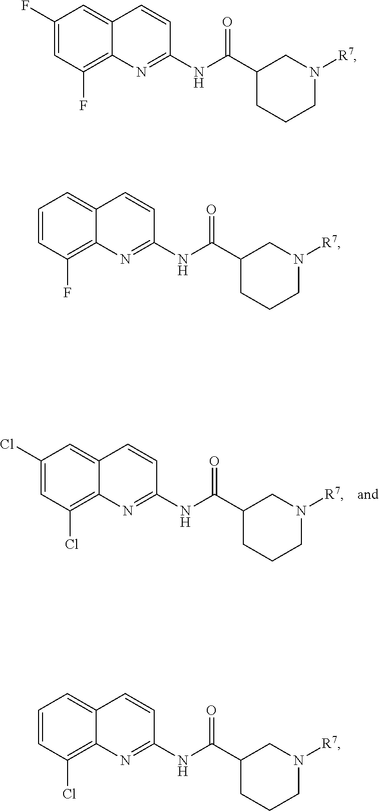

or a pharmaceutically acceptable salt thereof.

In some embodiments, the compound of Formula (X) has formula:

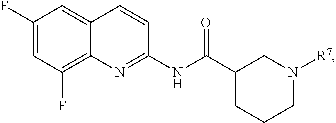

or a pharmaceutically acceptable salt thereof.

In some embodiments, the compound of Formula (X) has formula:

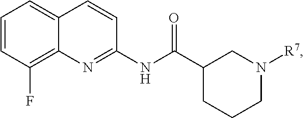

or a pharmaceutically acceptable salt thereof.

In some embodiments, the compound of Formula (X) has formula:

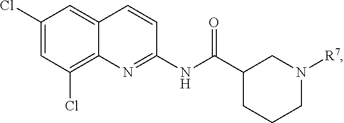

or a pharmaceutically acceptable salt thereof.

In some embodiments, the compound of Formula (X) has formula:

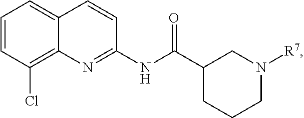

or a pharmaceutically acceptable salt thereof.

In some embodiments, R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}. In some embodiments, R⁷ is H. In some embodiments, R⁷ is Cy¹. In some embodiments, R⁷ is C(═O)Cy¹. In some embodiments, R⁷ is C(O)R^{b1}. In some embodiments, R⁷ is C(O)NR^{c1}R^{d1}. In some embodiments, R⁷ is C₁₋₆ alkyl substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹, OR^{a1}, C(O)NR^{c1}R^{d1}, C(O)OR^{a1}, NHC(═NH)NH₂, or NR^{c1}R^{d1}. In some embodiments, R⁷ is C₁₋₆ alkyl substituted with Cy¹.

In some embodiments, R⁷ is C₁₋₆ alkyl substituted with OR^{a1}. In some embodiments, R⁷ is C₁₋₆ alkyl substituted with C(O)NR^{c1}R^{d1}. In some embodiments, R⁷ is C₁₋₆ alkyl substituted with C(O)OR^{a1}. In some embodiments, R⁷ is C₁₋₆ alkyl substituted with NHC(═NH)NH₂. In some embodiments, R⁷ is selected from Cy¹, C(═O)Cy¹, and C₁₋₆ alkyl substituted with Cy¹. In some embodiments, R⁷ is C₁₋₆ alkyl substituted with NR^{c1}R^{d1}.

In some embodiments, R^{a1} is selected from H, C₁₋₃ alkyl, and C₁₋₃ haloalkyl. In some embodiments, R^{a1} is H or C₁₋₃ alkyl. In some embodiments, R^{a1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{a1} is Cy¹-C₁₋₃ alkylene. In some embodiments, R^{a1} is NR^{c2}R^{d2}—C₁₋₃ alkylene. In some embodiments, R^{a1} is C(O)OR^{a2}—C₁₋₃ alkylene. In some embodiments, R^{a1} is OR^{a2}—C₁₋₃ alkylene.

In some embodiments, R^{b1} is selected from C₁₋₃ alkyl and C₁₋₃ haloalkyl. In some embodiments, R^{b1} is C₁₋₃ alkyl. In some embodiments, R^{b1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{b1} is Cy¹-C₁₋₃ alkylene. In some embodiments, R^{b1} is NR^{c2}R^{d2}—C₁₋₃ alkylene. In some embodiments, R^{b1} is C(O)OR^{a2}—C₁₋₃ alkylene. In some embodiments, R^{b1} is OR^{a2}—C₁₋₃ alkylene.

In some embodiments, R^{c1} is selected from H, C₁₋₃ alkyl, and C₁₋₃ haloalkyl. In some embodiments, R^{c1} is H or C₁₋₃ alkyl. In some embodiments, R^{c1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{c1} is Cy¹-C₁₋₃ alkylene. In some embodiments, R^{c1} is NR^{c2}R^{d2}—C₁₋₃ alkylene. In some embodiments, R^{c1} is C(O)OR^{a2}—C₁₋₃ alkylene. In some embodiments, R^{c1} is OR^{a2}—C₁₋₃ alkylene.

In some embodiments, R^{d1} is selected from H, C₁₋₃ alkyl, and C₁₋₃ haloalkyl. In some embodiments, R^{d1} is H or C₁₋₃ alkyl. In some embodiments, R^{d1} is Cy¹-C₁₋₃ alkylene, NR^{c2}R^{d2}—C₁₋₃ alkylene, C(O)OR^{a2}—C₁₋₃ alkylene, or OR^{a2}—C₁₋₃ alkylene. In some embodiments, R^{d1} is Cy¹-C₁₋₃ alkylene. In some embodiments, R^{d1} is NR^{c2}R^{d2}—C₁₋₃ alkylene. In some embodiments, R^{d1} is C(O)OR^{a2}—C₁₋₃ alkylene. In some embodiments, R^{d1} is OR^{a2}—C₁₋₃ alkylene.

In some embodiments, Cy¹ is C₆₋₁₀ aryl, optionally substituted with 1, 2, or 3 substituents independently selected from R^{Cy1}. In some embodiments, Cy¹ is C₃₋₁₀ cycloalkyl, optionally substituted with 1, 2, or 3 substituents independently selected from R^{Cy1}. In some embodiments, Cy¹ is 5-14 membered heteroaryl, optionally substituted with 1, 2, or 3 substituents independently selected from R^{Cy1}. In some embodiments, Cy¹ is 4-10 membered heterocycloalkyl, optionally substituted with 1, 2, or 3 substituents independently selected from R^{Cy1}. In some embodiments, Cy¹ is C₆₋₁₀ aryl or C₃₋₁₀ cycloalkyl, each of which is optionally substituted with 1, 2, or 3 substituents independently selected from R^{Cy1}. In some embodiments, Cy¹ is C₆₋₁₀ aryl or 5-14 membered heteroaryl, each of which is optionally substituted with 1, 2, or 3 substituents independently selected from R^{Cy1}.

In some embodiments, R^{Cy1} is selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, S(O)₂R^{b2}, or S(O)₂NR^{c2}R^{d2}. In some embodiments, R^{Cy1} is selected from halo, C₁₋₆ alkyl, C₁₋₆ haloalkyl, C₂₋₆ alkenyl, C₂₋₆ alkynyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with CN, OR^{a2}, C(O)R^{b2}, C(O)NR^{c2}R^{d2}, C(O)OR^{a2}, NR^{c2}R^{d2}, or NR^{c2}C(O)R^{b2}. In some embodiments, R^{Cy1} is selected from halo, C₁₋₆ alkyl, C₂₋₆ alkenyl, OR^{a2}, C(O)R^{b2}, C(O)NR^{c2}R^{d2}, C(O)OR^{a2}, and NR^{c2}R^{d2}; wherein said C₁₋₆ alkyl is optionally substituted with OR^{a2}, C(O)R^{b2}, C(O)NR^{c2}R^{d2}, or C(O)OR^{a2}.

In some embodiments, each R^{a2}, R^{c2}, and R^{d2} is independently selected from H and C₁₋₆ alkyl. In some embodiments, R^{b2} is C₁₋₆ alkyl.

In some embodiments, the compound of Formula (X) is selected from any one of the following compounds:

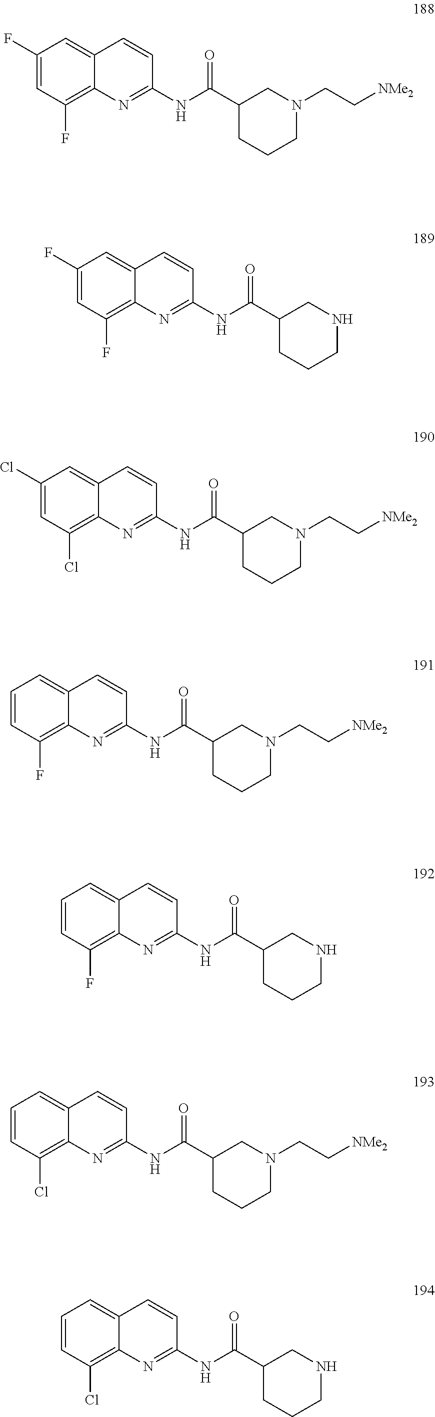

or a pharmaceutically acceptable salt thereof.

### Compounds of Formula (XI)

In some embodiments, the present disclosure provides a compound of Formula (XI):

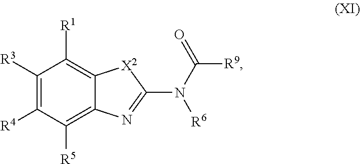

or a pharmaceutically acceptable salt thereof, wherein:
R⁹ is selected from any one of the following groups:

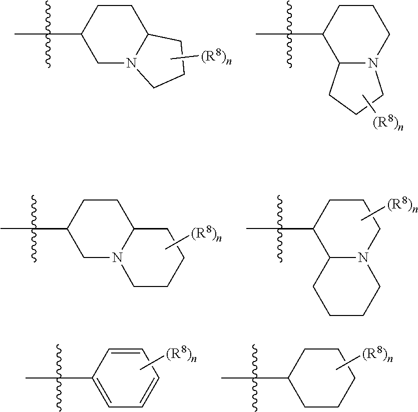

X² is selected from S, O, and NR²
R² is selected from H, C₁₋₃ alkyl, and C₁₋₃ haloalkyl;
R³ is halo;
R⁵ is halo;
R¹ and R⁴ are each independently selected from H, halo, CN, NO₂, C(O)OR^{a1}, OR^{a1}, NR^{c1}R^{d1}, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2} S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.

In some embodiments, R³ is F, Cl, or Br. In some embodiments, R⁵ is F, Cl, or Br.

In some embodiments, R⁵ is F or Cl, and R³ is F or Cl. In some embodiments, R⁵ is F, and R³ is F. In some embodiments, R⁵ is Cl, and R³ is Cl.

In some embodiments, R¹ and R⁴ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹. In some embodiments, R¹ and R⁴ are each H. In some embodiments, R¹ and R⁴ are each independently selected from H, halo, CN, OH, C₁₋₃ alkyl, C₁₋₃ haloalkyl, C₁₋₃ alkoxy, C₁₋₃ haloalkoxy, OR^{a}i-C₁₋₃ alkylene, and NR^{c1}R^{d1}—C₁₋₃ alkyl. In some embodiments, R¹ and R⁴ are each independently selected from halo, CN, OH, C₁₋₃ alkyl, C₁₋₃ haloalkyl, C₁₋₃ alkoxy, C₁₋₃ haloalkoxy, OR^{a}i-C₁₋₃ alkylene, and NR^{c1}R^{d1}—C₁₋₃ alkyl. In some embodiments, R¹ and R⁴ are each independently selected from H, halo, CN, OH, C₁₋₃ alkyl, C₁₋₃ haloalkyl, C₁₋₃ alkoxy, and C₁₋₃ haloalkoxy. In some embodiments, R¹ and R⁴ are each independently selected from halo, CN, OH, C₁₋₃ alkyl, C₁₋₃ haloalkyl, C₁₋₃ alkoxy, and C₁₋₃ haloalkoxy. In some embodiments, at least one of R¹ and R⁴ is H. In some embodiments, at least one of R¹ and R⁴ is other than H.

In some embodiments, R⁶ is H. In some embodiments, R⁶ is H or C₁₋₃ alkyl. In some embodiments, R⁶ is C₁₋₃ alkyl. In some embodiments, R⁶ is C₁₋₃ haloalkyl.

In some embodiments, X² is selected from S or O. In some embodiments, X² is S. In some embodiments, X² is O. In some embodiments, X² is NR². In some embodiments, R² is H. In some embodiments, R² is H or C₁₋₃ alkyl. In some embodiments, R² is C₁₋₃ alkyl. In some embodiments, R² is C₁₋₃ haloalkyl.

In some embodiments, the compound of Formula (XI) has formula:

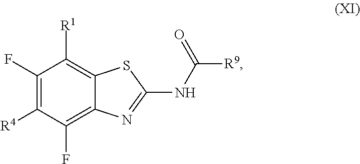

or a pharmaceutically acceptable salt thereof.

In some embodiments, R⁹ is:

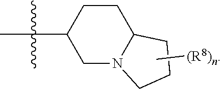

In some embodiments, R⁹ is:

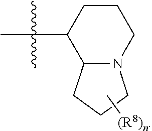

In some embodiments, R⁹ is:

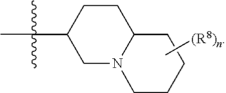

In some embodiments, R⁹ is:

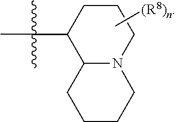

In some embodiments, R⁹ is:

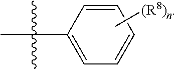

In some embodiments, R⁹ is:

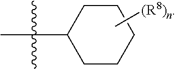

In some embodiments, R⁸ is selected from halo, OR^{a1}, NR^{c1}R^{d1}, C₁₋₃ alkyl, C₁₋₃ haloalkyl, and Cy¹, wherein said C₁₋₃ alkyl is optionally substituted with OR^{a1}, NR^{c1}R^{d1} or Cy¹. In some embodiments, R⁸ is selected from halo, OR^{a1}, NR^{c1}R^{d1} C₁₋₃ alkyl, and Cy¹, wherein said C₁₋₃ alkyl is optionally substituted with NR^{c1}R^{d1}.

In some embodiments, R⁸ is halo. In some embodiments, R⁸ is OR^{a1}. In some embodiments, R⁸ is NR^{c1}R^{d1}. In some embodiments, R⁸ is C₁₋₃ alkyl. In some embodiments, R⁸ is C₁₋₃ haloalkyl. In some embodiments, R⁸ is Cy¹. In some embodiments, R⁸ is C₁₋₃ alkyl substituted with OR^{a1}. In some embodiments, R⁸ is C₁₋₃ alkyl substituted with NR^{c1}R^{d1}. In some embodiments, R⁸ is C₁₋₃ alkyl substituted with Cy¹.

In some embodiments, n is 0, 1, or 2. In some embodiments, n is 0. In some embodiments, n is 1. In some embodiments, n is 2.

In some embodiments, the compound of Formula (XI) has formula:

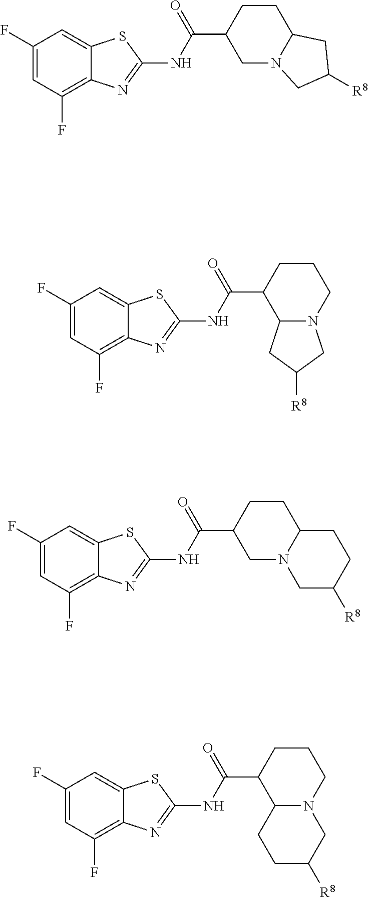

or a pharmaceutically acceptable salt thereof.

In some embodiments, R⁸ is selected from NR^{c1}R^{d1} and NR^{c1}R^{d1}—C₁₋₃ alkylene;
R^{c1} and R^{d1} is independently selected from H, C₁₋₃ alkyl, and NR^{c2}R^{d2}—C₁₋₃ alkylene; and
R^{c2} and R^{d2} are each independently selected from H and C₁₋₆ alkyl.

In some embodiments, the compound of Formula (XI) has formula

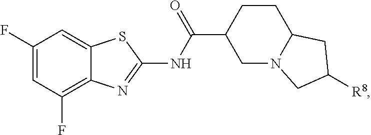

or a pharmaceutically acceptable salt thereof.

In some embodiments, the compound of Formula (XI) has formula

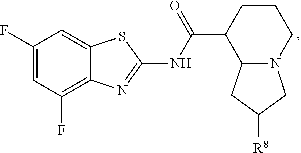

or a pharmaceutically acceptable salt thereof.

In some embodiments, the compound of Formula (XI) has formula

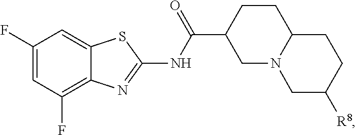

or a pharmaceutically acceptable salt thereof.

In some embodiments, the compound of Formula (XI) has formula

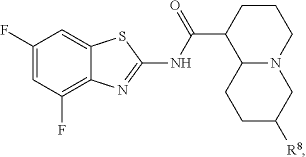

or a pharmaceutically acceptable salt thereof.

In some embodiments, the compound of Formula (XI) has any one of the following formulae:

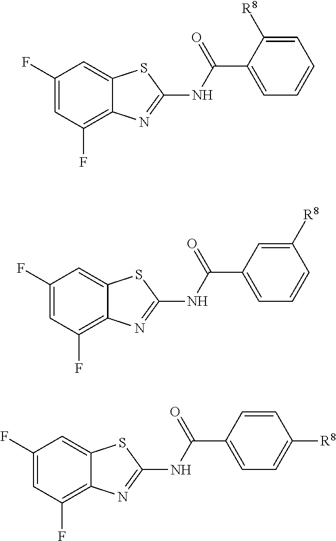

or a pharmaceutically acceptable salt thereof.

In some embodiments, each R⁸ is independently selected from halo, OR^{a1}, and Cy¹. In some embodiments, each R⁸ is independently selected from halo, C₁₋₃ alkoxy, and phenyl.

In some embodiments, the compound of Formula (XI) has formula

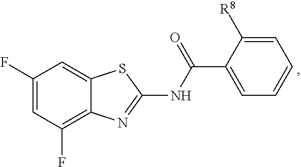

or a pharmaceutically acceptable salt thereof.

In some embodiments, the compound of Formula (XI) has formula

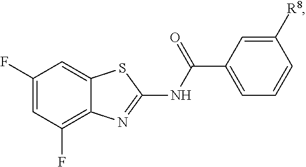

or a pharmaceutically acceptable salt thereof.

In some embodiments, the compound of Formula (XI) has formula

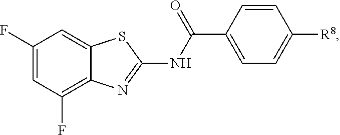

or a pharmaceutically acceptable salt thereof.

In some embodiments, the compound of Formula (XI) has any one of the following formulae:

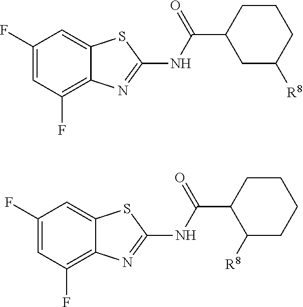

or a pharmaceutically acceptable salt thereof.

In some embodiments, R⁸ is selected from NR^{c1}R^{d1} and NR^{c1}R^{d1}—C₁₋₃ alkylene; R^{c1} and R^{d1} is independently selected from H, C₁₋₃ alkyl, and NR^{c2}R^{d2}—C₁₋₃ alkylene; and R^{c2} and R^{d2} are each independently selected from H and C₁₋₆ alkyl. In some embodiments, R⁸ is NR^{c1}R^{d1}.

In some embodiments, the compound of Formula (XI) has formula

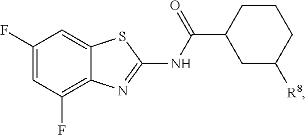

or a pharmaceutically acceptable salt thereof.

In some embodiments, the compound of Formula (XI) has formula

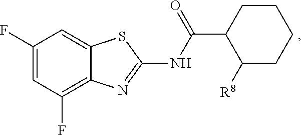

or a pharmaceutically acceptable salt thereof.

In some embodiments, the compound of Formula (XI) is selected from any one of the following compounds:

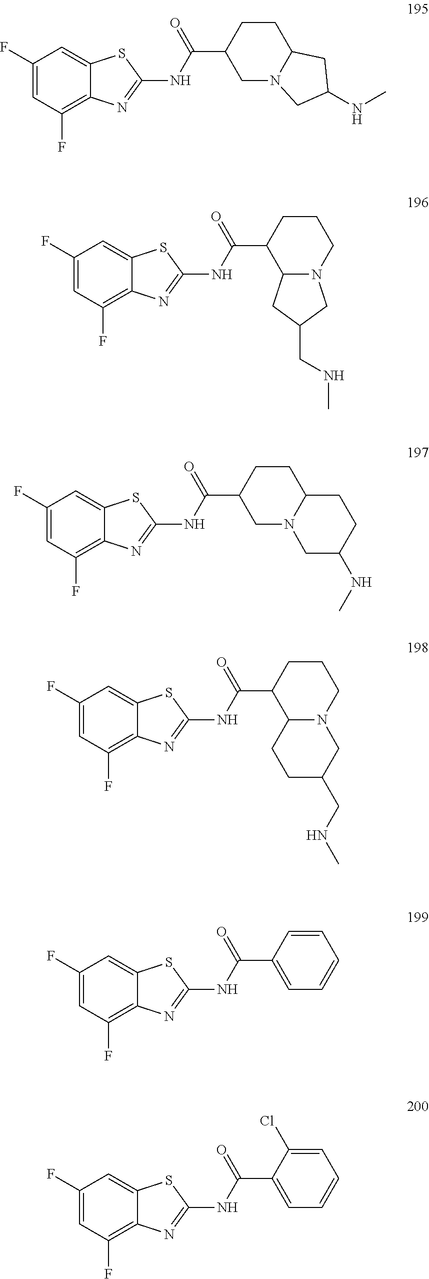 [SMILES: CNC1CC2CCC(C(=O)Nc3nc4c(F)cc(F)cc4s3)CN2C1 ; CNCC1CC2C(C(=O)Nc3nc4c(F)cc(F)cc4s3)CCCN2C1 ; CNC1CCC2CCC(C(=O)Nc3nc4c(F)cc(F)cc4s3)CN2C1 ; CNCC1CCC2C(C(=O)Nc3nc4c(F)cc(F)cc4s3)CCCN2C1 ; O=C(Nc1nc2c(F)cc(F)cc2s1)c1ccccc1 ; O=C(Nc1nc2c(F)cc(F)cc2s1)c1ccccc1Cl]

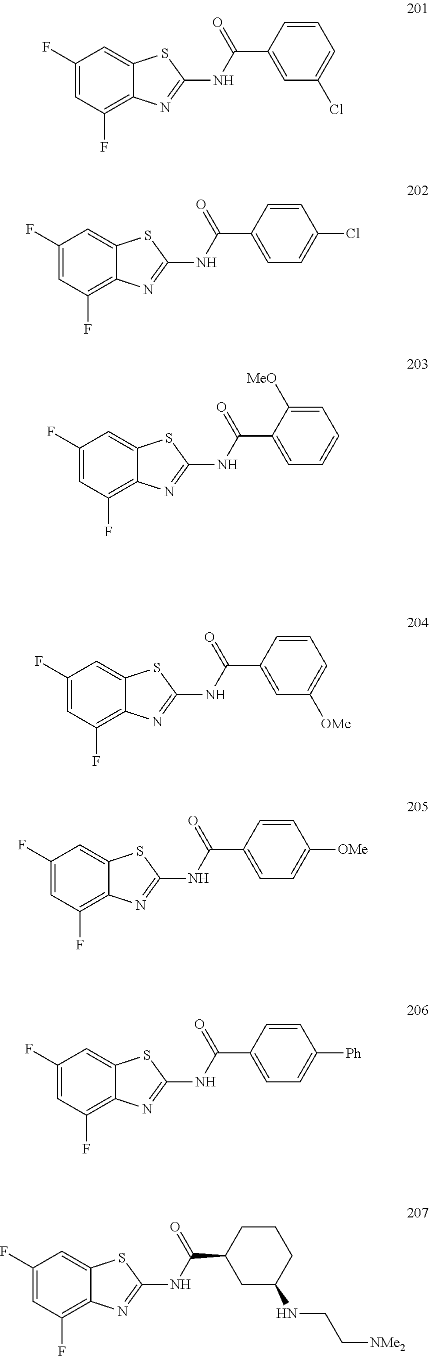

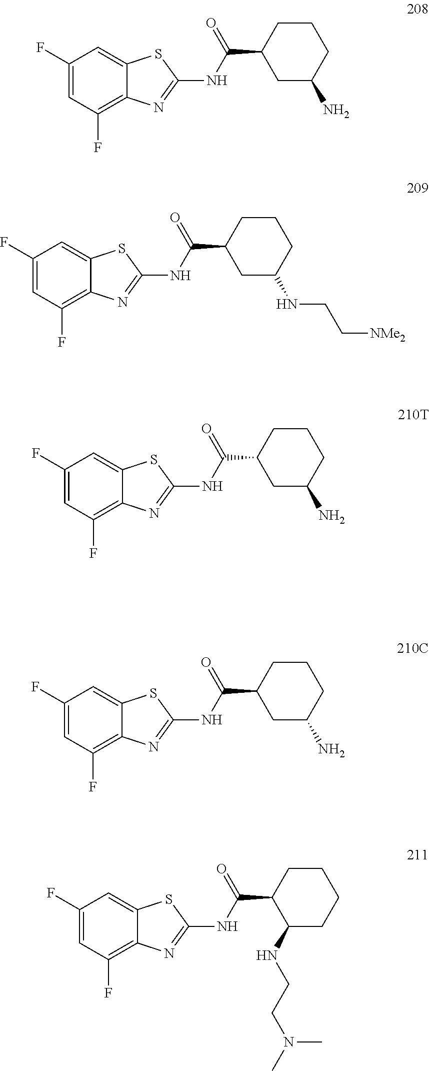

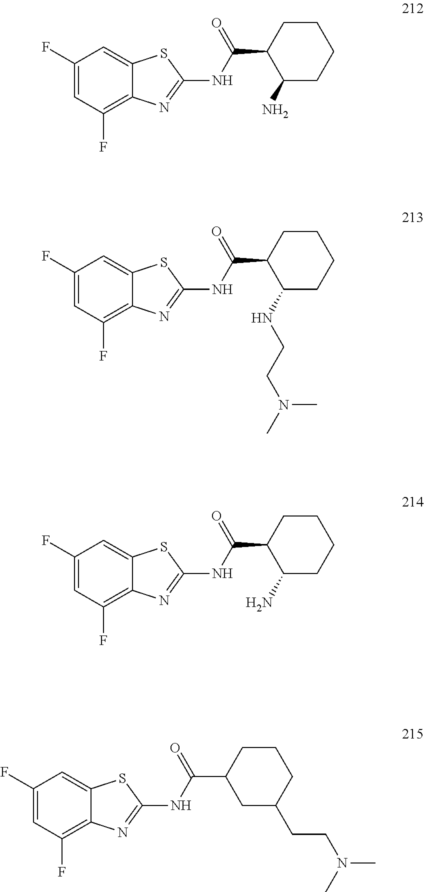 [SMILES: N[C@@H]1CCCC[C@@H]1C(=O)Nc1nc2c(F)cc(F)cc2s1 ; CN(C)CCN[C@H]1CCCC[C@@H]1C(=O)Nc1nc2c(F)cc(F)cc2s1 ; N[C@H]1CCCC[C@@H]1C(=O)Nc1nc2c(F)cc(F)cc2s1 ; CN(C)CCC1CCCC(C(=O)Nc2nc3c(F)cc(F)cc3s2)C1]

or a pharmaceutically acceptable salt thereof.

In some embodiments, the present disclosure provides a compound selected from any one of the following compounds

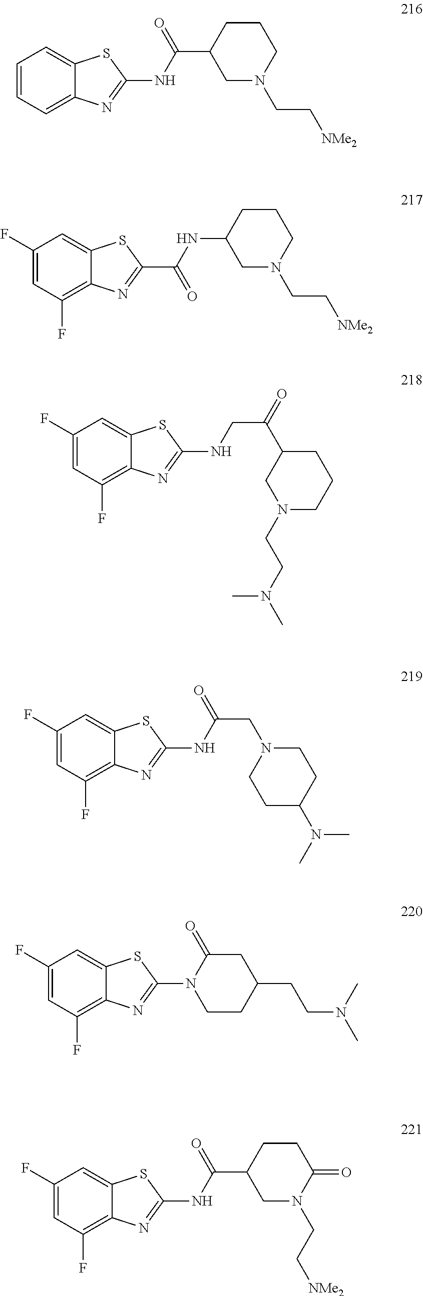 [SMILES: CN(C)CCN1CCCC(C(=O)Nc2nc3ccccc3s2)C1 ; CN(C)CCN1CCCC(NC(=O)c2nc3c(F)cc(F)cc3s2)C1 ; CN(C)CCN1CCCC(C(=O)CNc2nc3c(F)cc(F)cc3s2)C1 ; CN(C)C1CCN(CC(=O)Nc2nc3c(F)cc(F)cc3s2)CC1 ; CN(C)CCC1CCN(c2nc3c(F)cc(F)cc3s2)C(=O)C1 ; CN(C)CCN1CC(C(=O)Nc2nc3c(F)cc(F)cc3s2)CCC1=O]

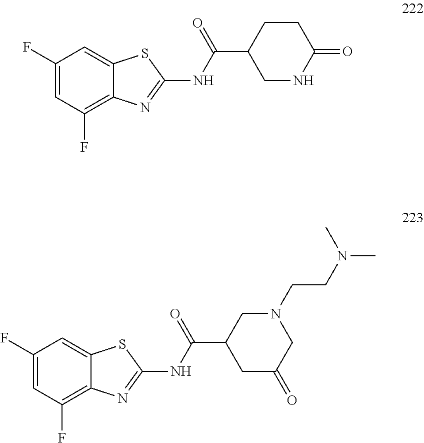 [SMILES: O=C1CCC(C(=O)Nc2nc3c(F)cc(F)cc3s2)CN1 ; CN(C)CCN1CC(=O)CC(C(=O)Nc2nc3c(F)cc(F)cc3s2)C1]

or a pharmaceutically acceptable salt thereof.

In some embodiments, the present disclosure provides a compound selected from any one of the following compounds

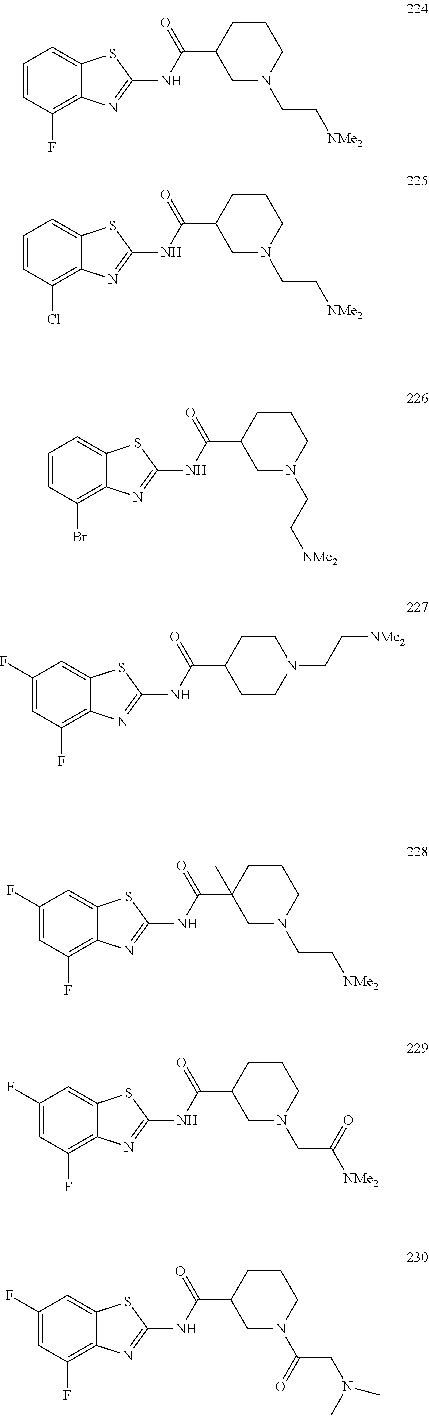

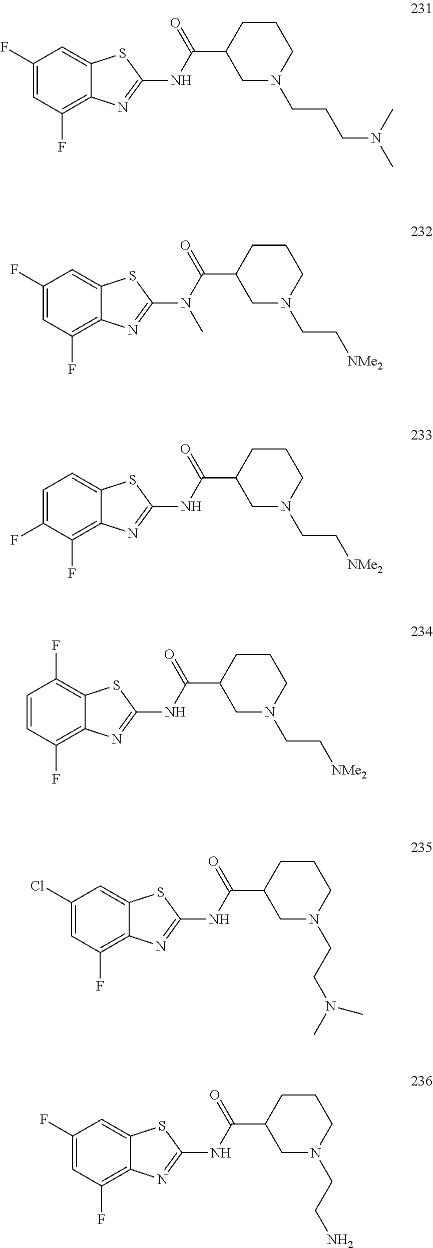 [SMILES: CN(C)CCCN1CCCC(C(=O)Nc2nc3c(F)cc(F)cc3s2)C1 ; CN(C)CCN1CCCC(C(=O)N(C)c2nc3c(F)cc(F)cc3s2)C1 ; CN(C)CCN1CCCC(C(=O)Nc2nc3c(F)c(F)ccc3s2)C1 ; CN(C)CCN1CCCC(C(=O)Nc2nc3c(F)ccc(F)c3s2)C1 ; CN(C)CCN1CCCC(C(=O)Nc2nc3c(F)cc(Cl)cc3s2)C1 ; NCCN1CCCC(C(=O)Nc2nc3c(F)cc(F)cc3s2)C1]

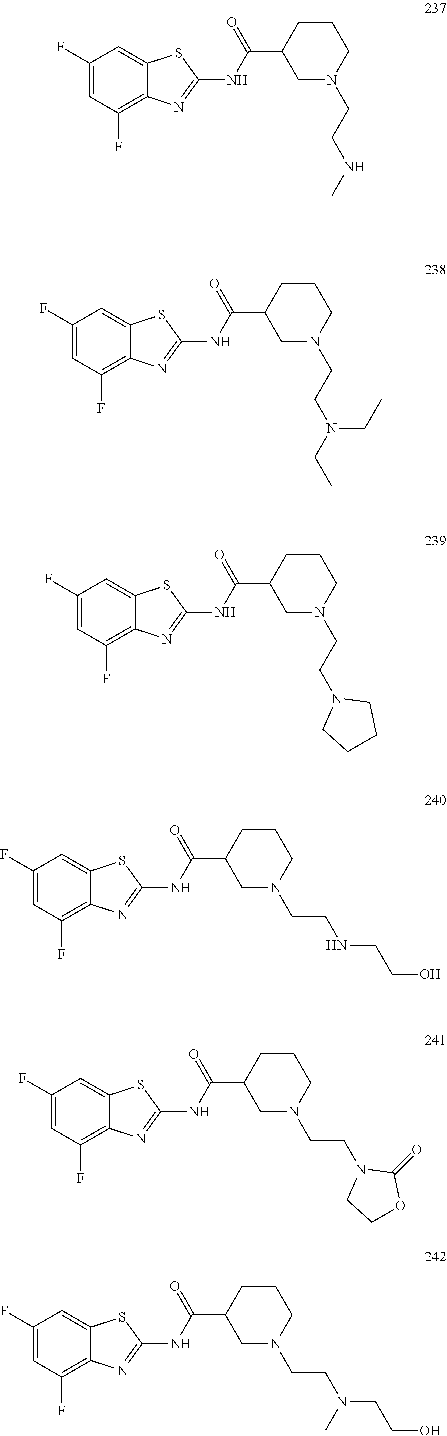 [SMILES: CNCCN1CCCC(C(=O)Nc2nc3c(F)cc(F)cc3s2)C1 ; CCN(CC)CCN1CCCC(C(=O)Nc2nc3c(F)cc(F)cc3s2)C1 ; O=C(Nc1nc2c(F)cc(F)cc2s1)C1CCCN(CCN2CCCC2)C1 ; O=C(Nc1nc2c(F)cc(F)cc2s1)C1CCCN(CCNCCO)C1 ; O=C(Nc1nc2c(F)cc(F)cc2s1)C1CCCN(CCN2CCOC2=O)C1 ; CN(CCO)CCN1CCCC(C(=O)Nc2nc3c(F)cc(F)cc3s2)C1]

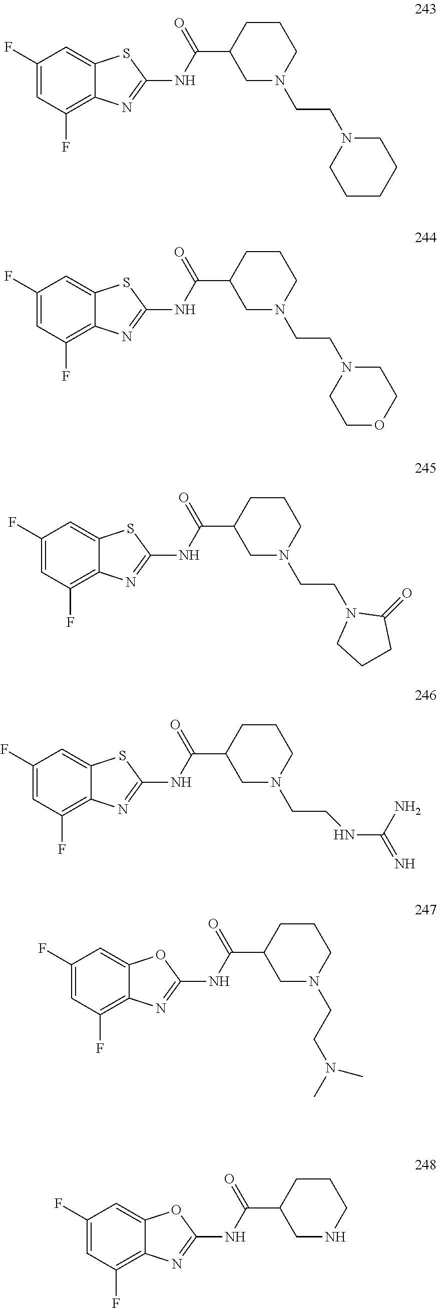 [SMILES: O=C(Nc1nc2c(F)cc(F)cc2s1)C1CCCN(CCN2CCCCC2)C1 ; O=C(Nc1nc2c(F)cc(F)cc2s1)C1CCCN(CCN2CCOCC2)C1 ; O=C(Nc1nc2c(F)cc(F)cc2s1)C1CCCN(CCN2CCCC2=O)C1 ; N=C(N)NCCN1CCCC(C(=O)Nc2nc3c(F)cc(F)cc3s2)C1 ; CN(C)CCN1CCCC(C(=O)Nc2nc3c(F)cc(F)cc3o2)C1 ; O=C(Nc1nc2c(F)cc(F)cc2o1)C1CCCNC1]

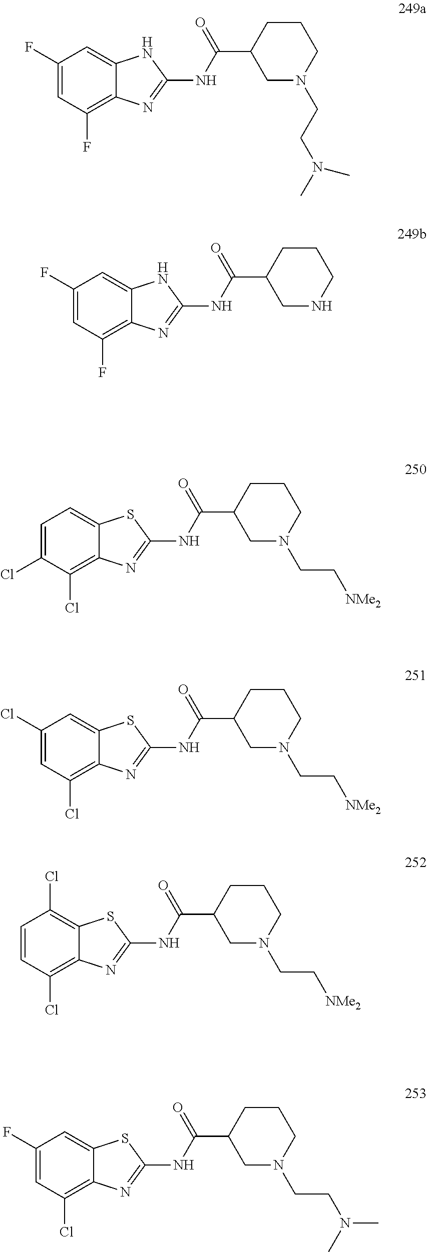 [SMILES: CN(C)CCN1CCCC(C(=O)Nc2nc3c(F)cc(F)cc3[nH]2)C1 ; O=C(Nc1nc2c(F)cc(F)cc2[nH]1)C1CCCNC1 ; CN(C)CCN1CCCC(C(=O)Nc2nc3c(Cl)c(Cl)ccc3s2)C1 ; CN(C)CCN1CCCC(C(=O)Nc2nc3c(Cl)cc(Cl)cc3s2)C1 ; CN(C)CCN1CCCC(C(=O)Nc2nc3c(Cl)ccc(Cl)c3s2)C1 ; CN(C)CCN1CCCC(C(=O)Nc2nc3c(Cl)cc(F)cc3s2)C1]

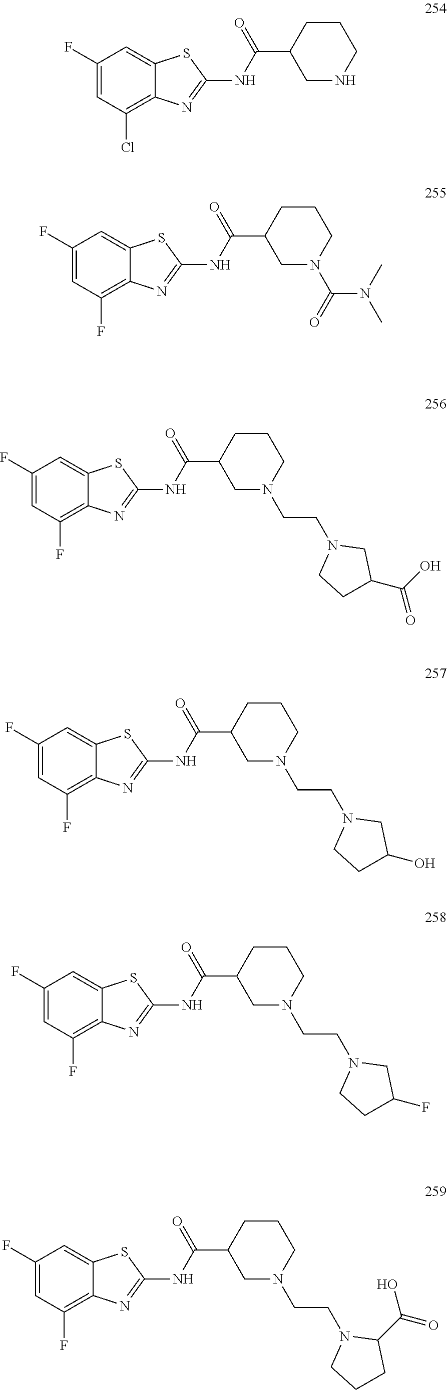 [SMILES: O=C(Nc1nc2c(Cl)cc(F)cc2s1)C1CCCNC1 ; CN(C)C(=O)N1CCCC(C(=O)Nc2nc3c(F)cc(F)cc3s2)C1 ; O=C(O)C1CCN(CCN2CCCC(C(=O)Nc3nc4c(F)cc(F)cc4s3)C2)C1 ; O=C(Nc1nc2c(F)cc(F)cc2s1)C1CCCN(CCN2CCC(O)C2)C1 ; O=C(Nc1nc2c(F)cc(F)cc2s1)C1CCCN(CCN2CCC(F)C2)C1 ; O=C(Nc1nc2c(F)cc(F)cc2s1)C1CCCN(CCN2CCCC2C(=O)O)C1]

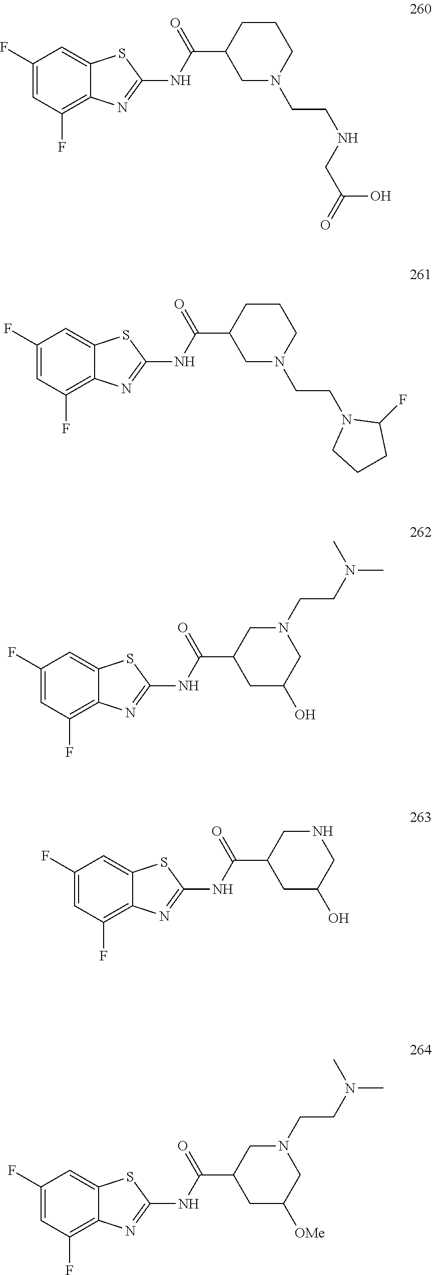 [SMILES: O=C(O)CNCCN1CCCC(C(=O)Nc2nc3c(F)cc(F)cc3s2)C1 ; O=C(Nc1nc2c(F)cc(F)cc2s1)C1CCCN(CCN2CCCC2F)C1 ; CN(C)CCN1CC(O)CC(C(=O)Nc2nc3c(F)cc(F)cc3s2)C1 ; O=C(Nc1nc2c(F)cc(F)cc2s1)C1CNCC(O)C1 ; COC1CC(C(=O)Nc2nc3c(F)cc(F)cc3s2)CN(CCN(C)C)C1]

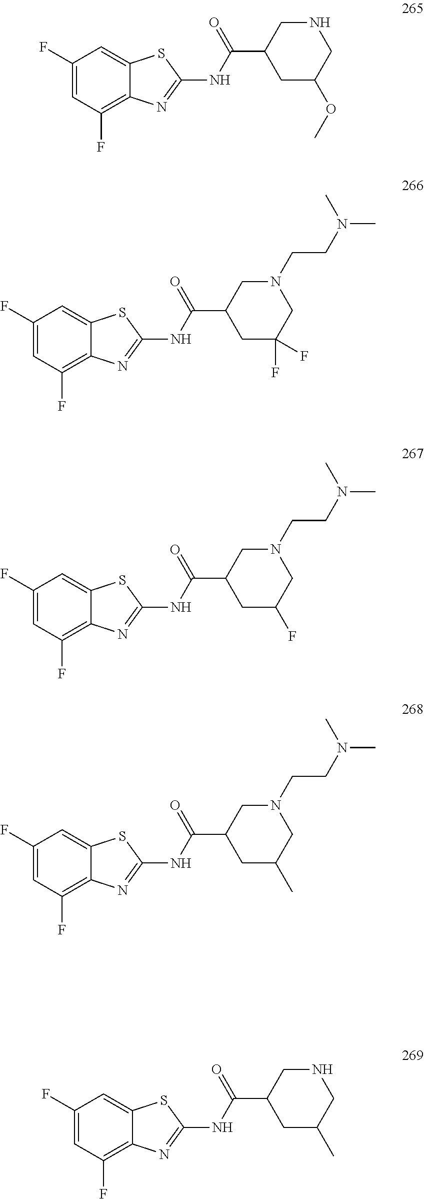 [SMILES: COC1CNCC(C(=O)Nc2nc3c(F)cc(F)cc3s2)C1 ; CN(C)CCN1CC(C(=O)Nc2nc3c(F)cc(F)cc3s2)CC(F)(F)C1 ; CN(C)CCN1CC(F)CC(C(=O)Nc2nc3c(F)cc(F)cc3s2)C1 ; CC1CC(C(=O)Nc2nc3c(F)cc(F)cc3s2)CN(CCN(C)C)C1 ; CC1CNCC(C(=O)Nc2nc3c(F)cc(F)cc3s2)C1]

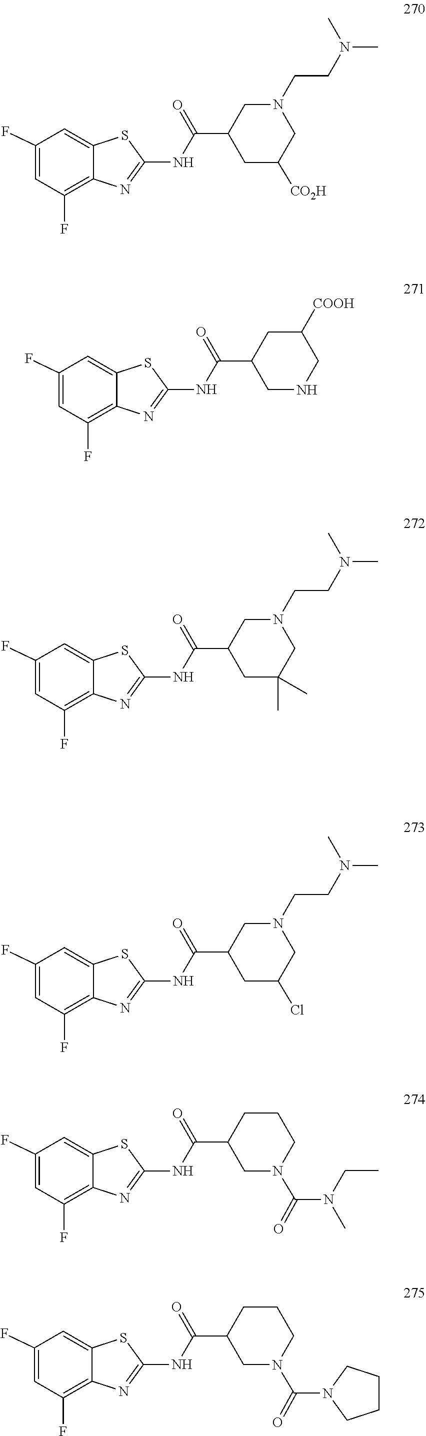 [SMILES: CN(C)CCN1CC(C(=O)O)CC(C(=O)Nc2nc3c(F)cc(F)cc3s2)C1 ; O=C(O)C1CNCC(C(=O)Nc2nc3c(F)cc(F)cc3s2)C1 ; CN(C)CCN1CC(C(=O)Nc2nc3c(F)cc(F)cc3s2)CC(C)(C)C1 ; CN(C)CCN1CC(Cl)CC(C(=O)Nc2nc3c(F)cc(F)cc3s2)C1 ; CCN(C)C(=O)N1CCCC(C(=O)Nc2nc3c(F)cc(F)cc3s2)C1 ; O=C(Nc1nc2c(F)cc(F)cc2s1)C1CCCN(C(=O)N2CCCC2)C1]

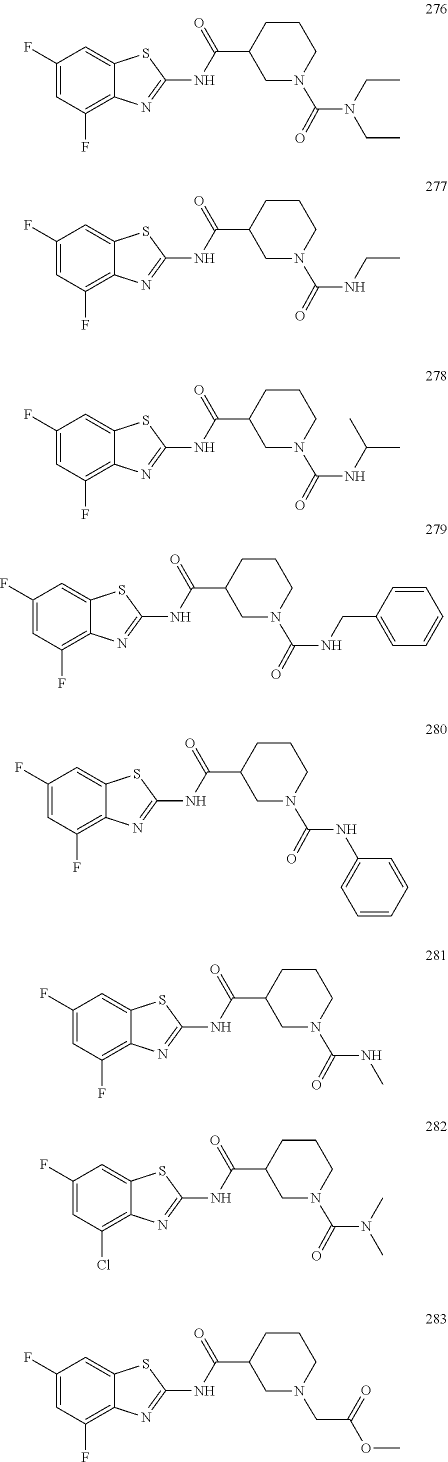

or a pharmaceutically acceptable salt thereof.

In some embodiments provided herein is a compound of Formula (XII):

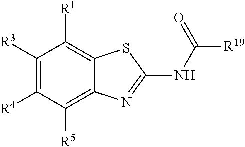

or a pharmaceutically acceptable salt thereof, wherein:
R¹=F, H, Cl, C₁-C₆ alkyl, OC₁-C₆ alkyl;
R⁴=H, C₁;
R³=F, H, C₁-C₆ alkyl, OC₁-C₆ alkyl;
R⁵=F, H, Cl, Br, CN;
R¹⁹ is

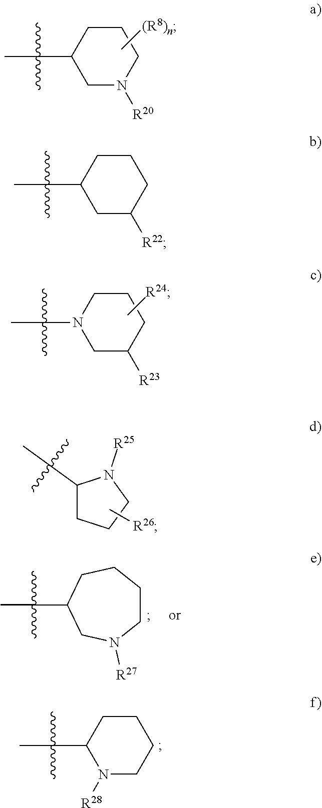

wherein n is 0, 1 or 2, wherein
when n is 1, R⁸ is OH;
and when n is 2,
each R⁸ is F, or
two R⁸ taken together with a ring carbon form C═O, or
two R⁸ taken together with the atoms connecting them form a 5-membered or 6-membered heterocycloalkane ring or a fused benzene ring;
R²⁰ is selected from
a) CON(C₁-C₆ alkyl)₂; b) CO-(4-, 5-, or 6-membered)-heterocycloalkyl optionally substituted at a ring carbon with C₁-C₆ alkyl; c) i) 5- or 6-membered heteroaryl optionally substituted with C₁-C₆ alkyl, NH₂, OC₁-C₆ alkyl, or halogen, or ii) phenyl substituted by N(C₁-C₆ alkyl)₂; d) C₁₋₄ alkyl substituted with R³⁰,
   wherein R³⁰ is COOC₁-C₆ alkyl, C(O)N(C₁-C₆ alkyl)₂, C(O)N(H)C₁-C₆ alkyl, C(O)CH₂N(C₁-C₆ alkyl)₂, N(C₁-C₆ alkyl)₂ wherein one alkyl is optionally substituted by OH, N(H)C₁-C₆ alkyl optionally substituted by OH, NH₂ optionally substituted by OH, 4-, 5-, or 6-membered heterocycloalkyl containing N and/or O and optionally substituted by ═O or by C₁-C₆ alkyl or by COOH, or 5- or 6-membered heteroaryl; provided that if R²⁰ is C₂ alkyl and R³⁰ is N(C₁-C₆ alkyl)₂, then R³ and R⁵ are not both F;
e) 5-membered cycloalkyl; and
f) 4-, 5-, or 6-membered heterocycloalkyl having NR²¹ and/or O and optionally substituted at a ring carbon with R³¹, wherein R³¹ is C₁-C₆ alkyl, ═O, OH, or OC₁-C₆ alkyl,
   wherein R²¹ is a) H; b) C₂-C₆ alkenyl; c) C₁-C₄ alkyl optionally substituted with R³², wherein R³² is halo, C₁-C₆ alkoxy, COOC₁-C₆ alkoxy, or CO-(4-, 5-, or 6-membered heterocycloalkyl); d) COC₁-C₆ alkyl; e) SO₂C₁-C₆ alkyl; or f) 5- or 6-membered heteroaryl optionally substituted with C₁-C₆ alkyl;
R²² is NH₂; C₁-C₆ alkyl substituted with N(C₁-C₆ alkyl)₂ or NH₂; NHC₁-C₆ alkyl substituted with N(C₁-C₆ alkyl)₂ or NH₂; or 4-, 5-, or 6-membered heterocycloalkyl containing NH or NC₁-C₆ alkyl;
R²³ is H or CON(C₁-C₆ alkyl)₂; or R²³ and R²⁴ form a 4-membered spirocyclic heterocycle optionally substituted by C₁-C₆ alkyl;
R²⁶ is H or OH;
R²⁵ is a 4-membered heterocycle containing NH or NC₁-C₆ alkyl;
R²⁷ is H or C₁-C₆ alkyl substituted with N(C₁-C₆ alkyl)₂ or NH₂; and
R²⁸ is C₁-C₆ alkyl substituted with N(C₁-C₆ alkyl)₂ or NH₂.

In some embodiments provided herein is a compound of Formula (XIII):

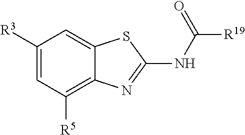

or a pharmaceutically acceptable salt thereof, wherein:
R³=F or H;
R⁵=F, Cl or Br;
R¹⁹ is

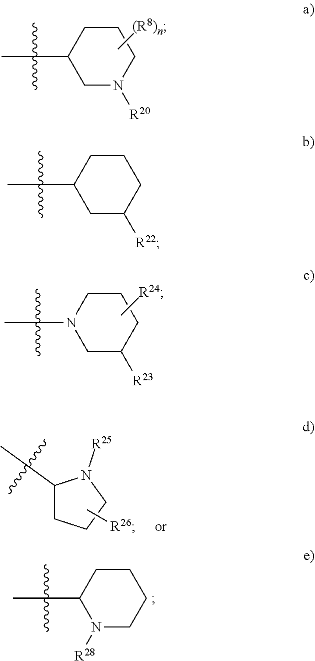

wherein n is 0, 1 or 2, wherein
when n is 1, R⁸ is OH;
and when n is 2,
each R⁸ is F, or
two R⁸ taken together with the atoms connecting them form a 5-membered or 6-membered heterocycloalkane ring;
R²⁰ is selected from
a) CO-(4-, 5-, or 6-membered)-heterocycloalkyl optionally substituted at a ring carbon with C₁-C₆ alkyl; b) 5- or 6-membered heteroaryl optionally substituted with C₁-C₆ alkyl, NH₂, OC₁-C₆ alkyl, or halogen; c) C₁₋₄ alkyl substituted with R³⁰,
wherein R³⁰ is COOC₁-C₆ alkyl, or 4-, 5-, or 6-m. heterocycloalkyl containing N and/or O;
and
d) 4-, 5-, or 6-membered heterocycloalkyl having NR²¹ and/or O and optionally substituted at a ring carbon with R³¹, wherein R³¹ is C₁-C₆ alkyl, ═O, or OH, wherein R²¹ is a) H; b) C₂-C₆ alkenyl; c) C₁-C₄ alkyl optionally substituted with R³², wherein R³² is halo, C₁-C₆ alkoxy, COOC₁-C₆ alkoxy, or CO-(4-, 5-, or 6-membered heterocycloalkyl); d) COC₁-C₆ alkyl; e) SO₂C₁-C₆ alkyl; or f) 5- or 6-membered heteroaryl optionally substituted with C₁-C₆ alkyl;
R²² is NH₂; C₁-C₆ alkyl substituted with N(C₁-C₆ alkyl)₂ or NH₂; or 4-, 5-, or 6-membered heterocycloalkyl containing NH or NC₁-C₆ alkyl;
R²³ is H or CON(C₁-C₆ alkyl)₂; or R²³ and R²⁴ form a 4-membered spirocyclic heterocycle optionally substituted by C₁-C₆ alkyl;
R²⁶ is H or OH;
R²⁵ is a 4-membered heterocycle containing NH or NC₁-C₆ alkyl; and
R²⁸ is C₁-C₆ alkyl substituted with N(C₁-C₆ alkyl)₂ or NH₂.

In some embodiments of a compound of Formula (XII) or Formula (XIII), R³ is F.

In some embodiments of a compound of Formula (XII) or Formula (XIII), R³ is H.

In some embodiments of a compound of Formula (XII) or Formula (XIII), R⁵ is F.

In some embodiments of a compound of Formula (XII) or Formula (XIII), R⁵ is Cl.

In some embodiments of a compound of Formula (XII) or Formula (XIII), R³ is Br.

In some embodiments of a compound of Formula (XII) or Formula (XIII), R¹⁹ is

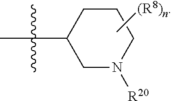

In some embodiments of a compound of Formula (XII) or Formula (XIII), R¹⁹ is

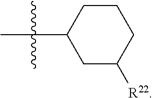

In some embodiments of a compound of Formula (XII) or Formula (XIII),

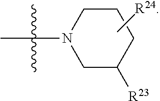

In some embodiments of a compound of Formula (XII) or Formula (XIII), R¹⁹ is

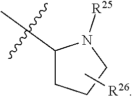

In some embodiments of a compound of Formula (XII) or Formula (XIII), R¹⁹ is

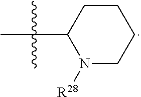

In some embodiments of a compound of Formula (XII) or Formula (XIII), R¹⁹ is

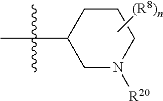

In some embodiments, R¹⁹ is

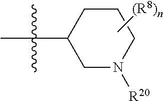

and R²⁰ is selected from
a)CO-(4-, 5-, or 6-membered)-heterocycloalkyl optionally substituted at a ring carbon with C₁-C₆ alkyl; b) 5- or 6-membered heteroaryl optionally substituted with C₁-C₆ alkyl; c) C₁₋₄ alkyl substituted with R³⁰,
   wherein R³⁰ is COOC₁-C₆ alkyl, or 4-, 5-, or 6-m. heterocycloalkyl containing N and/or O;
and
d) 4-, 5-, or 6-membered heterocycloalkyl having NR²¹ and/or O and optionally substituted at a ring carbon with R³¹, wherein R³¹ is C₁-C₆ alkyl, ═O, or OH,
   wherein R²¹ is a) H; b) C₂-C₆ alkenyl; c) C₁-C₄ alkyl optionally substituted with R³², wherein R³² is halo, C₁-C₆ alkoxy, COOC₁-C₆ alkoxy, or CO-(4-, 5-, or 6-membered heterocycloalkyl); d) COC₁-C₆ alkyl; e) SO₂C₁-C₆ alkyl; or f) 5- or 6-membered heteroaryl optionally substituted with C₁-C₆ alkyl.

In some embodiments, R¹⁹ is

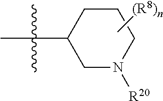

and R²⁰ is CO-(4-, 5-, or 6-membered)-heterocycloalkyl optionally substituted at a ring carbon with C₁-C₆ alkyl.

In some embodiments, R¹⁹ is

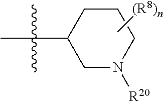

and R²⁰ is 5- or 6-membered heteroaryl optionally substituted with C₁-C₆ alkyl.

In some embodiments, R¹⁹ is

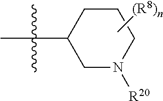

and R²⁰ is C₁₋₄ alkyl substituted with R³⁰, wherein R³⁰ is COOC₁-C₆ alkyl, or 4-, 5-, or 6-m. heterocycloalkyl containing N and/or O.

In some embodiments, R¹⁹ is

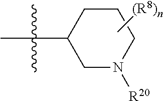

and R²⁰ is 4-, 5-, or 6-membered heterocycloalkyl having NR²¹ and/or O and optionally substituted at a ring carbon with R³¹, wherein R³¹ is C₁-C₆ alkyl, ═O, or OH,
wherein R²¹ is a) H; b) C₂-C₆ alkenyl; c) C₁-C₄ alkyl optionally substituted with R³², wherein R³² is halo, C₁-C₆ alkoxy, COOC₁-C₆ alkoxy, or CO-(4-, 5-, or 6-membered heterocycloalkyl); d) COC₁-C₆ alkyl; e) SO₂C₁-C₆ alkyl; or f) 5- or 6-membered heteroaryl optionally substituted with C₁-C₆ alkyl.

In some embodiments, R¹⁹ is

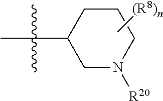

and R²⁰ is a 4-, 5-, or 6-membered heterocycloalkyl containing NR²¹ and/or 0.

In some embodiments, R¹⁹ is

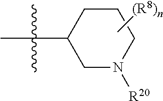

and R²⁰ is a 5-membered heterocycloalkyl containing NR²¹ and/or O and having (R) stereochemistry at the carbon bonded to the N of the

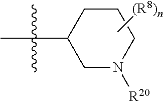

ring.

In some embodiments, R¹⁹ is

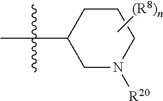

and R²⁰ is a 5-membered heterocycloalkyl containing NR²¹ and/or O and having (S) stereochemistry at the carbon bonded to the N of the

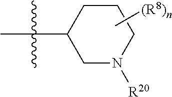

ring.

In some embodiments where R¹⁹ is

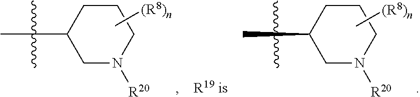

In some embodiments where R¹⁹ is

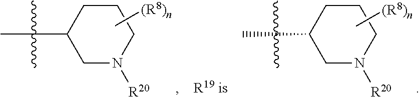

In some embodiments, the compound is selected from the group consisting of the following compounds, or a pharmaceutically acceptable salt thereof:

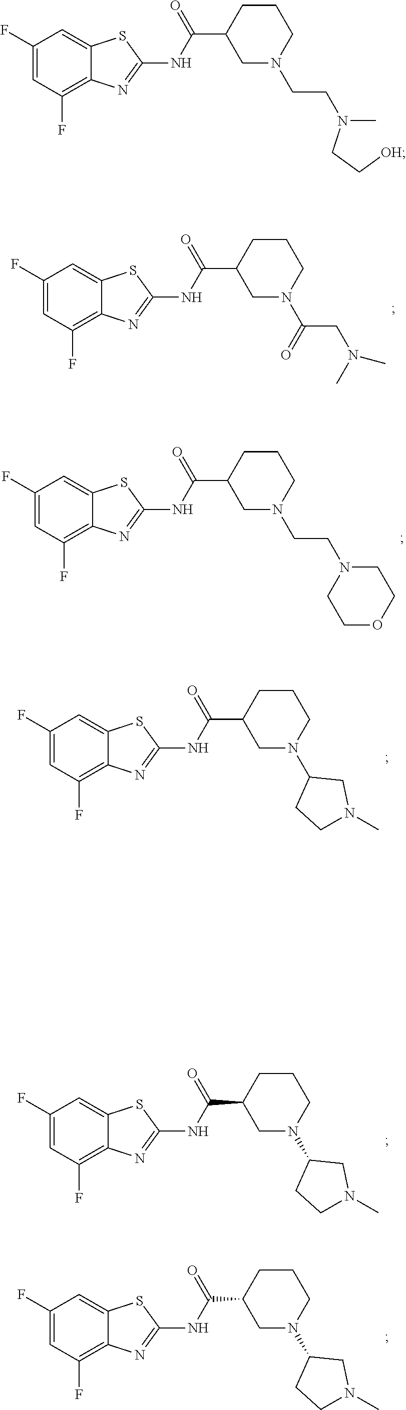 [SMILES: CN(CCO)CCN1CCCC(C(=O)Nc2nc3c(F)cc(F)cc3s2)C1 ; CN(C)CC(=O)N1CCCC(C(=O)Nc2nc3c(F)cc(F)cc3s2)C1 ; O=C(Nc1nc2c(F)cc(F)cc2s1)C1CCCN(CCN2CCOCC2)C1 ; CN1CCC(N2CCCC(C(=O)Nc3nc4c(F)cc(F)cc4s3)C2)C1 ; CN1CC[C@H](N2CCC[C@H](C(=O)Nc3nc4c(F)cc(F)cc4s3)C2)C1 ; CN1CC[C@H](N2CCC[C@@H](C(=O)Nc3nc4c(F)cc(F)cc4s3)C2)C1]

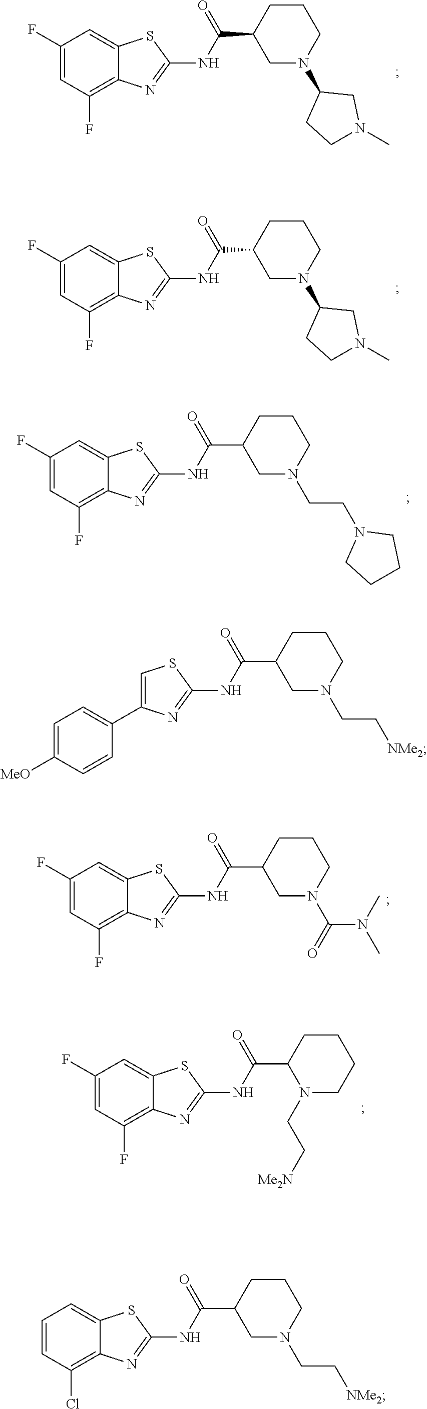

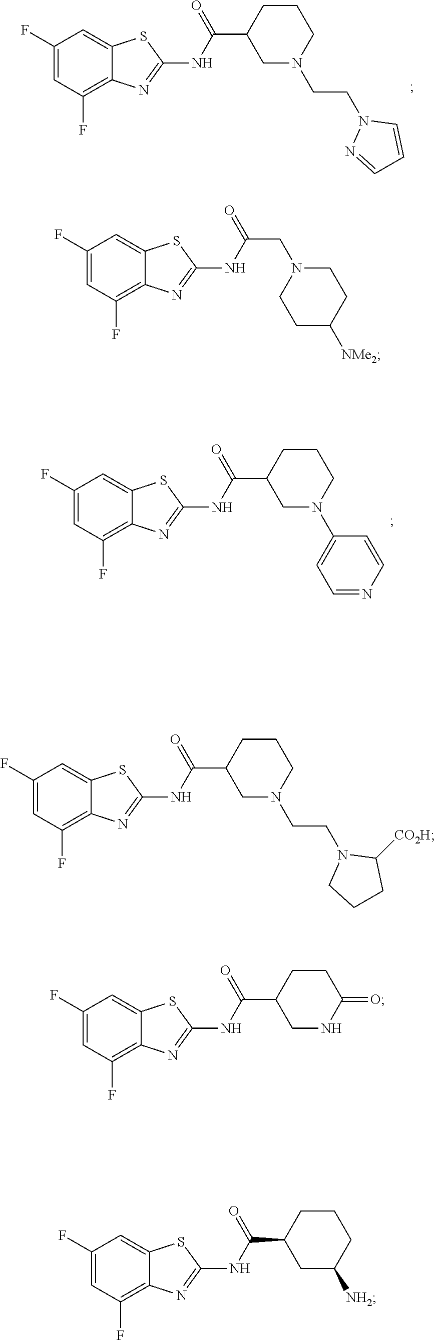 [SMILES: O=C(Nc1nc2c(F)cc(F)cc2s1)C1CCCN(CCn2cccn2)C1 ; CN(C)C1CCN(CC(=O)Nc2nc3c(F)cc(F)cc3s2)CC1 ; O=C(Nc1nc2c(F)cc(F)cc2s1)C1CCCN(c2ccncc2)C1 ; O=C(Nc1nc2c(F)cc(F)cc2s1)C1CCCN(CCN2CCCC2C(=O)O)C1 ; O=C1CCC(C(=O)Nc2nc3c(F)cc(F)cc3s2)CN1 ; N[C@@H]1CCC[C@H](C(=O)Nc2nc3c(F)cc(F)cc3s2)C1]

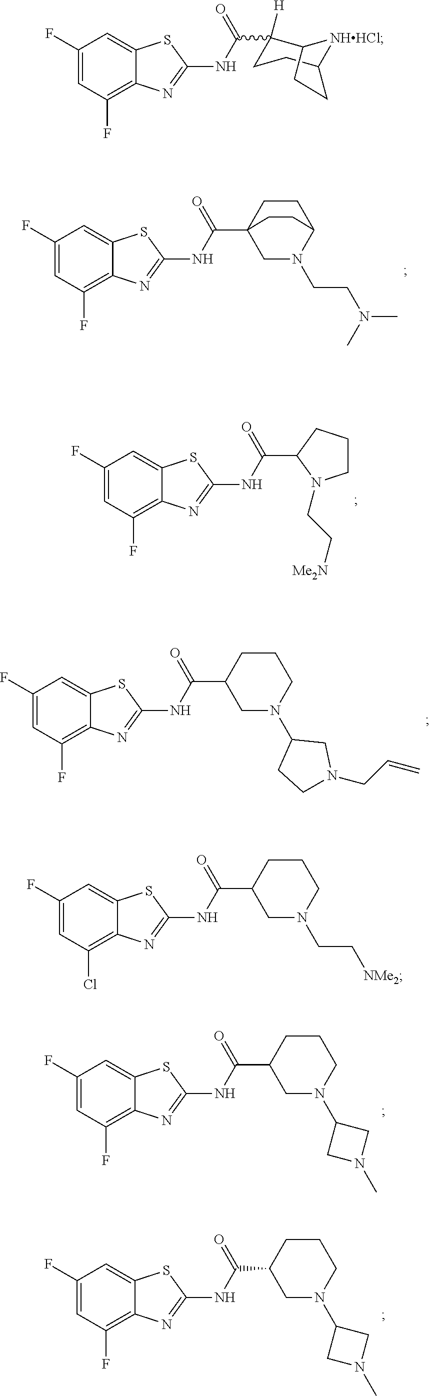

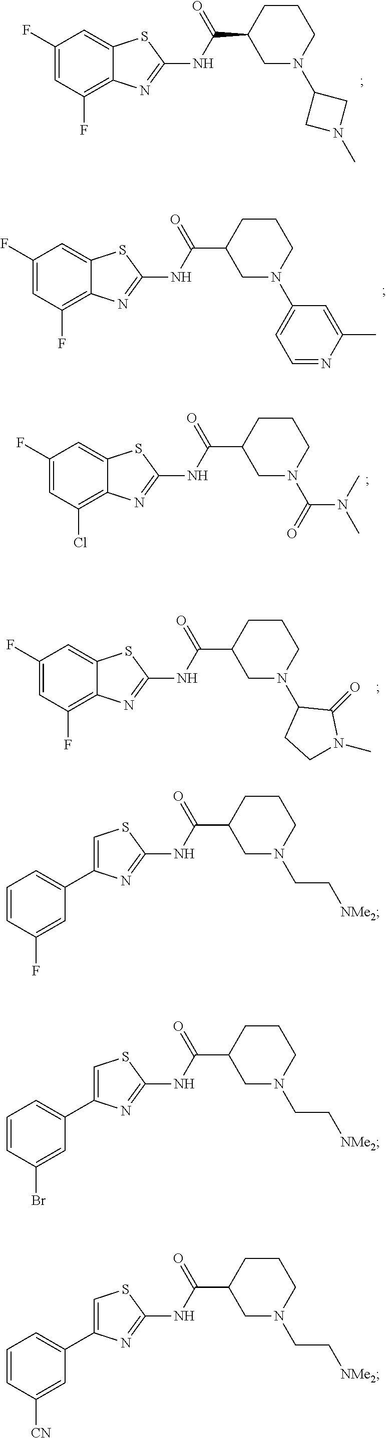

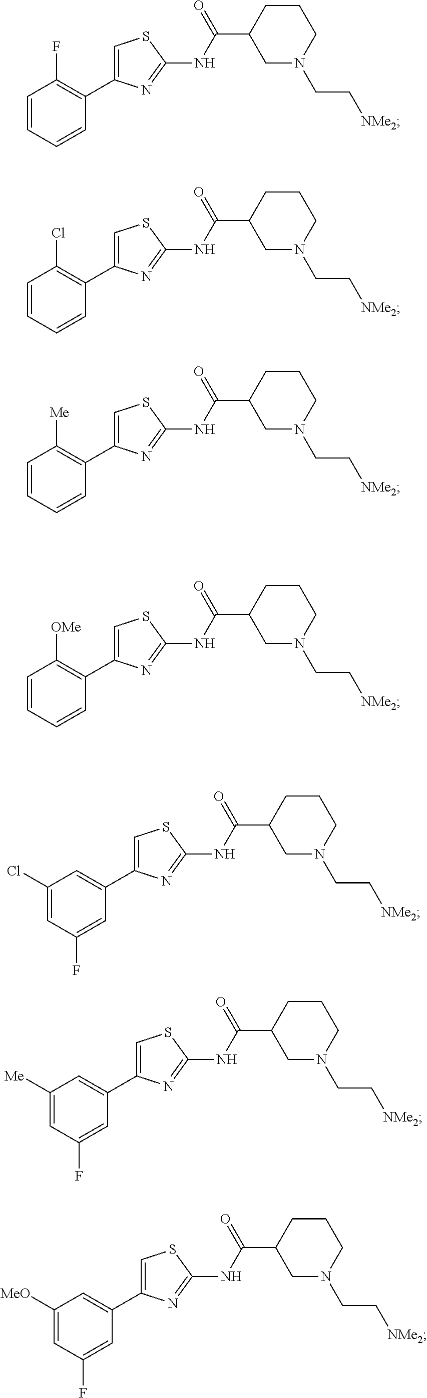

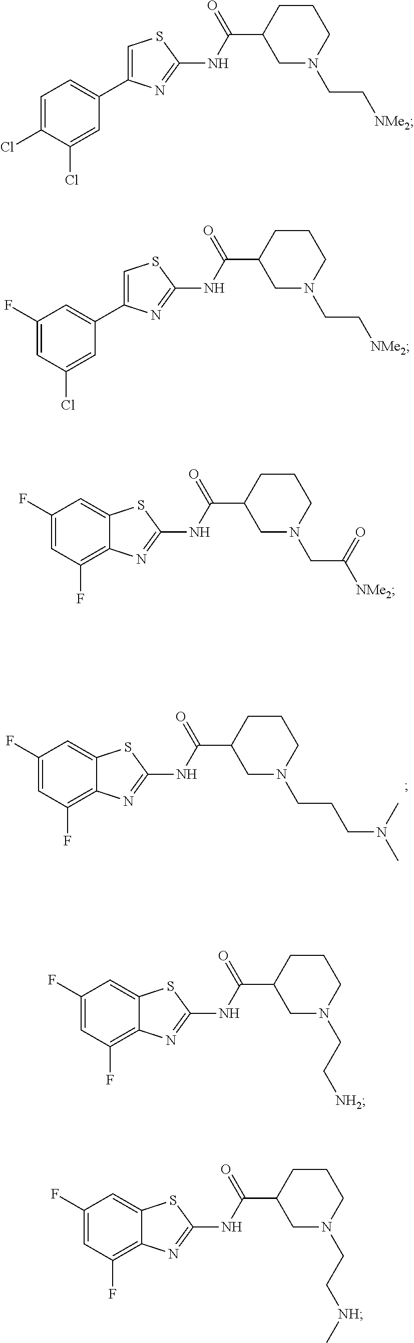 [SMILES: CN(C)CCN1CCCC(C(=O)Nc2nc(-c3ccc(Cl)c(Cl)c3)cs2)C1 ; CN(C)CCN1CCCC(C(=O)Nc2nc(-c3cc(F)cc(Cl)c3)cs2)C1 ; CN(C)C(=O)CN1CCCC(C(=O)Nc2nc3c(F)cc(F)cc3s2)C1 ; CN(C)CCCN1CCCC(C(=O)Nc2nc3c(F)cc(F)cc3s2)C1 ; NCCN1CCCC(C(=O)Nc2nc3c(F)cc(F)cc3s2)C1 ; CNCCN1CCCC(C(=O)Nc2nc3c(F)cc(F)cc3s2)C1]

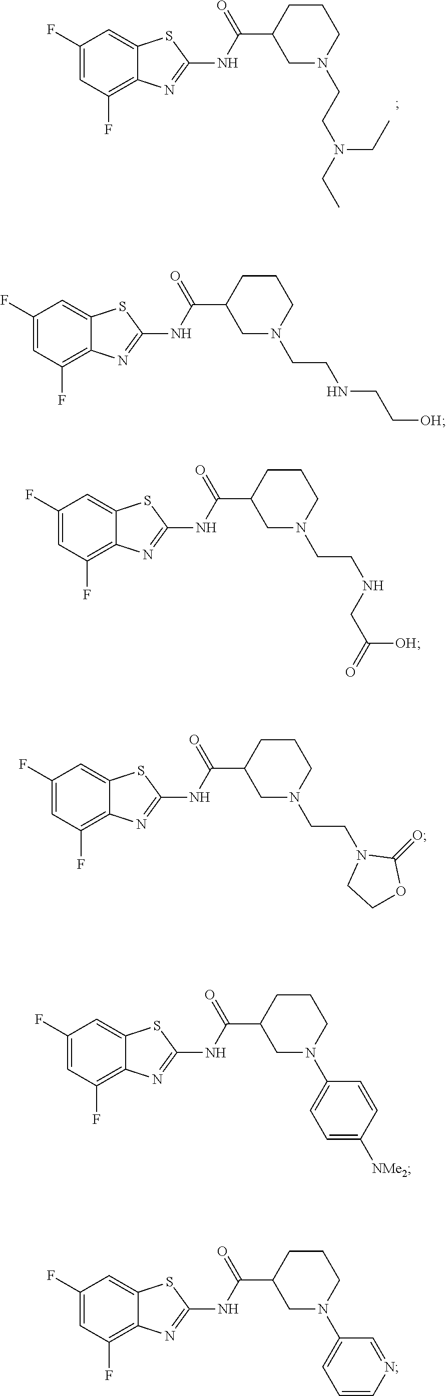 [SMILES: CCN(CC)CCN1CCCC(C(=O)Nc2nc3c(F)cc(F)cc3s2)C1 ; O=C(Nc1nc2c(F)cc(F)cc2s1)C1CCCN(CCNCCO)C1 ; O=C(O)CNCCN1CCCC(C(=O)Nc2nc3c(F)cc(F)cc3s2)C1 ; O=C(Nc1nc2c(F)cc(F)cc2s1)C1CCCN(CCN2CCOC2=O)C1 ; CN(C)c1ccc(N2CCCC(C(=O)Nc3nc4c(F)cc(F)cc4s3)C2)cc1 ; O=C(Nc1nc2c(F)cc(F)cc2s1)C1CCCN(c2cccnc2)C1]

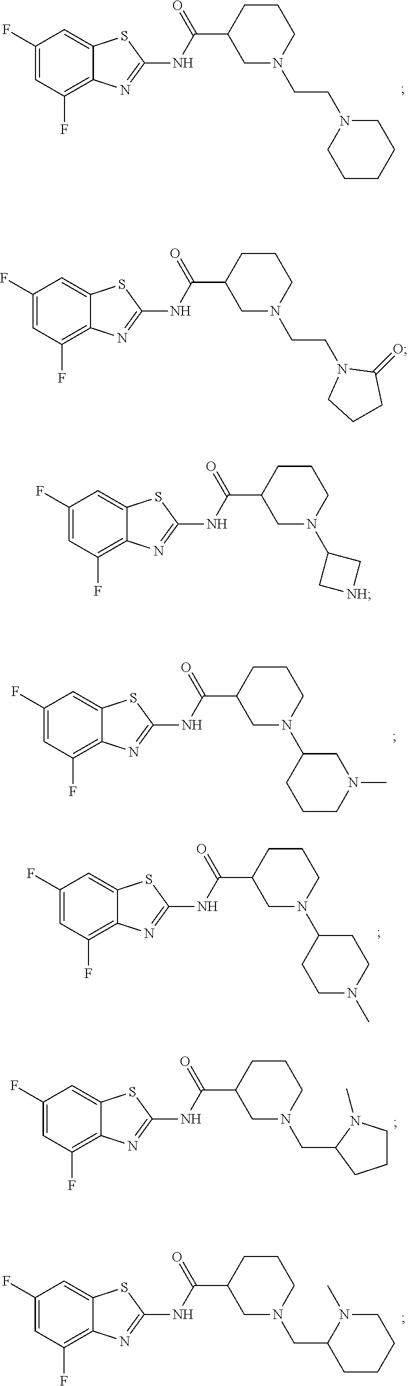

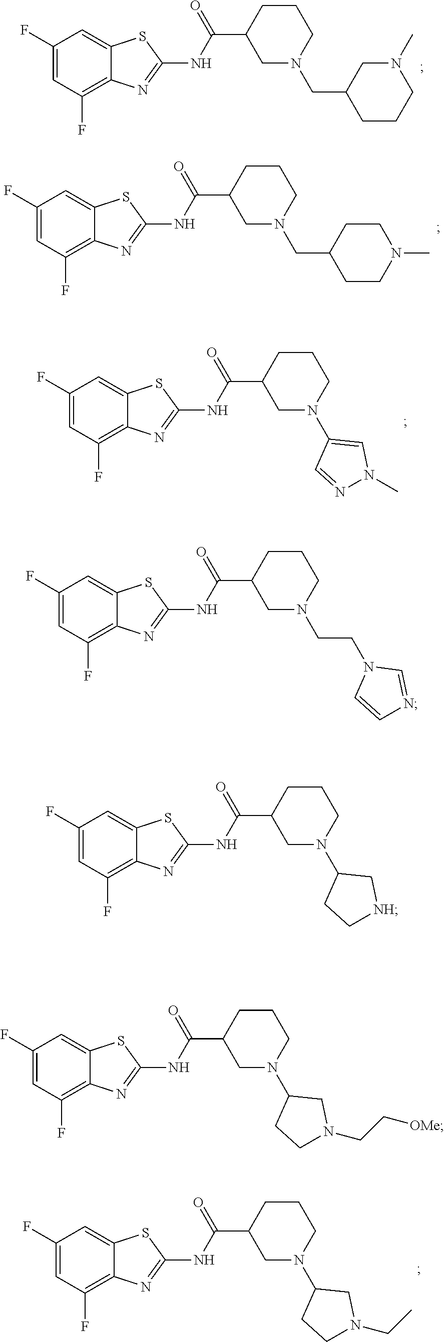

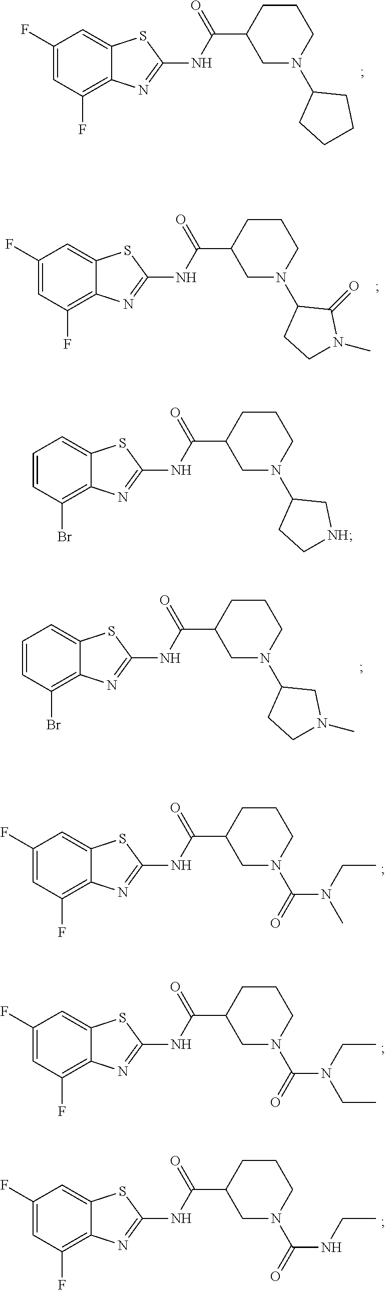

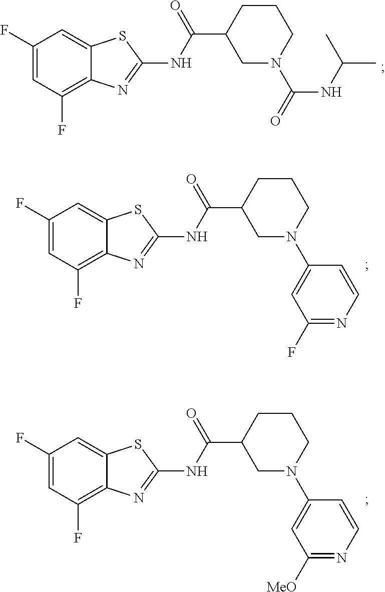 [SMILES: CC(C)NC(=O)N1CCCC(C(=O)Nc2nc3c(F)cc(F)cc3s2)C1 ; O=C(Nc1nc2c(F)cc(F)cc2s1)C1CCCN(c2ccnc(F)c2)C1 ; COc1cc(N2CCCC(C(=O)Nc3nc4c(F)cc(F)cc4s3)C2)ccn1]

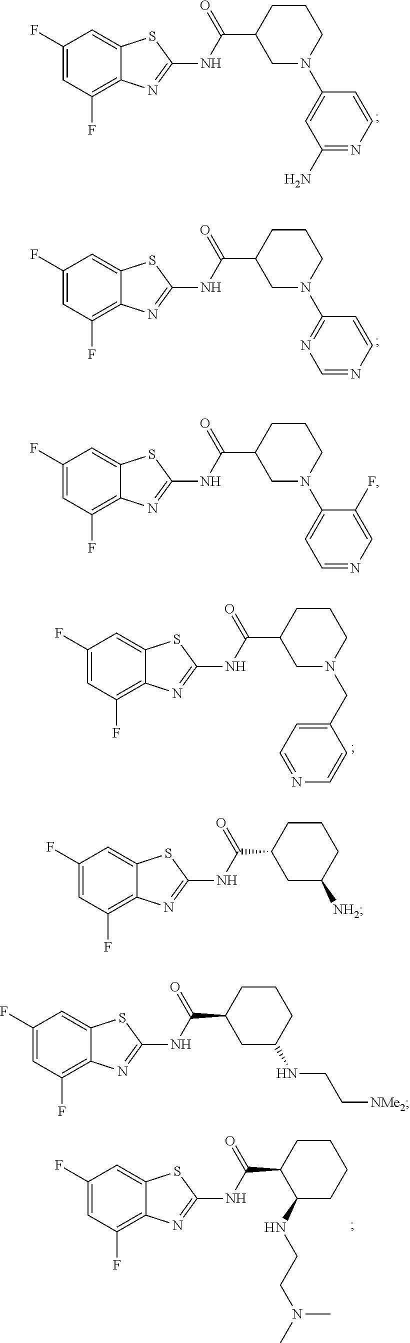

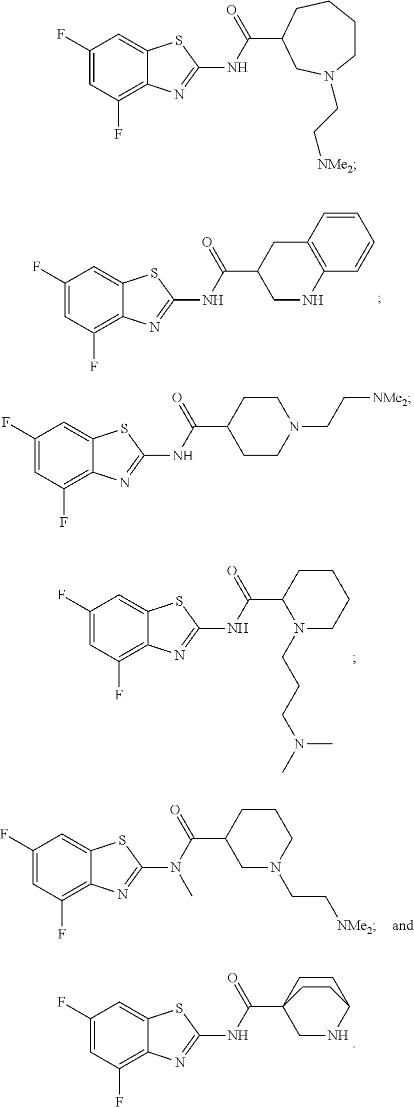

### Pharmaceutically Acceptable Salts

In some embodiments, a salt of a compound of this disclosure is formed between an acid and a basic group of the compound, such as an amino functional group, or a base and an acidic group of the compound, such as a carboxyl functional group. According to another embodiment, the compound is a pharmaceutically acceptable acid addition salt. In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid. In some embodiments, bases commonly employed to form pharmaceutically acceptable salts of the compounds of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds of this disclosure, or pharmaceutically acceptable salts thereof, are substantially isolated.

The compounds disclosed herein may have asymmetric centers. Compounds containing an asymmetrically substituted atom may be in optically active or racemic forms. It is well known in the art how to prepare optically active forms, such as by resolution of materials, for example by chiral chromatography. All forms, including enantiomers, diastereomers, racemic mixtures, scalemic mixtures, as well as mixtures of diastereomers, are within the scope of this disclosure, unless the specific stereochemistry or isomeric form is specified.

### Methods of Making Therapeutic Compounds

Compounds of this disclosure, including salts thereof, can be prepared using known organic synthesis techniques and can be synthesized according to any of numerous possible synthetic routes. A person skilled in the art knows how to select and implement appropriate synthetic protocols, and appreciates that the processes described are not the exclusive means by which compounds provided herein may be synthesized, and that a broad repertoire of synthetic organic reactions is available to be potentially employed in synthesizing compounds provided herein.

Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

### Methods of Using Therapeutic Compounds

The present disclosure provides, at least in part, that the pGC-A/cGMP pathway is a valuable molecular target for metabolic, cardiovascular (CV), renal, and anticancer therapeutics. As discussed above, the elevation of pGC-A's endogenous ligand ANP levels is associated with protection from obesity and metabolic syndrome, reduced blood pressure, decreased risk for hypertension as well as reduced incidence of myocardial infarction. Similarly, the elevation of levels of endogenous ligand BNP is associated with reduced risk for type II diabetes mellitus.

Accordingly, in a general aspect, the present disclosure provides a method of modulating particulate guanylyl cyclase receptor A (pGC-A) in a cell, the method comprising contacting the cell with an effective amount of the compound as described herein, or a pharmaceutically acceptable salt thereof. In some embodiments, the cell is contacted in vitro, in vivo, or ex vivo.

The present disclosure also provides a method of modulating particulate guanylyl cyclase receptor A (pGC-A) in a subject, the method comprising administering to the subject in need thereof an effective amount of the compound as described herein, or a pharmaceutically acceptable salt thereof, or the pharmaceutical composition comprising same.

In some embodiments of the methods of the present disclosure, modulating of the particulate guanylyl cyclase receptor A (pGC-A) comprises positive allosteric enhancement of activity of the particulate guanylyl cyclase receptor A (pGC-A) (e.g., the modulating comprises increased production cGMP in a cell (e.g., in a cell of the subject)). In some embodiments, the cell is a renal cell, a heart muscle cell or fat cell.

The present disclosure also provides a method of treating or preventing a disease or condition responsive to modulation of a particulate guanylyl cyclase receptor A (pGC-A) in a subject, the method comprising administering to the subject in need thereof a therapeutically effective amount of the compound as described herein, or a pharmaceutically acceptable salt thereof, or the pharmaceutical composition comprising same.

The present disclosure also provides a compound as described herein, or a pharmaceutically acceptable salt thereof, or the pharmaceutical composition comprising same, for use in a manufacture of a medicament for the treatment or prevention of a disease or condition responsive to modulation of a particulate guanylyl cyclase receptor A (pGC-A) in a subject.

The present disclosure also provides a compound as described herein, or a pharmaceutically acceptable salt thereof, or the pharmaceutical composition comprising same, for use the treatment or prevention of a disease or condition responsive to modulation of a particulate guanylyl cyclase receptor A (pGC-A) in a subject.

In some embodiments, the disease or condition responsive to modulation of a particulate guanylyl cyclase receptor A (pGC-A) is a metabolic disease or disorder. In some embodiments, the metabolic disorder is congenital. Suitable examples of such disorders include Fabry disease, phenylketonuria, Prader-Willi syndrome, galactosemia, Tay-Sachs's disease, porphyria, Pompe disease, Neimann-Pick disease, Morquio's syndrome, Morteaus-lamy syndrome, Hunter syndrome, Lesh-Nyhan syndrome, Hurler syndrome, homocystinuria, Hartnup disease, and Gaucher's disease. In some embodiments, the metabolic disorder is acquired. Suitable examples of such disorders include diabetes (e.g., type 1 diabetes, diabetes insipidus, or type II diabetes mellitus), obesity, metabolic syndrome, dyslipidemia, hipolipidemia (hyperlipoproteinemia), hyperthyroidism, hypoparathyroidism, hypothyroidism, Cushing's syndrome, hyperuricemia, hemochromatosis, and hyperparathyroidism. Other examples of metabolic disorders include glucose intolerance, insulin resistance, fibrinolysis disorder, endothelial dysfunction, atherosclerosis, impaired fasting glycemia, hyperinsulinemia, galactosemia, mucopolysaccaridose, tyrosinemia, methylmalonic aciduria, acidemia (e.g., propionic acidemia, isovaleric acidemia), and hyperammonemia. In some embodiments, the metabolic disease is selected from obesity, hypertriglyceridemia, metabolic syndrome, insulin resistance, hyperinsulinemia, diabetes, and acidemia.

In some embodiments, the disease or condition responsive to modulation of a particulate guanylyl cyclase receptor A (pGC-A) is a cardiovascular disease. Suitable examples of cardiovascular disorders include high blood pressure, myocardial infarction, abnormal heart rhythms (e.g., arrhythmia), aorta disease, Marfan syndrome, congenital heart disease, coronary artery disease (e.g., narrowing of the arteries), deep vein thrombosis, pulmonary embolism, heart attack, heart failure, heart muscle disease (e.g., cardiomyopathy), heart valve disease, pericardial disease, peripheral vascular disease, rheumatic heart disease, stroke, vascular disease (e.g., blood vessel disease), cardiomyopathies, hypertension, aortic stenosis, mitral valve insufficiency, mitral valve prolapse, pericarditis, rheumatic heart disease, and cardiorenal syndrome. In some embodiments, the cardiovascular disease is selected from heart failure, cardiomyopathy, hypertension, high blood pressure, and myocardial infarction.

In some embodiments, the disease or condition responsive to modulation of a particulate guanylyl cyclase receptor A (pGC-A) is kidney disease. Suitable examples of renal diseases include nephropathy, acute kidney injury, kidney failure, acute renal failure, kidney stones, glomerulonephritis, polycystic kidney disease, urinary tract infections, kidney infection (pyelonephritis), simple kidney cysts, diabetic kidney disease, nephropathy, lupus nephritis, Henoch-Schonlein purpura, goodpasture syndrome, ectopic kidney, amyloidosis, acquired cystic kidney disease, glomerular disease, kidney dysplasia, medullary sponge kidney, nephrotic syndrome, kidney damage, renal artery stenosis, renal tubular acidosis, and solitary kidney. In some embodiments, the kidney disease is selected from nephropathy, acute renal failure, chronic kidney disease, cardiorenal syndrome and diabetic kidney disease.

In some embodiments, the disease or condition responsive to modulation of a particulate guanylyl cyclase receptor A (pGC-A) is cancer. Suitable example of cancer include bladder cancer, brain cancer, breast cancer, colorectal cancer (e.g., colon cancer), rectal cancer, cervical cancer, gastrointestinal cancer, genitourinary cancer, head and neck cancer, lung cancer, oral cancer, ovarian cancer, pancreatic cancer (e.g., pancreatic neuroendocrine tumor), prostate cancer, endometrial cancer, renal cancer (kidney cancer) (e.g., advanced kidney cancer), skin cancer, liver cancer, thyroid cancer, leukemia, and testicular cancer.

### Pharmaceutical Compositions and Formulations

The present application also provides pharmaceutical compositions comprising an effective amount of a compound of as described herein, or a pharmaceutically acceptable salt thereof, and a pharmaceutically acceptable carrier. The pharmaceutical composition may also comprise any one of the additional therapeutic agents described herein, or a pharmaceutically acceptable salt thereof. In certain embodiments, the application also provides pharmaceutical compositions and dosage forms comprising any one the additional therapeutic agents described herein, or a pharmaceutically acceptable salt thereof, and a pharmaceutically acceptable carrier. The carrier(s) and excipient(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound as described herein is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a compound as described herein can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound as described herein is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent in a suitable amount or dosage.

### Definitions

At various places in the present specification, substituents of compounds of the present application are disclosed in groups or in ranges. It is specifically intended that various embodiments of the present application include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, the term “about” means “approximately” (e.g., plus or minus approximately 10% of the indicated value).

As used herein, the term “compound” as used herein is meant to include all stereoisomers, geometric isomers, tautomers, and isotopes of the structures named or depicted. Compounds herein identified by name or structure as one particular tautomeric form are intended to include other tautomeric forms unless otherwise specified.

As used herein, the term “tautomer” refers to compounds which are capable of existing in a state of equilibrium between two isomeric forms. Such compounds may differ in the bond connecting two atoms or groups and the position of these atoms or groups in the compound.

As used herein, the term “isomer” refers to structural, geometric and stereo isomers.

Throughout the definitions, the term “Cₙ₋ₘ” indicates a range which includes the endpoints, wherein n and m are integers and indicate the number of carbon atoms. Examples include C₁₋₄, C₁₋₆, and the like.

As used herein, the phrase “optionally substituted” means unsubstituted or substituted. As used herein, the term “substituted” means that a hydrogen atom is removed and replaced by a substituent. It is to be understood that substitution at a given atom is limited by valency.

As used herein, the term “Cn-m alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cn-m haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cn-m alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cn-m alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl.

Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen.

In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C=O), or attached to a heteroatom forming a sulfoxide or sulfone group.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system, an in vivo system, or an ex vivo system. For example, “contacting” the particulate guanylyl cyclase receptor A with a compound of the invention includes the administration of a compound of the present invention to an individual or patient, such as a human, having particulate guanylyl cyclase receptor A, as well as, for example, introducing a compound of the invention into a sample containing a cellular or purified preparation containing the particulate guanylyl cyclase receptor A.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

Assay: Generally, the assay monitors the production of cGMP, the second messenger generated by pGC-A, by Time-Resolved Florescence (HTRF) in HEK293 cells overexpressing the pGC-A. pGC-A suspension cells were stimulated in the presence of the test compound and an EC₂₀ concentration of ANP. The quantity of cGMP was detected by competitive immunoassay using Eu³⁺ cryptate-labeled anti-cGMP and d2-labeled cGMP and normalized to maximal amount produced by an EC₈₀ concentration of ANP. Compound EC₅₀ values were determined in the primary assay, in the presence or absence of ANP, to determine mode of action as positive modulators and tested for selectivity in the same assay platform but in HEK cells overexpressing the particulate guanylyl cyclase B receptor (pGC-B), of which CNP is the endogenous ligand.

Assay details: 20 nL of 10 μM test compound in DMSO was added to columns 5-48 of 1536 well white high base screening plates (Corning, New York, NY) cells using 550 ECHO acoustic dispenser (Labcyte, San Jose, CA). Alpha-atrial natriuretic peptide (ANP) (Phoenix Pharmaceutics) was prepared as stock aliquots at 5 μM in PBS with 0.1% BSA. An approximate EC₃₀ concentration of ANP (9 μM) in assay buffer (HBSS containing 5 mM HEPES and 0.05% BSA) was added to columns 3-48 at a volume of 1 μL. Assay buffer only was added to column 1 and assay buffer containing a saturating concentration of ANP (5 nM) was added to column 2. HEK293 cells overexpressing GC-A were resuspended in assay media (OptimMem media containing 2% Heat-inactivated Fetal bovine serum and L-glutamine) at a density of 6×10⁵ cells/mL and 2 μL were plated in screening plates (1200 cells/well) in suspension using a Bioraptr 2. Plates were spun at 1000 rpm for 1 min and incubated for 30 min at room temperature. 1.5 μL d2-labeled cGMP followed by 1.5 μL Eu³⁺ cryptate-labeled anti-cGMP cGMP detection kit (CiBio; #62GM2PEC) prepared according to manufacturer's protocol were added to all wells using a Bioraptr 2 and TR-FRET signal was detected on an EnVision detector (PerkinElmer). Wells treated with 0.3% DMSO only serve as blank controls (column 1); wells treated with 0.3% DMSO and 5 nM ANP (columns 2) serve as positive controls and wells treated with 0.3% DMSO and 9 μM ANP (columns 3-4) serve as negative controls. DMSO did not exceed 0.3% in all wells.

### Example 1

The following compounds provided in Table 1 were prepared using methods and procedures similar to those described in WO2021/243230, which is incorporated herein by reference in its entirety.

TABLE 1

| Cmpd. No. | Chemical structure |
|---|---|
| 216 | 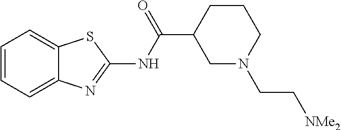 [SMILES: CN(C)CCN1CCCC(C(=O)Nc2nc3ccccc3s2)C1] |
| 224 | 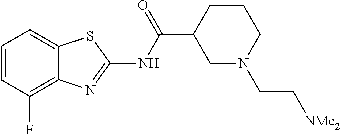 [SMILES: CN(C)CCN1CCCC(C(=O)Nc2nc3c(F)cccc3s2)C1] |
| 225 | 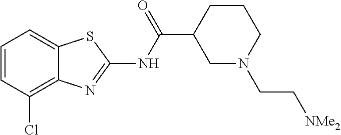 [SMILES: CN(C)CCN1CCCC(C(=O)Nc2nc3c(Cl)cccc3s2)C1] |
| 001 | 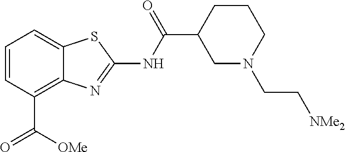 [SMILES: COC(=O)c1cccc2sc(NC(=O)C3CCCN(CCN(C)C)C3)nc12] |
| 002 | 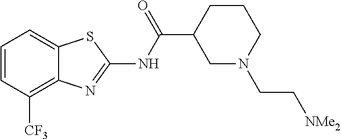 [SMILES: CN(C)CCN1CCCC(C(=O)Nc2nc3c(C(F)(F)F)cccc3s2)C1] |
| 003 | 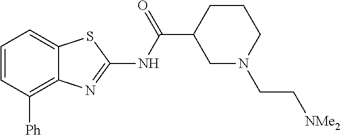 [SMILES: CN(C)CCN1CCCC(C(=O)Nc2nc3c(-c4ccccc4)cccc3s2)C1] |
| 004 | 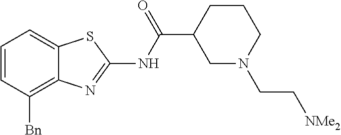 [SMILES: CN(C)CCN1CCCC(C(=O)Nc2nc3c(Cc4ccccc4)cccc3s2)C1] |
| 005 | 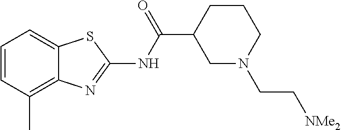 [SMILES: Cc1cccc2sc(NC(=O)C3CCCN(CCN(C)C)C3)nc12] |
| 006 | 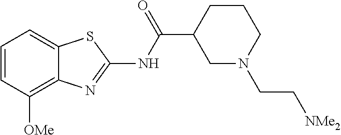 [SMILES: COc1cccc2sc(NC(=O)C3CCCN(CCN(C)C)C3)nc12] |
| 010 | 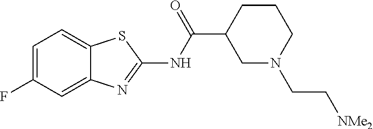 [SMILES: CN(C)CCN1CCCC(C(=O)Nc2nc3cc(F)ccc3s2)C1] |
| 011 | 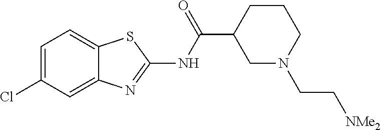 [SMILES: CN(C)CCN1CCCC(C(=O)Nc2nc3cc(Cl)ccc3s2)C1] |
| 012 | 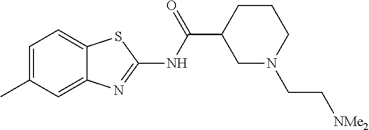 [SMILES: Cc1ccc2sc(NC(=O)C3CCCN(CCN(C)C)C3)nc2c1] |
| 013 | 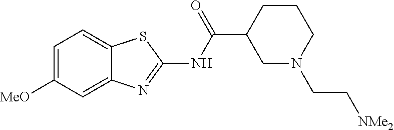 [SMILES: COc1ccc2sc(NC(=O)C3CCCN(CCN(C)C)C3)nc2c1] |
| 015 | 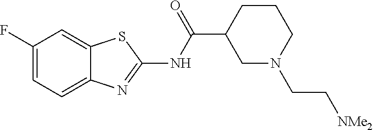 [SMILES: CN(C)CCN1CCCC(C(=O)Nc2nc3ccc(F)cc3s2)C1] |
| 016 | 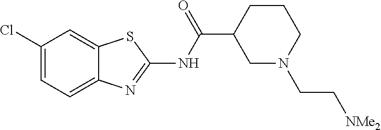 [SMILES: CN(C)CCN1CCCC(C(=O)Nc2nc3ccc(Cl)cc3s2)C1] |
| 017 | 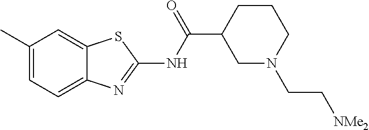 [SMILES: Cc1ccc2nc(NC(=O)C3CCCN(CCN(C)C)C3)sc2c1] |
| 018 | 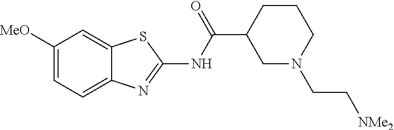 [SMILES: COc1ccc2nc(NC(=O)C3CCCN(CCN(C)C)C3)sc2c1] |
| 032 | 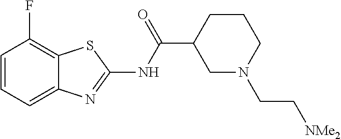 [SMILES: CN(C)CCN1CCCC(C(=O)Nc2nc3cccc(F)c3s2)C1] |
| 033 | 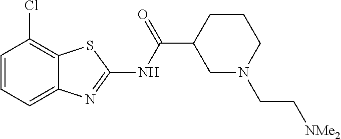 [SMILES: CN(C)CCN1CCCC(C(=O)Nc2nc3cccc(Cl)c3s2)C1] |
| 034 | 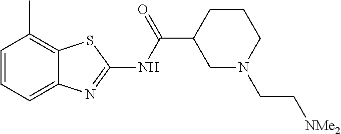 [SMILES: Cc1cccc2nc(NC(=O)C3CCCN(CCN(C)C)C3)sc12] |
| 035 | 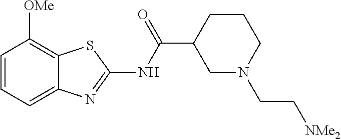 [SMILES: COc1cccc2nc(NC(=O)C3CCCN(CCN(C)C)C3)sc12] |
| 226 | 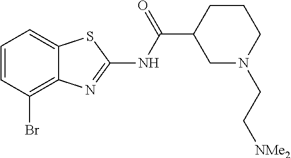 [SMILES: CN(C)CCN1CCCC(C(=O)Nc2nc3c(Br)cccc3s2)C1] |
| 007 | 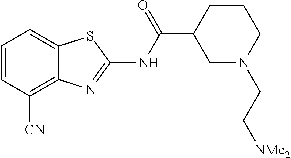 [SMILES: CN(C)CCN1CCCC(C(=O)Nc2nc3c(C#N)cccc3s2)C1] |
| 008 | 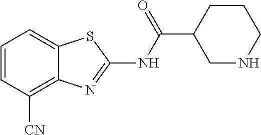 [SMILES: N#Cc1cccc2sc(NC(=O)C3CCCNC3)nc12] |
| 227 | 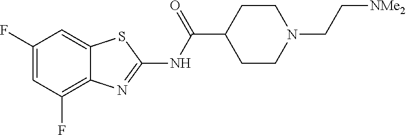 [SMILES: CN(C)CCN1CCC(C(=O)Nc2nc3c(F)cc(F)cc3s2)CC1] |
| 228 | 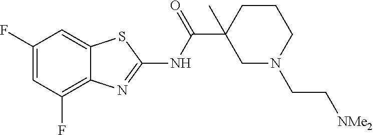 [SMILES: CN(C)CCN1CCCC(C)(C(=O)Nc2nc3c(F)cc(F)cc3s2)C1] |
| 037 | 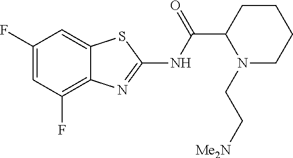 [SMILES: CN(C)CCN1CCCCC1C(=O)Nc1nc2c(F)cc(F)cc2s1] |
| 229 | 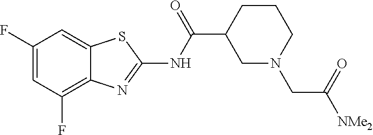 [SMILES: CN(C)C(=O)CN1CCCC(C(=O)Nc2nc3c(F)cc(F)cc3s2)C1] |
| 230 | 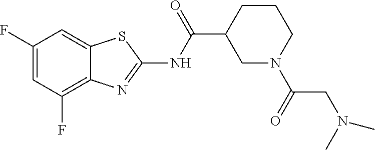 [SMILES: CN(C)CC(=O)N1CCCC(C(=O)Nc2nc3c(F)cc(F)cc3s2)C1] |
| 231 | 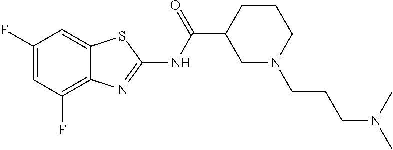 [SMILES: CN(C)CCCN1CCCC(C(=O)Nc2nc3c(F)cc(F)cc3s2)C1] |
| 232 | 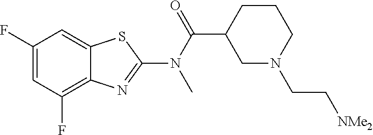 [SMILES: CN(C)CCN1CCCC(C(=O)N(C)c2nc3c(F)cc(F)cc3s2)C1] |
| 217 | 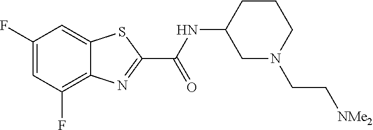 [SMILES: CN(C)CCN1CCCC(NC(=O)c2nc3c(F)cc(F)cc3s2)C1] |
| 038 | 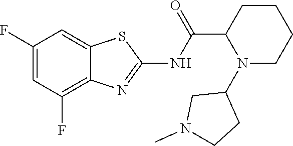 [SMILES: CN1CCC(N2CCCCC2C(=O)Nc2nc3c(F)cc(F)cc3s2)C1] |
| 039 | 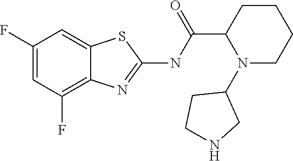 [SMILES: O=C(Nc1nc2c(F)cc(F)cc2s1)C1CCCCN1C1CCNC1] |
| 233 | 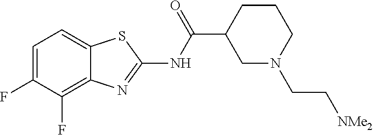 [SMILES: CN(C)CCN1CCCC(C(=O)Nc2nc3c(F)c(F)ccc3s2)C1] |
| 234 | 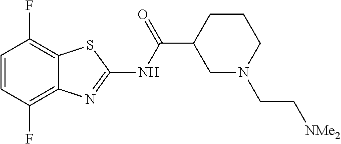 [SMILES: CN(C)CCN1CCCC(C(=O)Nc2nc3c(F)ccc(F)c3s2)C1] |
| 235 | 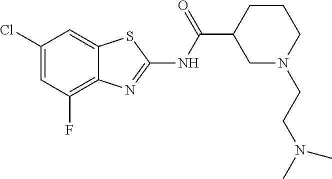 [SMILES: CN(C)CCN1CCCC(C(=O)Nc2nc3c(F)cc(Cl)cc3s2)C1] |
| 019 | 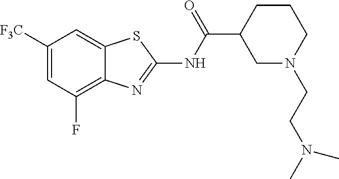 [SMILES: CN(C)CCN1CCCC(C(=O)Nc2nc3c(F)cc(C(F)(F)F)cc3s2)C1] |
| 020 | 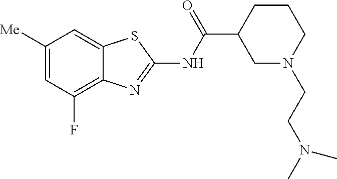 [SMILES: Cc1cc(F)c2nc(NC(=O)C3CCCN(CCN(C)C)C3)sc2c1] |
| 021 | 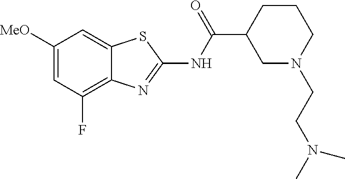 [SMILES: COc1cc(F)c2nc(NC(=O)C3CCCN(CCN(C)C)C3)sc2c1] |
| 022 | 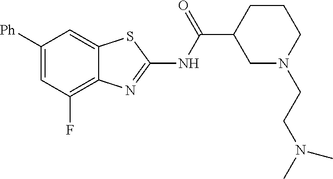 [SMILES: CN(C)CCN1CCCC(C(=O)Nc2nc3c(F)cc(-c4ccccc4)cc3s2)C1] |
| 023 | 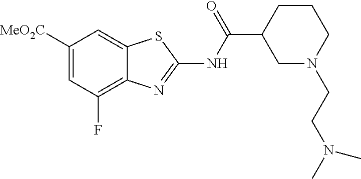 [SMILES: COC(=O)c1cc(F)c2nc(NC(=O)C3CCCN(CCN(C)C)C3)sc2c1] |
| 024 | 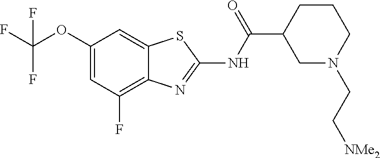 [SMILES: CN(C)CCN1CCCC(C(=O)Nc2nc3c(F)cc(OC(F)(F)F)cc3s2)C1] |
| 025 | 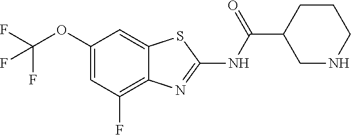 [SMILES: O=C(Nc1nc2c(F)cc(OC(F)(F)F)cc2s1)C1CCCNC1] |
| 026 | 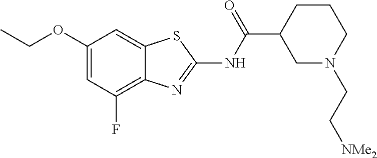 [SMILES: CCOc1cc(F)c2nc(NC(=O)C3CCCN(CCN(C)C)C3)sc2c1] |
| 027 | 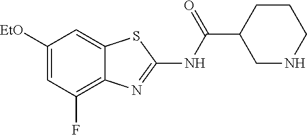 [SMILES: CCOc1cc(F)c2nc(NC(=O)C3CCCNC3)sc2c1] |
| 028 | 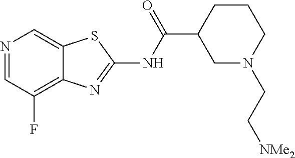 [SMILES: CN(C)CCN1CCCC(C(=O)Nc2nc3c(F)cncc3s2)C1] |
| 029 | 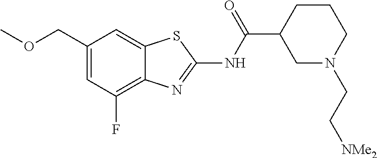 [SMILES: COCc1cc(F)c2nc(NC(=O)C3CCCN(CCN(C)C)C3)sc2c1] |
| 030 | 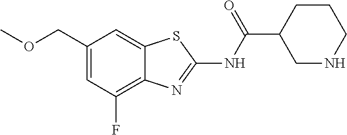 [SMILES: COCc1cc(F)c2nc(NC(=O)C3CCCNC3)sc2c1] |
| 236 | 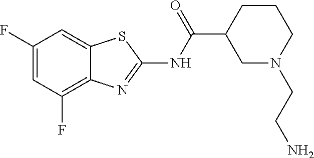 [SMILES: NCCN1CCCC(C(=O)Nc2nc3c(F)cc(F)cc3s2)C1] |
| 237 | 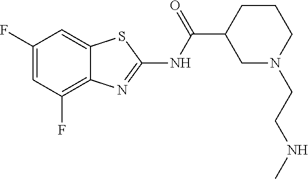 [SMILES: CNCCN1CCCC(C(=O)Nc2nc3c(F)cc(F)cc3s2)C1] |
| 238 | 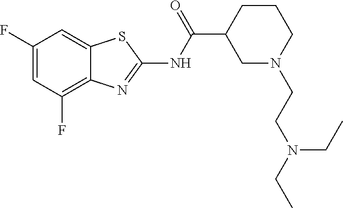 [SMILES: CCN(CC)CCN1CCCC(C(=O)Nc2nc3c(F)cc(F)cc3s2)C1] |
| 239 | 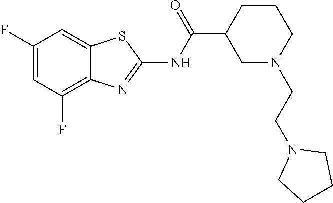 [SMILES: O=C(Nc1nc2c(F)cc(F)cc2s1)C1CCCN(CCN2CCCC2)C1] |
| 240 | 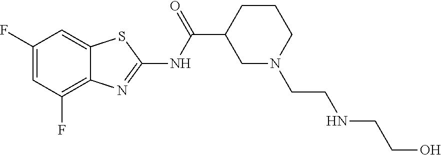 [SMILES: O=C(Nc1nc2c(F)cc(F)cc2s1)C1CCCN(CCNCCO)C1] |
| 241 | 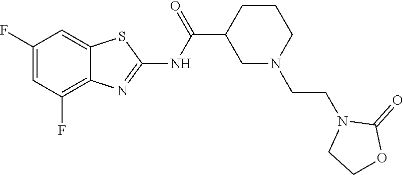 [SMILES: O=C(Nc1nc2c(F)cc(F)cc2s1)C1CCCN(CCN2CCOC2=O)C1] |
| 242 | 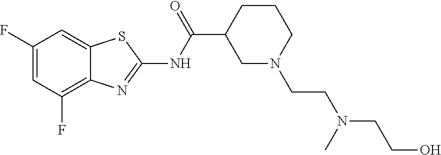 [SMILES: CN(CCO)CCN1CCCC(C(=O)Nc2nc3c(F)cc(F)cc3s2)C1] |
| 064 | 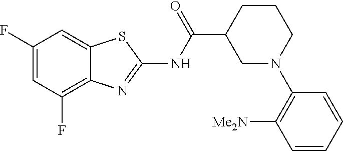 [SMILES: CN(C)c1ccccc1N1CCCC(C(=O)Nc2nc3c(F)cc(F)cc3s2)C1] |
| 065 | 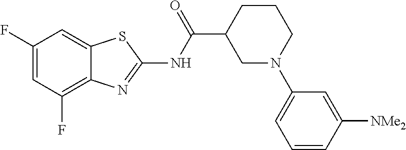 [SMILES: CN(C)c1cccc(N2CCCC(C(=O)Nc3nc4c(F)cc(F)cc4s3)C2)c1] |
| 066 | 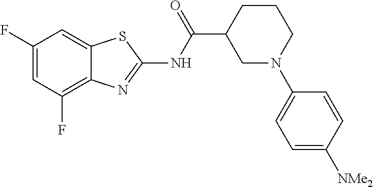 [SMILES: CN(C)c1ccc(N2CCCC(C(=O)Nc3nc4c(F)cc(F)cc4s3)C2)cc1] |
| 243 | 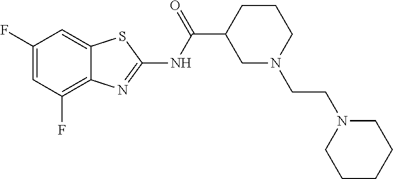 [SMILES: O=C(Nc1nc2c(F)cc(F)cc2s1)C1CCCN(CCN2CCCCC2)C1] |
| 244 | 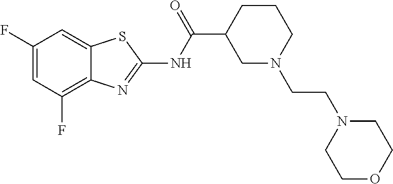 [SMILES: O=C(Nc1nc2c(F)cc(F)cc2s1)C1CCCN(CCN2CCOCC2)C1] |
| 067 | 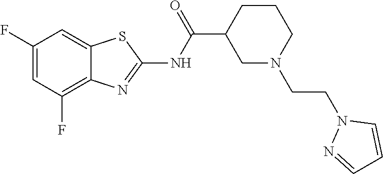 [SMILES: O=C(Nc1nc2c(F)cc(F)cc2s1)C1CCCN(CCn2cccn2)C1] |
| 068 | 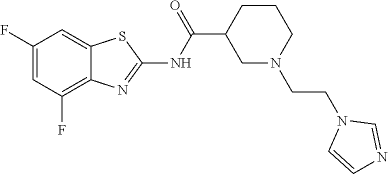 [SMILES: O=C(Nc1nc2c(F)cc(F)cc2s1)C1CCCN(CCn2ccnc2)C1] |
| 245 | 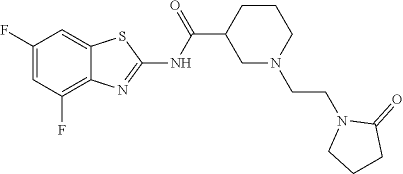 [SMILES: O=C(Nc1nc2c(F)cc(F)cc2s1)C1CCCN(CCN2CCCC2=O)C1] |
| 069 | 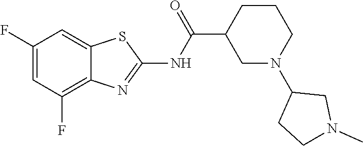 [SMILES: CN1CCC(N2CCCC(C(=O)Nc3nc4c(F)cc(F)cc4s3)C2)C1] |
| 070 | 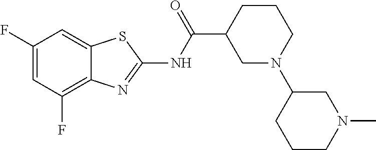 [SMILES: CN1CCCC(N2CCCC(C(=O)Nc3nc4c(F)cc(F)cc4s3)C2)C1] |
| 071 | 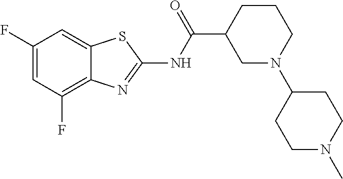 [SMILES: CN1CCC(N2CCCC(C(=O)Nc3nc4c(F)cc(F)cc4s3)C2)CC1] |
| 072 | 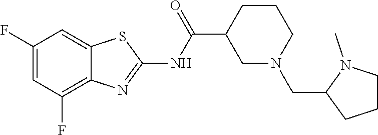 [SMILES: CN1CCCC1CN1CCCC(C(=O)Nc2nc3c(F)cc(F)cc3s2)C1] |
| 073 | 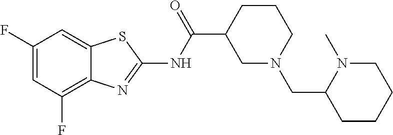 [SMILES: CN1CCCCC1CN1CCCC(C(=O)Nc2nc3c(F)cc(F)cc3s2)C1] |
| 074 | 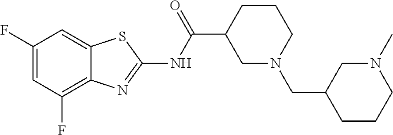 [SMILES: CN1CCCC(CN2CCCC(C(=O)Nc3nc4c(F)cc(F)cc4s3)C2)C1] |
| 075 | 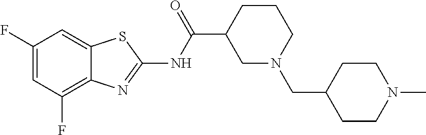 [SMILES: CN1CCC(CN2CCCC(C(=O)Nc3nc4c(F)cc(F)cc4s3)C2)CC1] |
| 076 | 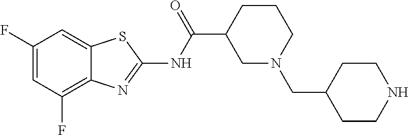 [SMILES: O=C(Nc1nc2c(F)cc(F)cc2s1)C1CCCN(CC2CCNCC2)C1] |
| 077 | 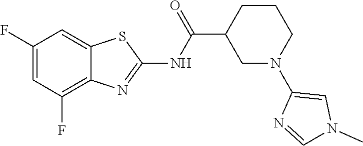 [SMILES: Cn1cnc(N2CCCC(C(=O)Nc3nc4c(F)cc(F)cc4s3)C2)c1] |
| 078 | 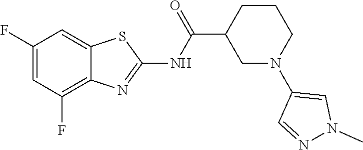 [SMILES: Cn1cc(N2CCCC(C(=O)Nc3nc4c(F)cc(F)cc4s3)C2)cn1] |
| 079 | 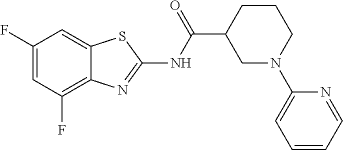 [SMILES: O=C(Nc1nc2c(F)cc(F)cc2s1)C1CCCN(c2ccccn2)C1] |
| 080 | 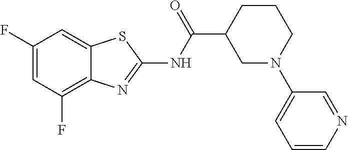 [SMILES: O=C(Nc1nc2c(F)cc(F)cc2s1)C1CCCN(c2cccnc2)C1] |
| 081 | 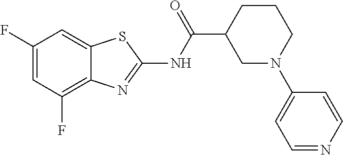 [SMILES: O=C(Nc1nc2c(F)cc(F)cc2s1)C1CCCN(c2ccncc2)C1] |
| 082 | 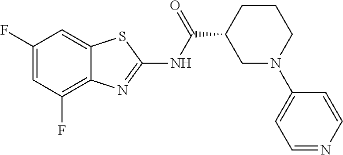 [SMILES: O=C(Nc1nc2c(F)cc(F)cc2s1)[C@@H]1CCCN(c2ccncc2)C1] |
| 083 | 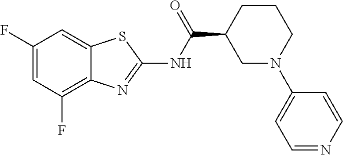 [SMILES: O=C(Nc1nc2c(F)cc(F)cc2s1)[C@H]1CCCN(c2ccncc2)C1] |
| 246 | 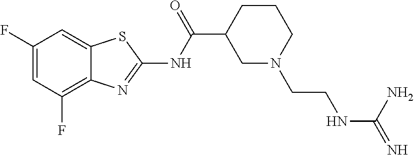 [SMILES: N=C(N)NCCN1CCCC(C(=O)Nc2nc3c(F)cc(F)cc3s2)C1] |
| 040 | 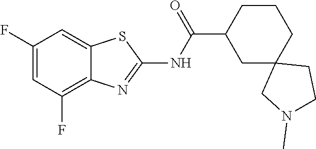 [SMILES: CN1CCC2(CCCC(C(=O)Nc3nc4c(F)cc(F)cc4s3)C2)C1] |
| 084 | 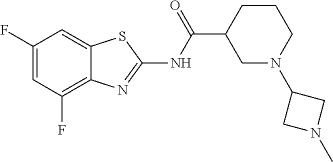 [SMILES: CN1CC(N2CCCC(C(=O)Nc3nc4c(F)cc(F)cc4s3)C2)C1] |
| 085 | 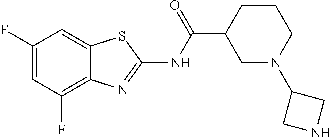 [SMILES: O=C(Nc1nc2c(F)cc(F)cc2s1)C1CCCN(C2CNC2)C1] |
| 195 | 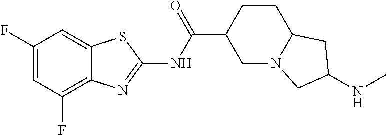 [SMILES: CNC1CC2CCC(C(=O)Nc3nc4c(F)cc(F)cc4s3)CN2C1] |
| 196 | 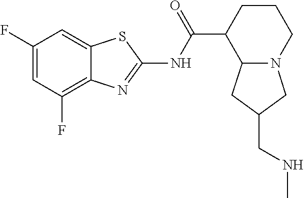 [SMILES: CNCC1CC2C(C(=O)Nc3nc4c(F)cc(F)cc4s3)CCCN2C1] |
| 197 | 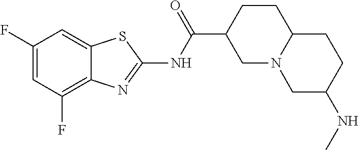 [SMILES: CNC1CCC2CCC(C(=O)Nc3nc4c(F)cc(F)cc4s3)CN2C1] |
| 198 | 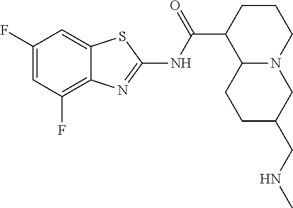 [SMILES: CNCC1CCC2C(C(=O)Nc3nc4c(F)cc(F)cc4s3)CCCN2C1] |
| 199 | 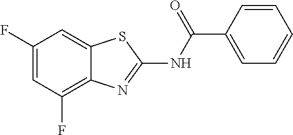 [SMILES: O=C(Nc1nc2c(F)cc(F)cc2s1)c1ccccc1] |
| 200 | 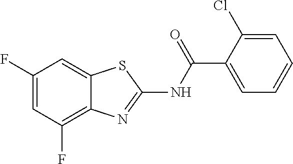 [SMILES: O=C(Nc1nc2c(F)cc(F)cc2s1)c1ccccc1Cl] |
| 201 | 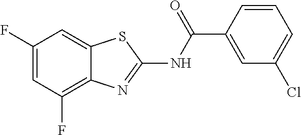 [SMILES: O=C(Nc1nc2c(F)cc(F)cc2s1)c1cccc(Cl)c1] |
| 202 | 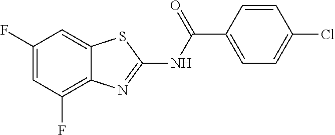 [SMILES: O=C(Nc1nc2c(F)cc(F)cc2s1)c1ccc(Cl)cc1] |
| 203 | 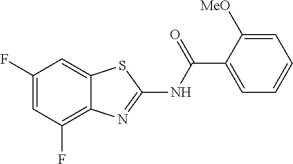 [SMILES: COc1ccccc1C(=O)Nc1nc2c(F)cc(F)cc2s1] |
| 204 | 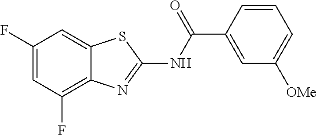 [SMILES: COc1cccc(C(=O)Nc2nc3c(F)cc(F)cc3s2)c1] |
| 205 | 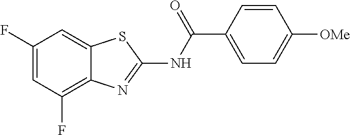 [SMILES: COc1ccc(C(=O)Nc2nc3c(F)cc(F)cc3s2)cc1] |
| 206 | 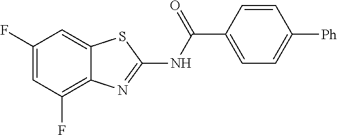 [SMILES: O=C(Nc1nc2c(F)cc(F)cc2s1)c1ccc(-c2ccccc2)cc1] |
| 247 | 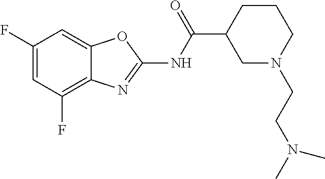 [SMILES: CN(C)CCN1CCCC(C(=O)Nc2nc3c(F)cc(F)cc3o2)C1] |
| 248 | 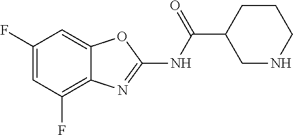 [SMILES: O=C(Nc1nc2c(F)cc(F)cc2o1)C1CCCNC1] |
| 249a | 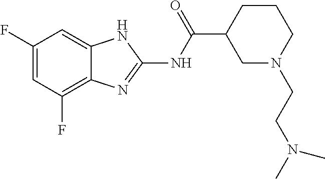 [SMILES: CN(C)CCN1CCCC(C(=O)Nc2nc3c(F)cc(F)cc3[nH]2)C1] |
| 249b | 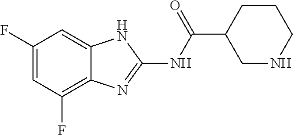 [SMILES: O=C(Nc1nc2c(F)cc(F)cc2[nH]1)C1CCCNC1] |
| 009 | 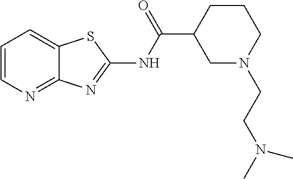 [SMILES: CN(C)CCN1CCCC(C(=O)Nc2nc3ncccc3s2)C1] |
| 014 | 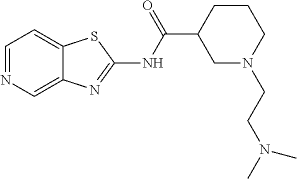 [SMILES: CN(C)CCN1CCCC(C(=O)Nc2nc3cnccc3s2)C1] |
| 031 | 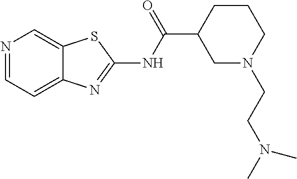 [SMILES: CN(C)CCN1CCCC(C(=O)Nc2nc3ccncc3s2)C1] |
| 036 | 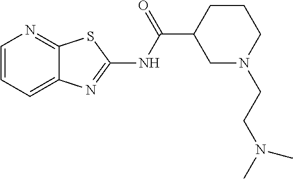 [SMILES: CN(C)CCN1CCCC(C(=O)Nc2nc3cccnc3s2)C1] |
| 144 | 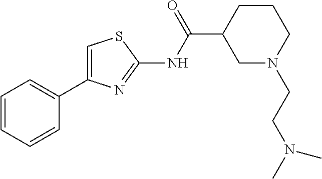 [SMILES: CN(C)CCN1CCCC(C(=O)Nc2nc(-c3ccccc3)cs2)C1] |
| 145 | 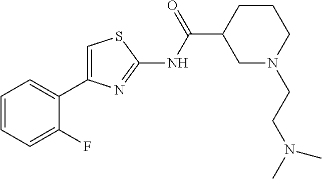 [SMILES: CN(C)CCN1CCCC(C(=O)Nc2nc(-c3ccccc3F)cs2)C1] |
| 146 | 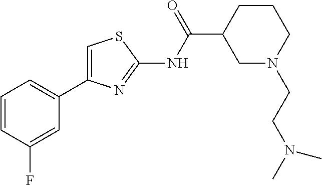 [SMILES: CN(C)CCN1CCCC(C(=O)Nc2nc(-c3cccc(F)c3)cs2)C1] |
| 147 | 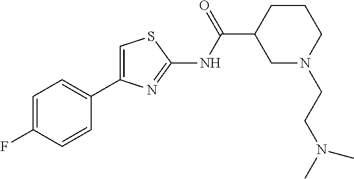 [SMILES: CN(C)CCN1CCCC(C(=O)Nc2nc(-c3ccc(F)cc3)cs2)C1] |
| 148 | 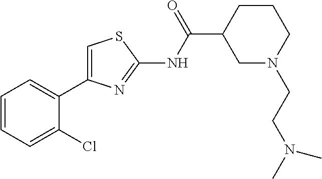 [SMILES: CN(C)CCN1CCCC(C(=O)Nc2nc(-c3ccccc3Cl)cs2)C1] |
| 149 | 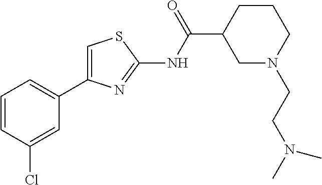 [SMILES: CN(C)CCN1CCCC(C(=O)Nc2nc(-c3cccc(Cl)c3)cs2)C1] |
| 150 | 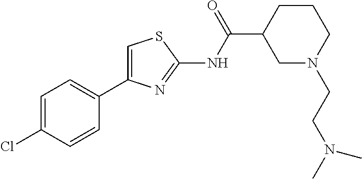 [SMILES: CN(C)CCN1CCCC(C(=O)Nc2nc(-c3ccc(Cl)cc3)cs2)C1] |
| 151 | 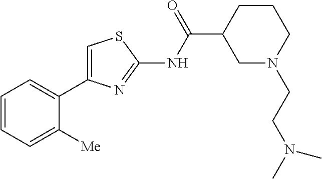 [SMILES: Cc1ccccc1-c1csc(NC(=O)C2CCCN(CCN(C)C)C2)n1] |
| 152 | 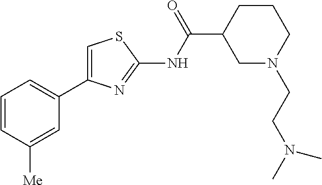 [SMILES: Cc1cccc(-c2csc(NC(=O)C3CCCN(CCN(C)C)C3)n2)c1] |
| 153 | 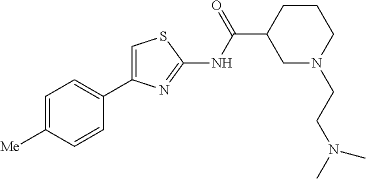 [SMILES: Cc1ccc(-c2csc(NC(=O)C3CCCN(CCN(C)C)C3)n2)cc1] |
| 154 | 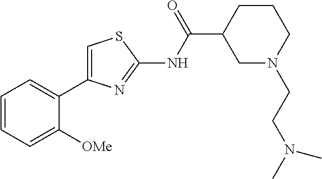 [SMILES: COc1ccccc1-c1csc(NC(=O)C2CCCN(CCN(C)C)C2)n1] |
| 155 | 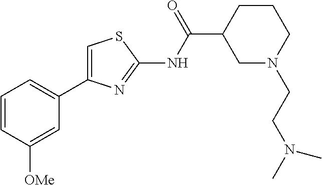 [SMILES: COc1cccc(-c2csc(NC(=O)C3CCCN(CCN(C)C)C3)n2)c1] |
| 156 | 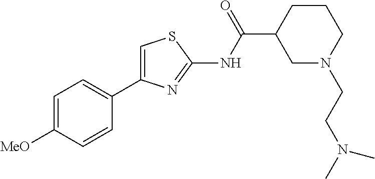 [SMILES: COc1ccc(-c2csc(NC(=O)C3CCCN(CCN(C)C)C3)n2)cc1] |
| 157 | 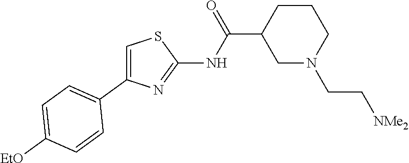 [SMILES: CCOc1ccc(-c2csc(NC(=O)C3CCCN(CCN(C)C)C3)n2)cc1] |
| 158 | 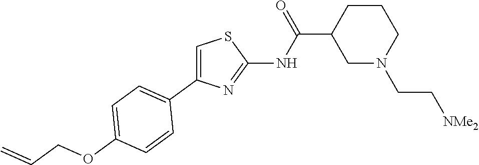 [SMILES: C=CCOc1ccc(-c2csc(NC(=O)C3CCCN(CCN(C)C)C3)n2)cc1] |
| 159 | 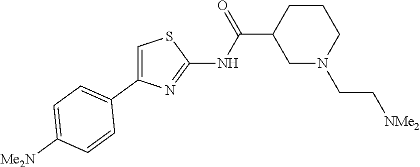 [SMILES: CN(C)CCN1CCCC(C(=O)Nc2nc(-c3ccc(N(C)C)cc3)cs2)C1] |
| 160 | 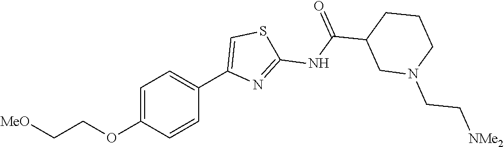 [SMILES: COCCOc1ccc(-c2csc(NC(=O)C3CCCN(CCN(C)C)C3)n2)cc1] |
| 161 | 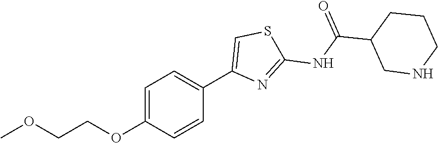 [SMILES: COCCOc1ccc(-c2csc(NC(=O)C3CCCNC3)n2)cc1] |
| 162 | 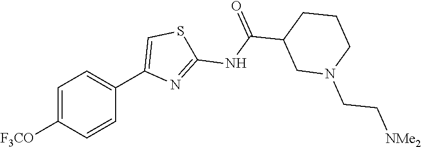 [SMILES: CN(C)CCN1CCCC(C(=O)Nc2nc(-c3ccc(OC(F)(F)F)cc3)cs2)C1] |
| 163 | 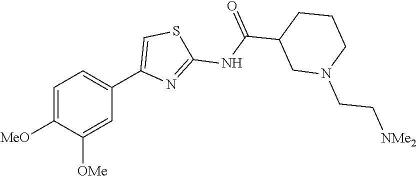 [SMILES: COc1ccc(-c2csc(NC(=O)C3CCCN(CCN(C)C)C3)n2)cc1OC] |
| 164 | 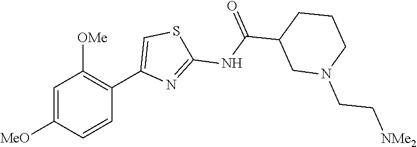 [SMILES: COc1ccc(-c2csc(NC(=O)C3CCCN(CCN(C)C)C3)n2)c(OC)c1] |
| 165 | 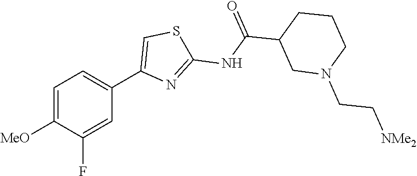 [SMILES: COc1ccc(-c2csc(NC(=O)C3CCCN(CCN(C)C)C3)n2)cc1F] |
| 166 | 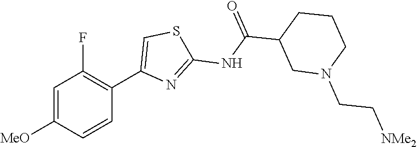 [SMILES: COc1ccc(-c2csc(NC(=O)C3CCCN(CCN(C)C)C3)n2)c(F)c1] |
| 169 | 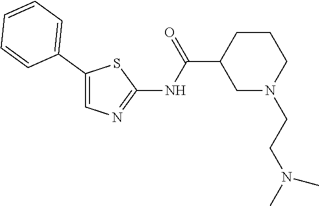 [SMILES: CN(C)CCN1CCCC(C(=O)Nc2ncc(-c3ccccc3)s2)C1] |
| 170 | 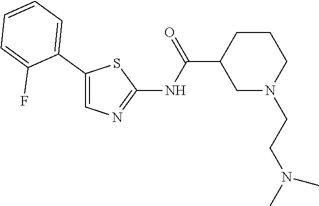 [SMILES: CN(C)CCN1CCCC(C(=O)Nc2ncc(-c3ccccc3F)s2)C1] |
| 171 | 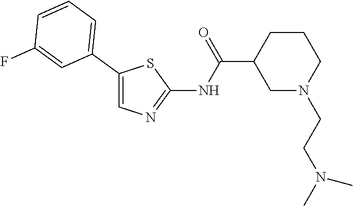 [SMILES: CN(C)CCN1CCCC(C(=O)Nc2ncc(-c3cccc(F)c3)s2)C1] |
| 172 | 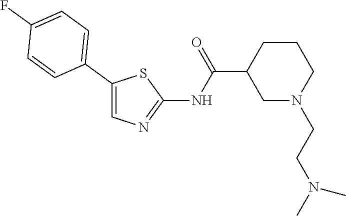 [SMILES: CN(C)CCN1CCCC(C(=O)Nc2ncc(-c3ccc(F)cc3)s2)C1] |
| 173 | 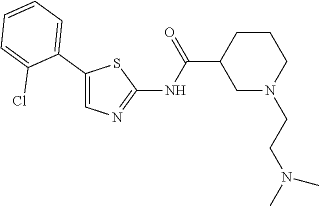 [SMILES: CN(C)CCN1CCCC(C(=O)Nc2ncc(-c3ccccc3Cl)s2)C1] |
| 174 | 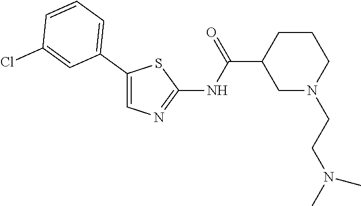 [SMILES: CN(C)CCN1CCCC(C(=O)Nc2ncc(-c3cccc(Cl)c3)s2)C1] |
| 175 | 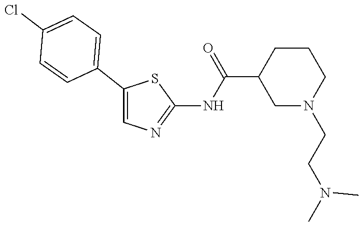 [SMILES: CN(C)CCN1CCCC(C(=O)Nc2ncc(-c3ccc(Cl)cc3)s2)C1] |
| 176 | 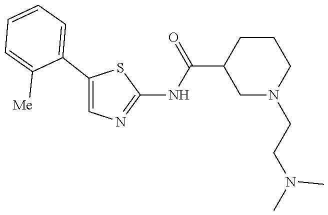 [SMILES: Cc1ccccc1-c1cnc(NC(=O)C2CCCN(CCN(C)C)C2)s1] |
| 177 | 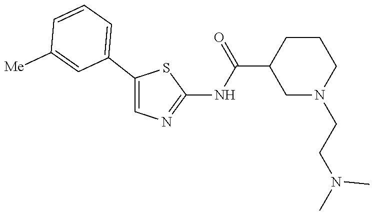 [SMILES: Cc1cccc(-c2cnc(NC(=O)C3CCCN(CCN(C)C)C3)s2)c1] |
| 178 | 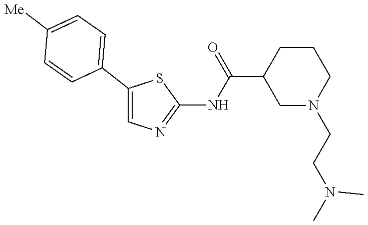 [SMILES: Cc1ccc(-c2cnc(NC(=O)C3CCCN(CCN(C)C)C3)s2)cc1] |
| 179 | 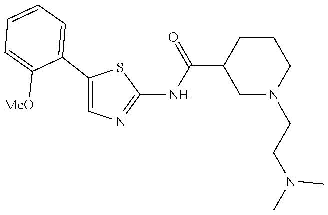 [SMILES: COc1ccccc1-c1cnc(NC(=O)C2CCCN(CCN(C)C)C2)s1] |
| 180 | 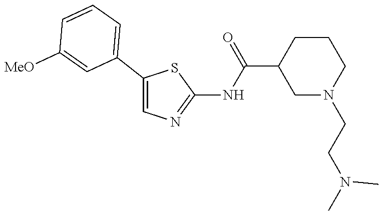 [SMILES: COc1cccc(-c2cnc(NC(=O)C3CCCN(CCN(C)C)C3)s2)c1] |
| 181 | 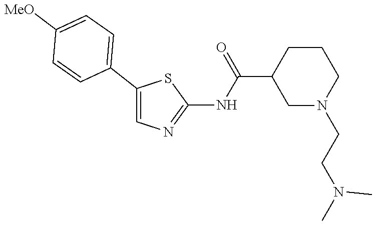 [SMILES: COc1ccc(-c2cnc(NC(=O)C3CCCN(CCN(C)C)C3)s2)cc1] |
| 250 | 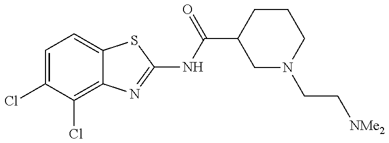 [SMILES: CN(C)CCN1CCCC(C(=O)Nc2nc3c(Cl)c(Cl)ccc3s2)C1] |
| 251 | 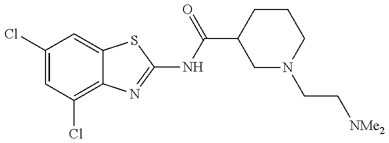 [SMILES: CN(C)CCN1CCCC(C(=O)Nc2nc3c(Cl)cc(Cl)cc3s2)C1] |
| 252 | 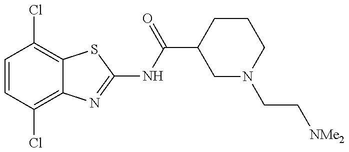 [SMILES: CN(C)CCN1CCCC(C(=O)Nc2nc3c(Cl)ccc(Cl)c3s2)C1] |
| 253 | 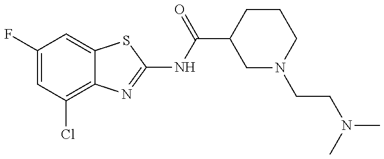 [SMILES: CN(C)CCN1CCCC(C(=O)Nc2nc3c(Cl)cc(F)cc3s2)C1] |
| 254 | 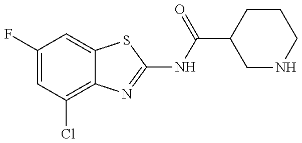 [SMILES: O=C(Nc1nc2c(Cl)cc(F)cc2s1)C1CCCNC1] |
| 255 | 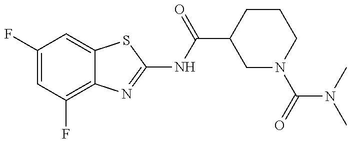 [SMILES: CN(C)C(=O)N1CCCC(C(=O)Nc2nc3c(F)cc(F)cc3s2)C1] |
| 218 | 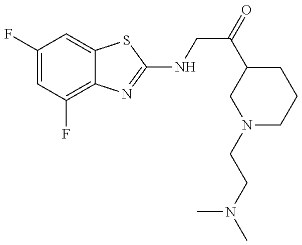 [SMILES: CN(C)CCN1CCCC(C(=O)CNc2nc3c(F)cc(F)cc3s2)C1] |
| 041 | 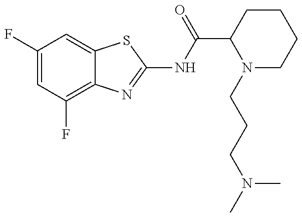 [SMILES: CN(C)CCCN1CCCCC1C(=O)Nc1nc2c(F)cc(F)cc2s1] |
| 219 | 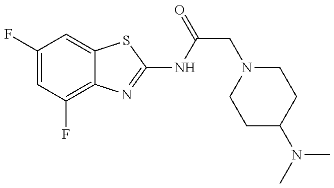 [SMILES: CN(C)C1CCN(CC(=O)Nc2nc3c(F)cc(F)cc3s2)CC1] |
| 220 | 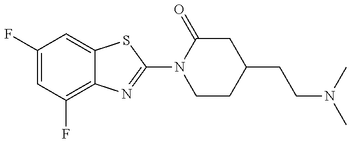 [SMILES: CN(C)CCC1CCN(c2nc3c(F)cc(F)cc3s2)C(=O)C1] |
| 221 | 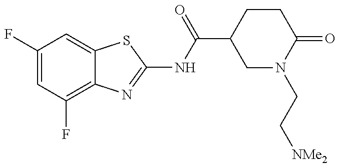 [SMILES: CN(C)CCN1CC(C(=O)Nc2nc3c(F)cc(F)cc3s2)CCC1=O] |
| 222 | 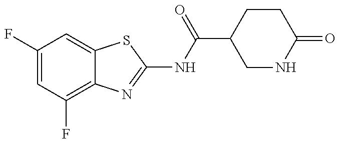 [SMILES: O=C1CCC(C(=O)Nc2nc3c(F)cc(F)cc3s2)CN1] |
| 042 | 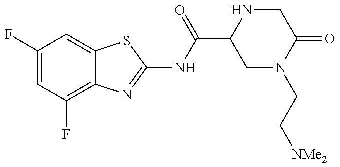 [SMILES: CN(C)CCN1CC(C(=O)Nc2nc3c(F)cc(F)cc3s2)NCC1=O] |
| 043 | 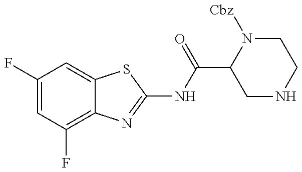 [SMILES: O=C(Nc1nc2c(F)cc(F)cc2s1)C1CNCCN1C(=O)OCc1ccccc1] |
| 044 | 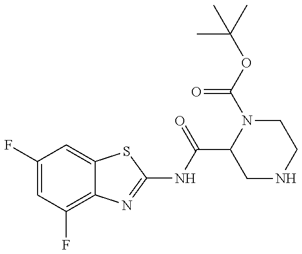 [SMILES: CC(C)(C)OC(=O)N1CCNCC1C(=O)Nc1nc2c(F)cc(F)cc2s1] |
| 045 | 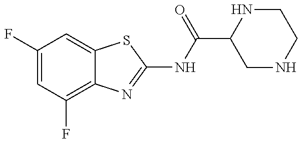 [SMILES: O=C(Nc1nc2c(F)cc(F)cc2s1)C1CNCCN1] |
| 046 | 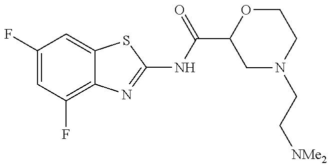 [SMILES: CN(C)CCN1CCOC(C(=O)Nc2nc3c(F)cc(F)cc3s2)C1] |
| 047 | 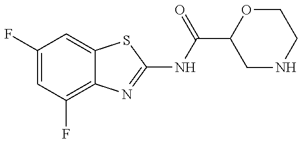 [SMILES: O=C(Nc1nc2c(F)cc(F)cc2s1)C1CNCCO1] |
| 207 | 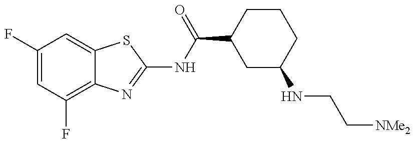 |
| 208 | 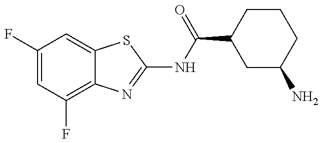 |
| 209 | 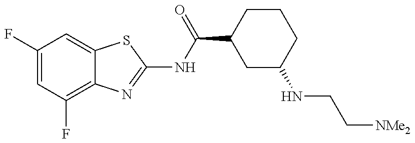 |
| 210T | 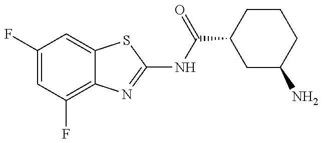 [SMILES: N[C@@H]1CCC[C@@H](C(=O)Nc2nc3c(F)cc(F)cc3s2)C1] |
| 210C | 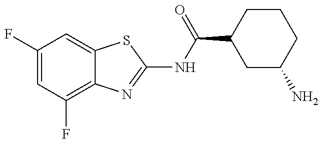 |
| 211 | 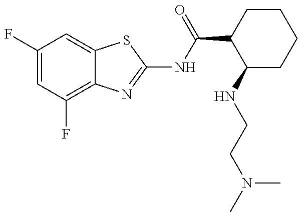 |
| 212 | 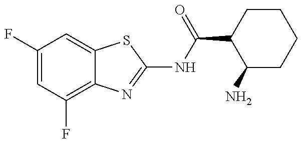 |
| 213 | 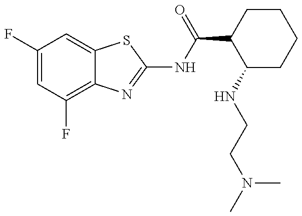 |
| 214 | 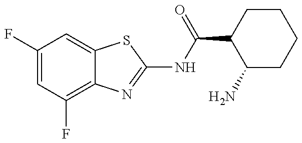 |
| 048 | 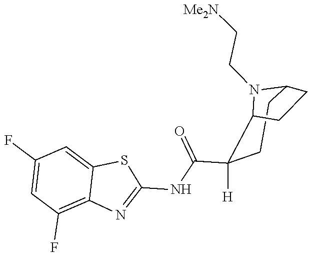 [SMILES: CN(C)CCN1C2CCC(C(=O)Nc3nc4c(F)cc(F)cc4s3)C1CC2] |
| 049 | 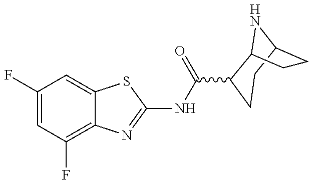 [SMILES: O=C(Nc1nc2c(F)cc(F)cc2s1)C1CCC2CCC1N2] |
| 050 | 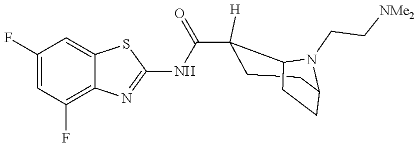 [SMILES: CN(C)CCN1C2CCC(C(=O)Nc3nc4c(F)cc(F)cc4s3)C1CC2] |
| 051 | 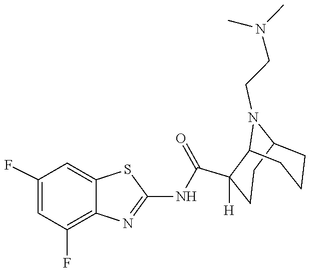 [SMILES: CN(C)CCN1C2CCCC1C(C(=O)Nc1nc3c(F)cc(F)cc3s1)CC2] |
| 053 | 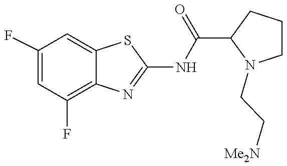 [SMILES: CN(C)CCN1CCCC1C(=O)Nc1nc2c(F)cc(F)cc2s1] |
| 054 | 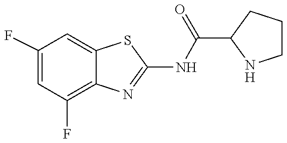 [SMILES: O=C(Nc1nc2c(F)cc(F)cc2s1)C1CCCN1] |
| 055 | 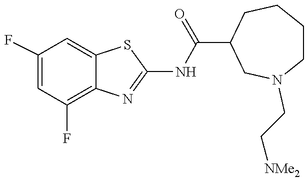 [SMILES: CN(C)CCN1CCCCC(C(=O)Nc2nc3c(F)cc(F)cc3s2)C1] |
| 056 | 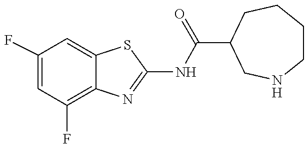 [SMILES: O=C(Nc1nc2c(F)cc(F)cc2s1)C1CCCCNC1] |
| 057 | 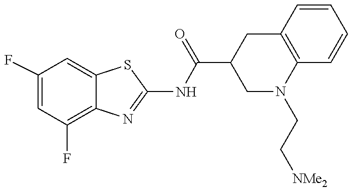 [SMILES: CN(C)CCN1CC(C(=O)Nc2nc3c(F)cc(F)cc3s2)Cc2ccccc21] |
| 058 | 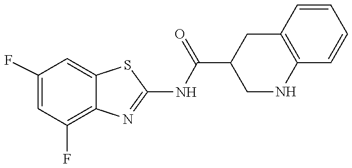 [SMILES: O=C(Nc1nc2c(F)cc(F)cc2s1)C1CNc2ccccc2C1] |
| 059 | 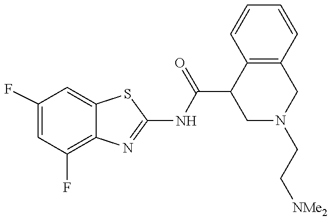 [SMILES: CN(C)CCN1Cc2ccccc2C(C(=O)Nc2nc3c(F)cc(F)cc3s2)C1] |
| 060 | 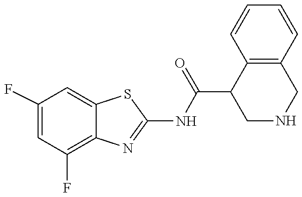 [SMILES: O=C(Nc1nc2c(F)cc(F)cc2s1)C1CNCc2ccccc21] |
| 182 | 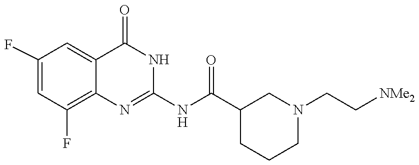 [SMILES: CN(C)CCN1CCCC(C(=O)Nc2nc3c(F)cc(F)cc3c(=O)[nH]2)C1] |
| 183 | 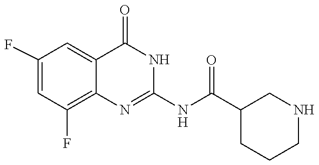 [SMILES: O=C(Nc1nc2c(F)cc(F)cc2c(=O)[nH]1)C1CCCNC1] |
| 184 | 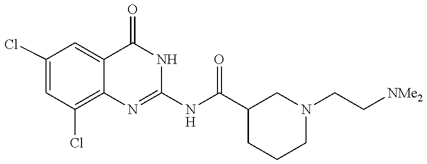 [SMILES: CN(C)CCN1CCCC(C(=O)Nc2nc3c(Cl)cc(Cl)cc3c(=O)[nH]2)C1] |
| 185 | 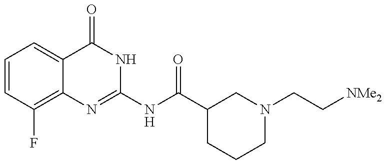 [SMILES: CN(C)CCN1CCCC(C(=O)Nc2nc3c(F)cccc3c(=O)[nH]2)C1] |
| 186 | 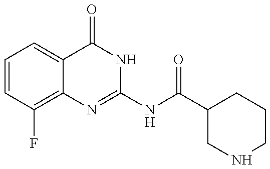 [SMILES: O=C(Nc1nc2c(F)cccc2c(=O)[nH]1)C1CCCNC1] |
| 187 | 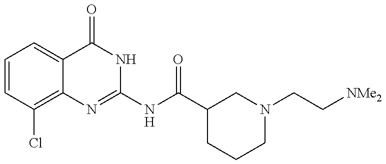 [SMILES: CN(C)CCN1CCCC(C(=O)Nc2nc3c(Cl)cccc3c(=O)[nH]2)C1] |
| 188 | 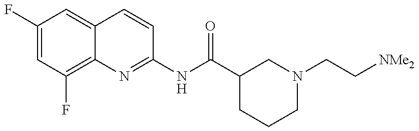 [SMILES: CN(C)CCN1CCCC(C(=O)Nc2ccc3cc(F)cc(F)c3n2)C1] |
| 189 | 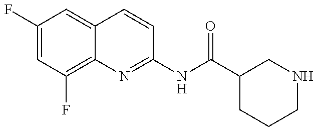 [SMILES: O=C(Nc1ccc2cc(F)cc(F)c2n1)C1CCCNC1] |
| 190 | 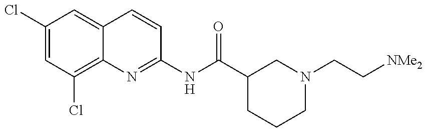 [SMILES: CN(C)CCN1CCCC(C(=O)Nc2ccc3cc(Cl)cc(Cl)c3n2)C1] |
| 191 | 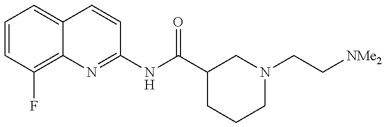 [SMILES: CN(C)CCN1CCCC(C(=O)Nc2ccc3cccc(F)c3n2)C1] |
| 192 | 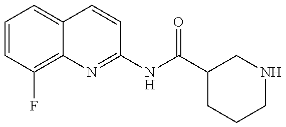 [SMILES: O=C(Nc1ccc2cccc(F)c2n1)C1CCCNC1] |
| 193 | 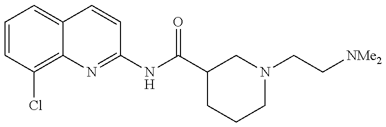 [SMILES: CN(C)CCN1CCCC(C(=O)Nc2ccc3cccc(Cl)c3n2)C1] |
| 194 | 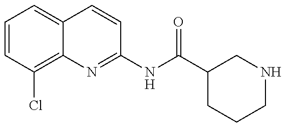 [SMILES: O=C(Nc1ccc2cccc(Cl)c2n1)C1CCCNC1] |
| 256 | 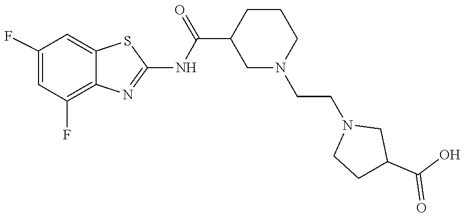 [SMILES: O=C(O)C1CCN(CCN2CCCC(C(=O)Nc3nc4c(F)cc(F)cc4s3)C2)C1] |
| 257 | 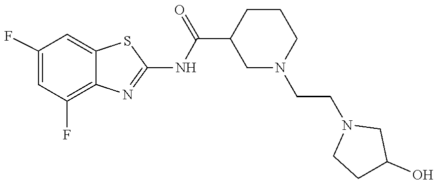 [SMILES: O=C(Nc1nc2c(F)cc(F)cc2s1)C1CCCN(CCN2CCC(O)C2)C1] |
| 258 | 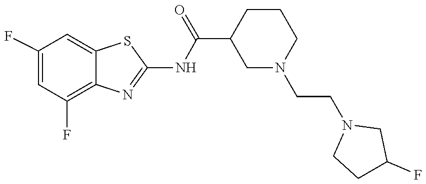 [SMILES: O=C(Nc1nc2c(F)cc(F)cc2s1)C1CCCN(CCN2CCC(F)C2)C1] |
| 259 | 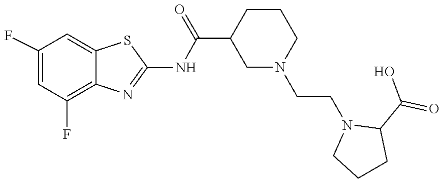 [SMILES: O=C(Nc1nc2c(F)cc(F)cc2s1)C1CCCN(CCN2CCCC2C(=O)O)C1] |
| 260 | 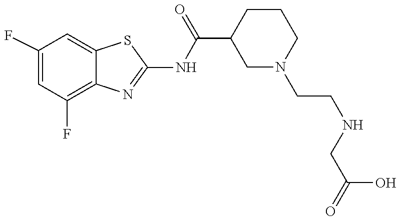 [SMILES: O=C(O)CNCCN1CCCC(C(=O)Nc2nc3c(F)cc(F)cc3s2)C1] |
| 261 | 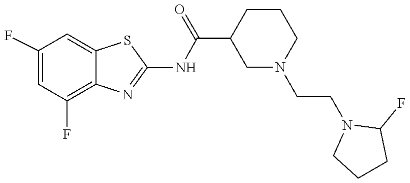 [SMILES: O=C(Nc1nc2c(F)cc(F)cc2s1)C1CCCN(CCN2CCCC2F)C1] |
| 223 | 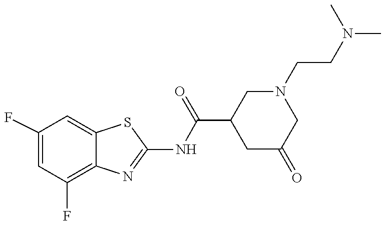 [SMILES: CN(C)CCN1CC(=O)CC(C(=O)Nc2nc3c(F)cc(F)cc3s2)C1] |
| 262 | 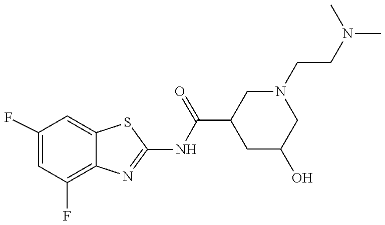 [SMILES: CN(C)CCN1CC(O)CC(C(=O)Nc2nc3c(F)cc(F)cc3s2)C1] |
| 263 | 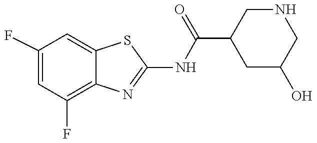 [SMILES: O=C(Nc1nc2c(F)cc(F)cc2s1)C1CNCC(O)C1] |
| 264 | 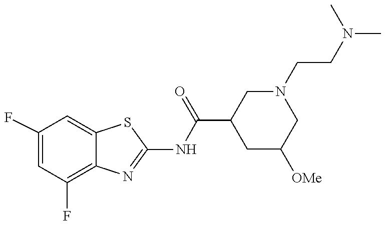 [SMILES: COC1CC(C(=O)Nc2nc3c(F)cc(F)cc3s2)CN(CCN(C)C)C1] |
| 265 | 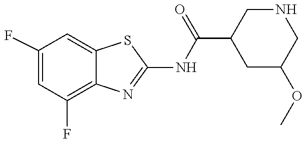 [SMILES: COC1CNCC(C(=O)Nc2nc3c(F)cc(F)cc3s2)C1] |
| 266 | 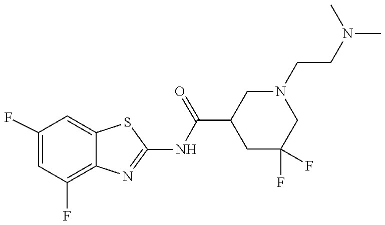 [SMILES: CN(C)CCN1CC(C(=O)Nc2nc3c(F)cc(F)cc3s2)CC(F)(F)C1] |
| 267 | 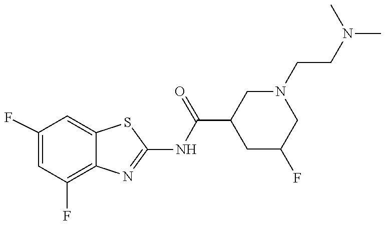 [SMILES: CN(C)CCN1CC(F)CC(C(=O)Nc2nc3c(F)cc(F)cc3s2)C1] |
| 268 | 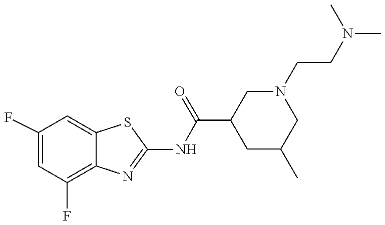 [SMILES: CC1CC(C(=O)Nc2nc3c(F)cc(F)cc3s2)CN(CCN(C)C)C1] |
| 269 | 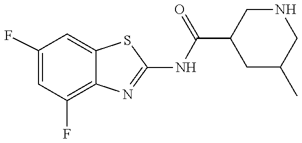 [SMILES: CC1CNCC(C(=O)Nc2nc3c(F)cc(F)cc3s2)C1] |
| 270 | 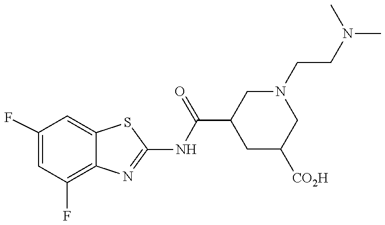 [SMILES: CN(C)CCN1CC(C(=O)O)CC(C(=O)Nc2nc3c(F)cc(F)cc3s2)C1] |
| 271 | 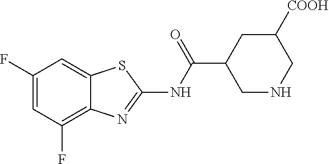 [SMILES: O=C(O)C1CNCC(C(=O)Nc2nc3c(F)cc(F)cc3s2)C1] |
| 272 | 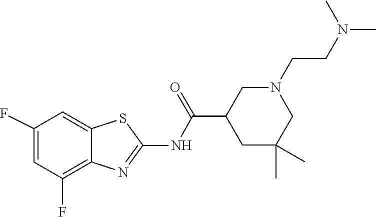 [SMILES: CN(C)CCN1CC(C(=O)Nc2nc3c(F)cc(F)cc3s2)CC(C)(C)C1] |
| 061 | 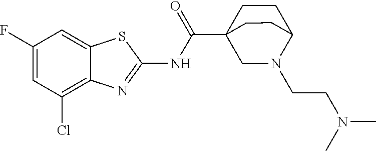 [SMILES: CN(C)CCN1CC2(C(=O)Nc3nc4c(Cl)cc(F)cc4s3)CCC1CC2] |
| 062 | 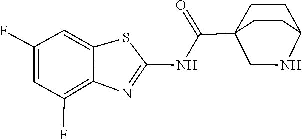 [SMILES: O=C(Nc1nc2c(F)cc(F)cc2s1)C12CCC(CC1)NC2] |
| 273 | 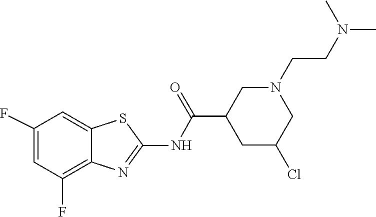 [SMILES: CN(C)CCN1CC(Cl)CC(C(=O)Nc2nc3c(F)cc(F)cc3s2)C1] |
| 086 | 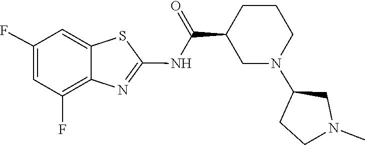 [SMILES: CN1CC[C@@H](N2CCC[C@H](C(=O)Nc3nc4c(F)cc(F)cc4s3)C2)C1] |
| 087 | 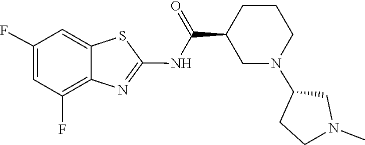 [SMILES: CN1CC[C@H](N2CCC[C@H](C(=O)Nc3nc4c(F)cc(F)cc4s3)C2)C1] |
| 088 | 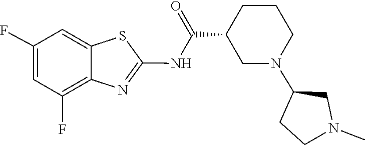 [SMILES: CN1CC[C@@H](N2CCC[C@@H](C(=O)Nc3nc4c(F)cc(F)cc4s3)C2)C1] |
| 089 | 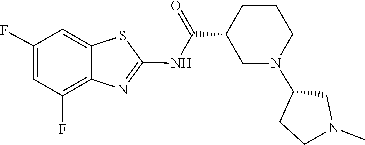 [SMILES: CN1CC[C@H](N2CCC[C@@H](C(=O)Nc3nc4c(F)cc(F)cc4s3)C2)C1] |
| 090 | 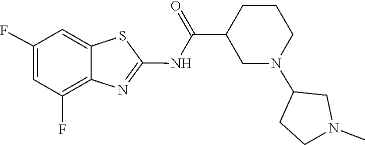 [SMILES: CN1CCC(N2CCCC(C(=O)Nc3nc4c(F)cc(F)cc4s3)C2)C1] |
| 091 | 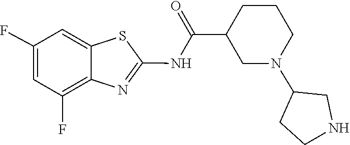 [SMILES: O=C(Nc1nc2c(F)cc(F)cc2s1)C1CCCN(C2CCNC2)C1] |
| 092 | 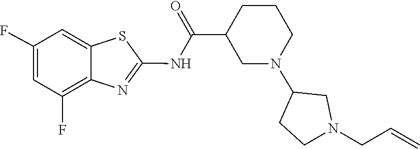 [SMILES: C=CCN1CCC(N2CCCC(C(=O)Nc3nc4c(F)cc(F)cc4s3)C2)C1] |
| 093 | 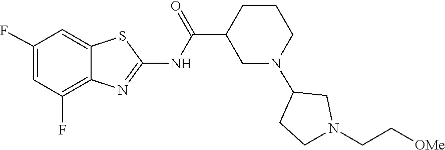 [SMILES: COCCN1CCC(N2CCCC(C(=O)Nc3nc4c(F)cc(F)cc4s3)C2)C1] |
| 094 | 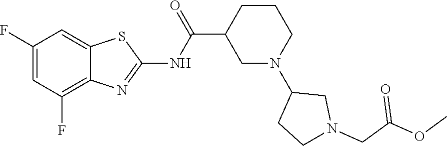 [SMILES: COC(=O)CN1CCC(N2CCCC(C(=O)Nc3nc4c(F)cc(F)cc4s3)C2)C1] |
| 095 | 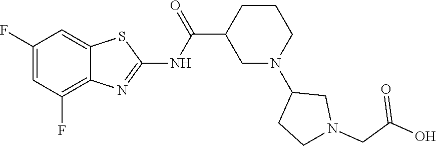 [SMILES: O=C(O)CN1CCC(N2CCCC(C(=O)Nc3nc4c(F)cc(F)cc4s3)C2)C1] |
| 096 | 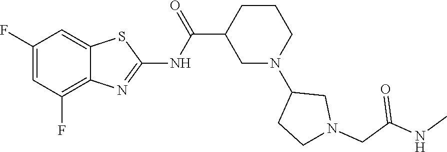 [SMILES: CNC(=O)CN1CCC(N2CCCC(C(=O)Nc3nc4c(F)cc(F)cc4s3)C2)C1] |
| 097 | 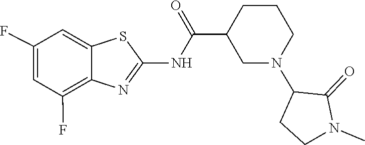 [SMILES: CN1CCC(N2CCCC(C(=O)Nc3nc4c(F)cc(F)cc4s3)C2)C1=O] |
| 098 | 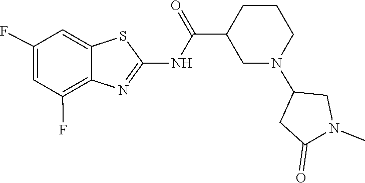 [SMILES: CN1CC(N2CCCC(C(=O)Nc3nc4c(F)cc(F)cc4s3)C2)CC1=O] |
| 099 | 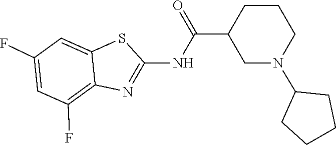 [SMILES: O=C(Nc1nc2c(F)cc(F)cc2s1)C1CCCN(C2CCCC2)C1] |
| 215 | 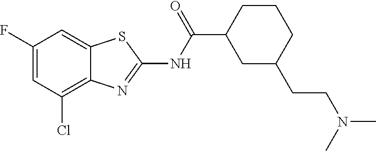 [SMILES: CN(C)CCC1CCCC(C(=O)Nc2nc3c(Cl)cc(F)cc3s2)C1] |
| 100 | 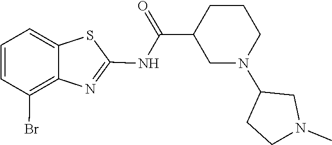 [SMILES: CN1CCC(N2CCCC(C(=O)Nc3nc4c(Br)cccc4s3)C2)C1] |
| 101 | 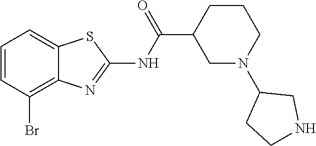 [SMILES: O=C(Nc1nc2c(Br)cccc2s1)C1CCCN(C2CCNC2)C1] |
| 102 | 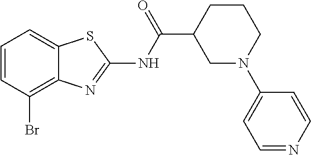 [SMILES: O=C(Nc1nc2c(Br)cccc2s1)C1CCCN(c2ccncc2)C1] |
| 167 | 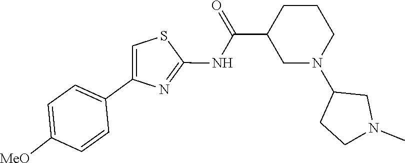 [SMILES: COc1ccc(-c2csc(NC(=O)C3CCCN(C4CCN(C)C4)C3)n2)cc1] |
| 168 | 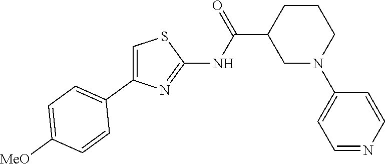 [SMILES: COc1ccc(-c2csc(NC(=O)C3CCCN(c4ccncc4)C3)n2)cc1] |
| 103 | 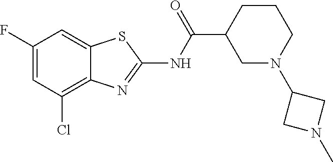 [SMILES: CN1CC(N2CCCC(C(=O)Nc3nc4c(Cl)cc(F)cc4s3)C2)C1] |
| 104 | 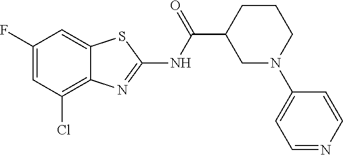 [SMILES: O=C(Nc1nc2c(Cl)cc(F)cc2s1)C1CCCN(c2ccncc2)C1] |
| 105 | 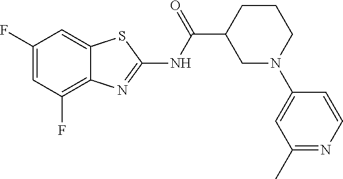 [SMILES: Cc1cc(N2CCCC(C(=O)Nc3nc4c(F)cc(F)cc4s3)C2)ccn1] |
| 106 | 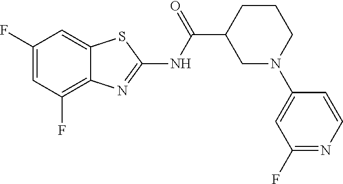 [SMILES: O=C(Nc1nc2c(F)cc(F)cc2s1)C1CCCN(c2ccnc(F)c2)C1] |
| 107 | 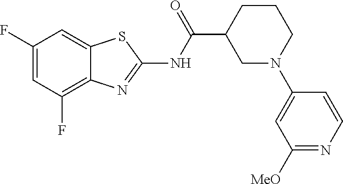 [SMILES: COc1cc(N2CCCC(C(=O)Nc3nc4c(F)cc(F)cc4s3)C2)ccn1] |
| 108 | 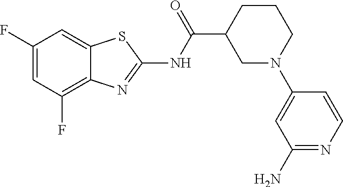 [SMILES: Nc1cc(N2CCCC(C(=O)Nc3nc4c(F)cc(F)cc4s3)C2)ccn1] |
| 109 | 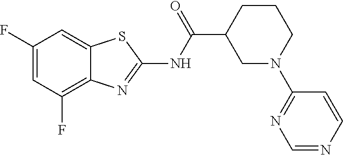 [SMILES: O=C(Nc1nc2c(F)cc(F)cc2s1)C1CCCN(c2ccncn2)C1] |
| 110 | 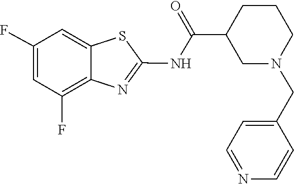 [SMILES: O=C(Nc1nc2c(F)cc(F)cc2s1)C1CCCN(Cc2ccncc2)C1] |
| 111 | 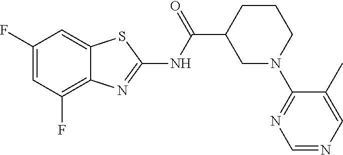 [SMILES: Cc1cncnc1N1CCCC(C(=O)Nc2nc3c(F)cc(F)cc3s2)C1] |
| 112 | 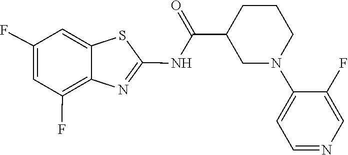 [SMILES: O=C(Nc1nc2c(F)cc(F)cc2s1)C1CCCN(c2ccncc2F)C1] |
| 113 | 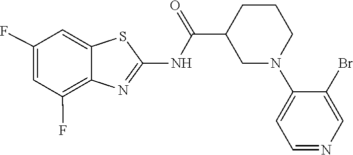 [SMILES: O=C(Nc1nc2c(F)cc(F)cc2s1)C1CCCN(c2ccncc2Br)C1] |
| 114 | 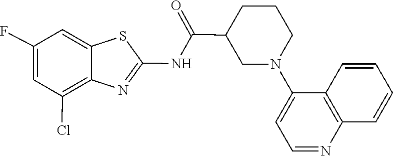 [SMILES: O=C(Nc1nc2c(Cl)cc(F)cc2s1)C1CCCN(c2ccnc3ccccc23)C1] |
| 115 | 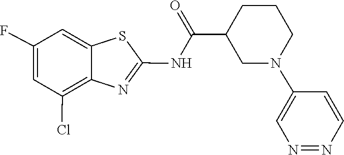 [SMILES: O=C(Nc1nc2c(Cl)cc(F)cc2s1)C1CCCN(c2ccnnc2)C1] |
| 116 | 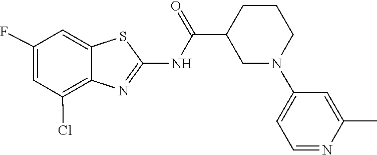 [SMILES: Cc1cc(N2CCCC(C(=O)Nc3nc4c(Cl)cc(F)cc4s3)C2)ccn1] |
| 117 | 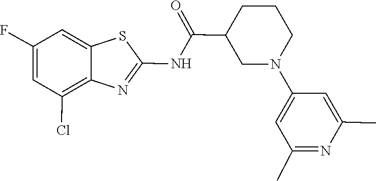 [SMILES: Cc1cc(N2CCCC(C(=O)Nc3nc4c(Cl)cc(F)cc4s3)C2)cc(C)n1] |
| 118 | 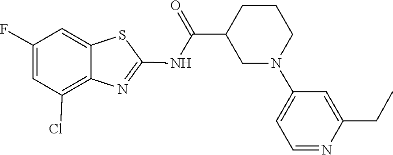 [SMILES: CCc1cc(N2CCCC(C(=O)Nc3nc4c(Cl)cc(F)cc4s3)C2)ccn1] |
| 119 | 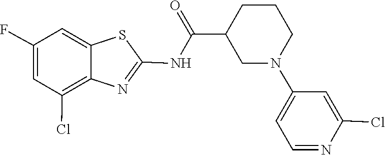 [SMILES: O=C(Nc1nc2c(Cl)cc(F)cc2s1)C1CCCN(c2ccnc(Cl)c2)C1] |
| 120 | 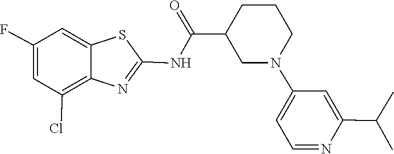 [SMILES: CC(C)c1cc(N2CCCC(C(=O)Nc3nc4c(Cl)cc(F)cc4s3)C2)ccn1] |
| 121 | 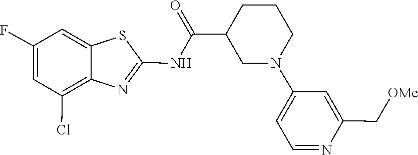 [SMILES: COCc1cc(N2CCCC(C(=O)Nc3nc4c(Cl)cc(F)cc4s3)C2)ccn1] |
| 122 | 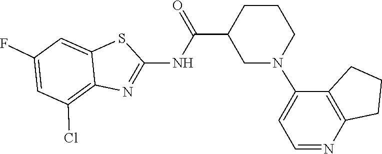 [SMILES: O=C(Nc1nc2c(Cl)cc(F)cc2s1)C1CCCN(c2ccnc3c2CCC3)C1] |
| 123 | 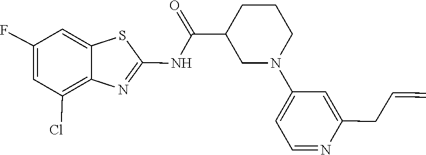 [SMILES: C=CCc1cc(N2CCCC(C(=O)Nc3nc4c(Cl)cc(F)cc4s3)C2)ccn1] |
| 124 | 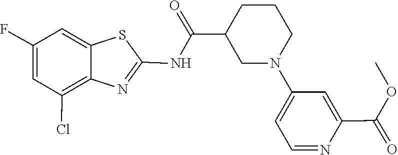 [SMILES: COC(=O)c1cc(N2CCCC(C(=O)Nc3nc4c(Cl)cc(F)cc4s3)C2)ccn1] |
| 125 | 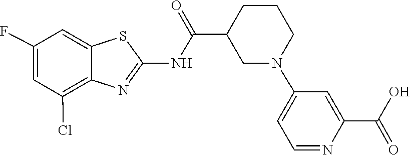 [SMILES: O=C(O)c1cc(N2CCCC(C(=O)Nc3nc4c(Cl)cc(F)cc4s3)C2)ccn1] |
| 126 | 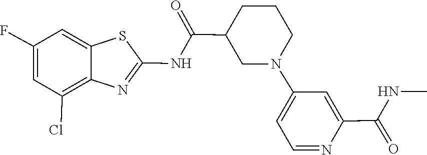 [SMILES: CNC(=O)c1cc(N2CCCC(C(=O)Nc3nc4c(Cl)cc(F)cc4s3)C2)ccn1] |
| 127 | 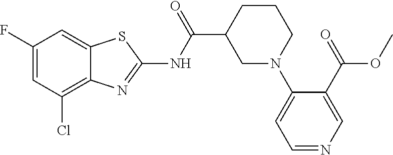 [SMILES: COC(=O)c1cnccc1N1CCCC(C(=O)Nc2nc3c(Cl)cc(F)cc3s2)C1] |
| 128 | 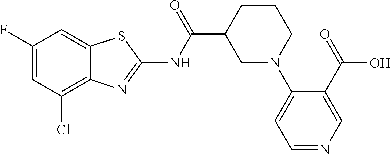 [SMILES: O=C(O)c1cnccc1N1CCCC(C(=O)Nc2nc3c(Cl)cc(F)cc3s2)C1] |
| 129 | 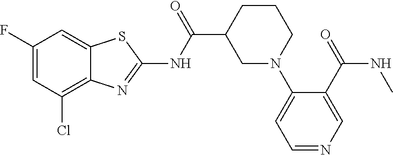 [SMILES: CNC(=O)c1cnccc1N1CCCC(C(=O)Nc2nc3c(Cl)cc(F)cc3s2)C1] |
| 274 | 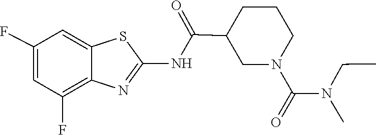 [SMILES: CCN(C)C(=O)N1CCCC(C(=O)Nc2nc3c(F)cc(F)cc3s2)C1] |
| 275 | 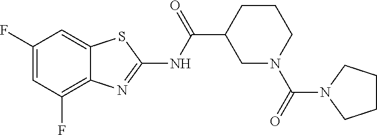 [SMILES: O=C(Nc1nc2c(F)cc(F)cc2s1)C1CCCN(C(=O)N2CCCC2)C1] |
| 276 | 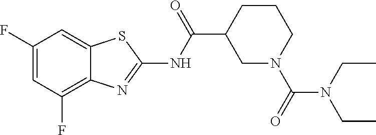 [SMILES: CCN(CC)C(=O)N1CCCC(C(=O)Nc2nc3c(F)cc(F)cc3s2)C1] |
| 277 | 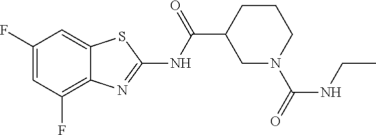 [SMILES: CCNC(=O)N1CCCC(C(=O)Nc2nc3c(F)cc(F)cc3s2)C1] |
| 278 | 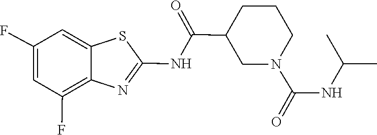 [SMILES: CC(C)NC(=O)N1CCCC(C(=O)Nc2nc3c(F)cc(F)cc3s2)C1] |
| 279 | 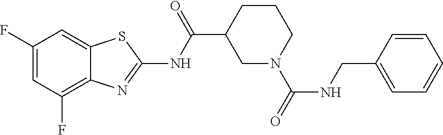 [SMILES: O=C(Nc1nc2c(F)cc(F)cc2s1)C1CCCN(C(=O)NCc2ccccc2)C1] |
| 280 | 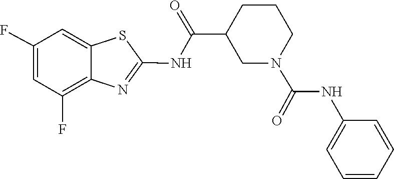 [SMILES: O=C(Nc1nc2c(F)cc(F)cc2s1)C1CCCN(C(=O)Nc2ccccc2)C1] |
| 281 | 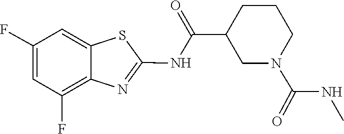 [SMILES: CNC(=O)N1CCCC(C(=O)Nc2nc3c(F)cc(F)cc3s2)C1] |
| 282 | 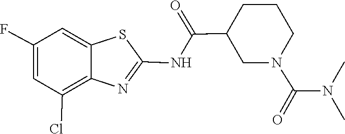 [SMILES: CN(C)C(=O)N1CCCC(C(=O)Nc2nc3c(Cl)cc(F)cc3s2)C1] |
| 130 | 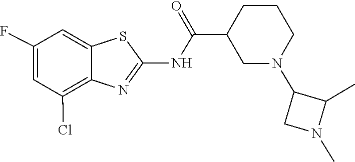 [SMILES: CC1C(N2CCCC(C(=O)Nc3nc4c(Cl)cc(F)cc4s3)C2)CN1C] |
| 131 | 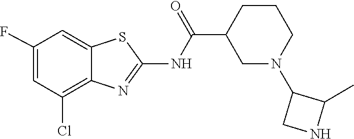 [SMILES: CC1NCC1N1CCCC(C(=O)Nc2nc3c(Cl)cc(F)cc3s2)C1] |
| 132 | 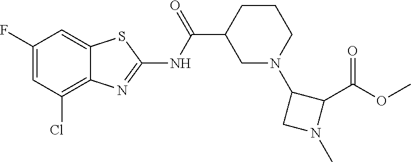 [SMILES: COC(=O)C1C(N2CCCC(C(=O)Nc3nc4c(Cl)cc(F)cc4s3)C2)CN1C] |
| 133 | 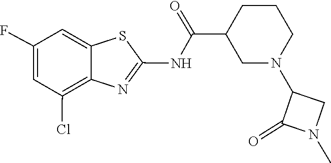 [SMILES: CN1CC(N2CCCC(C(=O)Nc3nc4c(Cl)cc(F)cc4s3)C2)C1=O] |
| 134 | 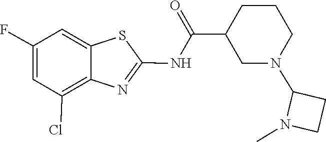 [SMILES: CN1CCC1N1CCCC(C(=O)Nc2nc3c(Cl)cc(F)cc3s2)C1] |
| 135 | 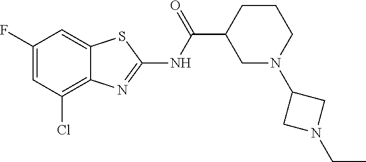 [SMILES: CCN1CC(N2CCCC(C(=O)Nc3nc4c(Cl)cc(F)cc4s3)C2)C1] |
| 136 | 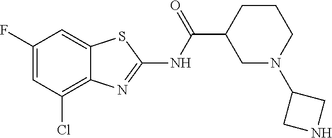 [SMILES: O=C(Nc1nc2c(Cl)cc(F)cc2s1)C1CCCN(C2CNC2)C1] |
| 137 | 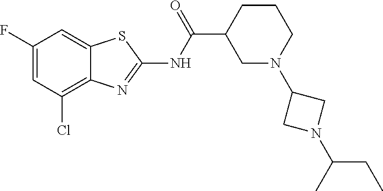 [SMILES: CCC(C)N1CC(N2CCCC(C(=O)Nc3nc4c(Cl)cc(F)cc4s3)C2)C1] |
| 138 | 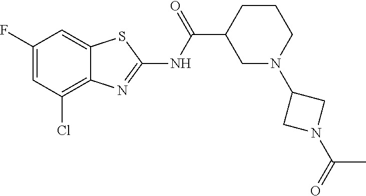 [SMILES: CC(=O)N1CC(N2CCCC(C(=O)Nc3nc4c(Cl)cc(F)cc4s3)C2)C1] |
| 139 | 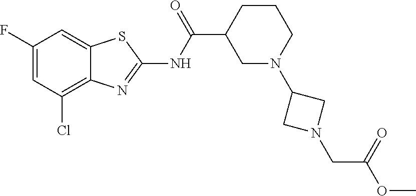 [SMILES: COC(=O)CN1CC(N2CCCC(C(=O)Nc3nc4c(Cl)cc(F)cc4s3)C2)C1] |
| 283 | 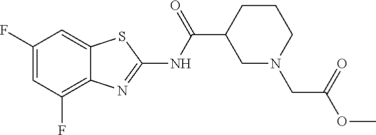 [SMILES: COC(=O)CN1CCCC(C(=O)Nc2nc3c(F)cc(F)cc3s2)C1] |
| 140 | 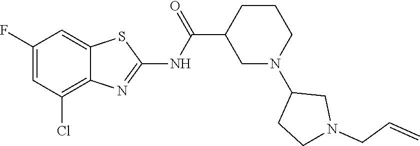 [SMILES: C=CCN1CCC(N2CCCC(C(=O)Nc3nc4c(Cl)cc(F)cc4s3)C2)C1] |
| 141 | 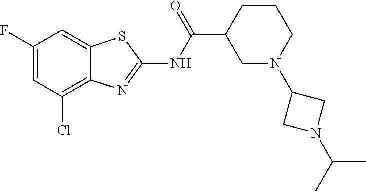 [SMILES: CC(C)N1CC(N2CCCC(C(=O)Nc3nc4c(Cl)cc(F)cc4s3)C2)C1] |
| 142 | 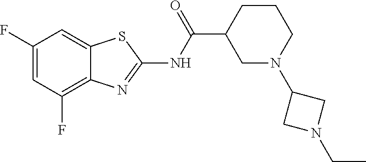 [SMILES: CCN1CC(N2CCCC(C(=O)Nc3nc4c(F)cc(F)cc4s3)C2)C1] |

Exemplary syntheses are shown below. Compounds disclosed herein may be made in analogous fashion.

### Example A

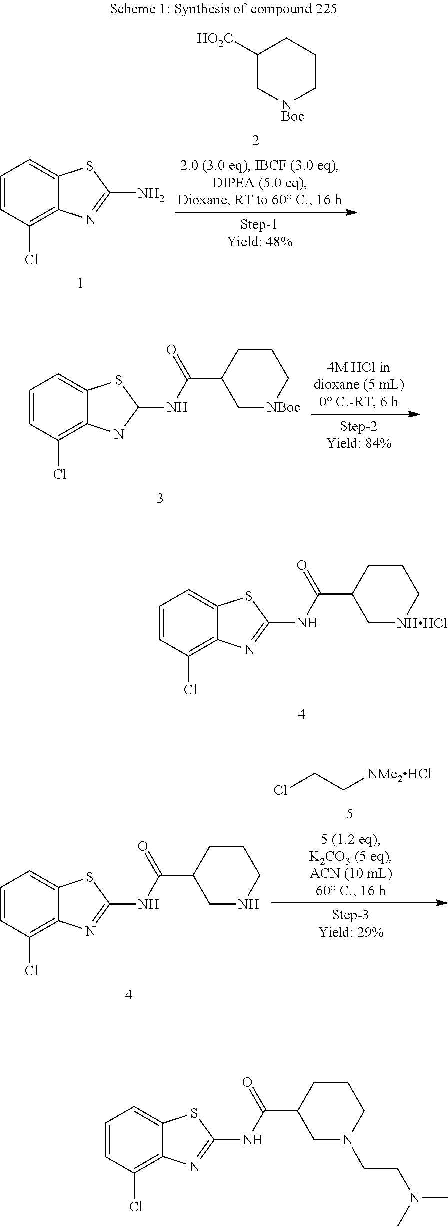

### Step-1: Synthesis of tert-butyl 3-[(4-chloro-1,3-benzothiazol-2-yl)carbamoyl]piperidine-1-carboxylate

To a stirred solution 1-[(tert-butoxy)carbonyl]piperidine-3-carboxylic acid (1.12 g, 4.87 mmol) and DIPEA (1.5 mL, 8.12 mmol) in 1,4-dioxane (10 mL) was added 2-methylpropyl carbonochloridate (666 mg, 4.87 mmol) and stirred for 30 min at room temperature. Then 4-chloro-1,3-benzothiazol-2-amine (0.3 g, 1.62 mmol) was added to the above suspension and reaction mixture was stirred at 60° C. for overnight. Progress of reaction was monitored by the TLC. Reaction mixture was diluted with water and extracted with ethyl acetate, organic layer was further washed with saturated NaHCO₃ solution followed by brine solution. The organic layer was dried over MgSO₄, filtered and concentrated in vacuo. The residue was purified by flash column chromatography to obtain tert-butyl 3-[(4-chloro-1,3-benzothiazol-2-yl)carbamoyl]piperidine-1-carboxylate (0.32 g) 48% Yield with 84% purity.

Analytical Data

```
  LCMS calculated: m/z 396.11 [M + H]⁺
  LCMS observed: m/z 396.23 [M + H]⁺
```

### Step-2: Synthesis of N-(4-chloro-1,3-benzothiazol-2-yl)piperidine-3-carboxamide

To a stirred solution tert-butyl 3-[(4-chloro-1,3-benzothiazol-2-yl)carbamoyl]piperidine-1-carboxylate (310 mg, 783 μmol) in 1,4-dioxane (3 mL) was added 4.0 M HCl in dioxane (2 mL) at 0° C. and further stirred at for 6 h. The progress of the reaction was monitored by TLC and LCMS. The reaction mixture was concentrated under reduced pressure and further triturated by diethyl ether afford N-(4-chloro-1,3-benzothiazol-2-yl)piperidine-3-carboxamide (220 mg) with 84% yield and 97% purity.

Analytical Data

| | |
|---|---|
| LCMS calculated: | m/z 296.05 [M + H]⁺ |
| LCMS observed: | m/z 296.07 [M + H]⁺ |

### Step-3: Synthesis of N-(4-chloro-1,3-benzothiazol-2-yl)-1-[2-(dimethylamino)ethyl]piperidine-3-carboxamide

Hydrogen (2-chloroethyl)dimethylamine chloride (80.3 mg, 558 μmol) was added to the stirred suspension of N-(4-chloro-1,3-benzothiazol-2-yl)piperidine-3-carboxamide (150 mg, 507 μmol) in acetonitrile (652 μL, 12.5 mmol), dipotassium carbonate (210 mg, 1.52 mmol), Resulting mixture was stirred at 60° C. for overnight and the progress of reaction was monitored by TLC and LCMS. Reaction mixture was filtered through celite and concentrated under vacuum to get 170 mg (60% purity) crude mixture which was subjected to the prep-HPLC to obtain the desired N-(4-chloro-1,3-benzothiazol-2-yl)-1-[2(dimethylamino)ethyl]piperidine-3-carboxamide (55 mg, 150 μmol) TFA salt as white in 29% yield with 99% purity.

Analytical Data

| | |
|---|---|
| LCMS calculated: | m/z 367.13 [M + H]⁺ |
| LCMS observed: | m/z 367.17 [M + H]⁺ |

¹H NMR (MeOD, 400 MHz) δ: 7.81 (d, 7.2 Hz, 1H), 7.47 (d, 7.2 Hz, 1H), 7.28 (t, 8 Hz, 1H), 3.43-3.31 (m, 2H), 3.10-2.90 (m, 12H), 2.22-2.12 (m, 1H), 2.10-2.0 (m, 1H), 1.90-1.75 (m, 3H) ppm.

### Example B

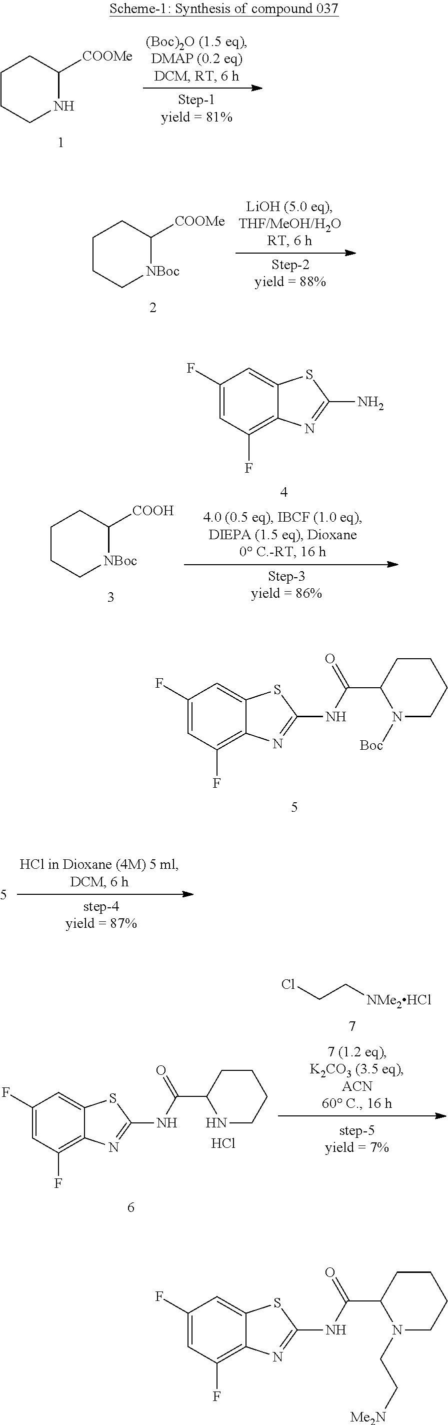

### Step-1: Synthesis of 1-tert-butyl 2-methyl piperidine-1,2-dicarboxylate

To the Solution of methyl piperidine-2-carboxylate hydrochloride (5 g, 27.8 mmol) and 4-(dimethylamino)pyridin-1-ium (4.11 g, 33.4 mmol) in dichloromethane (50 mL), di-tert-butyl dicarbonate (7.29 g, 33.4 mmol) was added dropwise at 0° C. After completion of the addition reaction mixture was stir at T for 6 h. After Complete consumption of Starting material mixture was diluted with water (20 ml) and extract the organic layer and dried over Na2SO4 followed by concentrated under reduced Pressure. Resulted Crude was subjected to the LCMS analysis and flash Chromatography to obtain 1-tert-butyl 2-methyl piperidine-1,2-dicarboxylate (5.5 g, 22.6 mmol) in 81% yield with 98% purity.

Analytical Data

| | |
|---|---|
| LCMS calculated: | m/z 244.15 [M + H]⁺ |
| LCMS observed: | m/z 244.17 [M + H]⁺ |

### Step-2: Synthesis of 1-[(tert-butoxy)carbonyl]piperidine-2-carboxylic acid

LiOH·H2O (2.07 g, 49.3 mmol) was added to the solution of 1-tert-butyl 2-methyl piperidine-1,2-dicarboxylate (3 g, 12.3 mmol) in THF (6 mL), H2O (1 mL) and MeOH (3 mL) at room temperature and resulting solution was stir for 6 h. Reaction was monitored with TLC and after complete consumption of starting material, the solution was concentrated under reduced pressure and diluted with DCM (20 ml) and washed with water (20 ml), aqueous layer was acidified with citric acid solution to PH-2 and extracted with 10% MeOH in DCM solution. organic layer was extracted and dried over Na2SO4 followed by concentrated under reduced pressure to get the desired 1-[(tert-butoxy)carbonyl]piperidine-2-carboxylic acid (2.5 g, 10.9 mmol) with 88% yield and 99% purity.

Analytical Data

| | |
|---|---|
| LCMS calculated: | m/z 228.12 [M − H]⁺ |
| LCMS observed: | m/z 228.20 [M − H]⁺ |

### Step-3: Synthesis of tert-butyl 3-[(4,6-difluoro-1,3-benzothiazol-2-yl)carbamoyl]-3-methylpiperidine-1-carboxylate

2-methylpropyl carbonochloridate (550 mg, 4.03 mmol) was added to a mixture of 1-[(tert-butoxy)carbonyl]piperidine-2-carboxylic acid (924 mg, 4.03 mmol) and DIPEA (1.2 mL, 6.71 mmol) in 1,4-dioxane (10 mL) at RT and resulted mixture was stirred for 15 min. Then 4,6-difluoro-1,3-benzothiazol-2-amine (250 mg, 1.34 mmol) was added to the solution and reaction mixture was warmed up to 60 0° C. and stirring continued for overnight. After completion of the reaction (monitored by TLC and LCMS), mixture was diluted ethyl acetate and washed with saturated NaHCO₃ solution followed by brine solution. Organic layer was extracted, dried over NaSO4 and concentrated under reduced pressure followed by the subjected to the silica get flash chromatography to obtain the desired tert-butyl 2-[(4,7-difluoro-1,3-benzothiazol-2-yl)carbamoyl]piperidine-1-carboxylate (0.1 g, 252 gmol) (97 yield) with 86% purity (LCMS)

Analytical Data

| | |
|---|---|
| LCMS calculated: | m/z 398.13 [M + H]⁺ |
| LCMS observed: | m/z 398.13 [M + H]⁺ |

### Step-4: Synthesis of N-(4,6-difluoro-1,3-benzothiazol-2-yl)piperidine-2-carboxamide hydrochloride

To a stirred solution of tert-butyl 2-[(4,6-difluoro-1,3-benzothiazol-2-yl)carbamoyl]piperidine-1-carboxylate (0.4 g, 1.01 mmol) in 1,4-dioxane (5 mL) was added 4M HCl in dioxane (5 mL) then stirred the mixture at room temperature after complete consumption of starting material (Monitored by TLC) reaction mixture was washed and triturated with ether to get N-(4,6-difluoro-1,3-benzothiazol-2-yl)piperidine-2-carboxamide hydrochloride (310 mg, 929 μmol) 92% yield with 87% Purity (LCMS).

Analytical Data

| | |
|---|---|
| LCMS calculated: | m/z 298.07 [M + H]⁺ |
| LCMS observed: | m/z 298.15 [M + H]⁺ |

### Step-5: Synthesis of N-(4,6-difluoro-1,3-benzothiazol-2-yl)-1-[2-(dimethylamino)ethyl]-3-methylpiperidine-2-carboxamide

To a stirred solution of N-(4,6-difluoro-1,3-benzothiazol-2-yl)-3-methyl-1-(2,2,2-trifluoroacetyl)piperidine-3-carboxamide (0.1 g, 245 μmol) and dipotassium carbonate (119 mg, 3.5 eq., 859 μmol) in acetonitrile (5 mL, 95.7 mmol), was added (2-chloroethyl)dimethylamine hydrochloride (42.4 mg, 1.2 eq., 295 μmol). and stirred at 60° C. for 16 h. Progress of reaction was monitored by TLC and LCMS. Reaction mixture was filtered through celite and concentrated under reduced pressure and further purified by prep-HPLC to afford N-(4,6-difluoro-1,3-benzothiazol-2-yl)-1-[2-(dimethylamino) ethyl]-3-methyl piperidine-3-carboxamide (6.5 mg, 17 μmol) with >95% purity and 7% yield.

Analytical Data

| | |
|---|---|
| LCMS calculated: | m/z 369.15 [M + H]⁺ |
| LCMS observed: | m/z 369.19 [M + H]⁺ |

¹H NMR (MeOD, 400 MHz) δ: 7.53-7.51 (m, 1H), 7.13-7.07 (m, 1H), 3.50-3.39 (m, 1H), 3.34-3.33 (m, 1H), 3.26 (s, 1H), 3.25-3.21 (m, 1H), 3.51-3.10 (s, 1H), 3.01-2.90 (m, 7H), 2.58 (td, J=10.4, 4.0 Hz, 1H), 2.28 (dt, J=11.6, 3.2 Hz, 1H), 2.04-1.99 (m, 1H), 1.90-1.58 (m, 4H) 1.54-1.45 (m, 1H) ppm.

### Example C

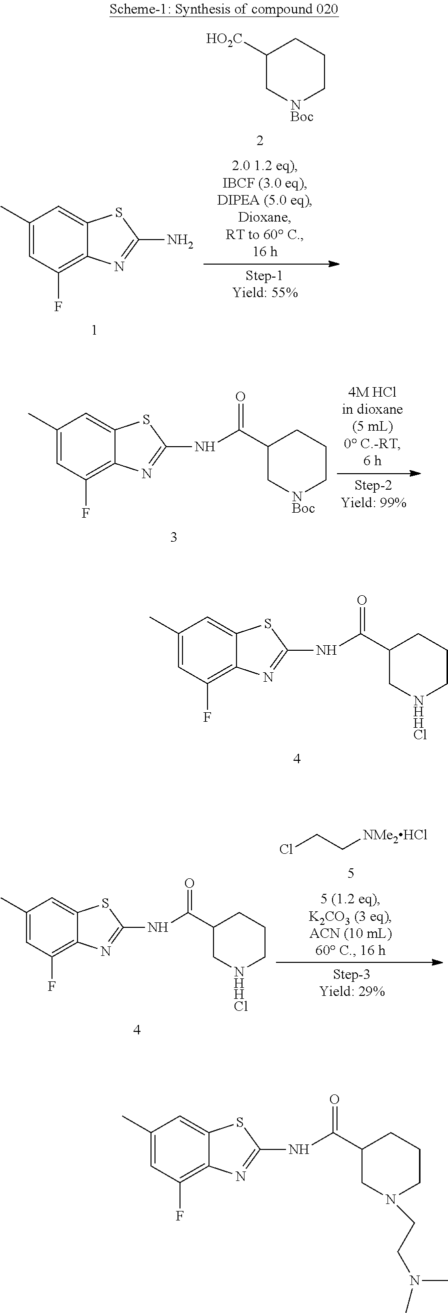

### Step-1: Synthesis of tert-butyl3-[(4-fluoro-6-methyl-1,3-benzothiazol-2-yl)carbamoyl]piperidine-1-carboxylate

To a stirred solution of 1-[(tert-butoxy)carbonyl]piperidine-3-carboxylic acid (377 mg, 1.2 eq., 1.65 mmol) and ethylbis(propan-2-yl)amine (887 mg, 5 eq., 6.86 mmol) in 1,4-dioxane (10 mL, 117 mmol), 2-methylpropyl carbonochloridate (562 mg, 3 eq., 4.12 mmol) added and stirred for 30 min at room temperature. Then 4-(3-chlorophenyl)-1,3-thiazol-2-amine (0.3 g, 1.42 mmol) was added to the above suspension and reaction mixture was stirred at 60° C. for overnight. Progress of reaction was monitored by the TLC. Reaction mixture was diluted with water and extracted with ethyl acetate, organic layer was further washed with saturated NaHCO₃ solution followed by brine solution. The organic layer was dried over MgSO₄, filtered and concentrated in vacuo. The residue was purified by flash column chromatography to obtain tert-butyl 3-[(4-fluoro-6-methyl-1,3-benzothiazol-2-yl)carbamoyl]piperidine-1-carboxylate (0.3 g, 762 μmol) (55% yield) with 71% purity (LCMS).

Analytical Data

| | |
|---|---|
| LCMS calculated: | m/z 394.15 [M + H]⁺ |
| LCMS observed: | m/z 394.40 [M + H]⁺ |

### Step-2: Synthesis of N-(4-fluoro-6-methyl-1,3-benzothiazol-2-yl)piperidine-3-carboxamide

To a stirred tert-butyl 3-[(4-fluoro-6-methyl-1,3-benzothiazol-2-yl)carbamoyl]piperidine-1-carboxylate (0.3 g, 762 μmol) HCl solution at 0° C. and further stirred at for 6 h. The progress of the reaction was monitored by TLC and LCMS. The reaction mixture was concentrated under reduced pressure and further triturated by diethyl ether N-(4-fluoro-6-methyl-1,3-benzothiazol-2-yl)piperidine-3-carboxamide (210 mg, 716 μmol) (93% Yield) with 93% purity as white solid.

Analytical Data

| | |
|---|---|
| LCMS calculated: | m/z 293.10 [M + H]⁺ |
| LCMS observed: | m/z 393.70 [M + H]⁺ |

### Step-3: Synthesis of 1-[2-(dimethylamino)ethyl]-N-(4-fluoro-6-methyl-1,3-benzothiazol-2-yl)piperidine-3-carboxamide

To a stirred solution N-(4-fluoro-6-methyl-1,3-benzothiazol-2-yl)piperidine-3-carboxamide (190 mg, 648 μmol) and dipotassium carbonate (269 mg, 3 eq., 1.94 mmol) in acetonitrile (8 mL, 153 mmol), was hydrogen (2-chloroethyl)dimethylamine chloride (112 mg, 1.2 eq., 777 μmol) and stirred at 60° C. for 16 h. Progress of reaction was monitored by TLC and LCMS. Reaction mixture was filtered through celite and concentrated under reduced pressure and further purified by prep-HPLC to afford 1-[2-(dimethylamino)ethyl]-N-(4-fluoro-6-methyl-1,3-benzothiazol-2-yl)piperidine-3-carboxamide (70 mg, 192 μmol) (29% Yield) with 99% purity.

Analytical Data

| | |
|---|---|
| LCMS calculated: | m/z 365.17 [M + H]⁺ |
| LCMS observed: | m/z 365.30 [M + H]⁺ |

¹H NMR (MeOD, 400 MHz) δ: 7.47 (s, 1H), 7.04-7.01 (dd, J=0.8 Hz, J=11.6 Hz, 1H), 3.52-3.40 (m, 2H), 3.25-3.0 (m, 6H), 2.96 (s, 6H), 2.85-2.72 (m, 1H), 2.45 (s, 3H), 2.12-2.0 (m, 1H), 1.95-1.78 (m, 3H).

### Example D

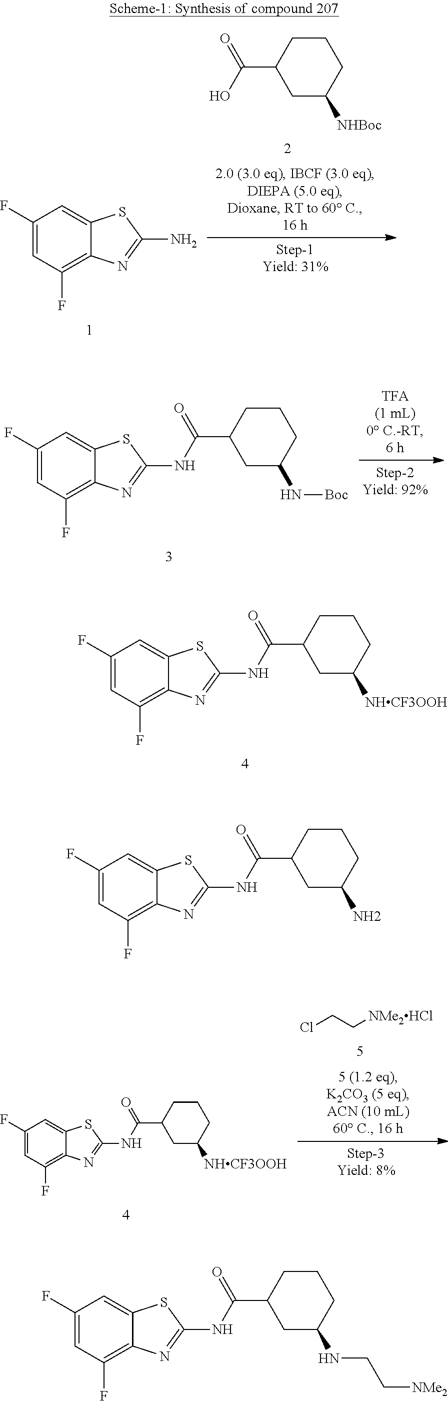

### Step-1: Synthesis of tert-butyl N-[(1R,3S)-3-[(4,6-difluoro-1,3-benzothiazol-2-yl)carbamoyl]cyclohexyl]carbamate

In a dried reaction vessel, to a mixture of (1R,3S)-3-{[(tert-butoxy)carbonyl]amino}cyclohexane-1-carboxylic acid (1.96 g, 8.06 mmol) and DIPEA (2.48 mL, 13.4 mmol) in 1,4-dioxane (15 mL) was added 2-methylpropyl carbonochloridate (1.1 g, 8.06 mmol) dropwise at RT. The resulted mixture was stirred for 30 min. Then 4,6-difluoro-1,3-benzothiazol-2-amine (0.5 g, 2.69 mmol) was added to the resulted white suspension and reaction mixture was warmed up to 60° C. and stirring continued for overnight. After completion of the reaction (monitored by TLC and LCMS), mixture was diluted ethyl acetate and washed with saturated NaHCO₃ solution followed by brine solution. The organic layer was extracted, dried over NaSO4 and concentrated under reduced pressure followed by the subjected to the silica get flash chromatography to obtain tert-butyl N-[(1R,3S)-3-[(4,6-difluoro-1,3-benzothiazol-2-yl)carbamoyl]cyclohexyl]carbamate (350 mg, 851 μmol) as a white solid. in 31% Yield with 94% purity.

Analytical Data

| | |
|---|---|
| LCMS calculated: | m/z 412.15 [M + H]⁺ |
| LCMS observed: | m/z 412.00 [M + H]⁺ |

### Step-2: Synthesis of (1S,3R)-3-amino-N-(4,6-difluoro-1,3-benzothiazol-2-yl)cyclohexane-1-carboxamide

To the solution of tert-butyl N-[(1R,3S)-3-[(4,6-difluoro-1,3-benzothiazol-2-yl)carbamoyl]cyclohexyl]carbamate (0.5 g, 1.22 mmol) in) was added and trifluoroacetic acid (186 μL, 2.43 mmol) at 0° C., resulting mixture was stirred at room temperature for 6 h. After completion of the reaction (Monitored by TLC), Mixture was concentrated under vacuum and triturated with diethyl ether to obtained (1S,3R)-3-amino-N-(4,6-difluoro-1,3-benzothiazol-2-yl)cyclohexane-1-carboxamide (350 mg, 1.12 mmol) as a white solid with 92% yield and 96% purity.

Analytical Data

| | |
|---|---|
| LCMS calculated: | m/z 312.09 [M + H]⁺ |
| LCMS observed: | m/z 311.88 [M + H]⁺ |

### Step-3: Synthesis of (1S,3R)—N-(4,6-difluoro-1,3-benzothiazol-2-yl)-3-{[2-(dimethylamino)ethyl]amino}cyclohexane-1-carboxamide

To a stirred solution of (1S,3R)—N-(4,6-difluoro-1,3-benzothiazol-2-yl)-3-(2,2,2-trifluoroacetamido)cyclohexane-1-carboxamide (80 mg, 196 μmol) and K2CO3 (81.4 mg, 589 μmol) in acetonitrile (7 mL) was added hydrogen (2-chloroethyl)dimethylamine chloride (31.1 mg, 216 μmol) and stirred at 60° C. for 16 h. Progress of reaction was monitored by TLC and LCMS. Reaction mixture was filtered through celite and concentrated under reduced pressure and further purified by prep-HPLC to afford (1S,3R)—N-(4,6-difluoro-1,3-benzothiazol-2-yl)-3-{[2-(dimethylamino)ethyl]amino}cyclohexane-1-carboxamide (6 mg, 15.7 μmol) as a white solid in 8% yield with 99% purity.

Analytical Data

| | |
|---|---|
| LCMS calculated: | m/z 383.17 [M + H]⁺ |
| LCMS observed: | m/z 383.20 [M + H]⁺ |

¹H NMR (MeOD, 400 MHz) δ: 7.54-7.52 (m, 1H), 7.31-7.25 (m, 1H), 4.98-4.90 (m, 2H), 3.75-3.62 (m, 2H), 3.25-3.20 (m, 1H), 3.07 (s, 6H), 2.75-2.68 (m, 1H), 2.28-2.35 (m, 1H), 2.15-2.22 (m, 1H), 2.10-1.98 (m, 2H), 1.65-1.45 (m, 2H), 1.42-1.28 (m, 3H) ppm.

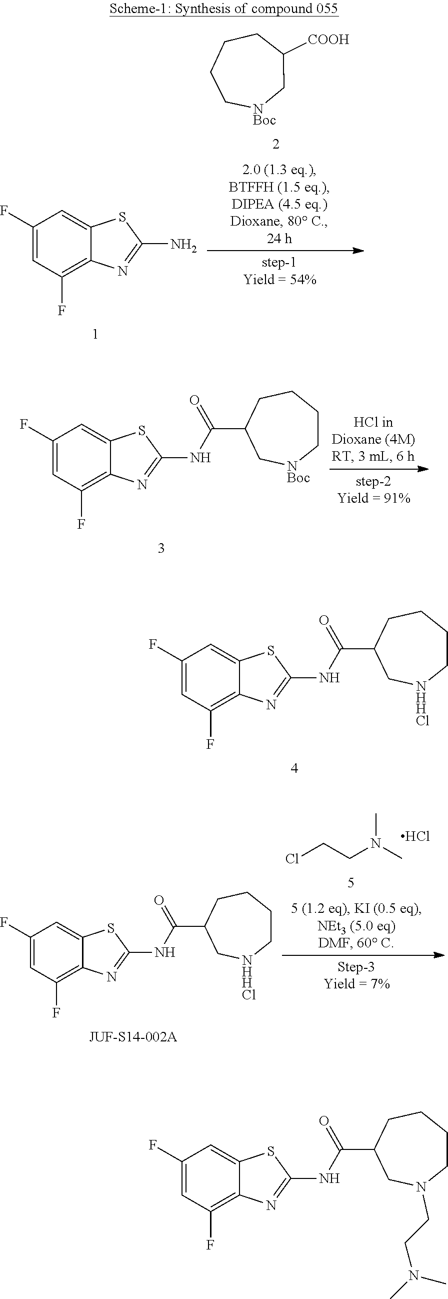

### Step-1: Synthesis of tert-butyl 3-((4,6-difluorobenzo[d]thiazol-2-yl)carbamoyl)azepane-1-carboxylate

1-[fluoro(pyrrolidin-1-yl)methylidene]-1IV-pyrrolidin-1-ylium; hexafluoro-λ⁵-phosphanuide (637 mg, 2.01 mmol) was added to the solution of 1-[(tert-butoxy)carbonyl]azepane-3-carboxylic acid (425 mg, 1.75 mmol) and DIPEA (1.15 mL 6.04 mmol) in dry DCM (6.09 mL) under N². Then reaction mixture was stirred at rt for 30 min. Then 4,6-difluoro-1,3-benzothiazol-2-amine (250 mg, 1.34 mmol) was added and the vial was sealed then heated at 80° C. for 24 h) (CAUTION: Heating CH₂Cl₂ over its boiling point causes overpressure in the reactor). The reaction mixture was cooled to room temperature and diluted with water and extracted with DCM. The collected organic phases were combined and washed with brine, dried over MgSO4 and concentrated and subjected to the Flash Chromatography to obtained tert-butyl 3-[(4,6-difluoro-1,3-benzothiazol-2-yl)carbamoyl]azepane-1-carboxylate (0.3 g, 729 μmol) in 54% yield and 95% Purity.

Analytical Data

| | |
|---|---|
| LCMS calculated: | m/z 410.13 [M − H]⁺ |
| LCMS observed: | m/z 410.05 [M − H]⁺ |

### Step-2: Synthesis of N-(4,6-difluorobenzo[d]thiazol-2-yl)azepane-3-carboxamide hydrochloride

To a stirred solution of tert-butyl 3-((4,6-difluorobenzo[d]thiazol-2-yl)carbamoyl)azepane-1-carboxylate (233 mg, 0.056 mmol) in DCM was added 4M (4 mL) was added and resulting mixture was stirred at room temperature for 6 h. After completion, the reaction mixture was concentrated under vacuum and triturated with diethyl ether to obtained Desired Product N-(4,6-difluoro-1,3-benzothiazol-2-yl)azepane-3-carboxamide hydrochloride (180 mg, 518 μmol) with 96% Purity and 91% yield.

Analytical Data

| | |
|---|---|
| LCMS calculated: | m/z 312.10 [M + H]⁺ |
| LCMS observed: | m/z 312.10 [M + H]⁺ |

### Step-3: Synthesis of N-(4,6-difluorobenzo[d]thiazol-2-yl)-1-(2-(dimethylamino)ethyl) azepane-3-carboxamide

(2-chloroethyl)dimethylamine (37.1 mg, 345 μmol) was added to the stirred suspension of N-(4,6-difluoro-1,3-benzothiazol-2-yl)azepane-3-carboxamide hydrochloride (0.1 g, 288 μmol) and K2CO3 (119 mg, 863 μmol) in acetonitrile (5 mL) at room temperature. Resulting mixture was stirred at 60° C. for overnight and the progress of reaction was monitored by TLC and LCMS. Reaction mixture was filtered through celite and concentrated under vacuum to get crude mixture which was subjected Prep-HPLC to obtain N-(4,6-difluoro-1,3-benzothiazol-2-yl)-1-[2-(dimethylamino)ethyl]azepane-3-carboxamide (8 mg, 20.9 μmol) in >95% Yield

Analytical Data

| | |
|---|---|
| LCMS calculated: | m/z 383.17 [M + H]⁺ |
| LCMS observed: | m/z 383.15 [M + H]⁺ |

¹H NMR (MeOD, 400 MHz) δ: 7.52-7.50 (dd, J=1.6, 8.0 Hz, 1H), 7.12-7.06 (m, 1H), 3.60-3.50 (m, 1H), 3.39-3.33 (m, 1H), 3.29-3.22 (m, 1H), 3.20-3.11 (m, 2H), 3.04-2.93 (m, 9H) 2.87-2.78 (m, 1H), 2.29-2.18 (m, 1H) 1.97-1.85 (m, 3H), 1.83-1.74 (m, 1H), 1.70-1.58 (m, 1H) ppm.

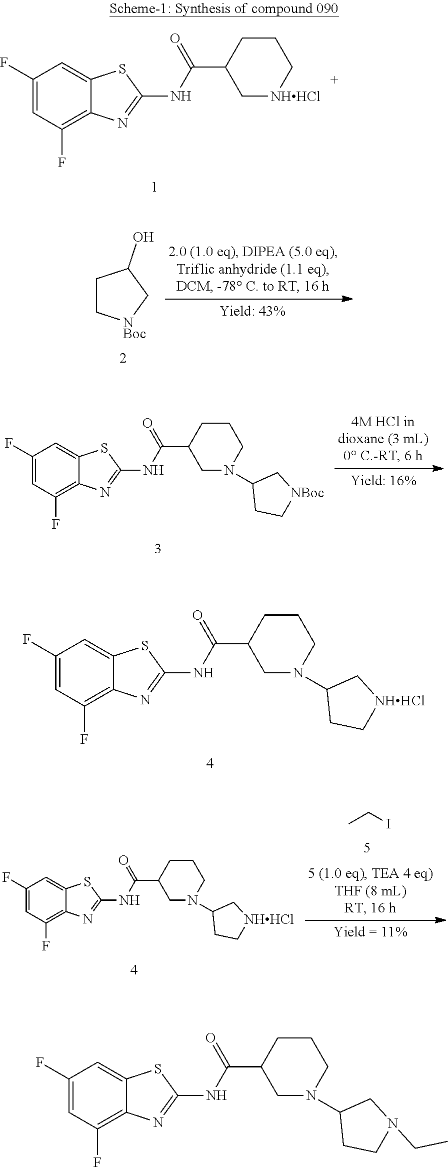

### Step-1: Synthesis of tert-butyl 3-{3-[(4,6-difluoro-1,3-benzothiazol-2-yl)carbamoyl]piperidin-1-yl}pyrrolidine-1-carboxylate

To a stirred solution of N-(4,6-difluoro-1,3-benzothiazol-2-yl)piperidine-3-carboxamide hydrochloride (3.74 g, 11.2 mmol) in dichloromethane (40 mL, 625 mmol) cool to −78° C., then added DIPEA (1.96 mL, 11.2 mmol) stirred 10 min. After trifluoromethanesulfonate (1.39 mL, 8.22 mmol) was added to reaction mixture then stirred 15 min at −10° C. The solution of (N-(4,6-difluoro-1,3-benzothiazol-2-yl)piperidine-3-carboxamide hydrochloride (3.74 g, 11.2 mmol) in DIPEA and DCM) was added to reaction mixture dropwise at 0° C. for 15 min. The reaction mixture was stirring continued at RT for 16 h. After completion of the reaction (monitored by TLC and LCMS), reaction mixture was diluted DCM and washed with brine solution. The organic layer was dried over NaSO4 and concentrated under reduced pressure followed by the subjected to the silica get flash chromatography to obtain the desired tert-butyl 3-{3-[(4,6-difluoro-1,3-benzothiazol-2-yl)carbamoyl]piperidin-1-yl}pyrrolidine-1-carboxylate (1.5 g, 3.22 mmol) 43% yield) with 75% purity (LCMS).

Analytical Data

| | |
|---|---|
| LCMS calculated: | m/z 465.19 [M − H]⁺ |
| LCMS observed: | m/z 465.06 [M − H]⁺ |

### Step-2: Synthesis of N-(4,6-difluoro-1,3-benzothiazol-2-yl)-1-(pyrrolidin-3-yl)piperidine-3-carboxamide hydrochloride

To a stirred solution of tert-butyl 3-{3-[(4,6-difluoro-1,3-benzothiazol-2-yl)carbamoyl]piperidin-1-yl}pyrrolidine-1-carboxylate (2 g, 4.29 mmol) in dichloromethane (20 mL) was added 4.0 M HCl in dioxane (5 mL) at 0° C. and further stirred at for 6 h. The progress of the reaction was monitored by TLC and LCMS. The reaction mixture was concentrated under reduced pressure and further triturated by diethyl ether afford N-(4,6-difluoro-1,3-benzothiazol-2-yl)-1-(pyrrolidin-3-yl)piperidine-3-carboxamide hydrochloride (290 mg, 720 mol) in 16% yield with 99% purity (LCMS).

Analytical Data

| | |
|---|---|
| LCMS calculated: | m/z 367.14 [M + H]⁺ |
| LCMS observed: | m/z 367.15 [M + H]⁺ |

### Step-3: Synthesis of N-(4,6-difluoro-1,3-benzothiazol-2-yl)-1-(1-ethylpyrrolidin-3-yl)piperidine-3-carboxamide

To a stirred solution of N-(4,6-difluoro-1,3-benzothiazol-2-yl)-1-(pyrrolidin-3-yl)piperidine-3-carboxamide hydrochloride (0.2 g, 496 μmol) and triethylamine (272 μL, 1.99 mmol) in tetrahydrofuran (10 mL, 123 mmol), was added iodoethane (40.7 μL, 496 μmol) and stirred at Rt for 16 h. Progress of reaction was monitored by TLC and LCMS. Reaction mixture was concentrated under reduced pressure and further purified by prep-HPLC to afford (11% yield) with 99% purity.

Analytical Data

| | |
|---|---|
| LCMS calculated: | m/z 395.17 [M + H]⁺ |
| LCMS observed: | m/z 395.20 [M + H]⁺ |

¹H NMR (MeOD, 400 MHz) δ: 7.52-7.50 (m, 1H), 7.11-7.06 (m, 1H), 7.23-7.18 (m, 1H), 3.90-3.52 (m, 4H), 3.45-3.35 (m, 1H), 3.27-3.12 (m, 1H), 3.10-2.85 (m, 2H), 2.82-2.70 (m, 1H), 2.55-2.45 (m, 1H), 2.35-2.20 (m, 1H), 2.18-2.08 (m, 1H), 1.90-1.70 (m, 2H), 1.36 (t, 7.2 Hz, 1H) ppm.

### Example G

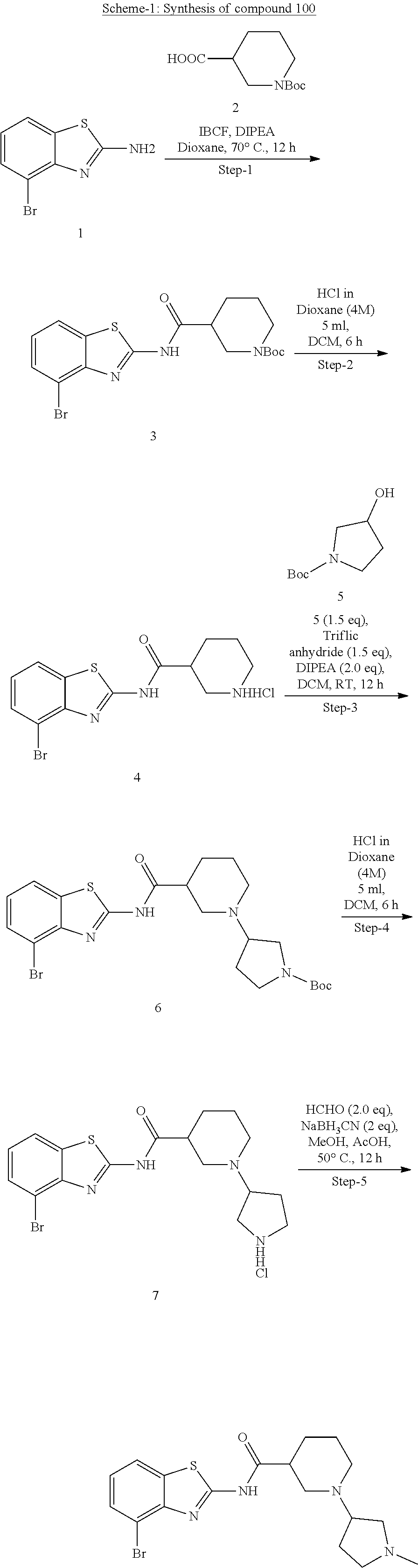

### Step-1: tert-butyl 3-((4-bromobenzo[d]thiazol-2-yl)carbamoyl)piperidine-1-carboxylate

To a stirred solution of 1-[(tert-butoxy)carbonyl]piperidine-3-carboxylic acid (4 g, 2 eq., 17.5 mmol) in 1,4-dioxane (40 mL, 469 mmol) was added DIPEA (4.57 mL, 26.2 mmol), followed by addition of 2-methylpropyl carbonochloridate (2.17 mL, 17.5 mmol) at 0° C. The reaction mixture was stirred for 30 min at that temperature. After 30 min 4-bromo-1,3-benzothiazol-2-amine (2 g, 8.73 mmol) was added and heated at 70° C. for 12 h. After 12 h TLC as well as LCMS monitoring indicated complete consumption of starting material with formation of new desired product mass. Then the reaction mixture was added to cold water and extracted with ethyl acetate. The organic portion was separated and concentrated to get the crude product. The crude was purified in combi flash chromatography to get the desired product tert-butyl 3-[(4-bromo-1,3-benzothiazol-2-yl)carbamoyl]piperidine-1-carboxylate (2.2 g, 5 mmol) as a colourless liquid.

Analytical Data

| | |
|---|---|
| LCMS calculated: | m/z 440.06 [M + H]⁺ |
| LCMS observed: | m/z 440.05 [M + H]⁺ |

### Step-2: N-(4-bromobenzo[d]thiazol-2-yl)piperidine-3-carboxamide hydrochloride

To a stirred solution of tert-butyl 3-[(4-bromo-1,3-benzothiazol-2-yl)carbamoyl]piperidine-1-carboxylate (2 g, 4.54 mmol) in 1,4-dioxane (10 mL) was added 4M hydrogen chloride in 1,4-dioxane (10 mL) at room temperature. The resulting reaction mixture was stirred for 6 h at room temperature. After 6 h TLC as well as LCMS monitoring indicated complete consumption of starting material with formation of new desired product mass. Then the reaction mixture was concentrated and triturated with diethyl ether to get desired compound N-(4-bromobenzo[d]thiazol-2-yl)piperidine-3-carboxamide hydrochloride (1.5 g, 82% yield) as a white solid

Analytical Data

| | |
|---|---|
| LCMS calculated: | m/z 340.0 [M + H]⁺ |
| LCMS observed: | m/z 339.90 [M + H]⁺ |

### Step-3: tert-butyl 3-{3-[(4-bromo-1,3-benzothiazol-2-yl)carbamoyl]piperidin-1-yl}pyrrolidine-1-carboxylate

To a stirred solution of tert-butyl 3-hydroxypyrrolidine-1-carboxylate (373 mg, 1.99 mmol) in dichloromethane (25 mL) was added DIPEA (695 μL, 3.98 mmol) at −78° C. and stirred for 15 min. Then trifluoromethanesulfonyl trifluoromethanesulfonate (562 mg, 1.99 mmol) was added to the resulting reaction mixture and again stirred for 30 min at that temperature. After 30 min N-(4-bromo-1,3-benzothiazol-2-yl)piperidine-3-carboxamide hydrochloride (0.5 g, 1.33 mmol) in DCM was added to the reaction mixture and stirred for 12 h at room temperature. After completion (Monitored by TLC as well as LCMS) The reaction mixture was added with cold water and extracted with DCM. The organic portion was dried in vacuum and purified in combi flash chromatography to get the pure product. tert-butyl 3-{3-[(4-bromo-1,3-benzothiazol-2-yl)carbamoyl]piperidin-1-yl}pyrrolidine-1-carboxylate as a light gum (0.3 g, 60% yield)

Analytical Data

| | |
|---|---|
| LCMS calculated: | m/z 509.11 [M + H]⁺ |
| LCMS observed: | m/z 509.15 [M + H]⁺ |

### Step-4: N-(4-bromo-1,3-benzothiazol-2-yl)-1-(pyrrolidin-3-yl) piperidine-3-carboxamide

To a stirred solution of tert-butyl 3-{3-[(4-bromo-1,3-benzothiazol-2-yl)carbamoyl]piperidin-1-yl}pyrrolidine-1-carboxylate (280 mg, 550 μmol) in 1,4-dioxane (2 mL) was added 4M hydrogen chloride in 1,4-dioxane (2 mL) at room temperature and stirred for 12 h at that temperature. After 12 h TLC as well as LCMS monitoring indicated consumption of starting material with formation of new desired mass peak. Then the reaction mixture was concentrated and triturated with diethyl ether to get the desired product N-(4-bromobenzo[d]thiazol-2-yl)-1-(pyrrolidin-3-yl)piperidine-3-carboxamide hydrochloride (0.2 g, 95% yield) as a white solid

Analytical Data

| | |
|---|---|
| LCMS calculated: | m/z 409.0 [M + H]⁺ |
| LCMS observed: | m/z 409.0 [M + H]⁺ |

### Step-5: N-(4-bromo-1,3-benzothiazol-2-yl)-1-(1-methylpyrrolidin-3-yl) piperidine-3-carboxamide

To a stirred solution of N-(4-bromo-1,3-benzothiazol-2-yl)-1-(pyrrolidin-3-yl)piperidine-3-carboxamide hydrochloride (0.2 g, 449 μmol) in methanol (4 mL) was added acetic acid (67.4 mg, 1.12 mmol) and stirred for 5 min. Then formaldehyde (53.9 mg, 1.79 mmol) was added and heated at 50° C. for 12 h. After 12 h boron(3+) sodium iminomethanide trihydride (84.6 mg, 1.35 mmol) was added and stirred for 1 h at that temperature. After completion (Monitored by LCMS) the reaction mixture was filtered through celite bed and concentrated in vacuum to get the crude product. The crude was purified in prep HPLC purification to get the pure product N-(4-bromo-1,3-benzothiazol-2-yl)-1-(1-methylpyrrolidin-3-yl)piperidine-3-carboxamide (25 mg, 59.1 μmol) as white solid.

Analytical Data

| | |
|---|---|
| LCMS calculated: | m/z 423.08 [M + H]⁺ |
| LCMS observed: | m/z 423.15 [M + H]⁺ |

¹H NMR (MeOD, 400 MHz) δ: 7.85 (d, J=8.0 Hz, 1H), 7.64 (d, J=7.6 Hz, 1H), 7.22-7.18 (m, 1H), 3.80-3.52 (m, 4H), 3.42-3.38 (m, 1H), 3.21-3.15 (m, 1H), 3.10-2.85 (m, 6H), 2.81-2.65 (m, 1H), 2.58-2.42 (m, 1H), 2.40-2.23 (m, 1H), 2.15-2.12 (m, 1H), 2.10-1.90 (m, 1H), 1.88-1.78 (m, 2H) ppm.

### Example H

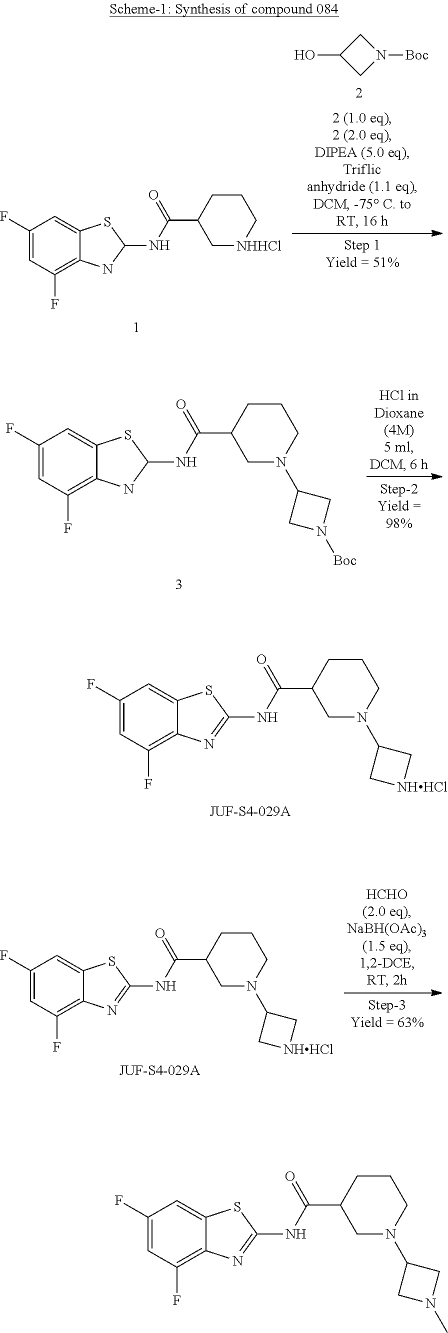

### Step-1: Synthesis of tert-butyl 3-{3-[(4,6-difluoro-1,3-benzothiazol-2-ylcarbamoyl]piperidin-1-yl}azetidine-1-carboxylate

trifluoromethanesulfonyl trifluoromethanesulfonate (465 mg, 1.65 mmol) was added to the solution of tert-butyl 3-hydroxyazetidine-1-carboxylate (259 mg, 1.5 mmol) and DIPEA (0.83 mL, 4.49 mmol) in DCM at −78° C. and resulted solution was stirred for 30 min. then allowed the reaction to warm up to −10° C. Then solution of DIPEA and N-(4,6-difluoro-1,3-benzothiazol-2-yl)piperidine-3-carboxamide hydrochloride (0.5 g, 1.5 mmol) was added to the mixture slowly and stirring continued for 8 h at room temperature. After completion of the reaction, mixture was diluted with water and extracted with DCM. The organic layer was concentrated under reduced pressure and subjected to the silica gel column chromatography to obtain tert-butyl 3-{3-[(4,6-difluoro-1,3-benzothiazol-2-yl)carbamoyl]piperidin-1-yl}azetidine-1-carboxylate (350 mg, 773 μmol) with 68% purity which was used as such for further reaction.

Analytical Data

| | |
|---|---|
| LCMS calculated: | m/z 453.18 [M + H]⁺ |
| LCMS observed: | m/z 453.10 [M + H]⁺ |

### Step-2: Synthesis of 1-(azetidin-3-yl)-N-(4,6-difluoro-1,3-benzothiazol-2-yl)piperidine-3-carboxamide hydrochloride

To the solution of tert-butyl 3-{3-[(4,6-difluoro-1,3-benzothiazol-2-yl)carbamoyl]piperidin-1-yl}azetidine-1-carboxylate (256 mg, 566 μmol) in DCM was added 4M HCl in 1,4-dioxane (4 mL) was added and resulting mixture was stirred at room temperature for 6 h. After completion of the reaction (Monitored by TLC), mixture was concentrated under vacuum and triturated with diethyl ether to obtained desired product N-1-(azetidin-3-yl)-N-(4,6-difluoro-1,3-benzothiazol-2-yl)piperidine-3-carboxamide hydrochloride (0.2 g, 514 μmol) with 83% purity and 98% yield.

Analytical Data

| | |
|---|---|
| LCMS calculated: | m/z 353.12 [M + H]⁺ |
| LCMS observed: | m/z 352.95 [M + H]⁺ |

### Step-3: Synthesis of N-(4,6-difluoro-1,3-benzothiazol-2-yl)-1-(1-methylazetidin-3-yl) piperidine-3-carboxamide

To a stirred solution of 1-(azetidin-3-yl)-N-(4,6-difluoro-1,3-benzothiazol-2-yl)piperidine-3-carboxamide hydrochloride (120 mg, 309 μmol) in Methanol (6 mL) was added formaldehyde (13.9 mg 463 μmol) and acetic acid (185 μg, 3.09 μmol) followed by stirred at 50° C. for 16 h. Then reaction mixture was cooled to 0° C. and NaBH4 (38.8 mg, 617 μmol) was added portion-wise and stir the mixture at room temperature for 2 h. After completion of the reaction (Monitored by TLC) mixture was concentrated under reduced pressure and diluted with DCM. washed the organic layer with Brine solution and extract the organic layer and concentrated under the reduced pressure. crude was subjected to the prep-HPLC to obtain N-(4,6-difluoro-1,3-benzothiazol-2-yl)-1-(1-methylazetidin-3-yl)piperidine-3-carboxamide (72 mg, 196 μmol) in 63% yield with >95% purity.

Analytical Data

| | |
|---|---|
| LCMS calculated: | m/z 367.14 [M + H]⁺ |
| LCMS observed: | m/z 367.20 [M + H]⁺ |

¹H NMR (MeOD, 400 MHz) δ: 7.53-7.46 (m, 1H), 7.12-7.04 (m, 1H), 4.40-4.05 (m, 4H), 3.50-3.43 (m, 1H), 2.98 (s, 3H), 2.97-2.93 (m, 1H), 2.90-2.82 (m, 1H), 2.80-2.74 (m, 1H), 2.44 (t, J=10.8 Hz, 1H), 2.28-2.18 (m, 1H), 2.03-2.00 (m, 1H), 1.93-1.85 (m, 1H), 1.78-1.63 (m, 2H) ppm.

### Example I

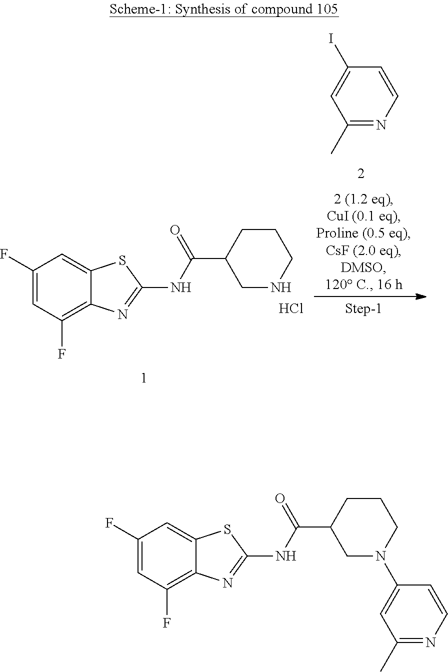

### Step-1: N-(4,6-difluorobenzo[d]thiazol-2-yl)-1-(2-methylpyridin-4-yl)piperidine-3-carboxamide

A mixture of N-(4,6-difluoro-1,3-benzothiazol-2-yl)piperidine-3-carboxamide (0.3 g, 901 μmol), 4-iodo-2-methylpyridine (197 mg, 901 μmol) and Cs2CO3 (881 mg, 2.7 mmol) in dimethyl sulfoxide (6.7 mL) was degassed with argon for 10 min. Then CuI (172 mg, 901 μmol), Proline (20.7 mg, 180 μmol), CsF (274 mg, 1.8 mmol) was added to reaction mixture and the reaction mixture was degassed with argon for 10 min. After the reaction mixture was stirred at 120° C. for 16 h. After completion, the reaction mixture was diluted with water and extracted with EtOAc. The organic layer were separated and dried over anhydrous Na₂SO₄ and concentrated under vacuo to get residue. The crude was submitted for prep HPLC. After HPLC purification N-(4,6-difluoro-1,3-benzothiazol-2-yl)-1-(2-methylpyridin-4-yl) piperidine-3-carboxamide (45 g, 116 mmol) as a white solid (13% yield).

Analytical Data

| | |
|---|---|
| LCMS calculated: | m/z 389.12 [M + H]⁺ |
| LCMS observed: | m/z 389.10 [M + H]⁺ |

¹HNMR (MeOD, 400 MHz) δ: 7.98 (d, J=8.0 Hz 1H), 7.52-7.49 (m, 1H), 7.11-7.05 (m, 3H), 4.29-4.25 (dd, J=3.6 Hz, 14.0 Hz, 1H), 4.11-4.05 (m, 1H), 3.70-3.65 (m, 1H), 3.47-3.40 (m, 1H), 2.90-2.84 (m, 1H), 2.50 (s, 3H), 2.24-2.18 (m, 1H), 2.06-1.95 (m, 2H), 1.76-1.67 (m, 1H) ppm.

### Example J

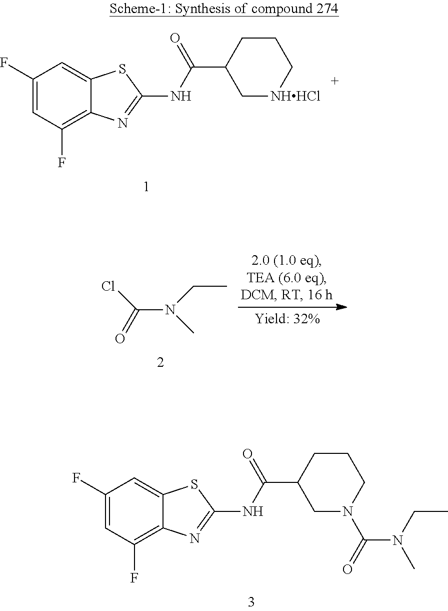

### Step-1: Synthesis of N3-(4,6-difluoro-1,3-benzothiazol-2-yl)-N¹-ethyl-N¹-methyl piperidine-1,3-dicarboxamide

To a stirred solution of N-(4,6-difluoro-1,3-benzothiazol-2-yl)piperidine-3-carboxamide hydrochloride (250 mg, 749 μmol), triethylamine (615 μL, 4.49 mmol) in dichloromethane (6 mL), was N-ethyl-N-methylcarbamoyl chloride (91 mg, 749 μmol) and stirred at RT for 16 h. Progress of reaction was monitored by TLC and LCMS. Reaction mixture was concentrated under reduced pressure and further purified by prep-HPLC to afford in N3-(4,6-difluoro-1,3-benzothiazol-2-yl)-N1-ethyl-N1-methylpiperidine-1,3-dicarboxamide (94 mg, 246 μmol) 32% yield with 99% purity.

Analytical Data

| | |
|---|---|
| LCMS calculated: | m/z 383.13 [M + H]⁺ |
| LCMS observed: | m/z 383.25 [M + H]⁺ |

¹H NMR (MeOD, 400 MHz) δ: 7.51-7.48 (m, 1H), 7.09-7.03 (m, 1H), 3.79-3.75 (m, 1H) 3.59-3.56 (m, 1H), 3.25-3.18 (m, 2H), 3.09-3.03 (m, 1H), 2.96-2.90 (m, 1H), 2.84 (s, 3H), 2.80-2.76 (m, 1H), 2.13-2.05 (m, 1H), 1.84-1.74 (m, 2H), 1.68-1.53 (m, 1H), 1.14 (t, J=7.2 Hz, 3H)ppm.

### Example 2

Test results (EC₅₀, max efficacy) for the exemplified compounds are shown in Table 2.

TABLE 2

| Cmpd. No. | EC₅₀ (μM) | Max efficacy (%) |
|---|---|---|
| 216 | >10 | |
| 224 | 5.8 | 105 |
| 225 | 0.36 | 97 |
| 002 | >10 | |
| 003 | >10 | |
| 004 | >10 | |
| 005 | >10 | |
| 006 | >10 | |
| 010 | >10 | |
| 011 | >10 | |
| 013 | >10 | |
| 015 | >10 | |
| 016 | 9.5 | 94 |
| 017 | >10 | |
| 018 | >10 | |
| 032 | 4.3 | 85 |
| 033 | 2.2, 1.2 | 82, 63 |
| 034 | 5.9 | 99 |
| 226 | 0.16, 0.9 | 62, 87 |
| 007 | 0.6 | 41 |
| 227 | 1.5 | 93 |
| 228 | >5 | |
| 037 | >5 | |
| 037 | 0.39 | 96 |
| 229 | 1.9 | 91 |
| 230 | 0.7 | 105 |
| 231 | 0.9 | 98 |
| 232 | 2.1 | 82 |
| 217 | >10 | |
| 038 | >5 | |
| 234 | >5 | |
| 235 | 1.4, 2.6 | 113, 113 |
| 019 | >5 | |
| 020 | 0.9 | 101.0 |
| 021 | 6.7, 1.7 | 112, 77 |
| 022 | >5 | |
| 023 | >5 | |
| 026 | >5 | |
| 029 | >5 | |
| 030 | >5 | |
| 236 | 0.7 | 85 |
| 237 | 0.39, 4.6 | 74, 51 |
| 238 | 0.21, 0.32 | 86, 92 |
| 239 | 0.22, 0.3 | 87, 94/77 |
| 240 | 0.7 | 105 |
| 241 | 1.6 | 93 |
| 242 | 0.7 | 105 |
| 066 | 1.9 | 77 |
| 243 | 0.47, 0.2 | 91, 77 |
| 244 | 0.28 | 94 |
| 067 | 0.7 | 88 |
| 068 | 0.9 | 89 |
| 245 | 4.5, 1.8 | 102, 105 |
| 069 | 0.03-0.2 | 89-98 |
| 070 | 0.7, 1.01, 0.1 | 71, 85, 97 |
| 071 | 0.2 | 93 |
| 072 | 1.2, 1.0 | 118, 94 |
| 073 | 0.4 | 94 |
| 074 | 1.1 | 80 |
| 075 | 1.5 | 92 |
| 078 | 4.9, 2.1 | 86, 81 |
| 079 | >5 | |
| 080 | 7.4, 2.5 | 135, 104 |
| 081 | 0.1, 0.07,0.3 | 87, 87, 87 |
| 084 | 0.07, 0.02 | 83, 67 |
| 085 | 0.08, 0.2 | 89, 84 |
| 199 | >10 | |
| 200 | >10 | |
| 201 | >10 | |
| 202 | >10 | |
| 203 | >10 | |
| 204 | >10 | |
| 205 | >10 | |
| 206 | >10 | |
| 248 | >5 | |
| 249a | >5 | |
| 249b | 22, 1.4 | 72 |
| 144 | >5 | |
| 145 | >5 | |
| 146 | >5 | |
| 147 | >5 | |
| 148 | >5 | |
| 149 | >5 | |
| 150 | >5 | |
| 151 | >5 | |
| 152 | >5 | |
| 153 | >5 | |
| 154 | >5 | |
| 155 | >5 | |
| 156 | >5 | |
| 160 | >5 | |
| 169 | >5 | |
| 250 | 1.4 | 60 |
| 251 | >5 | |
| 252 | >5 | |
| 253 | 0.05 | 72 |
| 255 | 0.6 | 86 |
| 041 | 1.53 | 101 |
| 219 | 1.5 | 91 |
| 220 | >5 | |
| 222 | 1.01 | 58 |
| 043 | >5 | |
| 046 | >5 | |
| 047 | >5 | |
| 207 | >5 | |
| 208 | 0.7 | 87 |
| 209 | >5 | |
| 210T | 0.5 | 86 |
| 210C | 1.9 | 92 |
| 211 | 1.53 | 101 |
| 049 | 0.3 | 98 |
| 053 | 0.8 | 92 |
| 055 | 0.6 | 89 |
| 057 | >5 | |
| 058 | 2.6 | 95 |
| 059 | >5 | |
| 060 | >5 | |
| 182 | >5 | |
| 183 | >5 | |
| 191 | >5 | |
| 259 | 1.3 | 74 |
| 260 | >5 | |
| 262 | 0.4 | 86 |
| 263 | 1.3 | 45 |
| 265 | 1.7 | 75 |
| 271 | >5 | |
| 061 | 1.02 | 94 |
| 062 | 3.4 | 92 |
| 090 | 0.16, 0.07 | 85, 69 |
| 091 | 1.3 | 72 |
| 092 | 0.05 | 89 |
| 093 | 0.09 | 86 |
| 094 | 1 | 93 |
| 095 | >5 | |
| 097 | 0.7 | 89 |
| 099 | 0.6 | 91 |
| 215 | 0.9 | 78 |
| 100 | 0.25 | 84 |
| 101 | 0.5 | 84 |
| 103 | 0.28 | 82 |
| 104 | 0.28 | 70 |
| 105 | 0.09 | 87 |
| 106 | 3.5 | 101 |
| 107 | 2.1 | 101 |
| 108 | 0.6 | 88 |
| 109 | 0.2, 1.08 | 82, 74 |
| 110 | 0.46, 1.1 | 95, 78 |
| 111 | 0.4 | 65 |
| 112 | 5.0 | 97 |
| 113 | >5 | |
| 114 | >5 | |
| 115 | 0.1 | 56 |
| 274 | 3.6, 1.2 | 62, 72 |
| 275 | >5 | |
| 276 | 1.6, >5 | 49 |
| 277 | >5 | |
| 278 | 2.6 | 70 |
| 279 | >5 | |
| 280 | >5 | |
| 281 | 1.3 | 53 |
| 282 | 0.18 | 61 |
| 130 | 0.12 | 94 |
| 131 | 0.13 | 81 |
| 135 | 0.11 | 85 |
| 136 | 0.3 | 46 |
| 137 | 0.08 | 80 |
| 138 | 1.1 | 46 |
| 139 | 0.2 | 79 |

### Exemplary Embodiments

1. A compound of Formula (I):

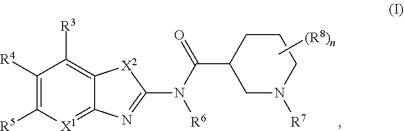

or a pharmaceutically acceptable salt thereof, wherein:
X¹ is selected from N and CR¹;
X² is selected from S, O, and NR²;
R¹ is selected from CN, NO₂, C(O)OR^{a1}, OR^{a1}, NR^{c1}R^{d1} Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹;
R² is selected from H, C₁₋₃ alkyl, and C₁₋₃ haloalkyl;
R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
2. The compound of embodiment 1, wherein R³, R⁴, and R⁵ are each independently selected from H, halo, CN, OH, C₁₋₃ alkyl, C₁₋₃ haloalkyl, C₁₋₃ alkoxy, and C₁₋₃ haloalkoxy.
3. The compound of embodiment 1, wherein R⁸ is selected from halo, CN, OH, NO₂, C₁₋₃ alkyl, C₁₋₃ haloalkyl, C₁₋₃ alkoxy, and C₁₋₃ haloalkoxy.
4. The compound of any one of embodiments 1-3, wherein n is 0, 1, or 2.
5. The compound of embodiment 1, wherein the compound of Formula (I) has formula:

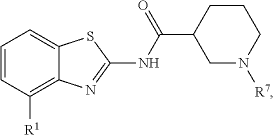

or a pharmaceutically acceptable salt thereof.
6. The compound of any one of embodiments 1-5, wherein R¹ is selected from CN, C(O)OR^{a1}, OR^{a1}, Cy¹, C₁₋₆ alkyl, and C₁₋₆ haloalkyl, wherein said C₁₋₆ alkyl is optionally substituted with Cy¹.
7. The compound of embodiment 6, wherein R¹ is C(O)O(C₁₋₃ alkyl).
8. The compound of embodiment 6, wherein R¹ is CN.
9. The compound of embodiment 6, wherein R¹ is C₁₋₃ alkoxy or C₁₋₃ haloalkoxy.
10. The compound of embodiment 6, wherein R¹ is C₁₋₆ alkyl or C₁₋₆ haloalkyl.
11. The compound of embodiment 6, wherein R¹ is phenyl or phenyl-C₁₋₆ alkylene.
12. The compound of embodiment 1, wherein the compound of Formula (I) has formula:

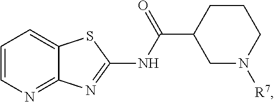

or a pharmaceutically acceptable salt thereof.
13. The compound of any one of embodiments 1-12, wherein R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1} NHC(═NH)NH₂, or NR^{c1}R^{d1}.
14. The compound of embodiment 13, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}.
15. The compound of embodiment 14, wherein R⁷ is C₁₋₆ alkyl substituted with NR^{c1}R^{d1}.
16. The compound of embodiment 1, wherein the compound is selected from any one of the following compounds:

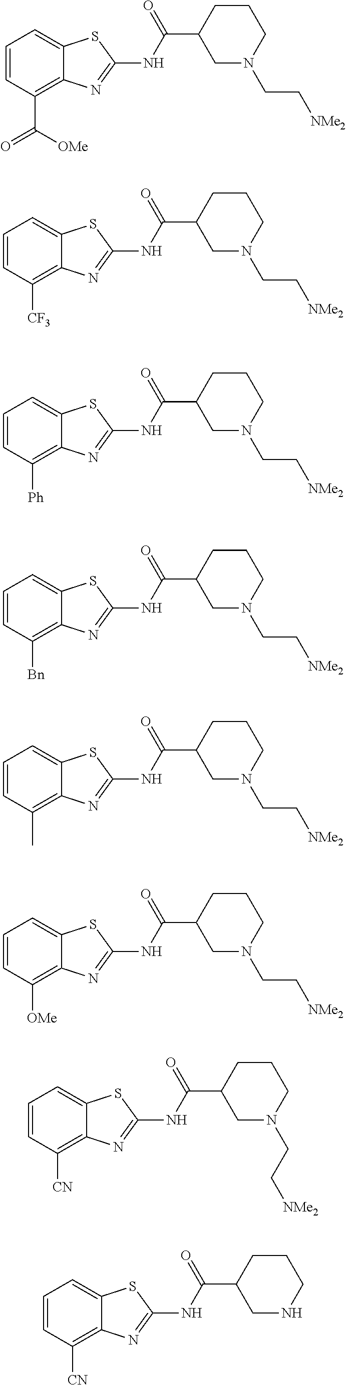

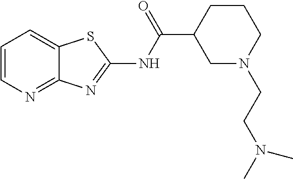 [SMILES: CN(C)CCN1CCCC(C(=O)Nc2nc3ncccc3s2)C1]

or a pharmaceutically acceptable salt thereof.
17. A compound of Formula (II):

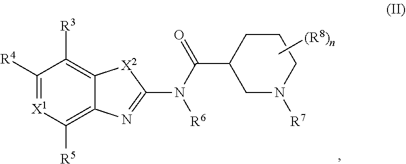

or a pharmaceutically acceptable salt thereof, wherein:
X¹ is selected from N and CR¹;
X² is selected from S, O, and NR²;
R¹ is selected from halo, CN, NO₂, C(O)OR^{a1}, OR^{a1}, NR^{c1}R^{d1} Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹;
R² is selected from H, C₁₋₃ alkyl, and C₁₋₃ haloalkyl;
R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
18. The compound of embodiment 17, wherein R³, R⁴, and R⁵ are each independently selected from H, halo, CN, OH, C₁₋₃ alkyl, C₁₋₃ haloalkyl, C₁₋₃ alkoxy, and C₁₋₃ haloalkoxy.
19. The compound of embodiment 17, wherein R⁸ is selected from halo, CN, OH, NO₂, C₁₋₃ alkyl, C₁₋₃ haloalkyl, C₁₋₃ alkoxy, and C₁₋₃ haloalkoxy.
20. The compound of any one of embodiments 17-19, wherein n is 0, 1, or 2.
21. The compound of embodiment 17, wherein the compound of Formula (I) has formula:

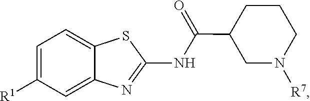

or a pharmaceutically acceptable salt thereof.
22. The compound of any one of embodiments 17-21, wherein R¹ is selected from halo, C₁₋₆ alkyl, C₁₋₆ haloalkyl, C₁₋₃ alkoxy, and C₁₋₃ haloalkoxy.
23. The compound of embodiment 22, wherein R¹ is halo 24. The compound of embodiment 22, wherein R¹ is C₁₋₆ alkyl.
25. The compound of embodiment 22, wherein R¹ is C₁₋₆ haloalkyl.
26. The compound of embodiment 22, wherein R¹ is C₁₋₃ alkoxy.
27. The compound of embodiment 22, wherein R¹ is C₁₋₃ haloalkoxy.
28. The compound of embodiment 17, wherein the compound of Formula (I) has formula:

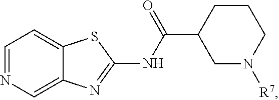

or a pharmaceutically acceptable salt thereof.
29. The compound of any one of embodiments 17-28, wherein R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}.
30. The compound of embodiment 29, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}.
31. The compound of embodiment 30, wherein R⁷ is C₁₋₆ alkyl substituted with NR^{c1}R^{d1}.
32. The compound of embodiment 17, wherein the compound is selected from any one of the following compounds:

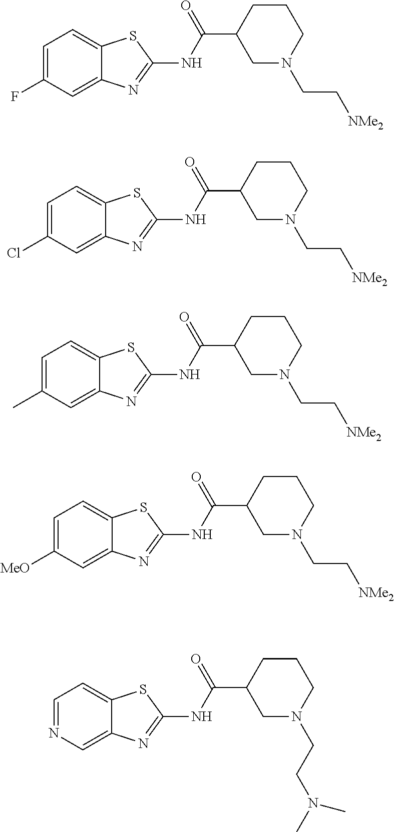 [SMILES: CN(C)CCN1CCCC(C(=O)Nc2nc3cc(F)ccc3s2)C1 ; CN(C)CCN1CCCC(C(=O)Nc2nc3cc(Cl)ccc3s2)C1 ; Cc1ccc2sc(NC(=O)C3CCCN(CCN(C)C)C3)nc2c1 ; COc1ccc2sc(NC(=O)C3CCCN(CCN(C)C)C3)nc2c1 ; CN(C)CCN1CCCC(C(=O)Nc2nc3cnccc3s2)C1]

or a pharmaceutically acceptable salt thereof.
33. A compound of Formula (III):

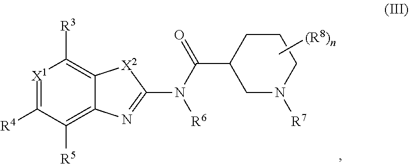

or a pharmaceutically acceptable salt thereof, wherein:
X¹ is selected from N and CR¹;
X² is selected from S, O, and NR²;
R¹ is selected from halo, CN, NO₂, C(O)OR^{a1}, OR^{a1}, NR^{c1}R^{d1}, Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹;
R² is selected from H, C₁₋₃ alkyl, and C₁₋₃ haloalkyl;
R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
34. The compound of embodiment 33, wherein R³, R⁴, and R⁵ are each independently selected from H, halo, CN, OH, C₁₋₃ alkyl, C₁₋₃ haloalkyl, C₁₋₃ alkoxy, and C₁₋₃ haloalkoxy.
35. The compound of embodiment 33, wherein R⁸ is selected from halo, CN, OH, NO₂, C₁₋₃ alkyl, C₁₋₃ haloalkyl, C₁₋₃ alkoxy, and C₁₋₃ haloalkoxy.
36. The compound of any one of embodiments 33-35, wherein n is 0, 1, or 2.
37. The compound of embodiment 33, wherein the compound of Formula (III) has formula:

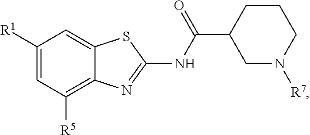

or a pharmaceutically acceptable salt thereof.
38. The compound of any one of embodiments 33-37, wherein R¹ is selected from halo, C(O)OR^{a1}, OR^{a1}, Cy¹, C₁₋₆ alkyl, and C₁₋₆ haloalkyl, wherein said C₁₋₆ alkyl is optionally substituted with OR^{a1}.
39. The compound of embodiment 38, wherein R¹ is halo.
40. The compound of embodiment 38, wherein R¹ is C(O)O(C₁₋₃ alkyl).
41. The compound of embodiment 38, wherein R¹ is C₁₋₃ alkoxy or C₁₋₃ haloalkoxy.
42. The compound of embodiment 38, wherein R¹ is C₁₋₆ alkyl or C₁₋₆ haloalkyl.
43. The compound of embodiment 38, wherein R¹ is C₆₋₁₀ aryl.
44. The compound of embodiment 38, wherein R¹ is C₁₋₆ alkyl substituted with C₁₋₃ alkoxy.
45. The compound of embodiment 33, wherein the compound of Formula (III) has formula:

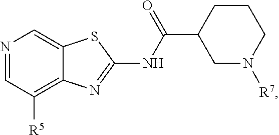

or a pharmaceutically acceptable salt thereof.
46. The compound of any one of embodiments 33-45, wherein R⁵ is selected from H and halo.
47. The compound of any one of embodiments 33-46, wherein R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}.
48. The compound of embodiment 47, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}.
49. The compound of embodiment 47, wherein R⁷ is C₁₋₆ alkyl substituted with NR^{c1}R^{d1}.
50. The compound of embodiment 33, wherein the compound is selected from any one of the following compounds:

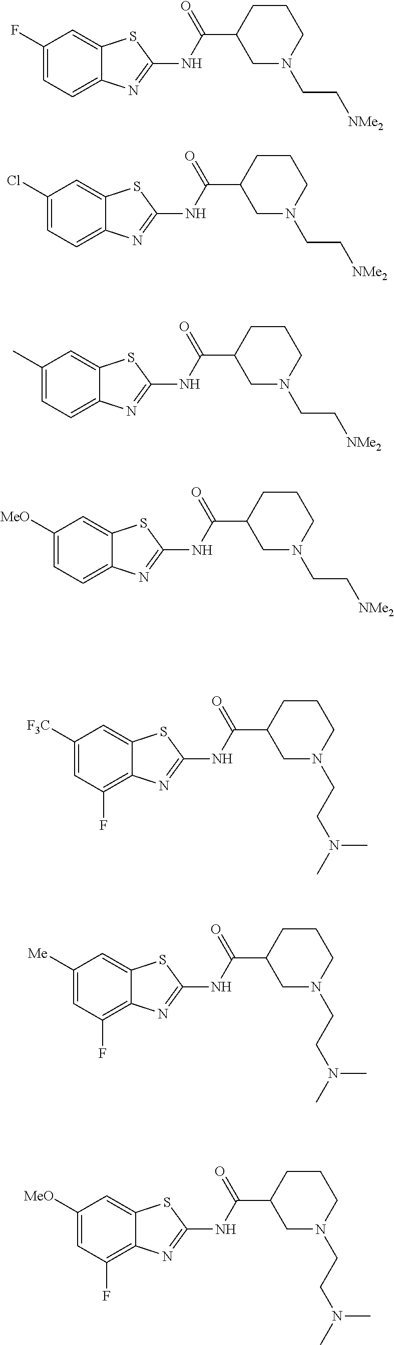

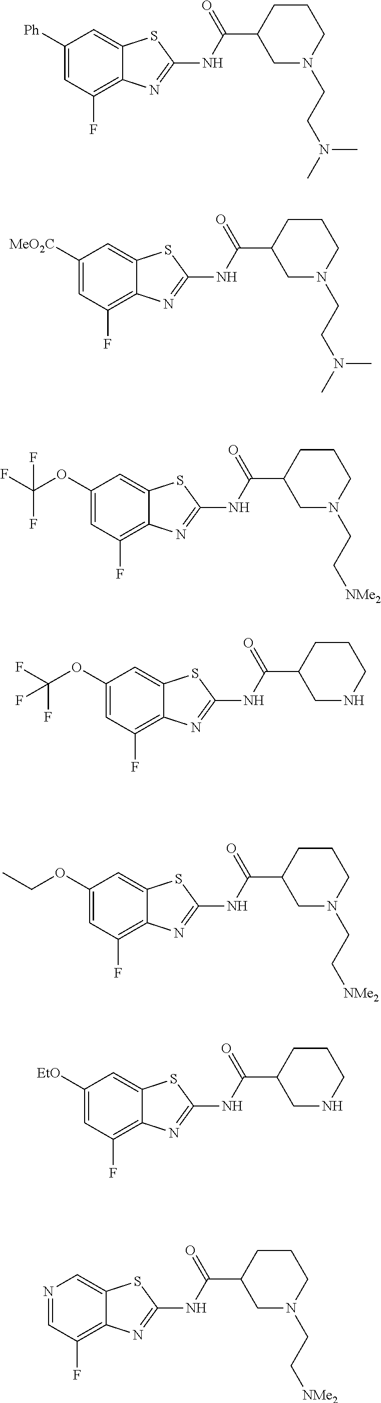

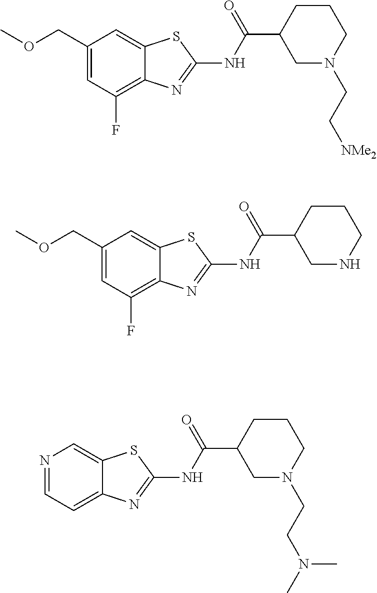 [SMILES: COCc1cc(F)c2nc(NC(=O)C3CCCN(CCN(C)C)C3)sc2c1 ; COCc1cc(F)c2nc(NC(=O)C3CCCNC3)sc2c1 ; CN(C)CCN1CCCC(C(=O)Nc2nc3ccncc3s2)C1]

or a pharmaceutically acceptable salt thereof.
51. A compound of Formula (IV):

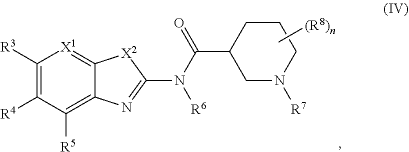

or a pharmaceutically acceptable salt thereof, wherein:
X¹ is selected from N and CR¹;
X² is selected from S, O, and NR²;
R¹ is selected from halo, CN, NO₂, C(O)OR^{a1}, OR^{a1}, NR^{c1}R^{d1}, Cy¹, C₁₋₆ alkyl, and C₁₋₆ haloalkyl, wherein said C₁₋₆ alkyl is optionally substituted with CN, C(O)OR^{a1}, OR^{a1}, NR^{c1}R^{d1}, or Cy¹;
R² is selected from H, C₁₋₃ alkyl, and C₁₋₃ haloalkyl;
R³, R⁴, and R⁵ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
52. The compound of embodiment 51, wherein R³, R⁴, and R⁵ are each independently selected from H, halo, CN, OH, C₁₋₃ alkyl, C₁₋₃ haloalkyl, C₁₋₃ alkoxy, and C₁₋₃ haloalkoxy.
53. The compound of embodiment 51, wherein R⁸ is selected from halo, CN, OH, NO₂, C₁₋₃ alkyl, C₁₋₃ haloalkyl, C₁₋₃ alkoxy, and C₁₋₃ haloalkoxy.
54. The compound of any one of embodiments 51-53, wherein n is 0, 1, or 2.
55. The compound of embodiment 51, wherein the compound of Formula (IV) has formula:

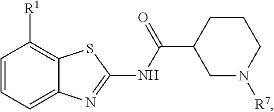

or a pharmaceutically acceptable salt thereof.
56. The compound of any one of embodiments 51-55, wherein R¹ is selected from halo, C₁₋₆ alkyl, C₁₋₆ haloalkyl, C₁₋₃ alkoxy, and C₁₋₃ haloalkoxy.
57. The compound of embodiment 56, wherein R¹ is halo 58. The compound of embodiment 56, wherein R¹ is C₁₋₆ alkyl.
59. The compound of embodiment 56, wherein R¹ is C₁₋₆ haloalkyl.
60. The compound of embodiment 56, wherein R¹ is C₁₋₃ alkoxy.
61. The compound of embodiment 56, wherein R¹ is C₁₋₃ haloalkoxy.
62. The compound of embodiment 51, wherein the compound of Formula (IV) has formula:

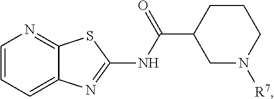

or a pharmaceutically acceptable salt thereof.
63. The compound of any one of embodiments 51-62, wherein R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}.
64. The compound of embodiment 63, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}.
65. The compound of embodiment 63, wherein R⁷ is C₁₋₆ alkyl substituted with NR^{c1}R^{d1}.
66. The compound of embodiment 51, wherein the compound is selected from any one of the following compounds

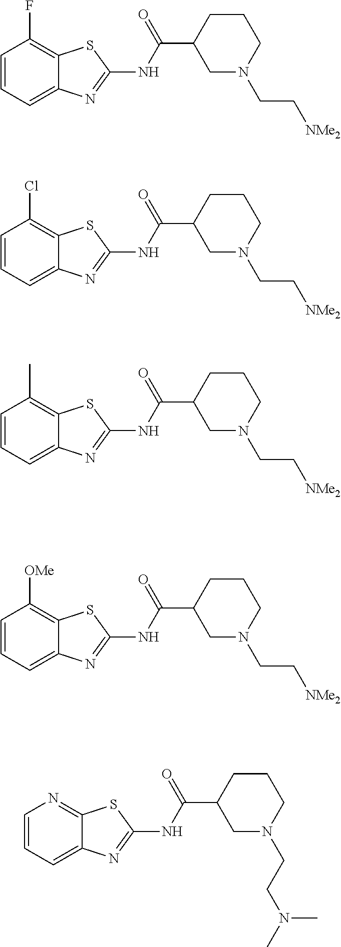 [SMILES: CN(C)CCN1CCCC(C(=O)Nc2nc3cccc(F)c3s2)C1 ; CN(C)CCN1CCCC(C(=O)Nc2nc3cccc(Cl)c3s2)C1 ; Cc1cccc2nc(NC(=O)C3CCCN(CCN(C)C)C3)sc12 ; COc1cccc2nc(NC(=O)C3CCCN(CCN(C)C)C3)sc12 ; CN(C)CCN1CCCC(C(=O)Nc2nc3cccnc3s2)C1]

or a pharmaceutically acceptable salt thereof.
67. A compound of Formula (V):

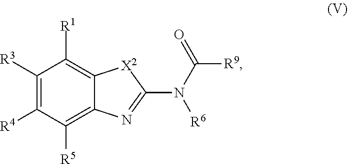

or a pharmaceutically acceptable salt thereof, wherein:
R⁹ is selected from any one of the following groups:

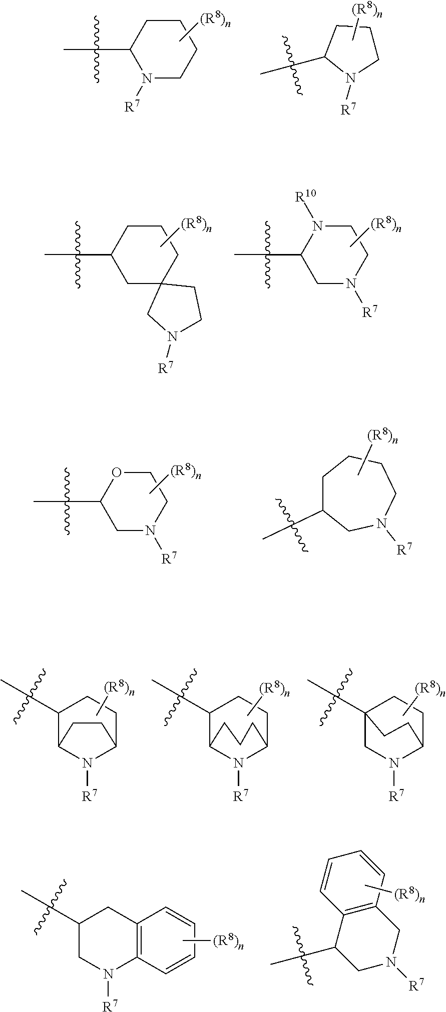

X² is selected from S, O, and NR²;
R² is selected from H, C₁₋₃ alkyl, and C₁₋₃ haloalkyl;
R³ is halo;
R⁵ is halo;
R¹ and R⁴ are each independently selected from H, halo, CN, NO₂, C(O)OR^{a1}, OR^{a1}, NR^{c1}R^{d1} C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
R¹⁰ is selected from H, C₁₋₃ alkyl, C₁₋₃ haloalkyl, C(O)OR^{a1}, C(O)R^{b1}, and C(O)NR^{c1}R^{d1};
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, N^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
68. The compound of embodiment 67, wherein R¹ and R⁴ are each independently selected from H, halo, CN, OH, C₁₋₃ alkyl, C₁₋₃ haloalkyl, C₁₋₃ alkoxy, and C₁₋₃ haloalkoxy.
69. The compound of embodiment 67, wherein the compound of Formula (V) has formula:

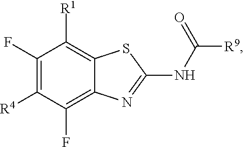

or a pharmaceutically acceptable salt thereof.
70. The compound of any one of embodiments 67-69, wherein R⁹ is:

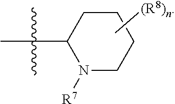

71. The compound of any one of embodiments 67-69, wherein R⁹ is:

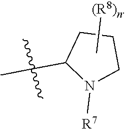

72. The compound of any one of embodiments 67-69, wherein R⁹ is:

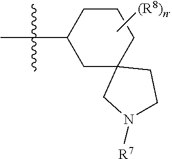

73. The compound of any one of embodiments 67-69, wherein R⁹ is.

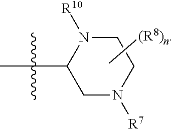

74. The compound of any one of embodiments 67-69, wherein R⁹ is:

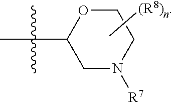

75. The compound of any one of embodiments 67-69, wherein R⁹ is:

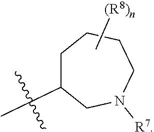

76. The compound of any one of embodiments 67-69, wherein R⁹ is:

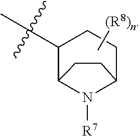

77. The compound of any one of embodiments 67-69, wherein R⁹ is:

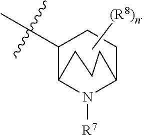

78. The compound of any one of embodiments 67-69, wherein R⁹ is:

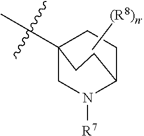

79. The compound of any one of embodiments 67-69, wherein R⁹ is:

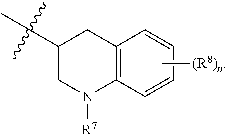

80. The compound of any one of embodiments 67-69, wherein R⁹ is

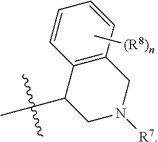

81. The compound of any one of embodiments 67-80, wherein R⁸ is selected from halo, CN, OH, NO₂, C₁₋₃ alkyl, C₁₋₃ haloalkyl, C₁₋₃ alkoxy, and C₁₋₃ haloalkoxy.
82. The compound of embodiment 81, wherein n is 0, 1, or 2.
83. The compound of embodiment 67, selected from any one of the following formulae:

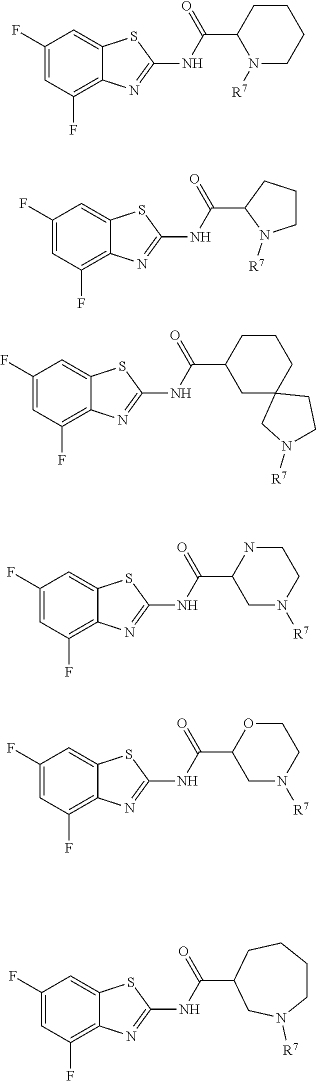

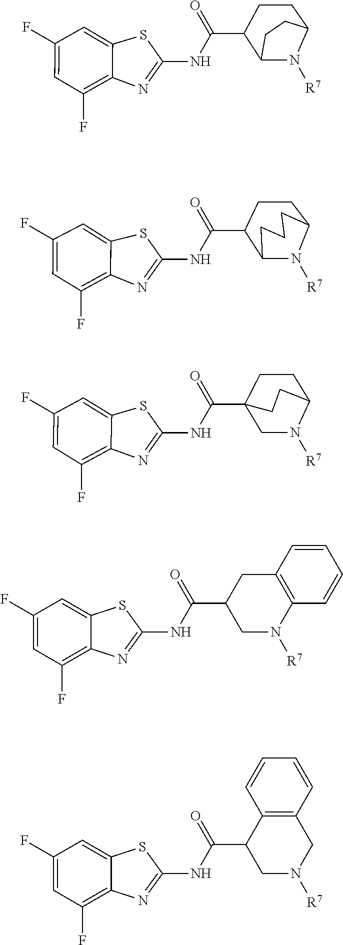

or a pharmaceutically acceptable salt thereof.
84. The compound of any one of embodiments 67-83, wherein R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1}.
85. The compound of embodiment 84, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}.
86. The compound of embodiment 84, wherein R⁷ is C₁₋₆ alkyl substituted with NR^{c1}R^{d1}.
87. The compound of embodiment 67, wherein the compound is selected from any one of the following compounds:

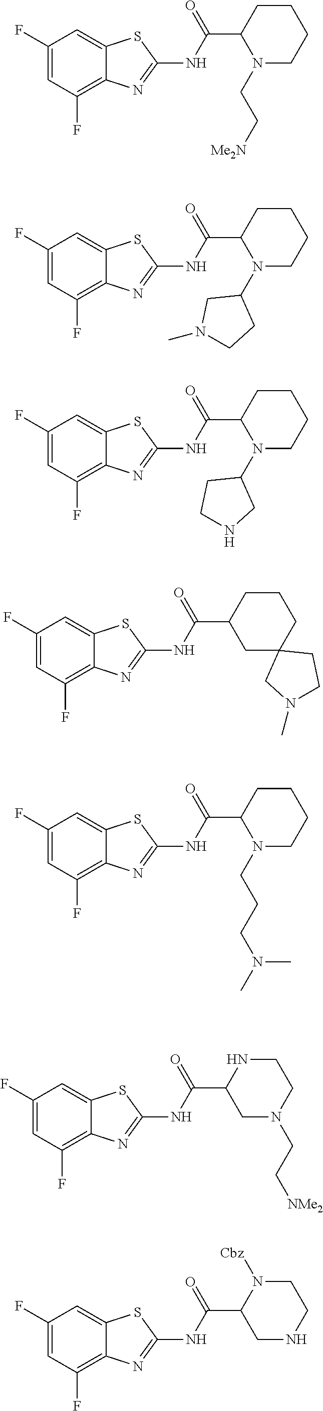

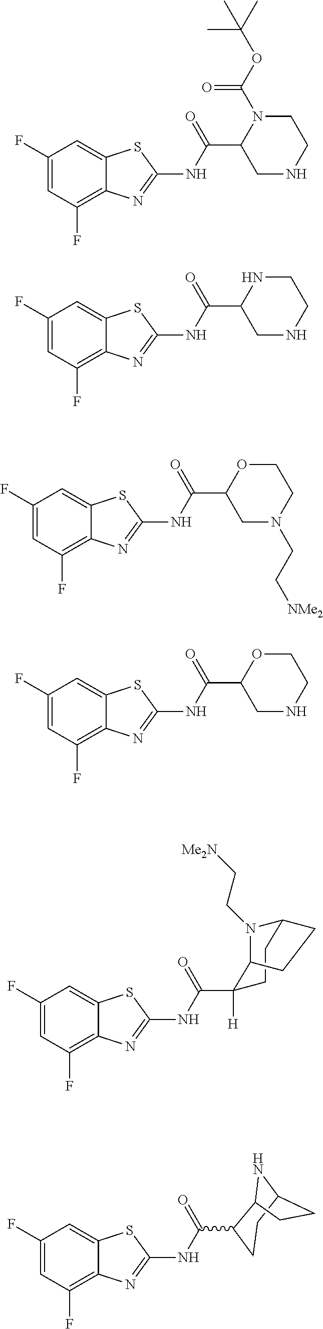

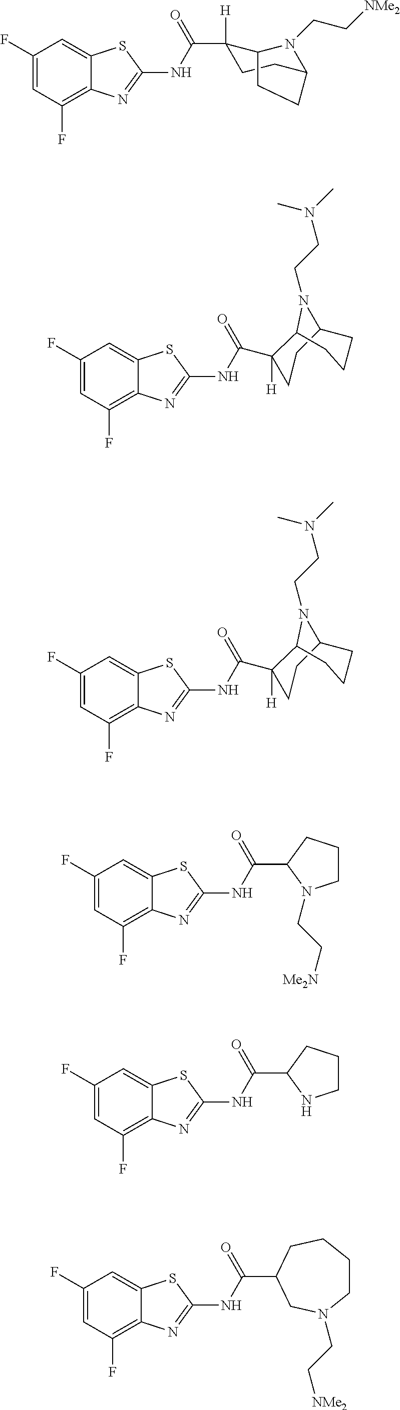 [SMILES: CN(C)CCN1C2CCC(C(=O)Nc3nc4c(F)cc(F)cc4s3)C1CC2 ; CN(C)CCN1C2CCCC1C(C(=O)Nc1nc3c(F)cc(F)cc3s1)CC2 ; CN(C)CCN1C2CCCC1C(C(=O)Nc1nc3c(F)cc(F)cc3s1)CC2 ; CN(C)CCN1CCCC1C(=O)Nc1nc2c(F)cc(F)cc2s1 ; O=C(Nc1nc2c(F)cc(F)cc2s1)C1CCCN1 ; CN(C)CCN1CCCCC(C(=O)Nc2nc3c(F)cc(F)cc3s2)C1]

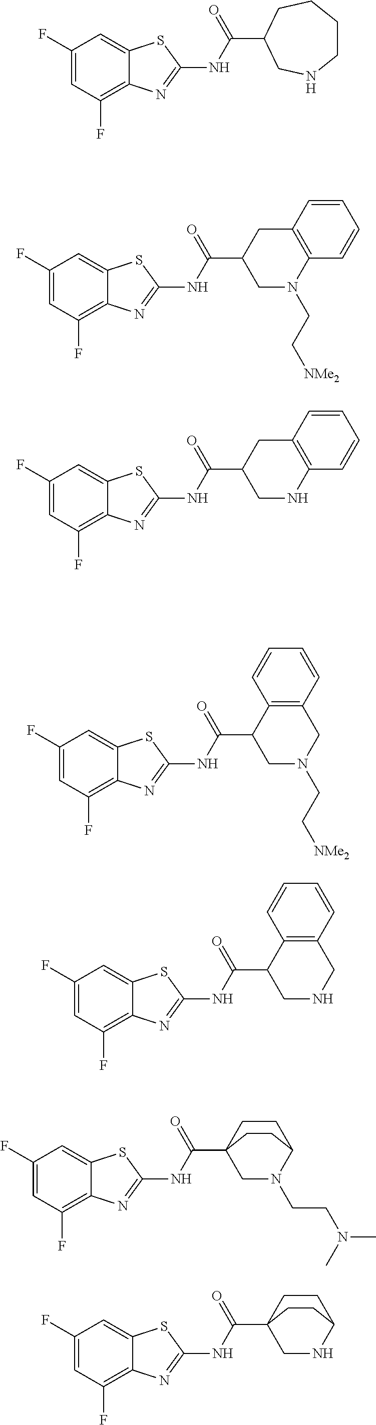

or a pharmaceutically acceptable salt thereof.
88. A compound of Formula (VI):

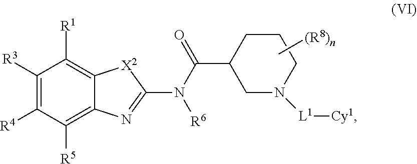

or a pharmaceutically acceptable salt thereof, wherein:
X² is selected from S, O, and NR²;
R² is selected from H, C₁₋₃ alkyl, and C₁₋₃ haloalkyl;
R³ is selected from H and halo;
R⁵ is halo;
R¹ and R⁴ are each independently selected from H, halo, CN, NO₂, C(O)OR^{a1}, OR^{a1}, NR^{c1}R^{d1} C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
L¹ is selected from a bond, C(═O), S(O)₂, and C₁₋₃ alkylene;
Cy¹ is selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, N^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
89. The compound of embodiment 88, wherein R¹ and R⁴ are each independently selected from H, halo, CN, OH, C₁₋₃ alkyl, C₁₋₃ haloalkyl, C₁₋₃ alkoxy, and C₁₋₃ haloalkoxy.
90. The compound of embodiment 88, wherein the compound of Formula (VI) is selected from:

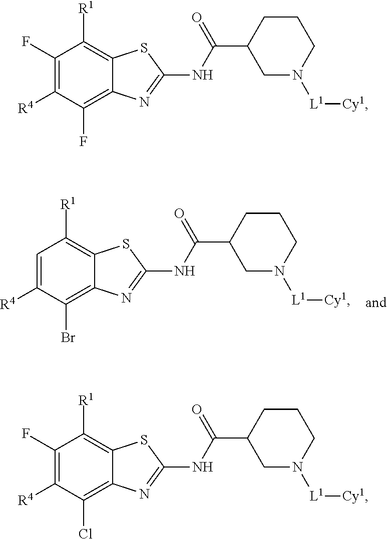

or a pharmaceutically acceptable salt thereof.
   91. The compound of any one of embodiments 88-90, wherein L¹ is C(═O).
92. The compound of any one of embodiments 88-90, wherein L¹ is a bond.
93. The compound of any one of embodiments 88-90, wherein L¹ is C₁₋₃ alkylene.
94. The compound of any one of embodiments 88-90, selected from any one of the following formulae:

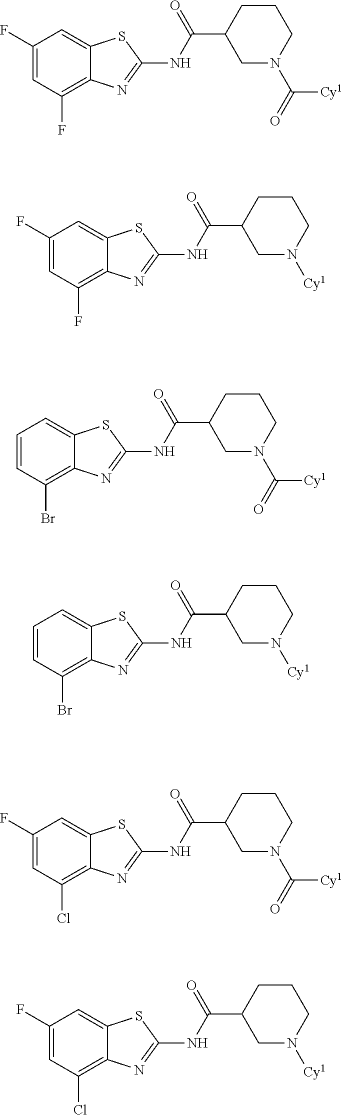

or a pharmaceutically acceptable salt thereof.
95. The compound of any one of embodiments 88-94, wherein Cy¹ is selected from C₆₋₁₀ aryl, 5-14 membered heteroaryl, and 4-10 membered heterocycloalkyl, each of which is optionally substituted with 1, 2, or 3 substituents independently selected from R^{Cy1}.
96. The compound of embodiment 95, wherein Cy¹ is C₆₋₁₀ aryl, optionally substituted with R^{Cy1}.
97. The compound of embodiment 95, wherein Cy¹ is 5-14 membered heteroaryl, optionally substituted with R^{Cy1}.
98. The compound of embodiment 95, wherein Cy¹ is 4-10 membered heterocycloalkyl, optionally substituted with R^{Cy1}.
99. The compound of any one of embodiments 88-98, wherein R^{Cy1} is selected from halo, C₁₋₆ alkyl, C₁₋₆ haloalkyl, C₂₋₆ alkenyl, OR^{a2}, C(O)R^{b2}, C(O)NR^{c2}R^{d2}, C(O)OR^{a2}, and NR^{c2}R^{d2}, wherein said C₁₋₆ alkyl is optionally substituted with OR^{a2}, C(O)NR^{c2}R^{d2} or C(O)OR^{a2}.
100. The compound of embodiment 88, wherein the compound is selected from any one of the following compounds:

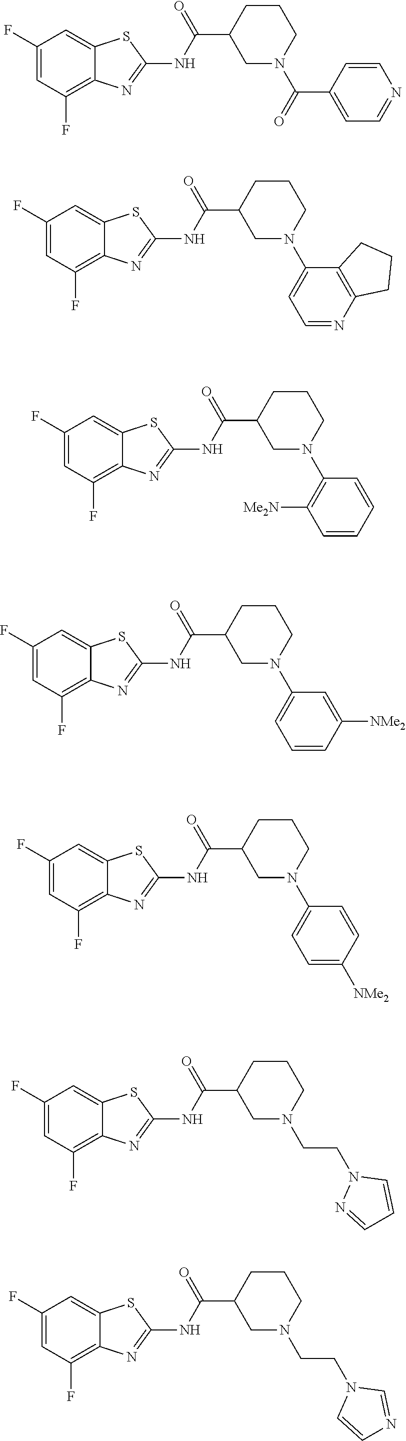

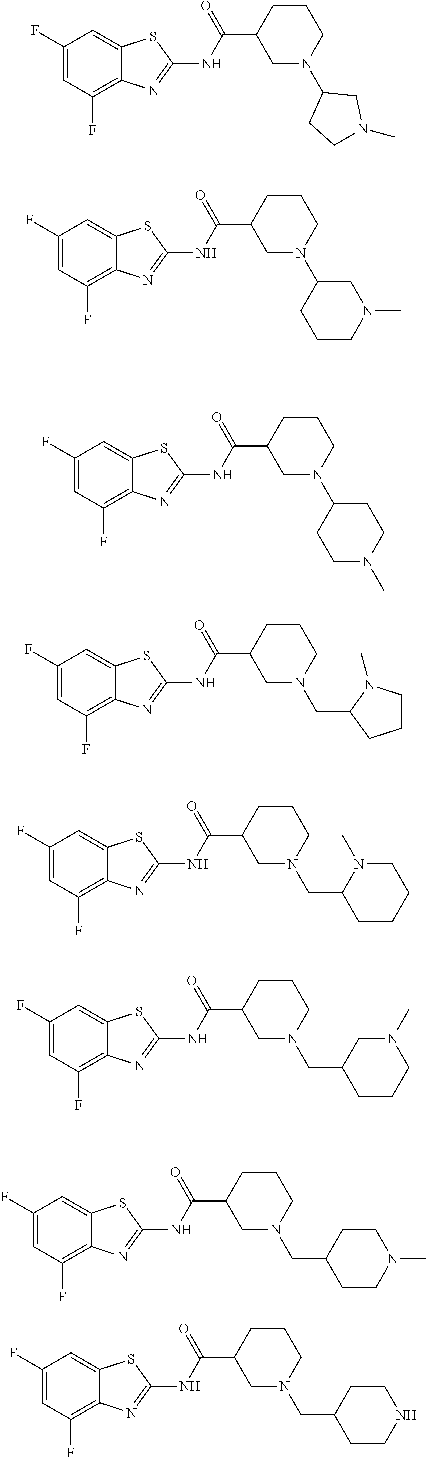

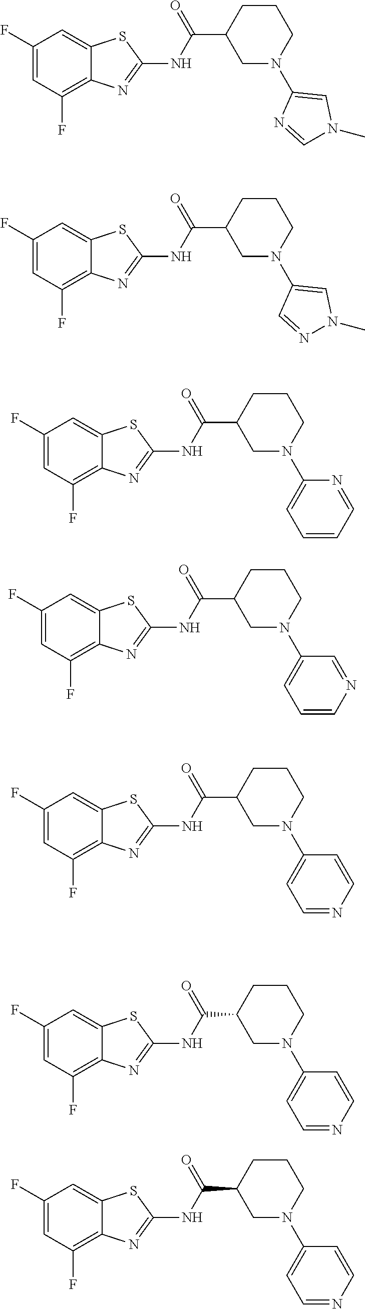

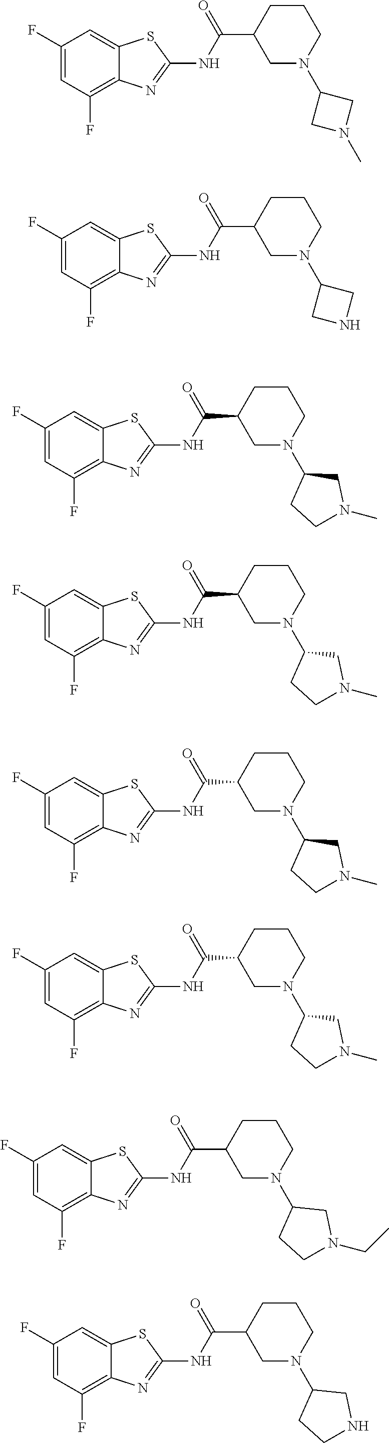

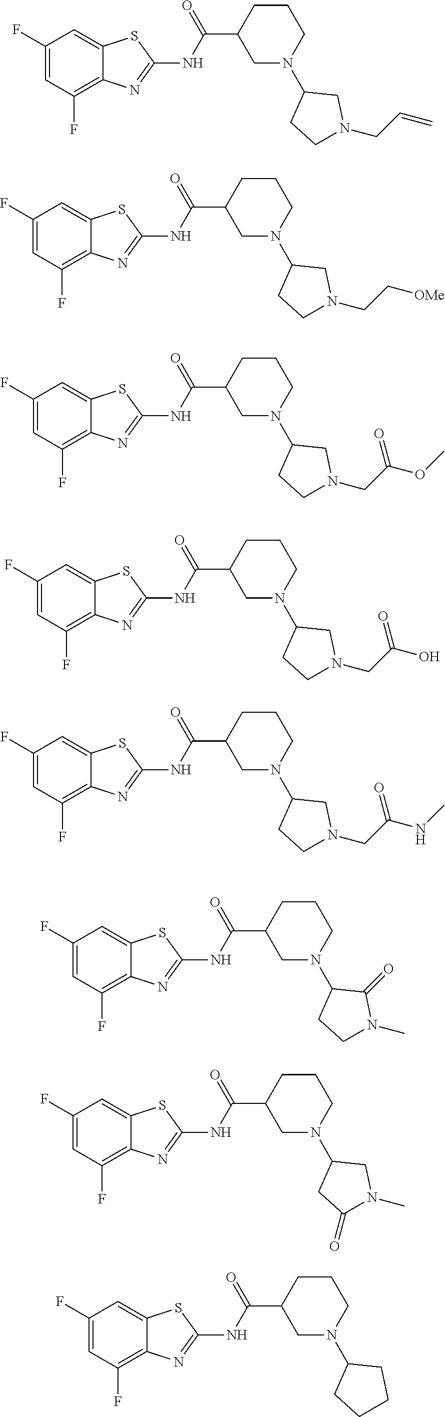

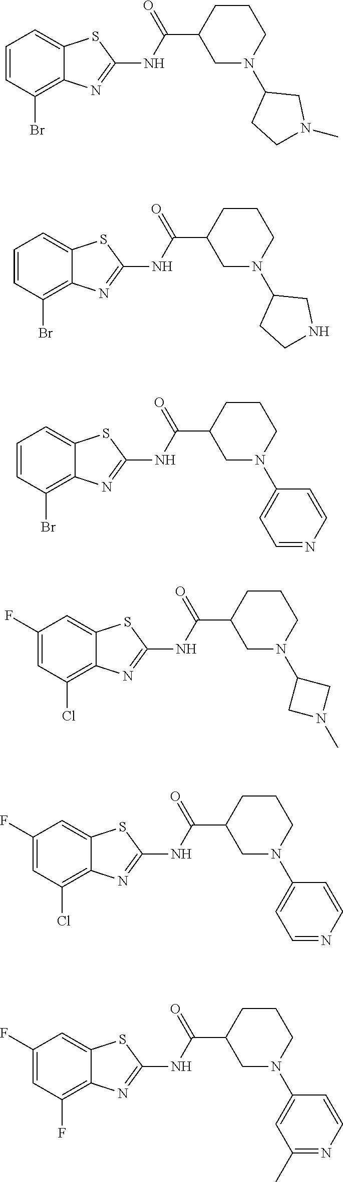

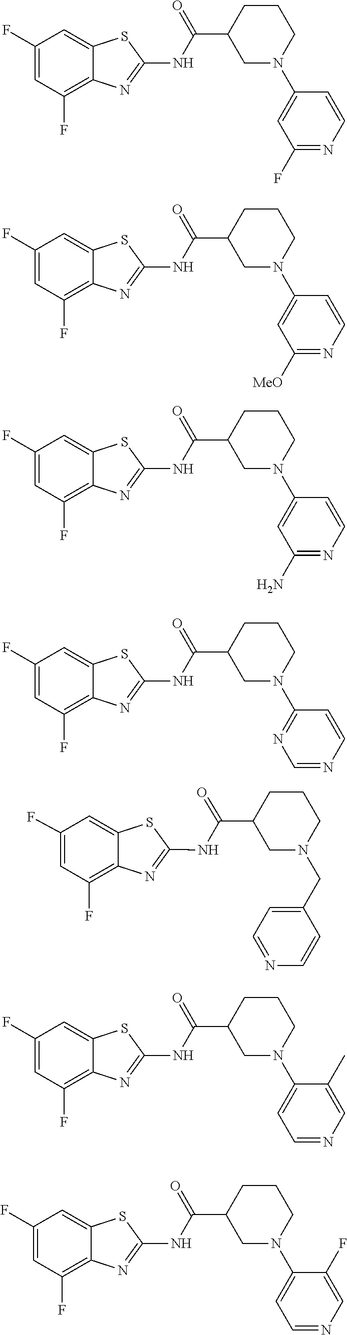

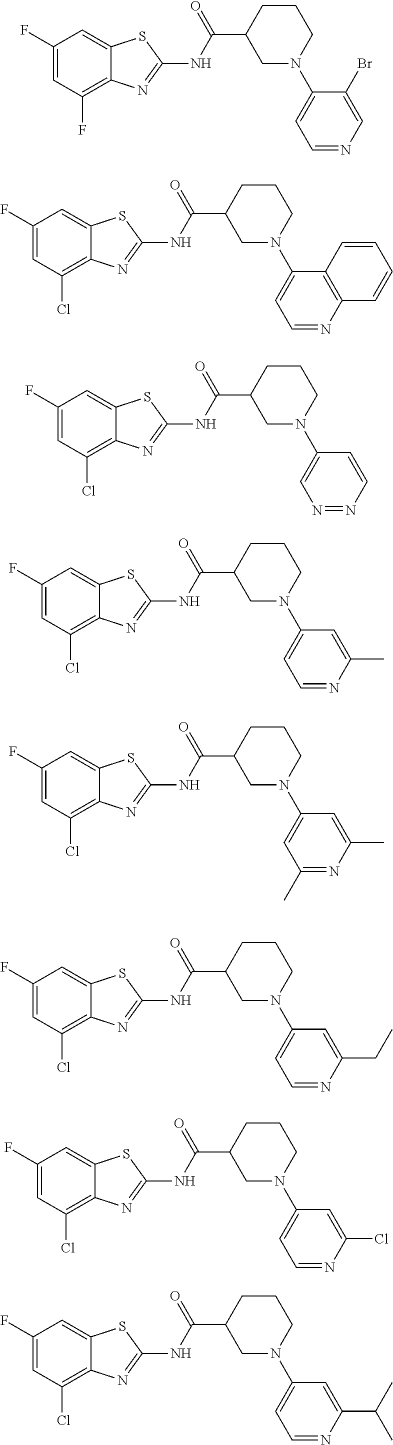

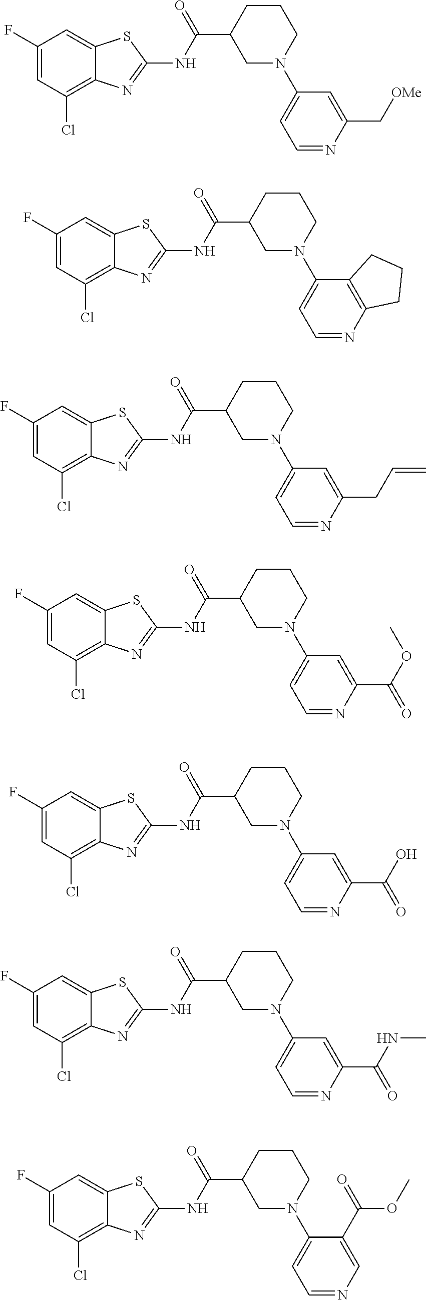

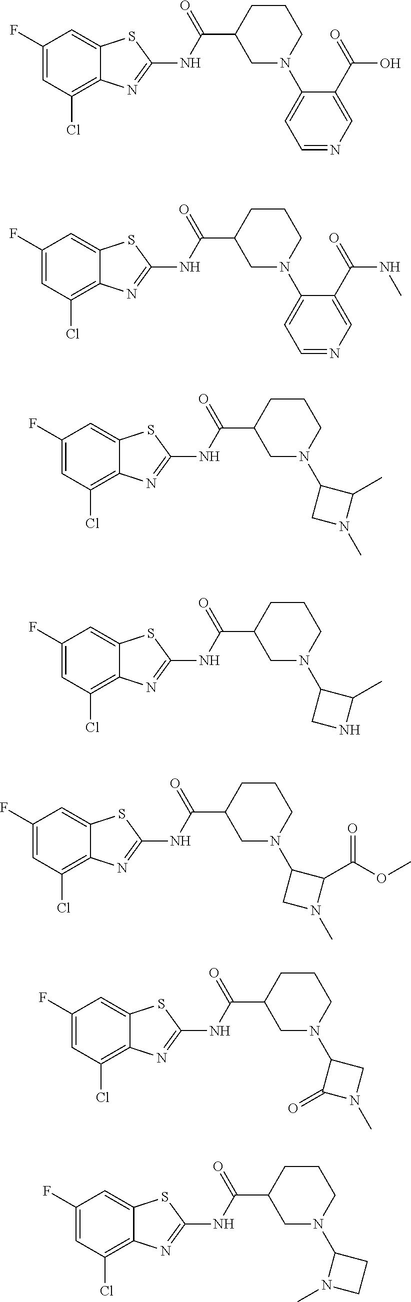

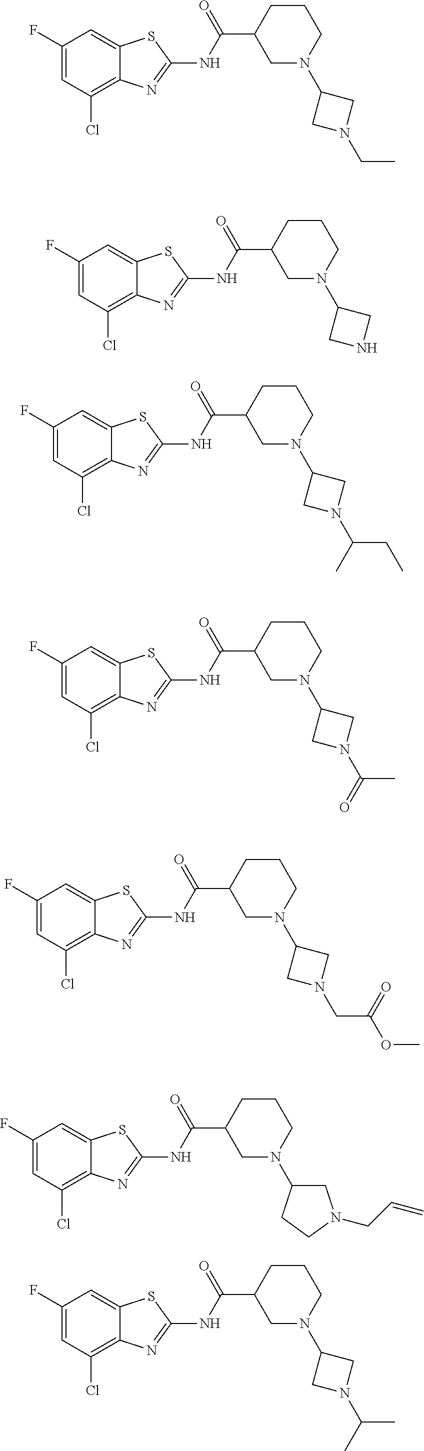

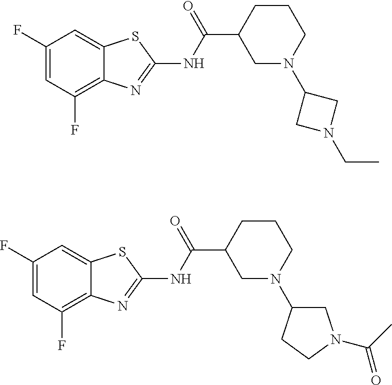 [SMILES: CCN1CC(N2CCCC(C(=O)Nc3nc4c(F)cc(F)cc4s3)C2)C1 ; CC(=O)N1CCC(N2CCCC(C(=O)Nc3nc4c(F)cc(F)cc4s3)C2)C1]

or a pharmaceutically acceptable salt thereof.
101. A compound of Formula (VII):

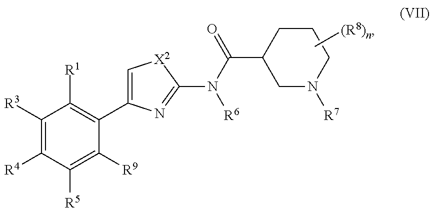

or a pharmaceutically acceptable salt thereof, wherein:
X² is selected from S, O, and NR²;
R² is selected from H, C₁₋₃ alkyl, and C₁₋₃ haloalkyl;
R¹, R³, R⁴, R⁵, and R⁹ are each independently selected from H, halo, CN, NO₂, C(O)OR^{a1}, OR^{a1}, NR^{c1}R^{d1} C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
102. The compound of embodiment 101, wherein R¹, R³, R⁴, R⁵, and R⁹ are each independently selected from H, halo, OR^{a1}, NR^{c1}R^{d1} and C₁₋₃ alkyl.
103. The compound of embodiment 102, wherein the compound of Formula (VII) has formula:

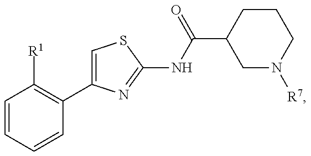

or a pharmaceutically acceptable salt thereof, wherein R¹ is selected from halo, OR^{a1}, NR^{c1}R^{d1} and C₁₋₃ alkyl.
104. The compound of embodiment 103, wherein R¹ is halo, C₁₋₃ alkyl, or C₁₋₃ alkoxy.
105. The compound of embodiment 102, wherein the compound of Formula (VII) has formula:

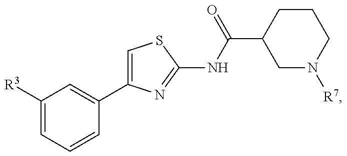

or a pharmaceutically acceptable salt thereof, wherein R³ is selected from halo, OR^{a1}, NR^{c1}R^{d1} and C₁₋₃ alkyl.
106. The compound of embodiment 105, wherein R³ is selected from halo, C₁₋₃ alkyl, or C₁₋₃ alkoxy.
107. The compound of embodiment 102, wherein the compound of Formula (VII) has formula:

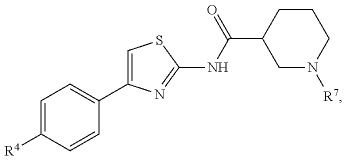

or a pharmaceutically acceptable salt thereof, wherein R⁴ is selected from halo, OR^{a1}, NR^{c1}R^{d1} and C₁₋₃ alkyl.
108. The compound of embodiment 102, wherein the compound of Formula (VII) has formula:

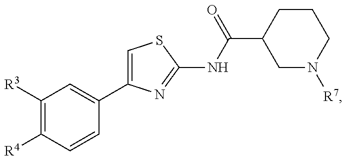

or a pharmaceutically acceptable salt thereof, wherein R³ and R⁴ are each independently selected from halo, OR^{a1}, NR^{c1}R^{d1} and C₁₋₃ alkyl.
109. The compound of embodiment 108, wherein R³ and R⁴ are each independently selected from halo and C₁₋₃ alkoxy.
110. The compound of embodiment 102, wherein the compound of Formula (VII) has formula:

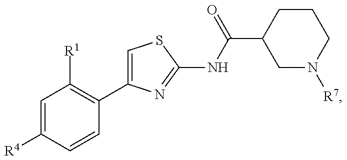

or a pharmaceutically acceptable salt thereof, wherein R¹ and R⁴ are each independently selected from halo, OR^{a1}, NR^{c1}R^{d1}, and C₁₋₃ alkyl.
111. The compound of embodiment 110, wherein R¹ and R⁴ are each independently selected from halo and C₁₋₃ alkoxy.
112. The compound of any one of embodiments 101-111, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}
113. The compound of embodiment 112, wherein R⁷ is selected from Cy¹, C(═O)Cy¹, and C₁₋₆ alkyl substituted with Cy¹.
114. The compound of embodiment 112, wherein R⁷ is C₁₋₆ alkyl substituted with NR^{c1}R^{d1}.
115. The compound of embodiment 101, wherein the compound is selected from any one of the following compounds:

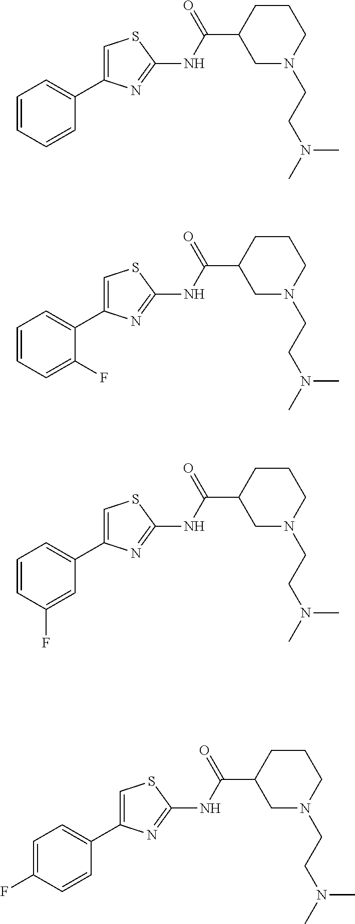 [SMILES: CN(C)CCN1CCCC(C(=O)Nc2nc(-c3ccccc3)cs2)C1 ; CN(C)CCN1CCCC(C(=O)Nc2nc(-c3ccccc3F)cs2)C1 ; CN(C)CCN1CCCC(C(=O)Nc2nc(-c3cccc(F)c3)cs2)C1 ; CN(C)CCN1CCCC(C(=O)Nc2nc(-c3ccc(F)cc3)cs2)C1]

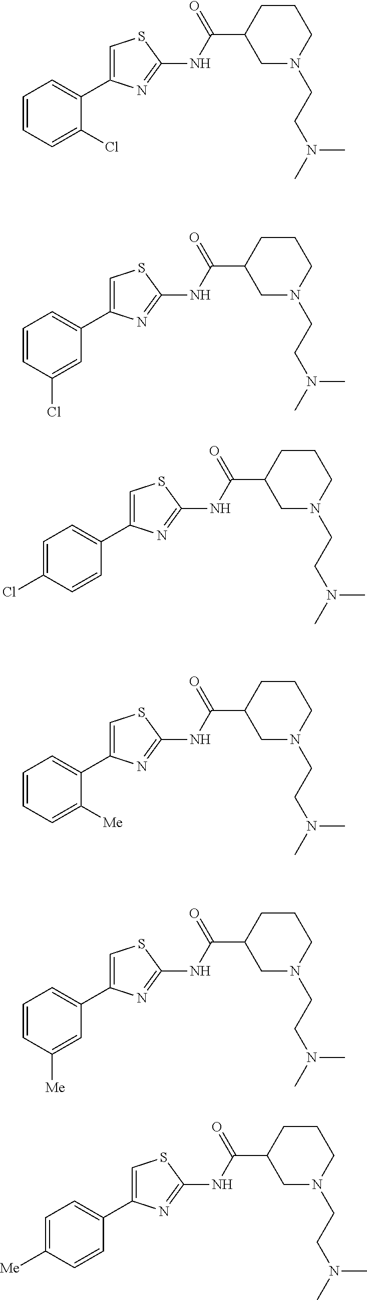

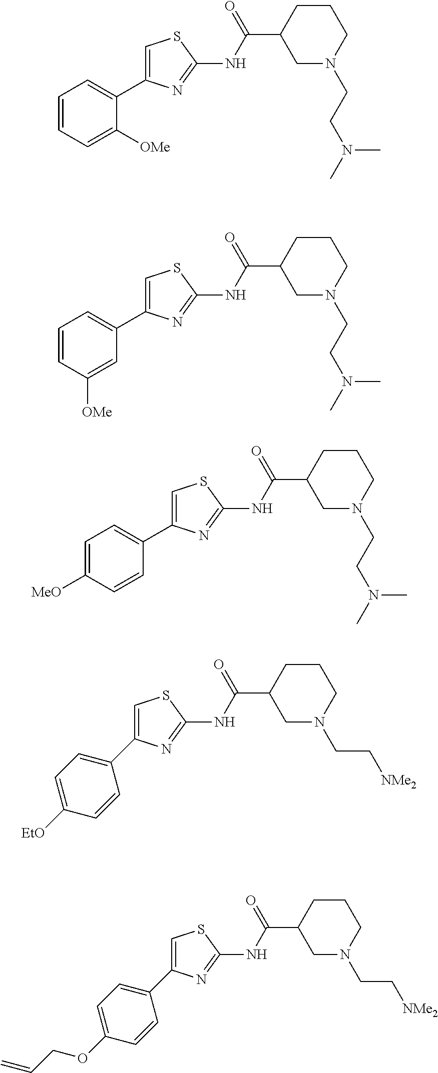

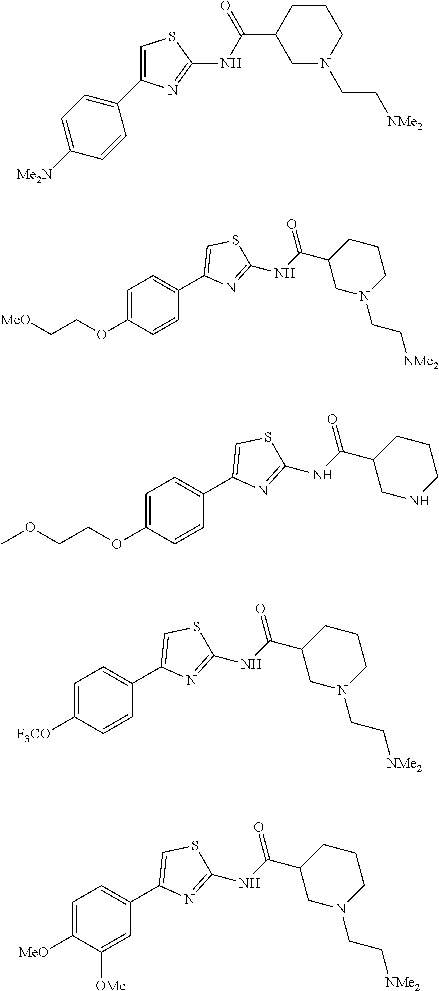 [SMILES: CN(C)CCN1CCCC(C(=O)Nc2nc(-c3ccc(N(C)C)cc3)cs2)C1 ; COCCOc1ccc(-c2csc(NC(=O)C3CCCN(CCN(C)C)C3)n2)cc1 ; COCCOc1ccc(-c2csc(NC(=O)C3CCCNC3)n2)cc1 ; CN(C)CCN1CCCC(C(=O)Nc2nc(-c3ccc(OC(F)(F)F)cc3)cs2)C1 ; COc1ccc(-c2csc(NC(=O)C3CCCN(CCN(C)C)C3)n2)cc1OC]

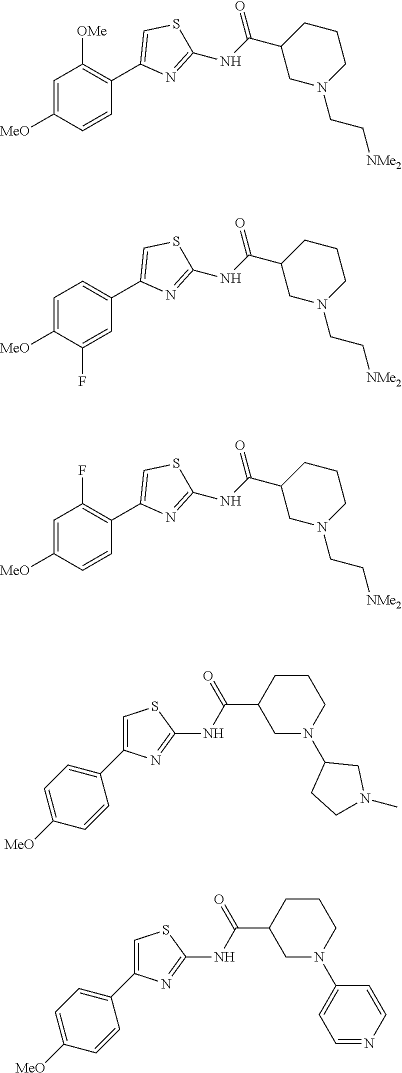 [SMILES: COc1ccc(-c2csc(NC(=O)C3CCCN(CCN(C)C)C3)n2)c(OC)c1 ; COc1ccc(-c2csc(NC(=O)C3CCCN(CCN(C)C)C3)n2)cc1F ; COc1ccc(-c2csc(NC(=O)C3CCCN(CCN(C)C)C3)n2)c(F)c1 ; COc1ccc(-c2csc(NC(=O)C3CCCN(C4CCN(C)C4)C3)n2)cc1 ; COc1ccc(-c2csc(NC(=O)C3CCCN(c4ccncc4)C3)n2)cc1]

or a pharmaceutically acceptable salt thereof.
116. A compound of Formula (VIII):

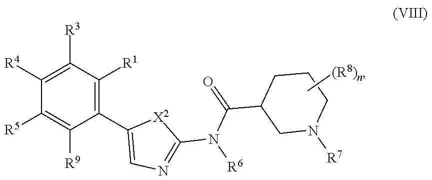

or a pharmaceutically acceptable salt thereof, wherein:
X² is selected from S, O, and NR²;
R² is selected from H, C₁₋₃ alkyl, and C₁₋₃ haloalkyl;
R¹, R³, R⁴, R⁵, and R⁹ are each independently selected from H, halo, CN, NO₂, C(O)OR^{a1}, OR^{a1}, NR^{c1}R^{d1} C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
117. The compound of embodiment 116, wherein R¹, R³, R⁴, R⁵, and R⁹ are each independently selected from H, halo, OR^{a1}, NR^{c1}R^{d1}, and C₁₋₃ alkyl.
118. The compound of embodiment 116, wherein R¹, R³, R⁴, R⁵, and R⁹ are each independently selected from H, halo, C₁₋₃ alkyl, and C₁₋₃ alkoxy.
119. The compound of embodiment 116, wherein the compound of Formula (VIII) has formula:

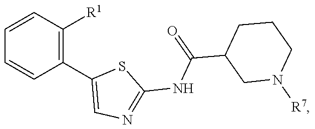

or a pharmaceutically acceptable salt thereof, wherein R¹ is selected from halo, C₁₋₃ alkyl, and C₁₋₃ alkoxy.
120. The compound of embodiment 116, wherein the compound of Formula (VIII) has formula:

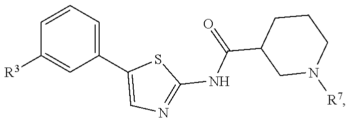

or a pharmaceutically acceptable salt thereof, wherein R³ is selected from halo, C₁₋₃ alkyl, and C₁₋₃ alkoxy.
121. The compound of embodiment 116, wherein the compound of Formula (VIII) has formula:

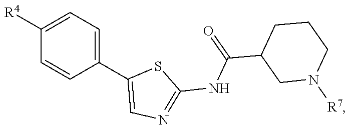

or a pharmaceutically acceptable salt thereof, wherein R⁴ is selected from halo, C₁₋₃ alkyl, and C₁₋₃ alkoxy.
122. The compound of any one of embodiments 116-121, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a}, or NR^{c1}R^{d1}.
123. The compound of any one of embodiments 116-121, wherein R⁷ is C₁₋₆ alkyl substituted with NR^{c1}R^{d1}.
124. The compound of embodiment 116, wherein the compound is selected from any one of the following compounds:

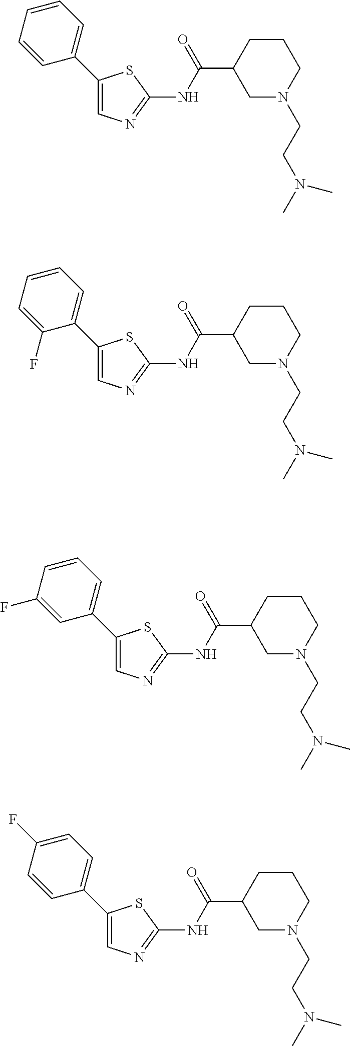 [SMILES: CN(C)CCN1CCCC(C(=O)Nc2ncc(-c3ccccc3)s2)C1 ; CN(C)CCN1CCCC(C(=O)Nc2ncc(-c3ccccc3F)s2)C1 ; CN(C)CCN1CCCC(C(=O)Nc2ncc(-c3cccc(F)c3)s2)C1 ; CN(C)CCN1CCCC(C(=O)Nc2ncc(-c3ccc(F)cc3)s2)C1]

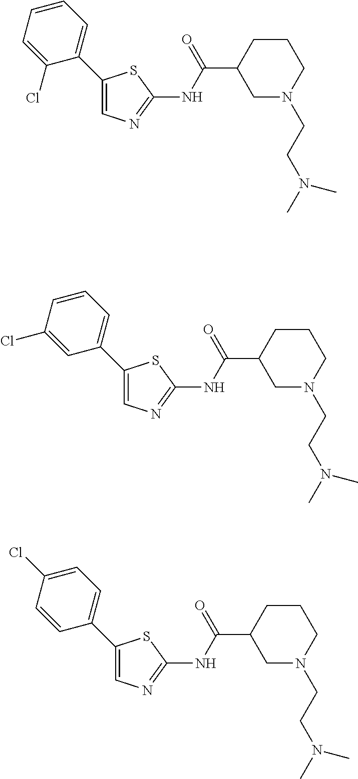 [SMILES: CN(C)CCN1CCCC(C(=O)Nc2ncc(-c3ccccc3Cl)s2)C1 ; CN(C)CCN1CCCC(C(=O)Nc2ncc(-c3cccc(Cl)c3)s2)C1 ; CN(C)CCN1CCCC(C(=O)Nc2ncc(-c3ccc(Cl)cc3)s2)C1]

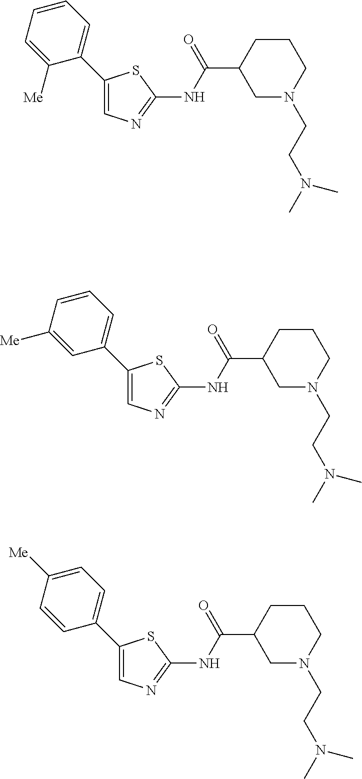 [SMILES: Cc1ccccc1-c1cnc(NC(=O)C2CCCN(CCN(C)C)C2)s1 ; Cc1cccc(-c2cnc(NC(=O)C3CCCN(CCN(C)C)C3)s2)c1 ; Cc1ccc(-c2cnc(NC(=O)C3CCCN(CCN(C)C)C3)s2)cc1]

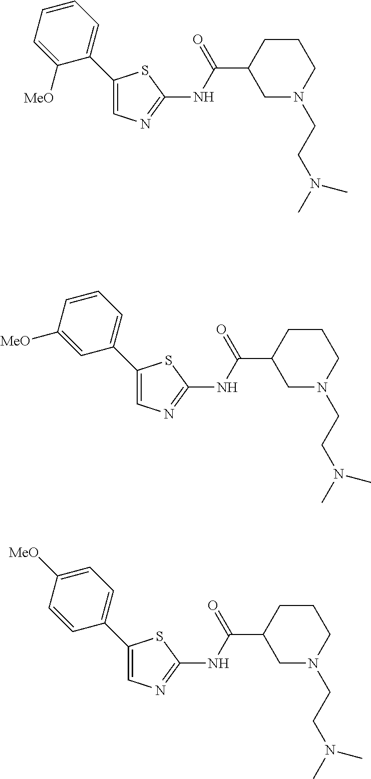 [SMILES: COc1ccccc1-c1cnc(NC(=O)C2CCCN(CCN(C)C)C2)s1 ; COc1cccc(-c2cnc(NC(=O)C3CCCN(CCN(C)C)C3)s2)c1 ; COc1ccc(-c2cnc(NC(=O)C3CCCN(CCN(C)C)C3)s2)cc1]

or a pharmaceutically acceptable salt thereof.
125. A compound of Formula (IX):

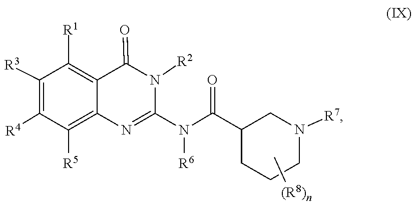

or a pharmaceutically acceptable salt thereof, wherein:
R² is selected from H, C₁₋₃ alkyl, and C₁₋₃ haloalkyl;
R⁶ is selected from H, C₁₋₃ alkyl, and C₁₋₃ haloalkyl;
R¹, R³, R⁴, and R⁵ are each independently selected from H, halo, CN, NO₂, C(O)OR^{a1}, OR^{a1}, NR^{c1}R^{d1} C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, N^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
126. The compound of embodiment 125, wherein:
   R³ is selected from H and halo;
   R⁵ is halo.
127. The compound of embodiment 126, wherein the compound of Formula (IX) has formula:

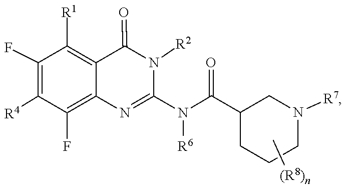

or a pharmaceutically acceptable salt thereof.
128. The compound of embodiment 126, wherein the compound of Formula (IX) has formula:

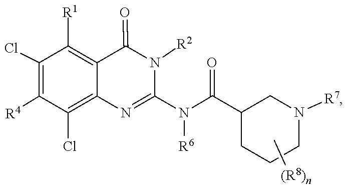

or a pharmaceutically acceptable salt thereof.
129. The compound of embodiment 126, wherein the compound of Formula (IX) has formula:

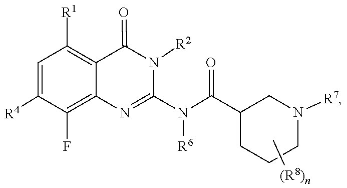

or a pharmaceutically acceptable salt thereof.
130. The compound of embodiment 126, wherein the compound of Formula (IX) has formula:

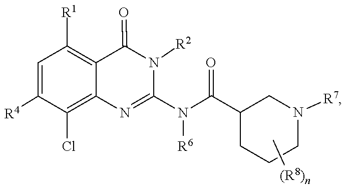

or a pharmaceutically acceptable salt thereof.
131. The compound of any one of embodiments 125-130, wherein n is 0, 1, or 2.
132. The compound of any one of embodiments 125-131, wherein R⁸ is selected from halo, CN, OH, NO₂, C₁₋₃ alkyl, C₁₋₃ haloalkyl, C₁₋₃ alkoxy, and C₁₋₃ haloalkoxy.
133. The compound of embodiment 132, wherein R⁸ is selected from halo, C₁₋₆ alkyl, C₁₋₆ haloalkyl, C₁₋₆ alkoxy, and C₁₋₆ haloalkoxy.
134. The compound of any one of embodiments 125-133, wherein R¹ and R⁴ are each independently selected from H, halo, CN, OH, C₁₋₃ alkyl, C₁₋₃ haloalkyl, C₁₋₃ alkoxy, and C₁₋₃ haloalkoxy.
135. The compound of embodiment 125, selected from any one of the following formulae:

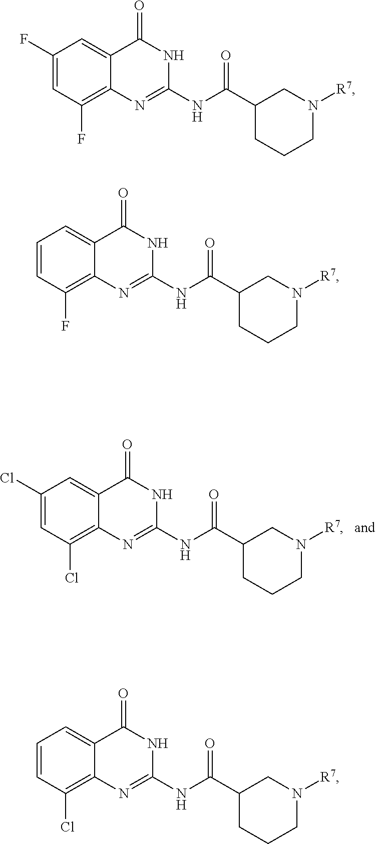

or a pharmaceutically acceptable salt thereof.
136. The compound of any one of embodiments 125-135, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}.
137. The compound of embodiment 136, wherein R⁷ is C₁₋₆ alkyl substituted with NR^{c1}R^{d1}.
138. The compound of embodiment 125, wherein the compound is selected from any one of the following compounds:

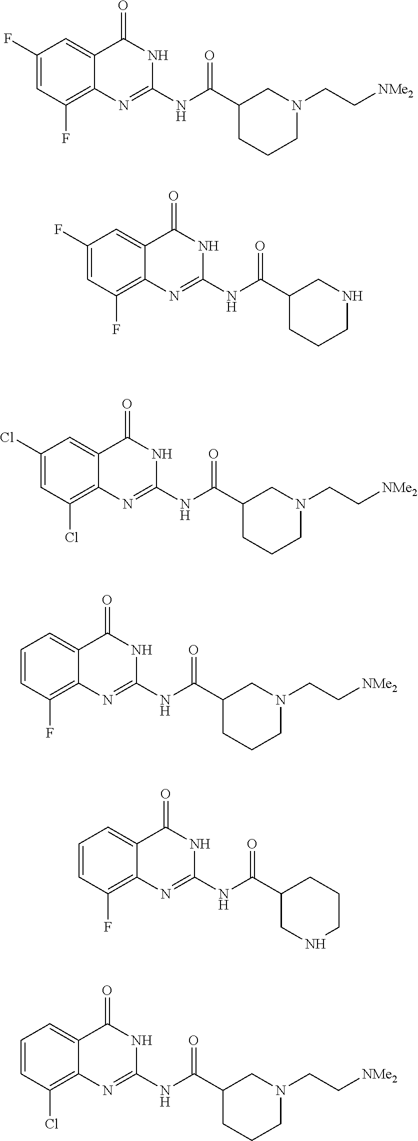 [SMILES: CN(C)CCN1CCCC(C(=O)Nc2nc3c(F)cc(F)cc3c(=O)[nH]2)C1 ; O=C(Nc1nc2c(F)cc(F)cc2c(=O)[nH]1)C1CCCNC1 ; CN(C)CCN1CCCC(C(=O)Nc2nc3c(Cl)cc(Cl)cc3c(=O)[nH]2)C1 ; CN(C)CCN1CCCC(C(=O)Nc2nc3c(F)cccc3c(=O)[nH]2)C1 ; O=C(Nc1nc2c(F)cccc2c(=O)[nH]1)C1CCCNC1 ; CN(C)CCN1CCCC(C(=O)Nc2nc3c(Cl)cccc3c(=O)[nH]2)C1]

or a pharmaceutically acceptable salt thereof.
139. A compound of Formula (X):

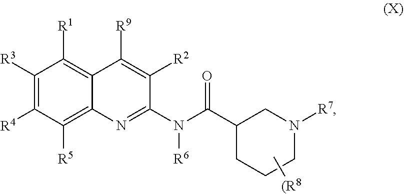

or a pharmaceutically acceptable salt thereof, wherein:
R¹, R², R³, R⁴, R⁵, and R⁹ are each independently selected from H, halo, CN, C(O)OR^{a1}, OR^{a1}, Cy¹, C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
R⁷ is selected from H, Cy¹, C(═O)Cy¹, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, CN, NO₂, OR^{a1}, C(O)R^{b1}, C(O)NR^{c1}R^{d1}, C(O)OR^{a1}, NHC(═NH)NH₂, or NR^{c1}R^{d1};
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each R^{b1} is independently selected from C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1}; and
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, N^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
140. The compound of embodiment 139, wherein:
   R³ is selected from H and halo;
   R⁵ is halo.
141. The compound of embodiment 140, wherein the compound of Formula (X) has formula:

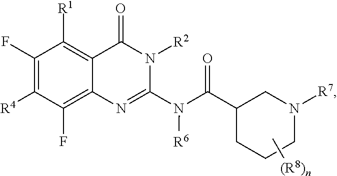

or a pharmaceutically acceptable salt thereof.
142. The compound of embodiment 140, wherein the compound of Formula (X) has formula:

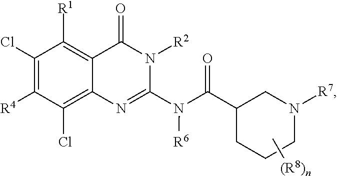

or a pharmaceutically acceptable salt thereof.
143. The compound of embodiment 140, wherein the compound of Formula (X) has formula:

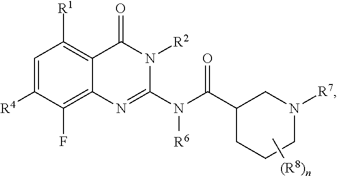

or a pharmaceutically acceptable salt thereof.
144. The compound of embodiment 140, wherein the compound of Formula (X) has formula:

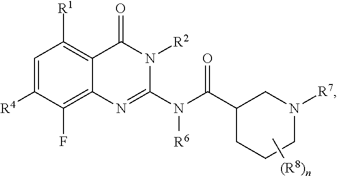

or a pharmaceutically acceptable salt thereof.
145. The compound of any one of embodiments 139-144, wherein n is 0, 1, or 2.
146. The compound of any one of embodiments 139-145, wherein R⁸ is selected from halo, CN, OH, NO₂, C₁₋₃ alkyl, C₁₋₃ haloalkyl, C₁₋₃ alkoxy, and C₁₋₃ haloalkoxy.
147. The compound of embodiment 146, wherein R⁸ is selected from halo, C₁₋₆ alkyl, C₁₋₆ haloalkyl, C₁₋₆ alkoxy, and C₁₋₆ haloalkoxy.
148. The compound of any one of embodiments 139-147, wherein R¹, R², R⁴, and R⁹ are each independently selected from H, halo, CN, OH, C₁₋₃ alkyl, C₁₋₃ haloalkyl, C₁₋₃ alkoxy, and C₁₋₃ haloalkoxy.
149. The compound of embodiment 139, selected from any one of the following formulae:

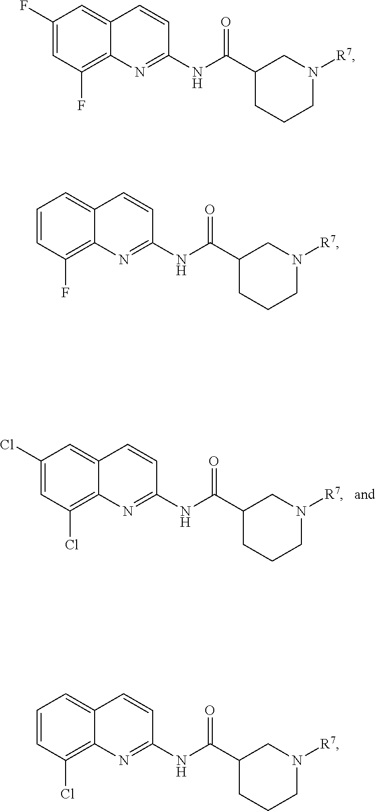

or a pharmaceutically acceptable salt thereof.
150. The compound of any one of embodiments 139-149, wherein R⁷ is selected from H, Cy¹, C(═O)Cy¹, C(O)NR^{c1}R^{d1}, C(O)R^{b1}, and C₁₋₆ alkyl, wherein said C₁₋₆ alkyl is optionally substituted with Cy¹, C(O)NR^{c1}R^{d1}, C(O)OR^{a1}, or NR^{c1}R^{d1}.
151. The compound of embodiment 150, wherein R⁷ is C₁₋₆ alkyl substituted with NR^{c1}R^{d1}.
152. The compound of embodiment 139, wherein the compound is selected from any one of the following compounds:

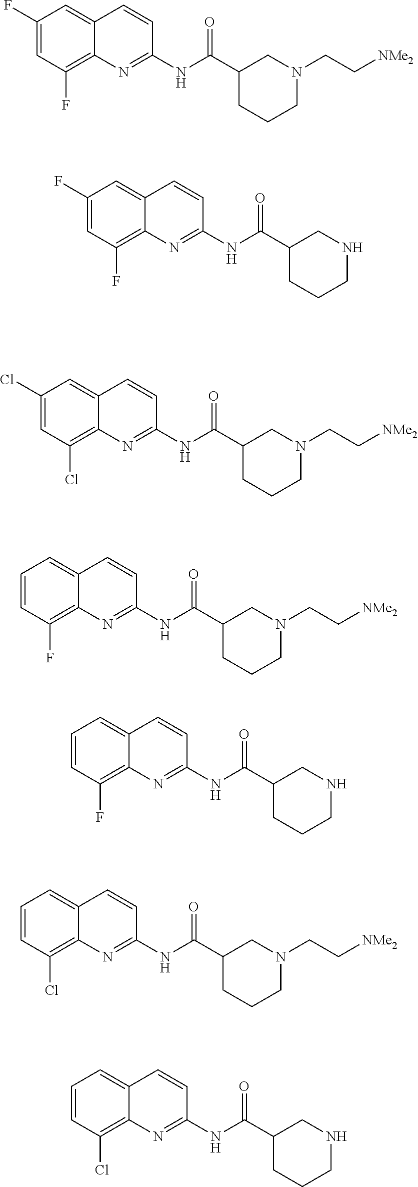

or a pharmaceutically acceptable salt thereof.
153. A compound of Formula (XI):

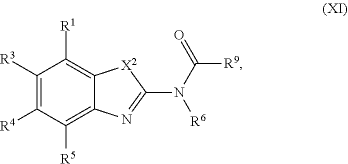

or a pharmaceutically acceptable salt thereof, wherein:
R⁹ is selected from any one of the following groups:

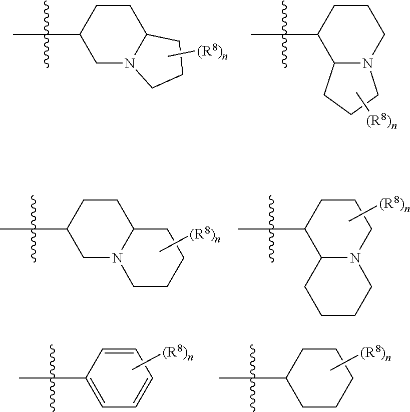

X² is selected from S, O, and NR²;
R² is selected from H, C₁₋₃ alkyl, and C₁₋₃ haloalkyl;
R³ is halo;
R⁵ is halo;
R¹ and R⁴ are each independently selected from H, halo, CN, NO₂, C(O)OR^{a1}, OR^{a1}, NR^{c1}R^{d1} C₁₋₃ alkyl, and C₁₋₃ haloalkyl, wherein said C₁₋₃ alkyl is optionally substituted with CN, C(O)OR^{a1}, OR^{a1}, NR^{c1}R^{d1}, or Cy¹;
R⁶ is selected from H, C₁₋₃ alkyl, and C₁₋₃ haloalkyl;
n is 0, 1, 2, or 3;
each R⁸ is independently selected from halo, CN, NO₂, C(O)OR^{a1}, OR^{a1}, NR^{c1}R^{d1}, C₁₋₃ alkyl, C₁₋₃ haloalkyl, and Cy¹, wherein said C₁₋₃ alkyl is optionally substituted with CN, C(O)OR^{a1}, OR^{a1}, NR^{c1}R^{d1}, or Cy¹;
each R^{a1}, R^{c1} and R^{d1} is independently selected from H, C₁₋₃ alkyl, C₁₋₃ haloalkyl, Cy¹, Cy¹-C₁₋₃ alkylene, NR^{c2}R^{d2}—C₁₋₃ alkylene, C(O)OR^{a2}—C₁₋₃ alkylene, and OR^{a2}—C₁₋₃ alkylene;
each Cy¹ is independently selected from C₆₋₁₀ aryl, C₃₋₁₀ cycloalkyl, 5-14 membered heteroaryl, and 4-10 membered heterocycloalkyl, each of which is optionally substituted with 1, 2, or 3 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, and S(O)₂NR^{c2}R^{d2}; wherein said C₁₋₆ alkyl is optionally substituted with CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, S(O)₂R^{b2}, or S(O)₂NR^{c2}R^{d2};
each R^{a2}, R^{c2}, and R^{d2} is independently selected from H, C₁₋₆ alkyl, and C₁₋₄ haloalkyl; and
each R^{b2} is independently selected from C₁₋₆ alkyl and C₁₋₄ haloalkyl.
154. The compound of embodiment 153, wherein R¹ and R⁴ are each independently selected from H, halo, CN, OH, C₁₋₃ alkyl, C₁₋₃ haloalkyl, C₁₋₃ alkoxy, and C₁₋₃ haloalkoxy.
155. The compound of embodiment 153, wherein the compound of Formula (XI) has formula:

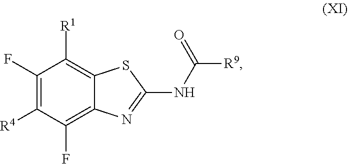

or a pharmaceutically acceptable salt thereof.
156. The compound of any one of embodiments 153-155, wherein R⁹ is:

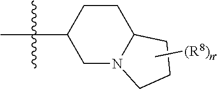

157. The compound of any one of embodiments 153-155, wherein R⁹ is:

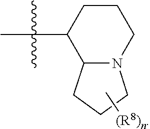

158. The compound of any one of embodiments 153-155, wherein R⁹ is:

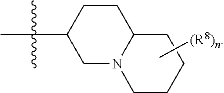

159. The compound of any one of embodiments 153-155, wherein R⁹ is:

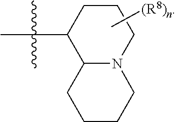

160. The compound of any one of embodiments 153-155, wherein R⁹ is:

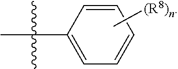

161. The compound of any one of embodiments 153-155, wherein R⁹ is:

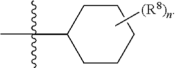

162. The compound of any one of embodiments 153-161, wherein R⁸ is selected from halo, OR^{a1}, NR^{c1}R^{d1}, C₁₋₃ alkyl, C₁₋₃ haloalkyl, and Cy¹, wherein said C₁₋₃ alkyl is optionally substituted with OR^{a1}, NR^{c1}R^{d1}, or Cy¹.
163. The compound of embodiment 162, wherein R⁸ is selected from halo, OR^{a1} NR^{c1}R^{d1} C₁₋₃ alkyl, and Cy¹, wherein said C₁₋₃ alkyl is optionally substituted with NR^{c1}R^{d1}.
164. The compound of any one of embodiments 153-163, wherein n is 0, 1, or 2.
165. The compound of embodiment 153, wherein the compound of Formula (XI) has formula:

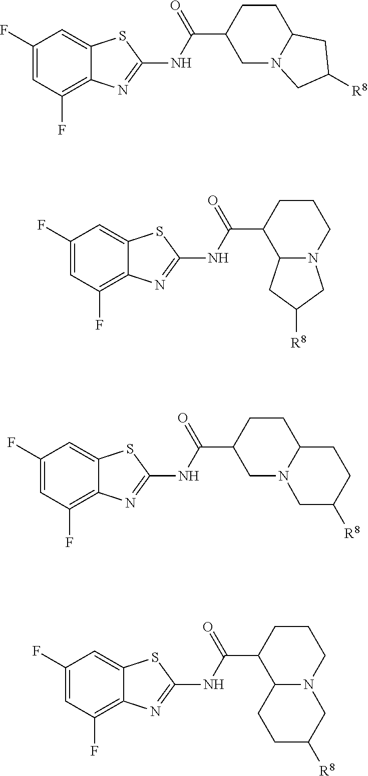

or a pharmaceutically acceptable salt thereof, wherein:
R⁸ is selected from NR^{c1}R^{d1} and NR^{c1}R^{d1}—C₁₋₃ alkylene;
R^{c1} and R^{d1} is independently selected from H, C₁₋₃ alkyl, and NR^{c2}R^{d2}—C₁₋₃ alkylene; and
R^{c2} and R^{d2} are each independently selected from H and C₁₋₆ alkyl.
166. The compound of embodiment 153, wherein the compound of Formula (XI) has any one of the following formulae:

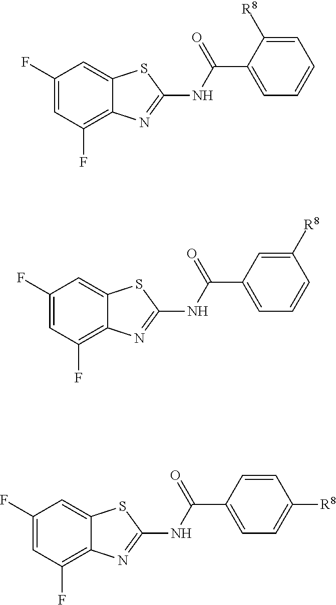

or a pharmaceutically acceptable salt thereof, wherein each R⁸ is independently selected from halo, OR^{a1}, and Cy¹.
167. The compound of embodiment 166, wherein each R⁸ is independently selected from halo, C₁₋₃ alkoxy, and phenyl.
168. The compound of embodiment 153, wherein the compound of Formula (XI) has any one of the following formulae:

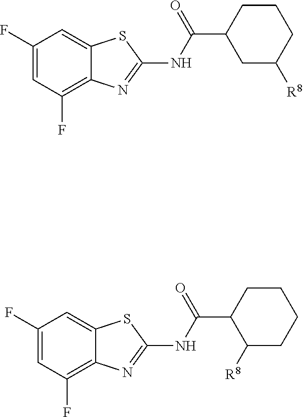

or a pharmaceutically acceptable salt thereof, wherein:
R⁸ is selected from NR^{c1}R^{d1} and NR^{c1}R^{d1}—C₁₋₃ alkylene;
R^{c1} and R^{d1} is independently selected from H, C₁₋₃ alkyl, and NR^{c2}R^{d2}—C₁₋₃ alkylene; and
R^{c2} and R^{d2} are each independently selected from H and C₁₋₆ alkyl.
169. The compound of embodiment 168, wherein R⁸ is NR^{c1}R^{d1}.
170. The compound of embodiment 153, wherein the compound is selected from any one of the following compounds:

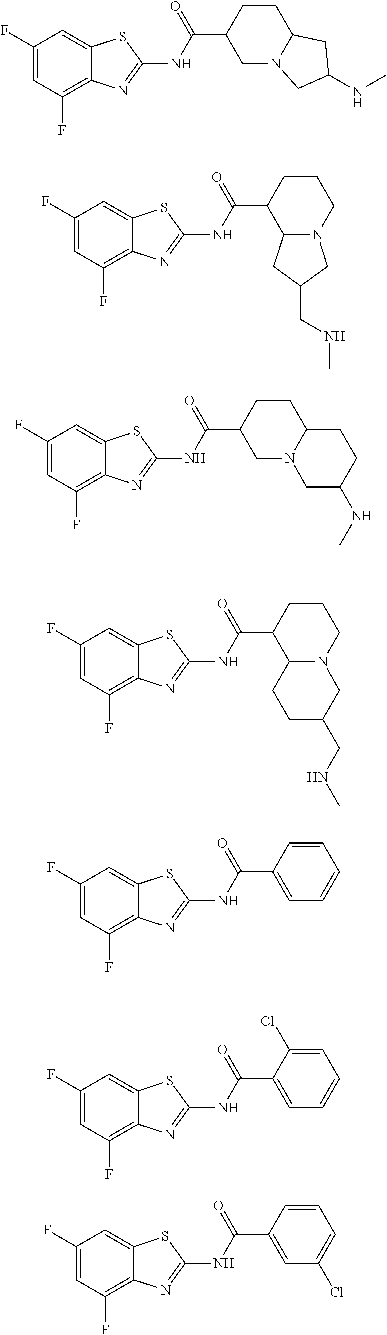

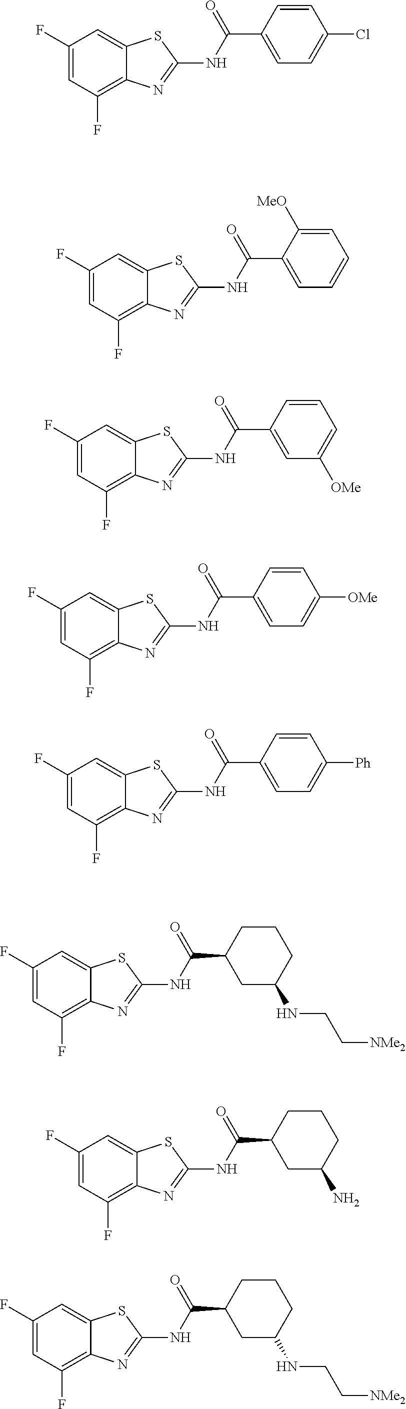

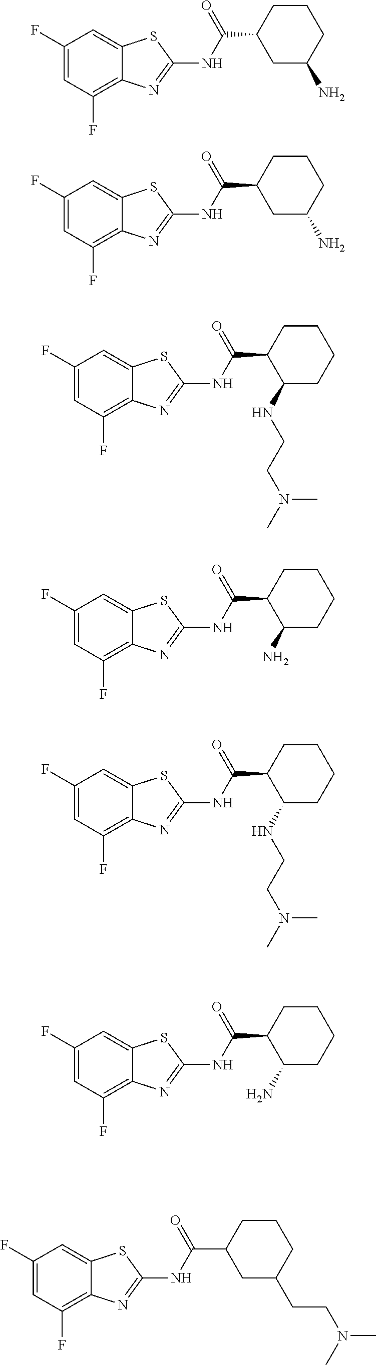

or a pharmaceutically acceptable salt thereof.
171. A compound selected from any one of the following compounds:

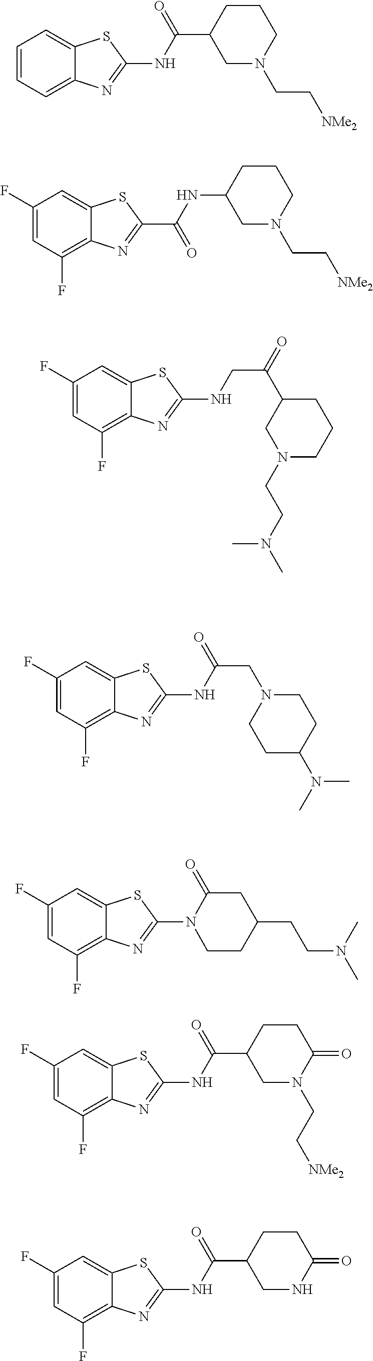

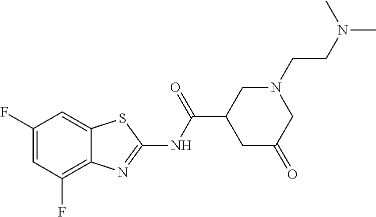 [SMILES: CN(C)CCN1CC(=O)CC(C(=O)Nc2nc3c(F)cc(F)cc3s2)C1]

or a pharmaceutically acceptable salt thereof.
172. A compound selected from any one of the following compounds:

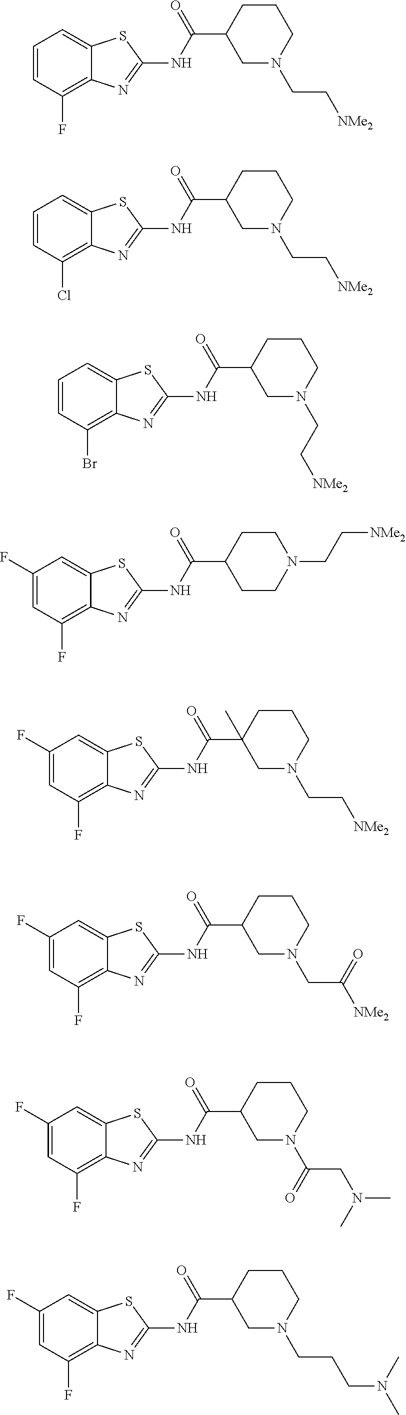

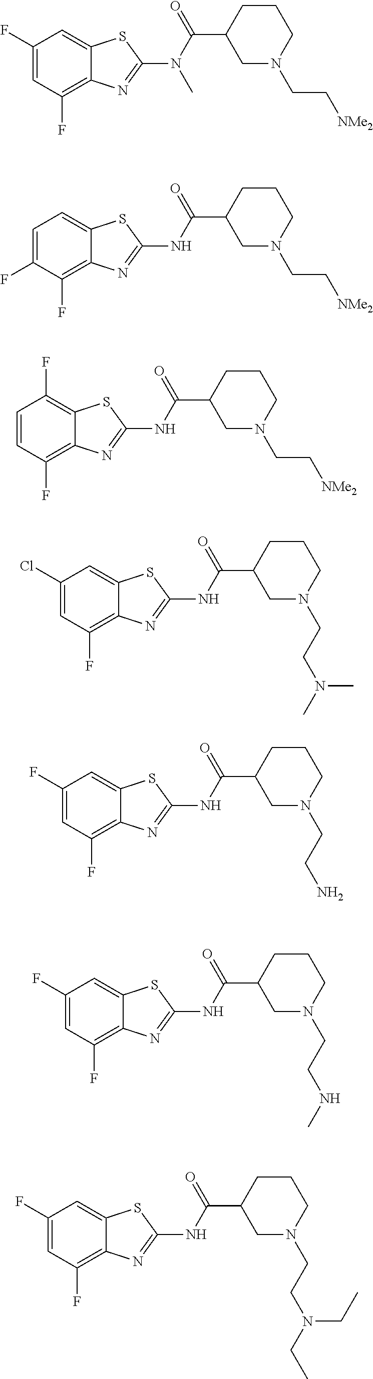

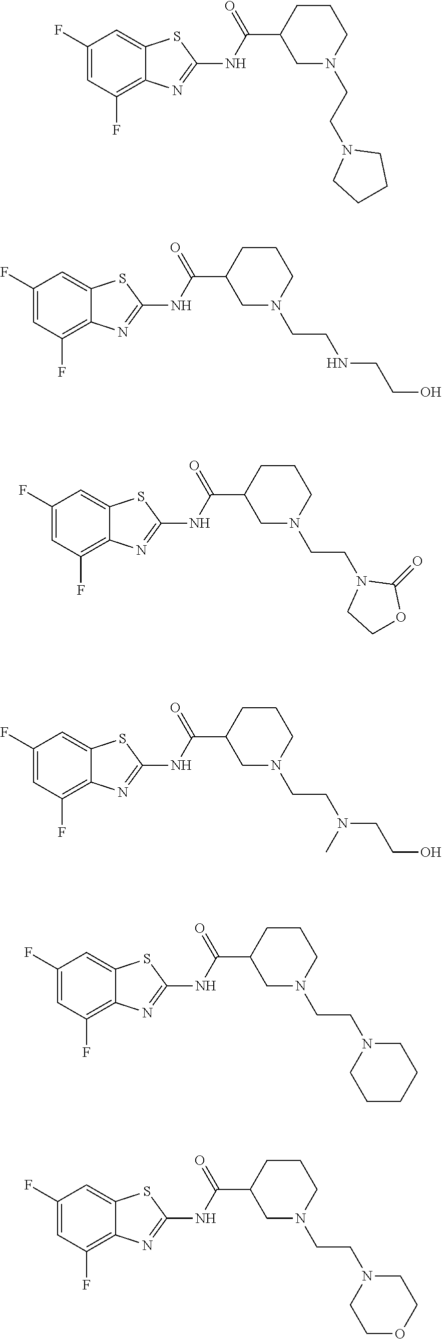 [SMILES: O=C(Nc1nc2c(F)cc(F)cc2s1)C1CCCN(CCN2CCCC2)C1 ; O=C(Nc1nc2c(F)cc(F)cc2s1)C1CCCN(CCNCCO)C1 ; O=C(Nc1nc2c(F)cc(F)cc2s1)C1CCCN(CCN2CCOC2=O)C1 ; CN(CCO)CCN1CCCC(C(=O)Nc2nc3c(F)cc(F)cc3s2)C1 ; O=C(Nc1nc2c(F)cc(F)cc2s1)C1CCCN(CCN2CCCCC2)C1 ; O=C(Nc1nc2c(F)cc(F)cc2s1)C1CCCN(CCN2CCOCC2)C1]

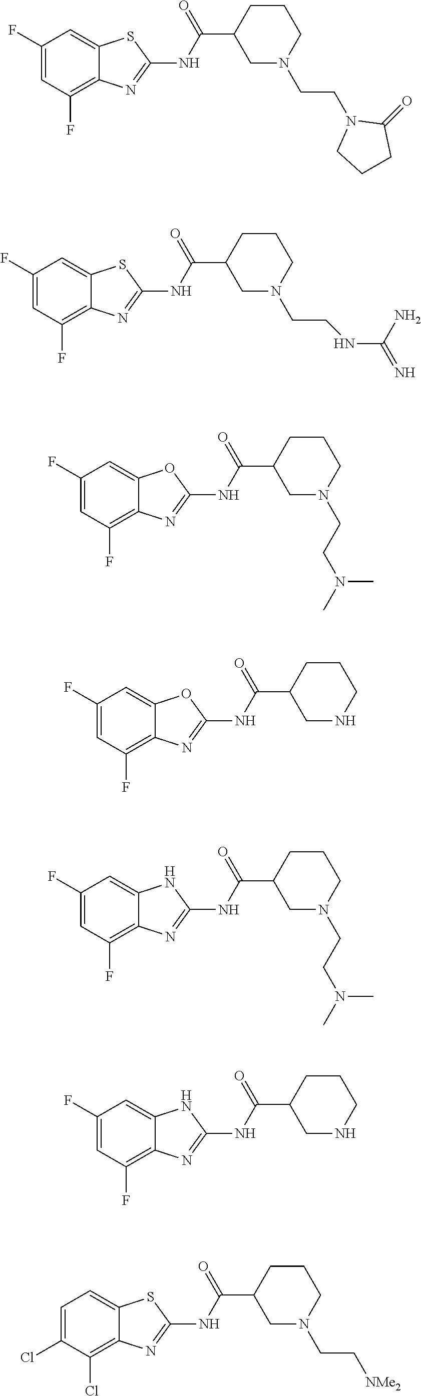

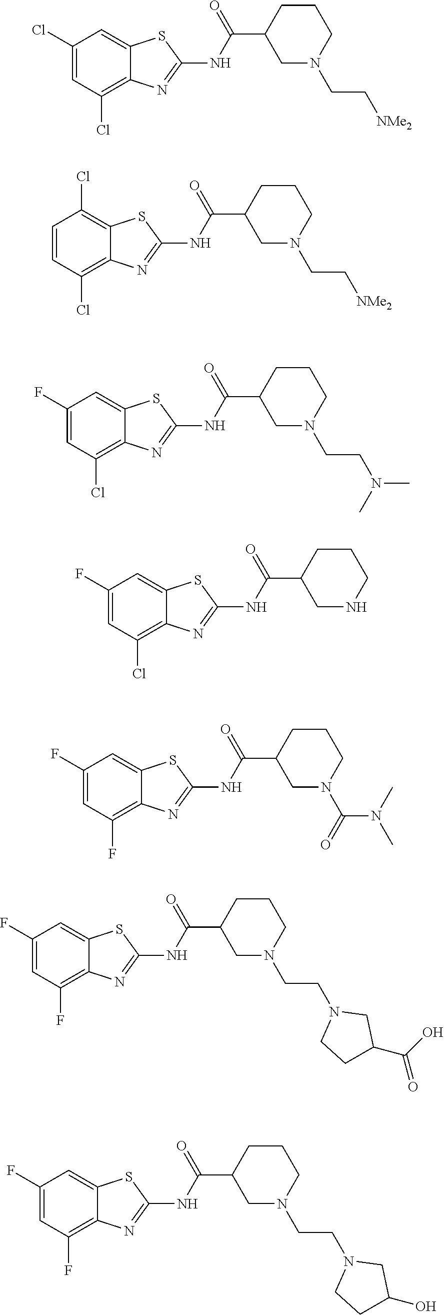

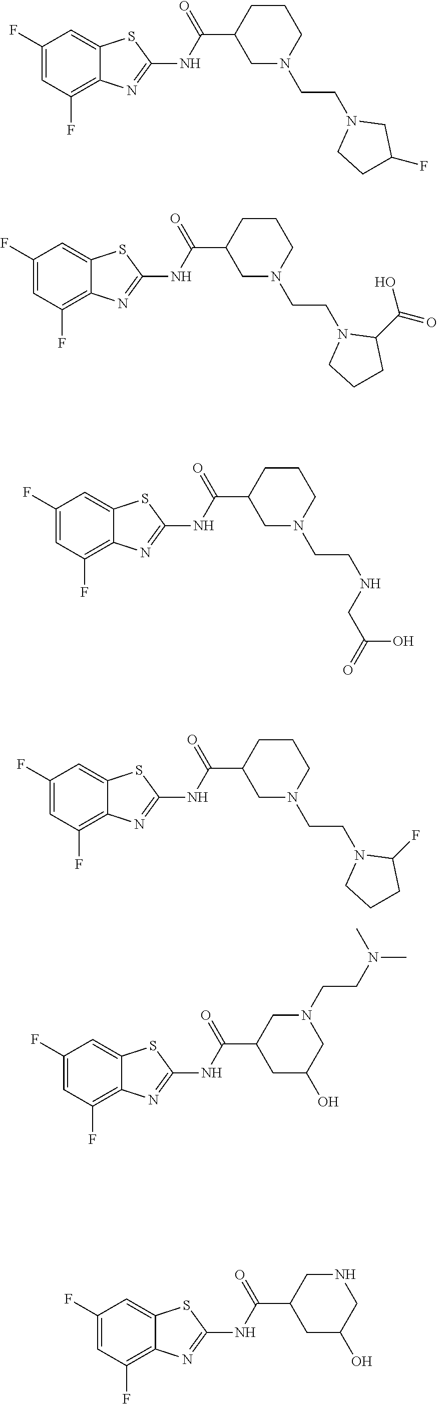

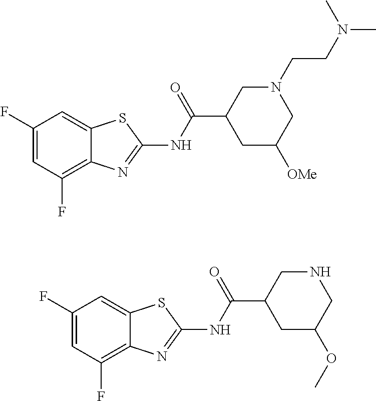 [SMILES: COC1CC(C(=O)Nc2nc3c(F)cc(F)cc3s2)CN(CCN(C)C)C1 ; COC1CNCC(C(=O)Nc2nc3c(F)cc(F)cc3s2)C1]

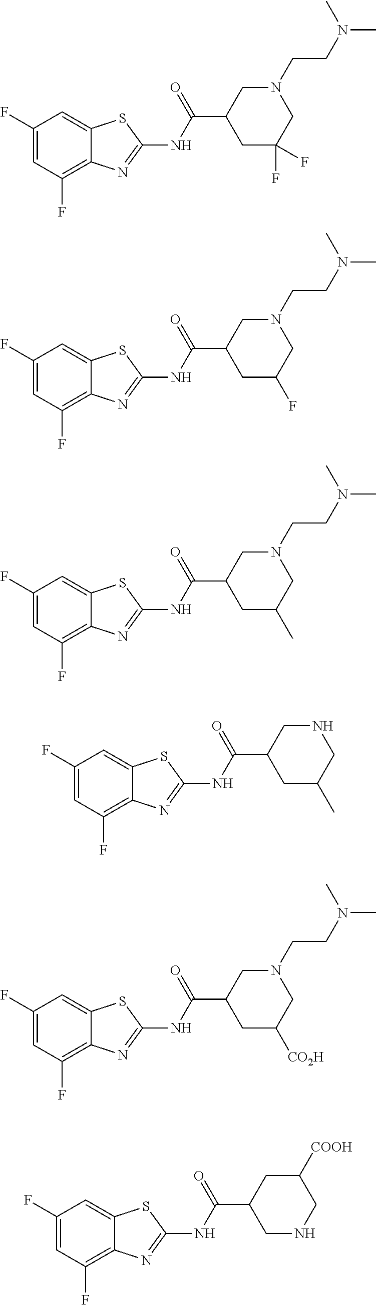 [SMILES: CN(C)CCN1CC(C(=O)Nc2nc3c(F)cc(F)cc3s2)CC(F)(F)C1 ; CN(C)CCN1CC(F)CC(C(=O)Nc2nc3c(F)cc(F)cc3s2)C1 ; CC1CC(C(=O)Nc2nc3c(F)cc(F)cc3s2)CN(CCN(C)C)C1 ; CC1CNCC(C(=O)Nc2nc3c(F)cc(F)cc3s2)C1 ; CN(C)CCN1CC(C(=O)O)CC(C(=O)Nc2nc3c(F)cc(F)cc3s2)C1 ; O=C(O)C1CNCC(C(=O)Nc2nc3c(F)cc(F)cc3s2)C1]

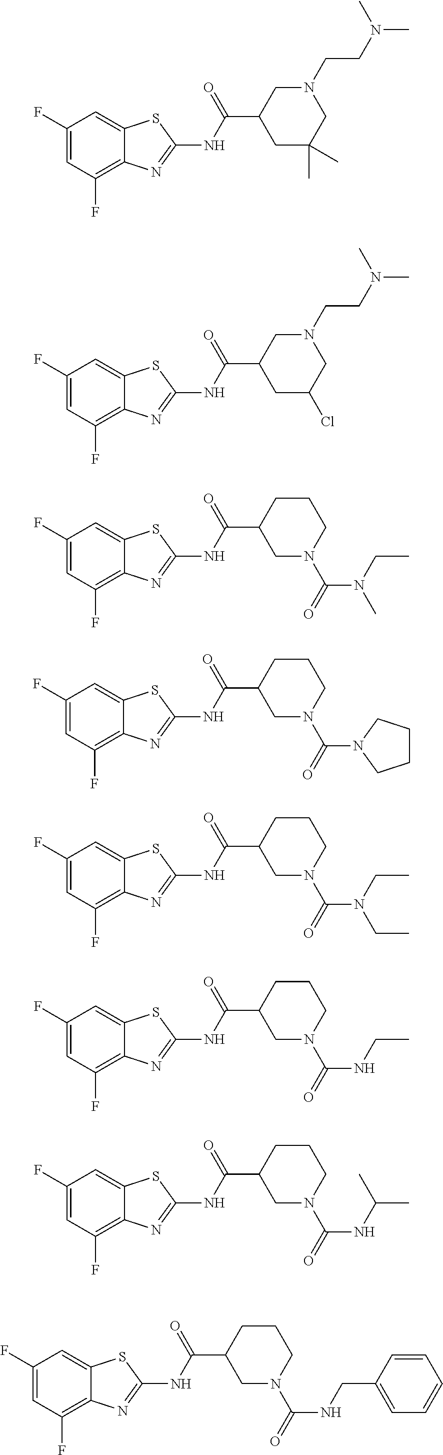

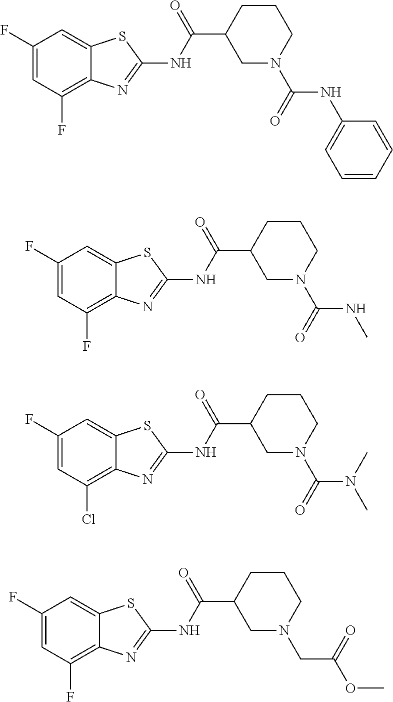 [SMILES: O=C(Nc1nc2c(F)cc(F)cc2s1)C1CCCN(C(=O)Nc2ccccc2)C1 ; CNC(=O)N1CCCC(C(=O)Nc2nc3c(F)cc(F)cc3s2)C1 ; CN(C)C(=O)N1CCCC(C(=O)Nc2nc3c(Cl)cc(F)cc3s2)C1 ; COC(=O)CN1CCCC(C(=O)Nc2nc3c(F)cc(F)cc3s2)C1]

or a pharmaceutically acceptable salt thereof.
173. A pharmaceutical composition comprising a compound of any one of embodiments 1-170, or a pharmaceutically acceptable salt thereof, and a pharmaceutically acceptable carrier.
174. A method of modulating particulate guanylyl cyclase receptor A (pGC-A) in a cell, the method comprising contacting the cell with an effective amount of the compound of any one of embodiments 1-170, or a pharmaceutically acceptable salt thereof.
175. The method of embodiment 174, comprising contacting the cell in vitro, in vivo, or ex vivo.
176. A method of modulating particulate guanylyl cyclase receptor A (pGC-A) in a subject, the method comprising administering to the subject in need thereof an effective amount of the compound of any one of embodiments 1-170, or a pharmaceutically acceptable salt thereof, or the pharmaceutical composition of embodiment 173.
177. The method of any one of embodiments 174-176, wherein the modulating of the particulate guanylyl cyclase receptor A (pGC-A) comprises positive allosteric enhancement of activity of the particulate guanylyl cyclase receptor A (pGC-A).
178. A method of treating or preventing a disease or condition responsive to modulation of a particulate guanylyl cyclase receptor A (pGC-A) in a subject, the method comprising administering to the subject in need thereof a therapeutically effective amount of the compound of any one of embodiments 1-170, or a pharmaceutically acceptable salt thereof, or the pharmaceutical composition of embodiment 173.
179. The method of embodiment 178, wherein the disease or condition is a metabolic disease.
180. The method of embodiment 179, wherein the metabolic disease is selected from obesity, hypertriglyceridemia, metabolic syndrome, insulin resistance, hyperinsulinemia, diabetes, and acidemia.
181. The method of embodiment 179, wherein the disease or condition is a cardiovascular disease.
182. The method of embodiment 181, wherein the cardiovascular disease is selected from heart failure, cardiomyopathy, hypertension, high blood pressure, and myocardial infarction.
183. The method of embodiment 178, wherein the disease or condition is a kidney disease.
184. The method of embodiment 183, wherein the kidney disease is selected from nephropathy, acute renal failure, chronic kidney disease, and diabetic kidney disease.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (XII): 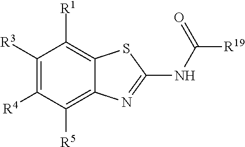
or a pharmaceutically acceptable salt thereof, wherein:
R¹=F, H, Cl, C₁-C₆ alkyl, OC₁-C₆ alkyl;
R⁴=H, C₁;
R³=F, H, C₁-C₆ alkyl, OC₁-C₆ alkyl;
R⁵=F, H, Cl, Br, CN;
R¹⁹ is 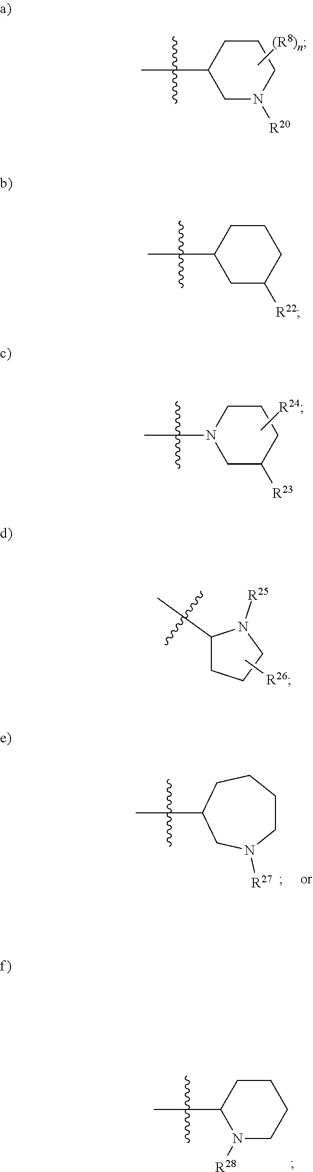
wherein n is 0, 1 or 2, wherein
when n is 1, R⁸ is OH;
and when n is 2,
each R⁸ is F, or
two R⁸ taken together with a ring carbon form C═O, or
two R⁸ taken together with the atoms connecting them form a 5-membered or 6-membered heterocycloalkane ring or a fused benzene ring;
R²⁰ is selected from
a) CON(C₁-C₆ alkyl)₂; b) CO-(4-, 5-, or 6-membered)-heterocycloalkyl optionally substituted at a ring carbon with C₁-C₆ alkyl; c) i) 5- or 6-membered heteroaryl optionally substituted with C₁-C₆ alkyl, NH₂, OC₁-C₆ alkyl, or halogen, or ii) phenyl substituted by N(C₁-C₆ alkyl)₂; d) C₁₋₄ alkyl substituted with R³⁰,
wherein R³⁰ is COOC₁-C₆ alkyl, C(O)N(C₁-C₆ alkyl)₂, C(O)N(H)C₁-C₆ alkyl, C(O)CH₂N(C₁-C₆ alkyl)₂, N(C₁-C₆ alkyl)₂ wherein one alkyl is optionally substituted by OH, N(H)C₁-C₆ alkyl optionally substituted by OH, NH₂ optionally substituted by OH, 4-, 5-, or 6-membered heterocycloalkyl containing N and/or O and optionally substituted by ═O or by C₁-C₆ alkyl or by COOH, or 5- or 6-membered heteroaryl; provided that if R²⁰ is C₂ alkyl and R³⁰ is N(C₁-C₆ alkyl)₂, then R³ and R⁵ are not both F;
e) 5-membered cycloalkyl; and sss
f) 4-, 5-, or 6-membered heterocycloalkyl having NR²¹ and/or O and optionally substituted at a ring carbon with R³¹, wherein R³¹ is C₁-C₆ alkyl, ═O, OH, or OC₁-C₆ alkyl,
wherein R²¹ is a) H; b) C₂-C₆ alkenyl; c) C₁-C₄ alkyl optionally substituted with R³², wherein R³² is halo, C₁-C₆ alkoxy, COOC₁-C₆ alkoxy, or CO-(4-, 5-, or 6-membered heterocycloalkyl); d) COC₁-C₆ alkyl; e) SO₂C₁-C₆ alkyl; or f) 5- or 6-membered heteroaryl optionally substituted with C₁-C₆ alkyl;
R²² is NH₂; C₁-C₆ alkyl substituted with N(C₁-C₆ alkyl)₂ or NH₂; NHC₁-C₆ alkyl substituted with N(C₁-C₆ alkyl)₂ or NH₂; or 4-, 5-, or 6-membered heterocycloalkyl containing NH or NC₁-C₆ alkyl;
R²³ is H or CON(C₁-C₆ alkyl)₂; or R²³ and R²⁴ form a 4-membered spirocyclic heterocycle optionally substituted by C₁-C₆ alkyl;
R²⁶ is H or OH;
R²⁵ is a 4-membered heterocycle containing NH or NC₁-C₆ alkyl;
R²⁷ is H or C₁-C₆ alkyl substituted with N(C₁-C₆ alkyl)₂ or NH₂; and
R²⁸ is C₁-C₆ alkyl substituted with N(C₁-C₆ alkyl)₂ or NH₂.

2. A compound of Formula (XIII): 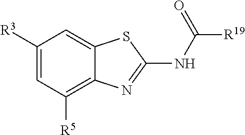
or a pharmaceutically acceptable salt thereof, wherein:
R³=F or H;
R⁵=F, Cl or Br;
R¹⁹ is 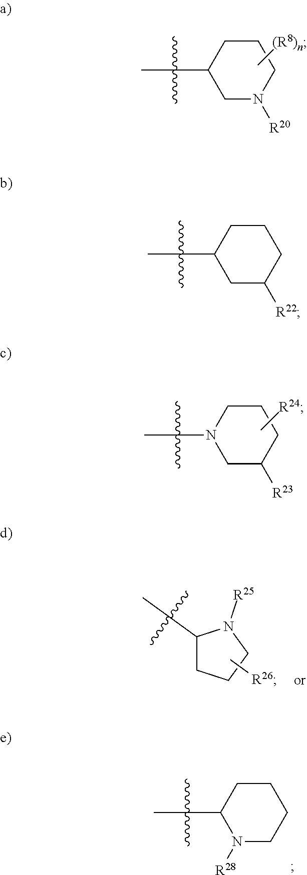
wherein n is 0, 1 or, wherein
when n is 1, R⁸ is OH;
and when n is 2,
each R⁸ is F, or
two R⁸ taken together with the atoms connecting them form a 5-membered or 6-membered heterocycloalkane ring;
R²⁰ is selected from
a) CO-(4-, 5-, or 6-membered)-heterocycloalkyl optionally substituted at a ring carbon with C₁-C₆ alkyl; b) 5- or 6-membered heteroaryl optionally substituted with C₁-C₆ alkyl, NH₂, OC₁-C₆ alkyl, or halogen; c) C₁₋₄ alkyl substituted with R³⁰,
wherein R³⁰ is COOC₁-C₆ alkyl, or 4-, 5-, or 6-m. heterocycloalkyl containing N and/or O;
and
d) 4-, 5-, or 6-membered heterocycloalkyl having NR²¹ and/or O and optionally substituted at a ring carbon with R³¹, wherein R³¹ is C₁-C₆ alkyl, ═O, or OH,
wherein R²¹ is a) H; b) C₂-C₆ alkenyl; c) C₁-C₄ alkyl optionally substituted with R³², wherein R³² is halo, C₁-C₆ alkoxy, COOC₁-C₆ alkoxy, or CO-(4-, 5-, or 6-membered heterocycloalkyl); d) COC₁-C₆ alkyl; e) SO₂C₁-C₆ alkyl; or f) 5- or 6-membered heteroaryl optionally substituted with C₁-C₆ alkyl;
R²² is NH₂; C₁-C₆ alkyl substituted with N(C₁-C₆ alkyl)₂ or NH₂; or 4-, 5-, or 6-membered heterocycloalkyl containing NH or NC₁-C₆ alkyl;
R²³ is H or CON(C₁-C₆ alkyl)₂; or R²³ and R²⁴ form a 4-membered spirocyclic heterocycle optionally substituted by C₁-C₆ alkyl;
R²⁶ is H or OH;
R²⁵ is a 4-membered heterocycle containing NH or NC₁-C₆ alkyl; and
R²⁸ is C₁-C₆ alkyl substituted with N(C₁-C₆ alkyl)₂ or NH₂.

3. The compound of claim 1 or 2, wherein R³ is F.

4. The compound of claim 1 or 2, wherein R³ is H.

5. The compound of claim 1, wherein R⁵ is F.

6. The compound of claim 1, wherein R⁵ is Cl.

7. The compound of claim 1, wherein R³ is Br.

8. The compound of claim 1, wherein R¹⁹ is

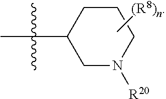

9. The compound of claim 1, wherein R¹⁹ is

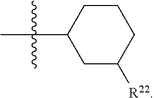

10. The compound of claim 1, wherein R¹⁹

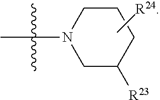

11. The compound of claim 1, wherein R¹⁹ is

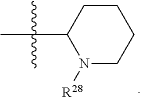

12. The compound of claim 8, wherein n is 0.

13. The compound of claim 8, wherein R²⁰ is selected from
a) CO-(4-, 5-, or 6-membered)-heterocycloalkyl optionally substituted at a ring carbon with C₁-C₆ alkyl; b) 5- or 6-membered heteroaryl optionally substituted with C₁-C₆ alkyl;
c) C₁₋₄ alkyl substituted with R³⁰,
wherein R³⁰ is COOC₁-C₆ alkyl, or 4-, 5-, or 6-m. heterocycloalkyl containing N and/or O;
and
d) 4-, 5-, or 6-membered heterocycloalkyl having NR²¹ and/or O and optionally substituted at a ring carbon with R³¹, wherein R³¹ is C₁-C₆ alkyl, ═O, or OH,
wherein R²¹ is a) H; b) C₂-C₆ alkenyl; c) C₁-C₄ alkyl optionally substituted with R³², wherein R³² is halo, C₁-C₆ alkoxy, COOC₁-C₆ alkoxy, or CO-(4-, 5-, or 6-membered heterocycloalkyl); d) COC₁-C₆ alkyl; e) SO₂C₁-C₆ alkyl; or f) 5- or 6-membered heteroaryl optionally substituted with C₁-C₆ alkyl.

14. The compound of claim 8, wherein R²⁰ is CO-(4-, 5-, or 6-membered)-heterocycloalkyl optionally substituted at a ring carbon with C₁-C₆ alkyl.

15. The compound of claim 8, wherein R²⁰ is 5- or 6-membered heteroaryl optionally substituted with C₁-C₆ alkyl.

16. The compound of claim 8, wherein R²⁰ is C₁₋₄ alkyl substituted with R³⁰, wherein R³⁰ is COOC₁-C₆ alkyl, or 4-, 5-, or 6-m. heterocycloalkyl containing N and/or O.

17. The compound of claim 8, wherein R²⁰ is 4-, 5-, or 6-membered heterocycloalkyl having NR²¹ and/or O and optionally substituted at a ring carbon with R³¹, wherein R³¹ is C₁-C₆ alkyl, ═O, or OH,
wherein R²¹ is a) H; b) C₂-C₆ alkenyl; c) C₁-C₄ alkyl optionally substituted with R³², wherein R³² is halo, C₁-C₆ alkoxy, COOC₁-C₆ alkoxy, or CO-(4-, 5-, or 6-membered heterocycloalkyl); d) COC₁-C₆ alkyl; e) SO₂C₁-C₆ alkyl; or f) 5- or 6-membered heteroaryl optionally substituted with C₁-C₆ alkyl.

18. The compound of claim 8, wherein R¹⁹ is

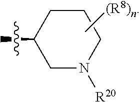

19. The compound of claim 8, wherein R¹⁹ is

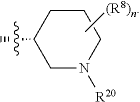

20. The compound of claim 8, wherein R²⁰ is a 4-, 5-, or 6-membered heterocycloalkyl containing NR²¹ and/or O.

21. The compound of claim 18, wherein R²⁰ is a 4-, 5-, or 6-membered heterocycloalkyl containing NR²¹ and/or O.

22. The compound of claim 19, wherein R²⁰ is a 4-, 5-, or 6-membered heterocycloalkyl containing NR²¹ and/or O.

23. The compound of claim 20 wherein R²⁰ is a 5-membered heterocycloalkyl containing NR²¹ and/or O and having (R) stereochemistry at the carbon bonded to the N of the 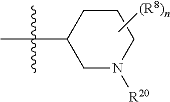
ring.

24. The compound of claim 20 wherein R²⁰ is a 5-membered heterocycloalkyl containing NR²¹ and/or O and having (S) stereochemistry at the carbon bonded to the N of the 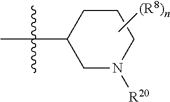
ring.

25. A compound selected from the group consisting of the following compounds, or a pharmaceutically acceptable salt thereof:

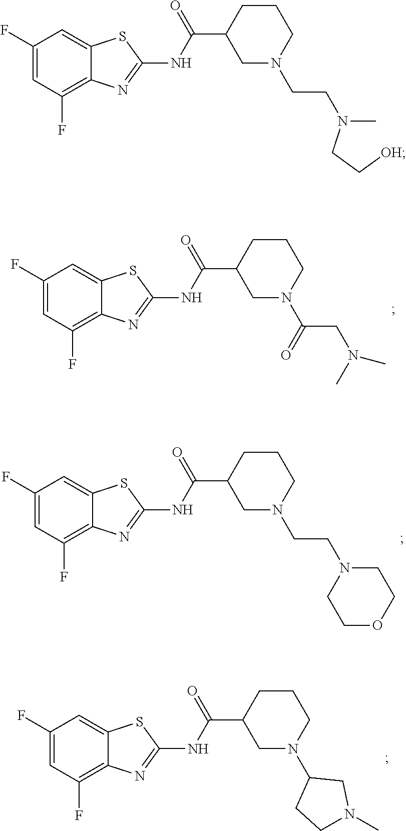 [SMILES: CN(CCO)CCN1CCCC(C(=O)Nc2nc3c(F)cc(F)cc3s2)C1 ; CN(C)CC(=O)N1CCCC(C(=O)Nc2nc3c(F)cc(F)cc3s2)C1 ; O=C(Nc1nc2c(F)cc(F)cc2s1)C1CCCN(CCN2CCOCC2)C1 ; CN1CCC(N2CCCC(C(=O)Nc3nc4c(F)cc(F)cc4s3)C2)C1]

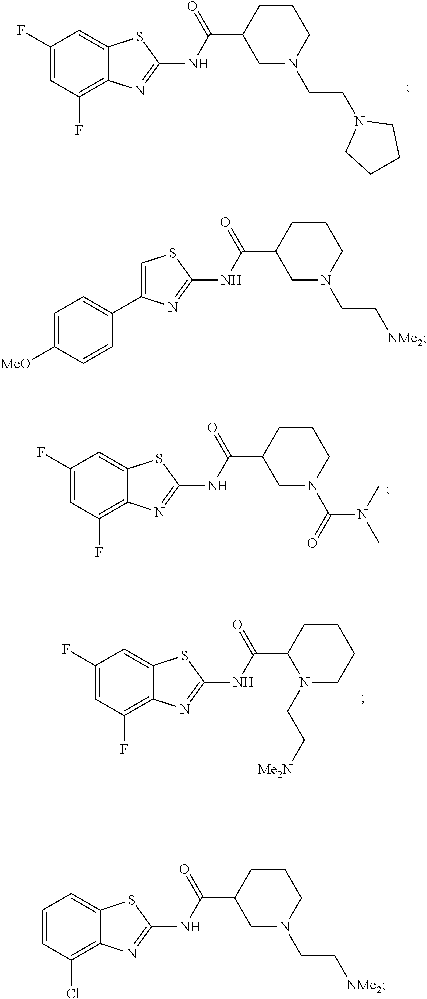 [SMILES: O=C(Nc1nc2c(F)cc(F)cc2s1)C1CCCN(CCN2CCCC2)C1 ; COc1ccc(-c2csc(NC(=O)C3CCCN(CCN(C)C)C3)n2)cc1 ; CN(C)C(=O)N1CCCC(C(=O)Nc2nc3c(F)cc(F)cc3s2)C1 ; CN(C)CCN1CCCCC1C(=O)Nc1nc2c(F)cc(F)cc2s1 ; CN(C)CCN1CCCC(C(=O)Nc2nc3c(Cl)cccc3s2)C1]

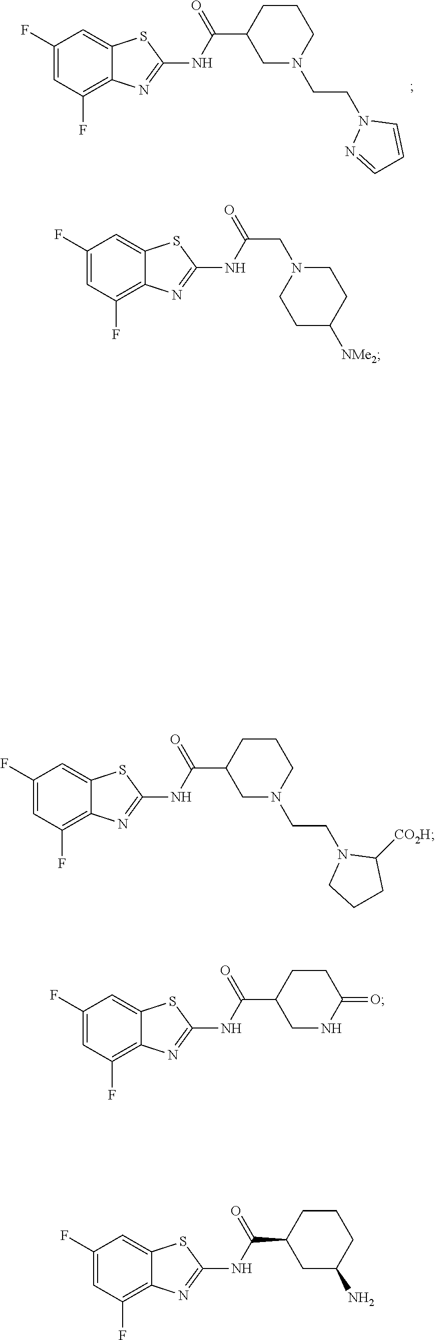 [SMILES: O=C(Nc1nc2c(F)cc(F)cc2s1)C1CCCN(CCn2cccn2)C1 ; CN(C)C1CCN(CC(=O)Nc2nc3c(F)cc(F)cc3s2)CC1 ; O=C(Nc1nc2c(F)cc(F)cc2s1)C1CCCN(CCN2CCCC2C(=O)O)C1 ; O=C1CCC(C(=O)Nc2nc3c(F)cc(F)cc3s2)CN1 ; N[C@@H]1CCC[C@H](C(=O)Nc2nc3c(F)cc(F)cc3s2)C1]

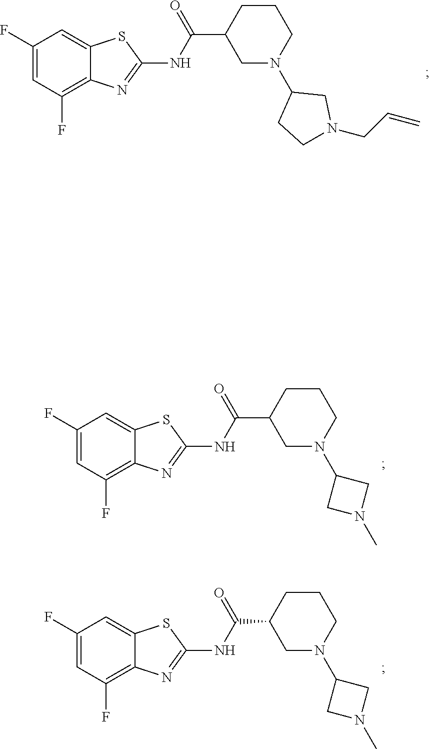 [SMILES: C=CCN1CCC(N2CCCC(C(=O)Nc3nc4c(F)cc(F)cc4s3)C2)C1 ; CN1CC(N2CCCC(C(=O)Nc3nc4c(F)cc(F)cc4s3)C2)C1 ; CN1CC(N2CCC[C@@H](C(=O)Nc3nc4c(F)cc(F)cc4s3)C2)C1]

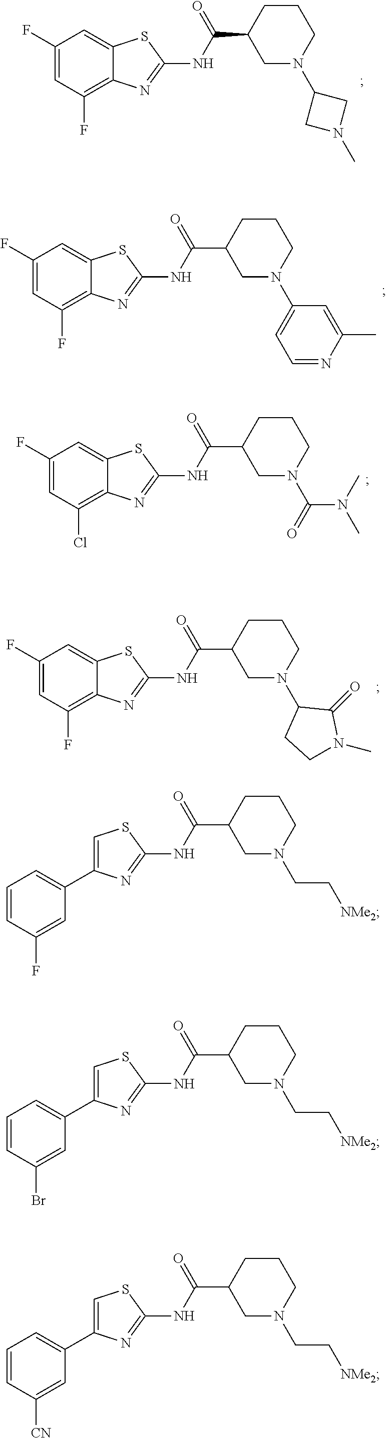

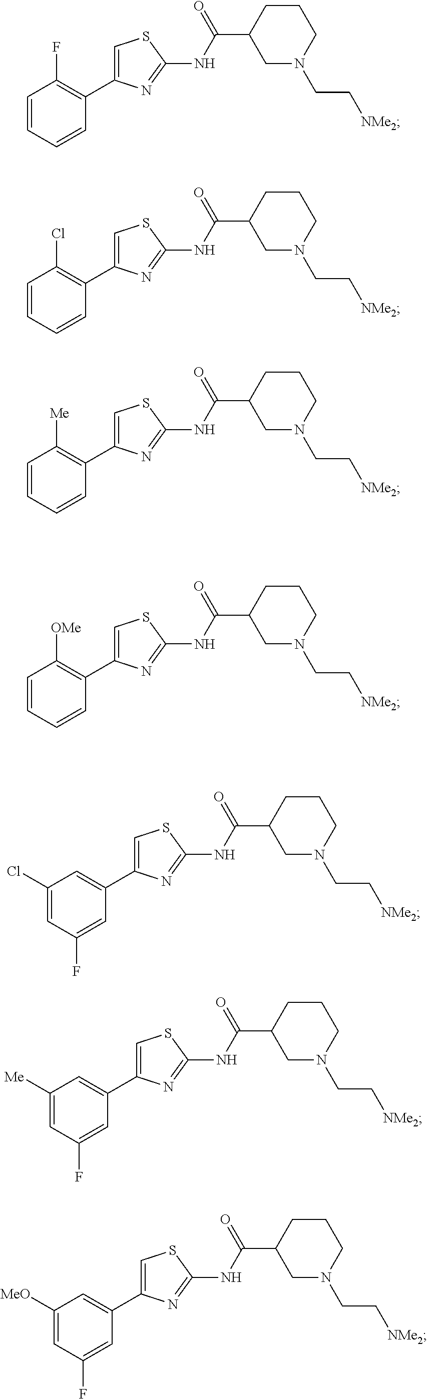

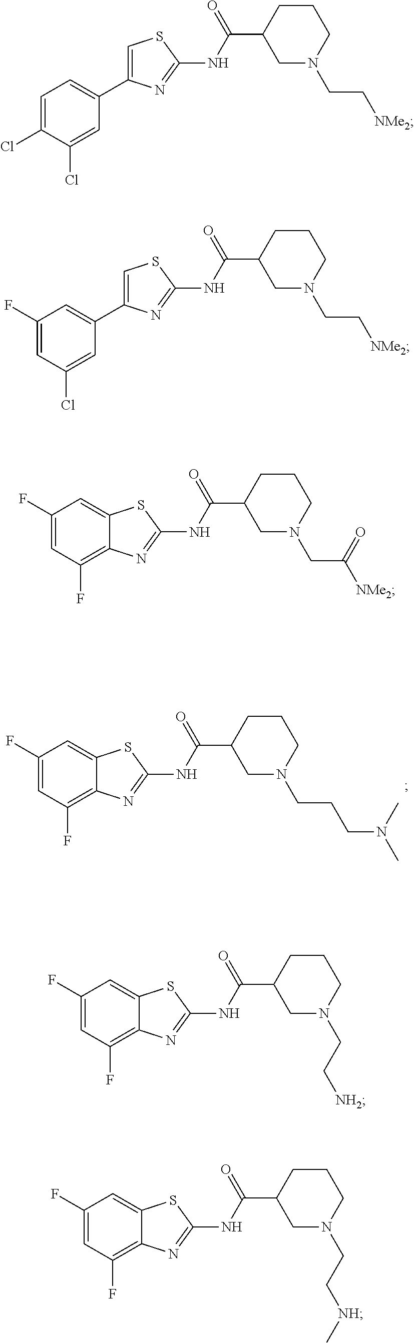 [SMILES: CN(C)CCN1CCCC(C(=O)Nc2nc(-c3ccc(Cl)c(Cl)c3)cs2)C1 ; CN(C)CCN1CCCC(C(=O)Nc2nc(-c3cc(F)cc(Cl)c3)cs2)C1 ; CN(C)C(=O)CN1CCCC(C(=O)Nc2nc3c(F)cc(F)cc3s2)C1 ; CN(C)CCCN1CCCC(C(=O)Nc2nc3c(F)cc(F)cc3s2)C1 ; NCCN1CCCC(C(=O)Nc2nc3c(F)cc(F)cc3s2)C1 ; CNCCN1CCCC(C(=O)Nc2nc3c(F)cc(F)cc3s2)C1]

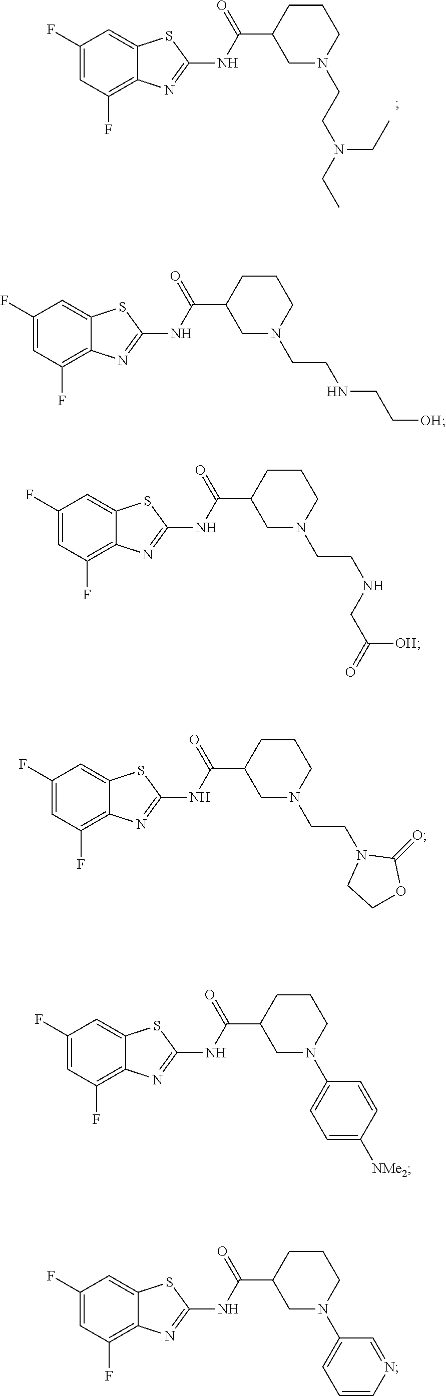 [SMILES: CCN(CC)CCN1CCCC(C(=O)Nc2nc3c(F)cc(F)cc3s2)C1 ; O=C(Nc1nc2c(F)cc(F)cc2s1)C1CCCN(CCNCCO)C1 ; O=C(O)CNCCN1CCCC(C(=O)Nc2nc3c(F)cc(F)cc3s2)C1 ; O=C(Nc1nc2c(F)cc(F)cc2s1)C1CCCN(CCN2CCOC2=O)C1 ; CN(C)c1ccc(N2CCCC(C(=O)Nc3nc4c(F)cc(F)cc4s3)C2)cc1 ; O=C(Nc1nc2c(F)cc(F)cc2s1)C1CCCN(c2cccnc2)C1]

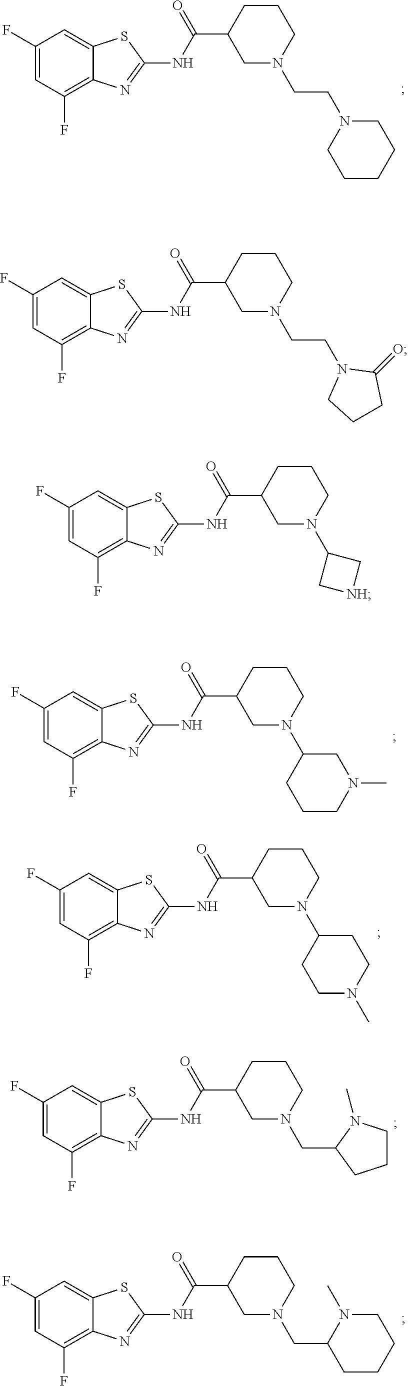

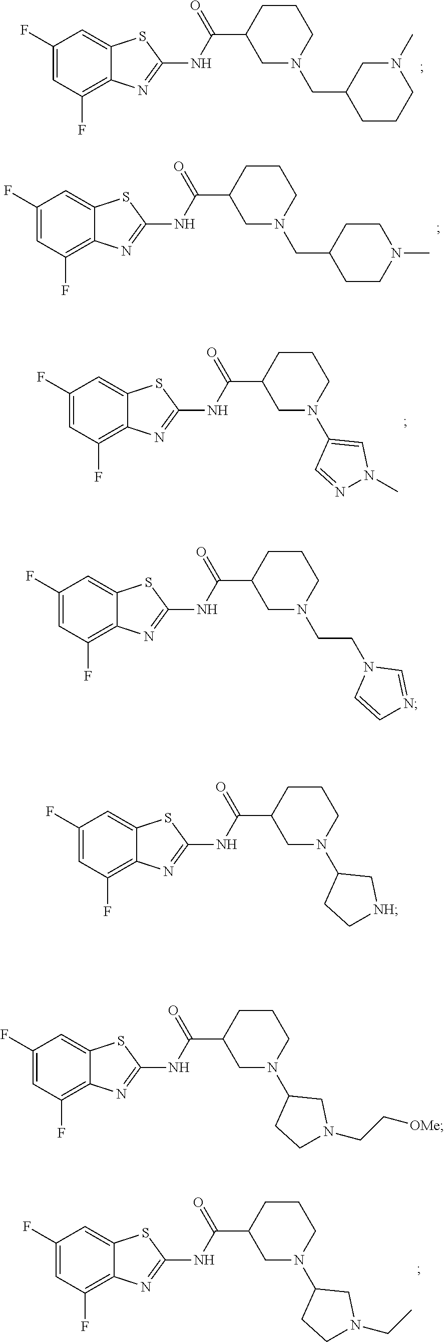

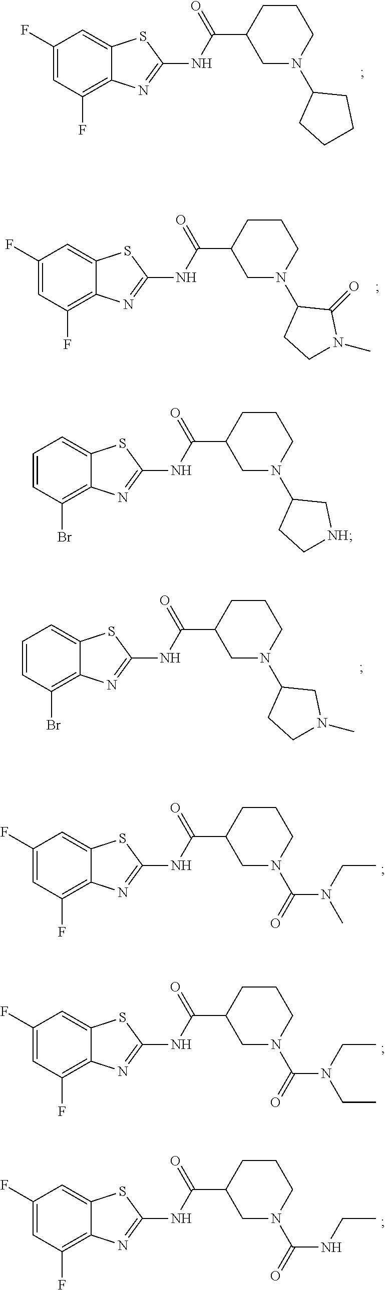

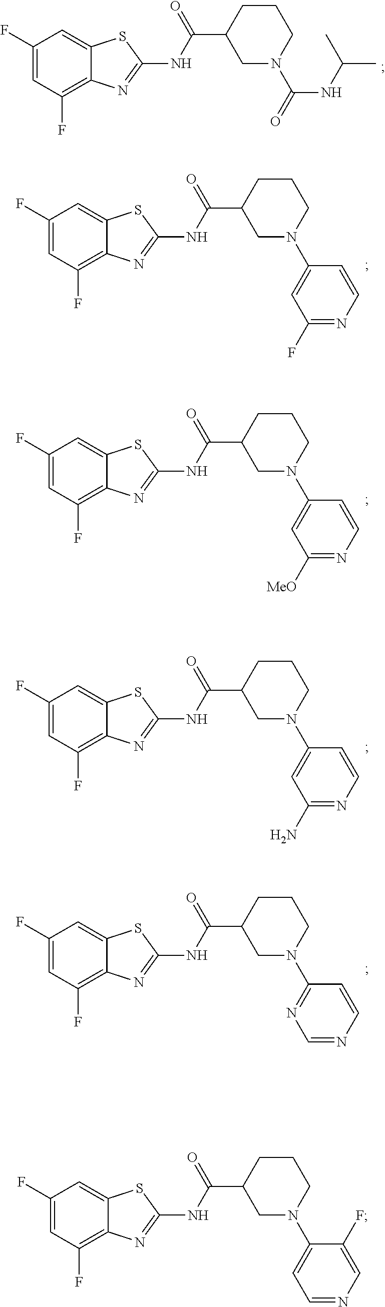 [SMILES: CC(C)NC(=O)N1CCCC(C(=O)Nc2nc3c(F)cc(F)cc3s2)C1 ; O=C(Nc1nc2c(F)cc(F)cc2s1)C1CCCN(c2ccnc(F)c2)C1 ; COc1cc(N2CCCC(C(=O)Nc3nc4c(F)cc(F)cc4s3)C2)ccn1 ; Nc1cc(N2CCCC(C(=O)Nc3nc4c(F)cc(F)cc4s3)C2)ccn1 ; O=C(Nc1nc2c(F)cc(F)cc2s1)C1CCCN(c2ccncn2)C1 ; O=C(Nc1nc2c(F)cc(F)cc2s1)C1CCCN(c2ccncc2F)C1]

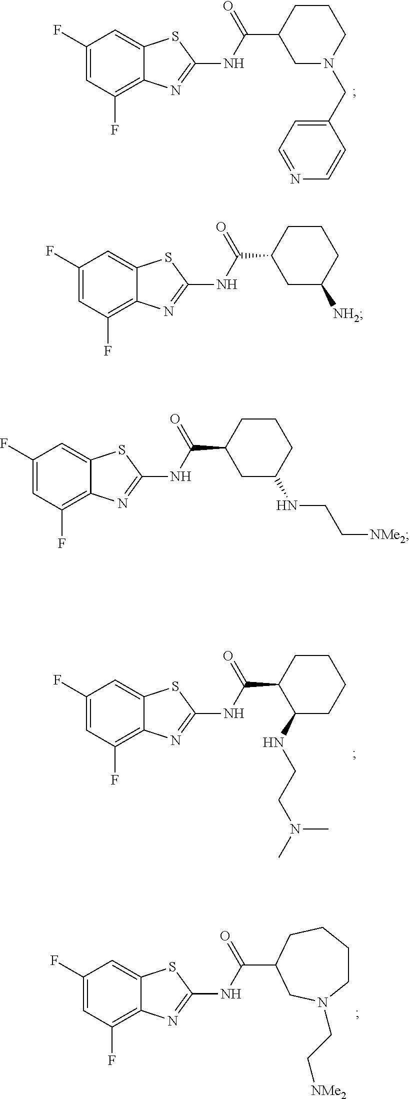

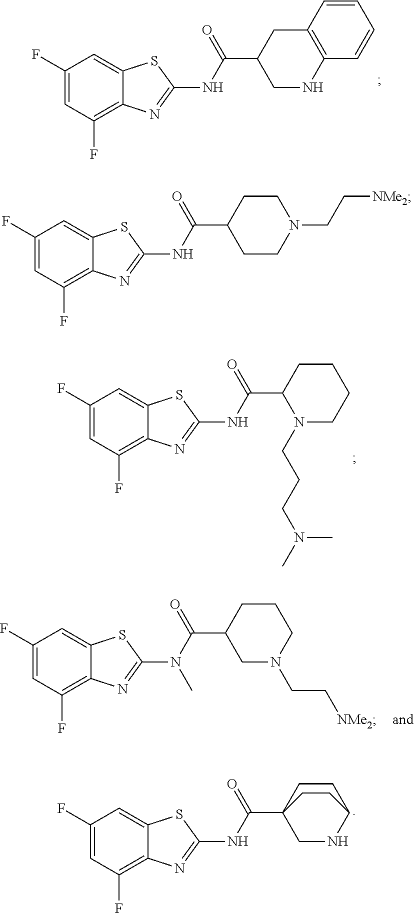

26. A pharmaceutical composition comprising a compound of any one of the preceding claims, or a pharmaceutically acceptable salt thereof, and a pharmaceutically acceptable carrier.

27. A method of modulating particulate guanylyl cyclase receptor A (pGC-A) in a cell, the method comprising contacting the cell with an effective amount of the compound of claim 1, or the pharmaceutical composition of claim 26.

28. A method of modulating particulate guanylyl cyclase receptor A (pGC-A) in a subject, the method comprising administering to the subject in need thereof an effective amount of the compound of claim 1, or the pharmaceutical composition of claim 26.

29. A method of treating or preventing a disease or condition responsive to modulation of a particulate guanylyl cyclase receptor A (pGC-A) in a subject, the method comprising administering to the subject in need thereof a therapeutically effective amount of the compound of claim 1, or the pharmaceutical composition of claim 26.
